(19) United States
(12) Patent Application Publication
(10) Pub. No.: US 2019/0381099 A1
(43) Pub. Date: Dec. 19, 2019

(54) T CELL RECEPTORS FROM THE HIV-SPECIFIC REPERTOIRE, MEANS FOR THEIR PRODUCTION AND THERAPEUTIC USES THEREOF

(71) Applicants: INSTITUT PASTEUR, Paris (FR); INSTITUT NATIONAL DE LA SANTE ET DE LA RECHERCHE MEDICALE, Paris (FR)

(72) Inventors: Lisa Amita CHAKRABARTI, Paris (FR); Daniela BENATI, Reggio Emilia (IT); Moran GALPERIN, Neuilly-Sur-Seine (FR)

(21) Appl. No.: 16/078,508

(22) PCT Filed: Feb. 23, 2017
(86) PCT No.: PCT/EP2017/054249

§ 371 Date: Aug. 21, 2018

(30) Foreign Application Priority Data
Feb. 24, 2016 (EP) 16305218.6

(51) Int. Cl. A61K 35/17; C07K 14/725; C07K 14/16; C07K 16/10; C12N 5/0783; C12N 15/86

(52) U.S. Cl. CPC A61K 35/17; C07K 14/7051; C07K 14/161; A61K 38/00; C12N 5/0636; C12N 15/86; C07K 16/1054

(57) ABSTRACT

The present invention pertains to the field of T Cell receptors (TCR) identification and clonotyping, and especially concerns particular TCRs identified by clonotyping of a HIV-specific TCR repertoire, or fragments thereof. The invention relates especially to TCRs recognizing Gag peptide located between positions 293-312 in the GAG protein of HIV-1. The present invention further relates to nucleic acid constructs suitable as means for cloning or expressing nucleic acid molecules or TCRs of the invention, such as plasmids, vectors, especially lentiviraltransfer vectors. The invention is of particular interest in the context of therapeutic treatment of human beings seropositive for HIV.

The present invention pertains to the field of T Cell receptors (TCR) identification and clonotyping, and especially concerns particular TCRs identified by clonotyping of a HIV-specific TCR repertoire, or fragments thereof.

The present invention further relates to chimeric TCRs derived from the TCRs of the invention or fragments thereof, and to nucleic acid molecules encoding the TCRs or fragments thereof of the invention.

The present invention further relates to nucleic acid constructs suitable as means for cloning or expressing nucleic acid molecules or TCRs of the invention, such as plasmids, vectors, especially lentiviral transfer vectors.

The present invention also provides recombinant lentiviral vector particles and methods and means for producing recombinant lentiviral vector particles, as well as a collection of recombinant human cells presenting TCRs of the invention or fragments thereof and methods for obtaining the same.

The invention is of particular interest in the context of therapeutic treatment of human beings.

The means of the invention can be especially formulated for administration to a human host, more particularly used as a medicament in the immunotherapeutic treatment of a human patient seropositive for HIV, in particular HIV-1.

The invention also relates to methods for the treatment of a patient infected with HIV, in particular HIV-1, especially for active control of HIV infection.

Such patients may be under antiretroviral therapy following a HIV infection, in particular a HIV-1 infection, especially undergoing HAART therapy (highly active antiretroviral therapy).

HIV remains one of the most devastating infectious agents, with an estimated 37 million people currently living with the virus worldwide (UNAIDS. Global AIDS update. http://wwwunaidsorg/en/resources/documents/2016/2016_GARPR_FAQ. 2016). Antiretroviral therapy (ART) has dramatically improved the health and life expectancy of people living with HIV. However, immune system dysfunction associated with chronic immune activation persists in treated patients, resulting in a significant non-AIDS related chronic disease burden that needs to be addressed. In addition, the economical and logistical challenges involved in providing lifelong antiretroviral treatment to the 37 million people living with HIV remain considerable. Therefore, there is an urgent need of identifying the means to control HIV in the absence of ART.

HIV relentlessly destroys CD4+ T cells in the course of infection and causes functional impairment in the remaining CD4+ T cell population. This leads to the progressive loss of adaptive responses to opportunistic pathogens and ultimately to the collapse of the immune system characteristic of AIDS. Chronic immune activation is thought to drive dysfunction of the remaining CD4+ T cell population, with both persistent viral antigenic stimulation and microbial translocation conspiring to exhaust T cell responses (1). Another parameter contributing to the loss of helper function may be the poor quality of CD4+ T cells that escape depletion. Early studies of the repertoire of T cell receptors (TCRs) documented a general loss of CD4+ T cell diversity in HIV infected patients (2, 3), while further studies highlighted a preferential depletion of HIV-specific CD4+ T cells (4-6), suggesting that the HIV-specific repertoire was especially prone to diversity contraction. To date, the HIV-specific CD4 repertoire remains mostly uncharacterized at the molecular level, even though this information would be critical to define the potential for immune reconstitution in treated patients.

Rare cases of spontaneously controlled HIV-1 infection provide a unique opportunity to study the molecular characteristics of a fully functional CD4+ T cell response directed at HIV. Patients who maintain an undetectable viral load in standard assays (<50 copies HIV-1 RNA/mL) represent fewer than 0.5% of seropositive individuals but have a remarkably low risk of progressing to AIDS (7). These rare patients, called HIV Controllers, or alternatively Elite Controllers, show signs of particularly efficient cellular responses that actively control the infected cell population (8). Controller CD8+ T cells have the capacity to potently inhibit HIV replication when added to cultures of infected autologous CD4+ T cells, and are thought to play a key role in HIV control (9, 10). Recent evidence suggest that particular TCR clonotypes expressed by Controller CD8+ T cells are responsible for their efficient cytotoxic responses, while HLA-matched non-Controller patients show clonotypes of lower efficacy (11, 12). The dominant Gag-specific clonotypes from Controllers appear more efficient at lyzing HIV-infected target cells through the rapid release of cytolytic granule content (12-14). In addition, clonotypes from Controllers are able to maintain cross-recognition of dominant epitope variants, thus preventing the emergence viral escape mutants (11, 12, 15). There is in the field a particular need to be in a position to target potential viral escape mutants.

However, the role of the CD4 response in HIV control remains more debated. Controllers maintain a population of HIV-specific CD4+ T cells endowed with a strong proliferative capacity, which has been linked to autocrine IL-2 production and the upregulation of anti-apoptotic molecules (16-18). A significant fraction of IL-2 producing CD4+ T cells in Controllers retain a central memory phenotype, while the HIV-specific central memory population is depleted in progressor patients (16, 19). The presence of long-lived central memory T cells in Controllers allows the persistence of immunological memory, but does not appear sufficient to ensure HIV control. Indeed, patients treated early in the course of primary HIV infection also maintain central memory CD4+ T cells with strong proliferative capacity, but in most cases fail to control HIV replication upon treatment interruption (20). A contributing factor may be the preferential infection and depletion of HIV-specific CD4+ T cells upon viral rebound, as these cells activate upon HIV antigen recognition and become highly susceptible target cells for viral replication (5). How HIV-specific CD4+ T cells escape depletion in Controllers is not entirely understood, though some studies suggest a lower susceptibility of Controller CD4+ T cells to HIV replication (21). In addition, multiple lines of evidence indicate that the antiviral CD4 response in Controllers is qualitatively different from that of efficiently treated patients, and is not just a consequence of a very low viremia. In particular, Controller CD4+ T cells preferentially target Gag rather than Env epitopes, suggesting differences in the repertoire of specific CD4+ T cells (22). Controller CD4+ T cells are more polyfunctional, as indicated by the capacity to produce multiple cytokines and chemokines simultaneously upon antigenic stimulation (23). A key difference lies in the persistence of specific CD4+ T cells with an effector differentiation status in controlled HIV infection, even though the amount of HIV antigens available to drive these responses is minimal. We reported that Gag-specific CD4+ T cells in Controllers maintain a Th1 effector profile with IFN-γ production and degranulation capacity, while such effectors disappear in patients treated in the long term (24). Controller CD4+ T cells express very low levels of negative costimulatory molecules such as CTLA-4 and PD-1, compatible with preserved effector functions (25, 26). Taken together, these findings suggest that Th1 effectors escape depletion and retain optimal functions to sustain the antiviral response in Controlled HIV infection.

An explanation for the remarkable properties of HIV-specific CD4 responses in Controllers may lie in the nature of the TCRs that mediate these responses, which remains unexplored so far. The inventors previously identified a population of CD4+ T cells with a high antigen sensitivity for immunoprevalent Gag peptides in HIV Controllers, while this population was absent in treated patients (27). The sensitive detection of Gag antigens depended on the TCR expressed by Controller CD4+ T cells, as indicated by a high TCR avidity measured in MHC II tetramer dilution assays. These experiments revealed intrinsic differences in the set of TCRs expressed by Controller CD4+ T cells as compared to those of treated patients. High TCR avidity is a hallmark of viral control in several models of chronic viral infections (28), and has been associated with antiviral efficacy in HIV-specific CD8+ T cells (10, 14, 29). In contrast, there are little data available on the impact of TCR avidity on human CD4+ T cell antiviral responses. The inventors reasoned that the presence of high avidity TCRs may explain why Controllers maintained HIV-specific CD4+ T cells with an effector differentiation status in spite of their very low viremia, and they set to characterize these TCRs at the molecular and functional level. The study reported herein was focused on TCRs specific for the most immunoprevalent CD4 epitope in Gag, located at position 293-312 in the capsid major homology region (MHR). This epitope, designated Gag293, is exceptionally immunoprevalent, as it induces an ELISPOT response in close to half of HIV-1 infected patients irrespective of genetic background (22, 30), and gives a >70% response rate in Controllers of the ANRS CO21 CODEX cohort (27). The inventors could thus compare the repertoire of Gag293-specific clonotypes in patients of varied HLA backgrounds, who controlled HIV infection either naturally or pharmacologically, through antiretroviral therapy. Clonotypes corresponding to both the TCRα and TCRβ chains were analyzed, so that patient-derived TCRs could be engineered and functionally tested.

The CD4+ T cell response in HIV control appears to remain an important field of investigation, at the source of therapeutic achievements. In this respect, there is a need for effective therapeutic strategies, which would be applicable to human patients having HIV infection, especially, but not only, those under long-term antiretroviral therapy, in order to circumvent or alleviate the drawbacks associated with such a treatment.

In particular, there is a need for immunotherapeutic approaches that aim at mimicking the antiviral responses seen in patients, HIV Controllers, who spontaneously control HIV replication in the absence of therapy. HIV Controllers develop particularly efficient antiviral T cell responses (82, 83). The inventors' findings, as detailed herein, pave the way to harnessing such efficient responses for cellular adoptive therapy targeting HIV, in order to trigger a full set of antiviral functions.

The present invention emanates from experiments, whose conclusions are that particular TCRs based on public clonotypes may confer high-avidity CD4+ T cell responses to HIV Controllers, as in particular defined herein.

Therefore, the invention concerns a T-cell receptor (TCR) which is specific for the epitope located between positions 293-312 in the GAG protein of HIV-1 (Gag293 epitope herein), said TCR comprising an alpha chain and a beta chain, wherein the sequence of the alpha variable domain of the alpha chain comprises a gene product from the human TRAV24 gene and the sequence of the beta variable domain of the beta chain comprises a gene product from the human TRBV2 gene.

By “Gag293 epitope”, it is meant the most immunoprevalent CD4 epitope in Gag, located at position 293-312 in the capsid major homology region (MHR) of the HIV-1, especially the epitope encoded by the nucleic acid sequence TTTAGAGACTATGTAGACCGGTTCTATAAAACTCTAAGAGCCGAGCAAGCTTCACAGGAG (SEQ ID NO: 54), corresponding to nucleotides 1212-1271 from HIV-1 HXB2 sequence with the GeneBank accession number AF033819.

The nucleotide sequence encoding the Gag 293-312 epitope according to the present disclosure is variable, given HIV extensive genetic diversity. The skilled person in the art is however aware of these variations.

TCRs alpha and beta chains each consist of a variable domain and a constant domain. The variable domain is at the amino-terminal end of the TCR molecule. Within a TCR variable domain, there are six complementarity determining regions (CDRs) that mediate recognition with an epitope, i.e. three CDRs found in the sequence of the alpha variable domain of the alpha chain, and three CDRs found in the sequence of the beta variable domain of the beta chain. The CDR1 and CDR2 regions found in the variable regions are encoded by germline TRAV and TRBV gene segments.

The diversity of the TCR repertoire produced by an individual relies on two phenomena known as 1) combinatorial diversity and 2) junctional diversity.

Combinatorial diversity results from the many possible different permutations and combinations of variable (V—TRAV or TRBV), diversity (D—TRBD) and joining (J—TRAJ or TRBJ) gene segments that can be chosen amongst the numerous V, D and J gene segments available in the genome of an individual, which can be recruited when TCRs are generated by the human body. Junctional diversity results from the further rearrangement, known as V(D)J recombination or V(D)J rearrangement, within the selected V and J gene segments, and also the selected D gene segment for the beta chain, during which germline-encoded nucleotide(s) may be deleted and random non-contemplated nucleotide(s) may be added.

The CDR3 regions on each variable chain of a TCR emanate from rearranged junction(s) of different V(D)J gene segments. The amino-acid sequence or corresponding nucleotide sequence of a CDR3 region (also termed CDR3 junction herein) is therefore particularly hypervariable, especially at the level of the rearranged junction(s), where junctional diversity events (rearrangement events or V(D)J recombination events) occurred.

A TCR of the invention, which is specific for the Gag293 epitope, therefore comprises three CDRs on its alpha variable domain and three CDRs on its variable beta domain, that are the result of a gene product based on the human TRAV24 and TRBV2 genes, wherein these genes are rearranged with TRAJ or TRBJ genes respectively for the constitution of the CDR3 domains.

By “gene product based on the human TRAV24 and TRBV2 genes”, it is meant a protein or polypeptide the amino acid sequence of which is produced by the combinatorial selection of these particular gene segments, which defines the amino-acid sequence of the alpha and beta chains, respectively, including the CDR1 and CDR2 regions. This selection also further sets the basis for the definition of the amino-acid sequence of the CDR3 region, which is then submitted to V(D)J recombination. According to a particular embodiment, the sequences of the alpha variable domain and the beta variable domains of a TCR of the invention comprise a rearranged gene product at the level of their respective CDR3 regions, which is at least partly based on the human TRAV24 and TRBV2 genes, respectively.

TRAV24 gene is located on chromosome 14 at 14q1.2.

TRBV2 gene is located on chromosome 7 at 7q34.

Part of the present disclosure, sequences for germline TRAV24 and TRBV2 genes are respectively disclosed under:

a. SEQ ID NO: 55 for the TRAV24-01 allelic sequence corresponding to the accession number AE000660 in the IMGT database at www.imgt.org

ATGGAGAAGAATCCTTTGGCAGCCCCATTACTAATCCTCTGGTTTCATCT
TGACTGCGTGAGCAGCATACTGAACGTGGAACAAAGTCCTCAGTCACTGC
ATGTTCAGGAGGGAGACAGCACCAATTTCACCTGCAGCTTCCCTTCCAGC
AATTTTTATGCCTTACACTGGTACAGATGGGAAACTGCAAAAAGCCCCGA
GGCCTTGTTTGTAATGACTTTAAATGGGGATGAAAAGAAGAAAGGACGAA
TAAGTGCCACTCTTAATACCAAGGAGGGTTACAGCTATTTGTACATCAAA
GGATCCCAGCCTGAAGACTCAGCCACATACCTCTGTGCCTTTA

b. SEQ ID NO: 56 for the TRBV2-01 allelic sequence corresponding to the accession number L36092 in the IMGT;

GAACCTGAAGTCACCCAGACTCCCAGCCATCAGGTCACACAGATGGGACA
GGAAGTGATCTTGCGCTGTGTCCCCATCTCTAATCACTTATACTTCTATT
GGTACAGACAAATCTTGGGGCAGAAAGTCGAGTTTCTGGTTTCCTTTTAT
AATAATGAAATCTCAGAGAAGTCTGAAATATTCGATGATCAATTCTCAGT
TGAAAGGCCTGATGGATCAAATTTCACTCTGAAGATCCGGTCCACAAAGC
TGGAGGACTCAGCCATGTACTTCTGTGCCAGCAGTGAAGC

The present disclosure also encompasses other alleles which have been reported for these genes. For example one allelic variant has been reported for the TRAV24 gene: TRAV24-02, accession number M17661 in the IMGT database. Two allelic variants have been reported for the TRBV2 gene: TRBV2-02 (accession M62379) and TRBV2-03 (accession M64351) in the IMGT database. These allelic variants are also part of the present disclosure in order to define the TCR rearranged coding sequences.

According to a particular embodiment, the CDR1 and CDR2 amino-acid sequences of a TCR of the invention are those encoded by the corresponding coding regions of the TRAV24 and TRBV2 germline genes defined herein, or a variant thereof.

In the following part of the description, in the absence of a different definition or meaning, reference to a “gene” is equivalent to a reference to a “germline gene”, when the context indicates that the CDR3 region of a TCR is not involved or considered. By definition, the sequence of the CDR3 region of a TCR (amino-acid sequence or nucleotide sequence) results from a rearrangement providing structural diversity with respect to the information directly derivable from germline genes.

The skilled person knows how to identify the amino-acid sequence (and corresponding encoding nucleotide sequence) of a CDR1 or CDR2 of a TCR. The nomenclature used herein is that of the IMGT database (www.imgt.org). In this respect, reference is made to Front Genet. 2012 May 23; 3:79. doi: 10.3389/fgene.2012.00079. eCollection 2012, “IMGT-ONTOLOGY 2012”, Giudicelli V, Lefranc MP which indicates that the numbering nomenclature used in the IMGT database provides a standardized delimitation of the complementarity determining regions (CDR), and therefore allows to correlate each position (amino-acid or codon) with the structure and the function of a variable domain of a TCR. Reference is also made to Cold Spring Harb Protoc. 2011 Jun. 1; 2011(6):633-42. doi: 10.1101/pdb.ip85. “IMGT unique numbering for the variable (V), constant (C), and groove (G) domains of IG, TR, MH, IgSF, and MhSF.” Lefranc MP, which, in particular, discloses in FIG. 2 an “IMGT Protein display for V domain” providing a delimitation of CDR1 and CDR2 domains with respect to the IMGT numbering. IMGT numbering allows the inclusion of gaps and additional positions. Using these references and possibly IMGT tools, the skilled in the art can therefore identify the amino-acid sequence (and corresponding encoding nucleotide sequence) of a CDR1 or CDR2 of a TCR upon visualization of a given sequence or sequences alignments. By “variant”, it is meant a polypeptide resulting from limited variations in the sequence of the polypeptide of reference, variant polypeptides encompassing polypeptides having at least 80% identity with the sequence of reference. According to a particular embodiment, identity percentages reach 85%, 86%, 87%, 88%, 89%, 90% or more, including 91%, 92%, 93%, 94%, 95%, 96%, 97%, 98% or 99%. In a particular embodiment, identity percentages are at least of 95%. By extension, as defined herein, “variant” also applies to nucleic acid molecules, including nucleic acid molecules encoding polypeptide(s) as defined herein, or as described herein.

By “% identity” it is meant that result is obtained when sequences of amino-acids or nucleotides are compared over the entire length of the considered reference sequence through a local alignment algorithm, for example the Smith and Waterman algorithm, allowing amino-acids or nucleotides substitution(s), addition(s) or deletion(s). According to another particular embodiment, the identity percentage is calculated through a global alignment, for example the Needleman and Wunsch algorithm. In this case, the modifications of amino-acids or nucleotides are especially substitutions.

TCRs of interest for immunotherapy can confer efficient T cell functions to the cells expressing them i.e., cells displaying TCRs of the invention at their surface. It is an aspect of the present invention that the TCRs described herein can confer highly efficient T cell biological functions.

Such biological functions may be assessed by evaluation of TCR antigen (and/or epitope) sensitivity.

As defined herein, the expressions “sensitivity” or “antigen sensitivity” encompass and/or can be alternatively be defined by the notions of “avidity” and/or “functional avidity”.

“Avidity” describes the ability of a TCR to recognize an antigen, and more particularly refers to assessment of the strength of the interaction between a TCR and its cognate antigen, taking into account multiple interactions influences in the context of a TCR displayed at the surface of a cell, more particularly T-cell(s), more particularly when presented by a MHC molecule, in particular a class II MHC molecule. A conventional way to determine avidity is to measure the fluorescent intensity of pMHC multimers bound to the TCR or also through tetramer titration experiments, in particular MHC II tetramer titration experiments as illustrated in the Examples herein.

“Functional avidity” is determined by exposure of the TCR in the context of cell(s), more particularly T-cell(s), more particularly when presented by a MHC molecule, in particular a class II MHC molecule, to different amounts of antigen, and is a biological measure aimed at assessing T-cells(s) response to antigen stimulation. Therefore, functional avidity inversely correlates with the antigen dose that is needed to trigger a T-cell response. Functional avidity can be determined by ex vivo, in particular in vitro, quantification of biological functions such as induction of T cell activation marker such as CD69, cytokine production, especially IFN-gamma production, cytotoxic activity (ability to lyse and/or induce the lysis of target cells, especially HIV1-infected cells), or proliferation. According to a particular embodiment, TCRs of the invention as disclosed herein show functional avidity for their cognate antigen, especially functional avidity quantified according to any one or several of the biological function(s) described herein.

Accordingly, antigen sensitivity of a TCR of the invention for its cognate antigen can be assessed, according to a particular embodiment, by the measure of the capability of CD4+ T cells which express a TCR of the invention, to proliferate and to differentiate into TNF-alpha and/or IFN-gamma secreting effectors upon stimulation with said epitope with half-maximal responses for TNF-alpha or IFN-gamma production (EC50) being achieved in particular with less than 10⁻⁷ M of Gag293 epitope concentration.

Alternatively, the antigen sensitivity may be assessed in transduced J76 cells wherein EC₅₀ for the Gag293 peptide concentration able to induce half maximal CD69 induction is assessed.

“Cytotoxicity” assessment refers to the ability of TCRs of the invention, in particular when displayed at the surface of a cell, especially a T-cell according to the embodiments described herein, to lyse and/or induce the lysis of target cells, especially HIV1-infected cells, in particular CD4+ HIV1-infected cells. The skilled person is aware of methods enabling such a determination. A particular example is shown in the experimental section herein. HIV suppression can especially be evaluated, in particular quantified, by the decrease of fluorescent signal, compared to a mock experiment.

According to a particular embodiment, the TCRs of the invention have cytotoxic capability, especially when displayed at the surface of cells, such as T-cells, in particular CD4+ and/or CD8+ cells. According to a particular embodiment, cytotoxicity can be shown at any degree of target cells lysis (HIV suppression). According to other particular embodiments, the percentage of lysis (HIV suppression) is as disclosed in the embodiments described herein.

“Polyfunctionality” of a TCR is, as detailed herein, indicated by its capacity to produce multiple cytokines and/or chemokines simultaneously upon antigenic stimulation. Polyfunctionality is in particular a well-established indicator describing the ability of a cell TCR to control a virus infection.

Therefore, “sensitivity”, “antigen sensitivity”, “avidity”, “functional avidity” and/or “polyfunctionality” as defined herein, are relevant parameters to characterize the functionality, especially the biological functionality, of the TCRs of the invention according to any one of the embodiments described herein.

The invention therefore relates to a TCR which is specific for the epitope located between positions 293-312 in the GAG protein of HIV-1 having the amino-acid sequence FRDYVDRF[Y/F]KTLRAEQA[S/T]QE (SEQ ID NO: 1), comprising an alpha chain and a beta chain whose variable domains each comprise three complementarity determining regions (CDR1, CDR2, CDR3), wherein

a. the amino-acid sequence of the CDR1 and CDR2 on the alpha variable chain is encoded by the human TRAV24 gene, and

b. the amino-acid sequence of the CDR1 and CDR2 on the beta variable chain is encoded by the human TRBV2 gene, and

c. the TCR has sensitivity, especially high sensitivity, for the epitope located between positions 293-312 of the GAG protein of HIV-1 as defined herein, in particular a Gag293 epitope that is presented by a MHC molecule, in particular a class II MHC molecule, as disclosed herein.

Sensitivity may be assessed or measured:
i. as the capability of CD4+ T cells which express the TCR, to proliferate and to differentiate into TNF-alpha and/or IFN-gamma secreting effectors upon stimulation with said epitope, defined through the epitope concentration required for achieving half-maximal responses (EC50) for TNF-alpha or IFN-gamma production, and/or
ii. by monitoring the induction of the early activation marker CD69 by cells expressing the TCRs, especially transduced J76 cells, upon stimulation with said epitope, as defined through the epitope concentration required for achieving half-maximal responses (EC50) for CD69 induction, and/or
iii. by MHC-class II tetramers binding/titration experiment, especially as shown in the Examples, sensitivity being defined by half-maximal tetramer binding values (EC50) in cells transduced with the TCR, in particular J76 cells, and/or
iv. by assessing with TCR-transduced cells, upon antigenic stimulation using the Gag293 peptide, markers production including at least one of the following markers: cytokines such as TNF-alpha, IL-2, IFN-gamma, chemokines such as MIP-1beta/CCL4, degranulation marker such as CD107a, and/or
v. by assessing the polyfunctionality of TCR-transduced cells such as CD4+ or CD8+ cells, in particular by intracellular cytokine staining (ICS), upon antigenic stimulation using the Gag293 peptide, the induction of at least 3 cytokines, in particular 5 cytokines in the group of TNF-alpha, IL-2, IFN-gamma, MIP-1beta and degranulation marker CD107a, and/or
vi. by assessing the cytotoxicity of TCR-transduced cells, such as CD4+ or CD8+ cells, in particular as shown in the Examples, in the presence of HIV-infected autologous target cells, in particular dendritic cells, especially by evaluating the percentage of viral suppression.

According to particular embodiments, “high” sensitivity as introduced in c) above is reached when any one or several of the parameters i. to vi. defined herein are achieved independently or in combination thereof, such that:

i. the EC50 value (minimal epitope concentration required to achieve half-maximal response(s)) for TNF-alpha or IFN-gamma production is in the range of 10E-8 to 10E-7 M, in particular is less than 10-7 M of Gag293 epitope concentration, and/or

ii. the EC50 value for CD69 induction is in the range of 10-5 M to 10-7 M, in particular in the range of 10-6 M to 10-7 M (depending upon the restricting HLA-DR allele—see Table 10 herein), more particularly is in the 10-7 M range, for example is about 4×10⁻⁷ M, and/or

iii. the EC50 tetramer binding value is in the range of 10E-9 to 10E-7 M (depending upon the restricting HLA-DR allele—see FIG. 10E herein), more particularly is in the 10-8 M range, and/or

v. the TCR is polyfunctional as measured by the induction of at least 3 cytokines, in particular 5 cytokines in the group of TNF-alpha, IL-2, IFN-gamma, MIP-1beta and degranulation marker CD107a, when the TCR is expressed on CD4+ and/or CD8+ T cells. According to a particular embodiment, polyfunctionality for the 5 above-mentioned cytokines is achieved through their detectable (or specific) expression until peptide dose below 10E-7 M, in particular below 10E-8 M in CD4+ cells. According to another particular embodiment, EC50 value when measuring TNF-alpha, CD107a or MIP-1beta is in the range of 10E-9 to 10E-7 M in CD4+ cells. According to another particular embodiment, EC50 value when measuring IFN-gamma is in the range of 10E-8 to 10E-7 M in CD4+ cells. According to another particular embodiment, polyfunctionality for the 5 above-mentioned cytokines is achieved through their detectable expression in CD8+ T cells. According to another particular embodiment, EC50 value when measuring TNF-alpha, CD107a or MIP-1beta is in the range of 10E-7 to 10E-5 M, or 10E-7 to 10E-6 M, or 10E-6 to 10E-5 M, in CD8+ cells, in particular is within the 10E-6 M range, and/or

vi. the viral suppression observed in HIV-infected target cells, especially in the presence of transduced CD4+ T cells, is in the range of about 40% to 100%, or about 50% to 100%, or about 60% to 100%, or about 70% to 100%, or about 40% to 90%, or about 50% to 90%, or about 60% to 90%, or about 70% to 90%, or about 40% to 80%, or about 50% to 80%, or about 60% to 80%, or about 70% to 80%, or about 40% to 70%, or about 50% to 70%, or about 60% to 70%, or about 40% to 60%, or about 50% to 60%, in particular is above 50%, more particularly is above 60%, especially at the E:T (Effector:Target) ratio of 5. According to a particular or another embodiment, viral suppression can be detected in HIV-infected target cells at an effector to target ratio selected amongst: 0.25, 0.5, 1, 2, 5, or any ratio in between or within any range defined between any one of these values. According to a particular, viral suppression can be detected in HIV-infected target cells at an effector to target ratio below 2 or below 1, or below 0.5, in particular below 0.25. According to a particular embodiment, effector cells are (transduced) CD4+ cells, or CD8+ cells, or both. According to a particular or another embodiment, the viral suppression observed in HIV-infected target cells, especially in the presence of transduced CD8+ T cells, is in the range of 40% to 100%, or about 50% to 100%, or about 60% to 100%, or about 70% to 100%, or about 40% to 90%, or about 50% to 90%, or about 60% to 90%, or about 70% to 90%, or about 40% to 80%, or about 50% to 80%, or about 60% to 80%, or about 70% to 80%, or about 40% to 70%, or about 50% to 70%, or about 60% to 70%, or about 40% to 60%, or about 50% to 60%, in particular is above 50%, more particularly is above 90%. Effector to target ratio(s) may as indicated above.

According to a particular embodiment, the viral suppression observed in HIV-infected target cells in the presence of transduced CD4+ T cells, is in the range of about 40% to 100%, or about 50% to 100%, or about 60% to 100%, or about 70% to 100%, or about 40% to 90%, or about 50% to 90%, or about 60% to 90%, or about 70% to 90%, or about 40% to 80%, or about 50% to 80%, or about 60% to 80%, or about 70% to 80%, or about 40% to 70%, or about 50% to 70%, or about 60% to 70%, or about 40% to 60%, or about 50% to 60%, in particular is above 50%, more particularly is above 60%, at the E:T (Effector:Target) ratio of 5.

According to a particular or another embodiment, the viral suppression observed in HIV-infected target cells in the presence of transduced CD8+ T cells, is in the range of 40% to 100%, or about 50% to 100%, or about 60% to 100%, or about 70% to 100%, or about 40% to 90%, or about 50% to 90%, or about 60% to 90%, or about 70% to 90%, or about 40% to 80%, or about 50% to 80%, or about 60% to 80%, or about 70% to 80%, or about 40% to 70%, or about 50% to 70%, or about 60% to 70%, or about 40% to 60%, or about 50% to 60%, in particular is above 50%, more particularly is above 90%, at an effector to target ratio below 2 or below 1, or below 0.5, in particular below 0.25.

More specifically, according to a particular embodiment, the invention relates to a TCR which is specific for the epitope located between positions 293-312 in the GAG protein of HIV-1 having the amino-acid sequence FRDYVDRF[Y/F]KTLRAEQA[S/T]QE (SEQ ID NO: 1), comprising an alpha chain and a beta chain whose variable domains each comprise three complementarity determining regions (CDR1, CDR2, CDR3), wherein

a. the amino-acid sequence of the CDR1 and CDR2 on the alpha variable chain is encoded by the human TRAV24 gene, and

b. the amino-acid sequence of the CDR1 and CDR2 on the beta variable chain is encoded by the human TRBV2 gene, and

c. the TCR has high sensitivity for the epitope located between positions 293-312 of the GAG protein of HIV-1 as defined herein, in particular a Gag293 epitope that is presented by a MHC molecule, in particular a class II MHC molecule, as disclosed herein, that may be measured as the capability of CD4+ T cells which express the TCR, to proliferate and to differentiate into TNF-alpha and/or IFN-gamma secreting effectors upon stimulation with said epitope with half-maximal responses for TNF-alpha or IFN-gamma production (EC50) being achieved in particular with less than 10⁻⁷ M of Gag293 epitope concentration.

Alternatively the antigen sensitivity may be assessed in transduced J76 cells wherein EC₅₀ for the Gag293 peptide concentration able to induce half maximal CD69 is observed.

High antigen sensitivity may be assayed according to the protocols disclosed in the Examples and the EC₅₀ may be in the range of the values disclosed in the Examples.

In a particular embodiment, antigen sensitivity of the TCR may be assessed in CD4+ and/or CD8+ T cell expressing an assayed TCR by determining the EC₅₀ value, e.g., measured by intracellular cytokine staining (ICS). Accordingly the minimal Gag293 concentration required to achieve half-maximal expression of the assayed cytokine(s) provides the EC₅₀ value. According to a particular embodiment, the TCR of the invention has EC₅₀ value in the range of 10E-9 to 10E-8 M when measuring one of TNF-alpha, MIP-1beta and degranulation marker CD107a production and has EC₅₀ value in the range of 10E-8 to 10E-7 M when measuring IFN-gamma production and/or has intermediate EC₅₀ value when measuring IL-2 production.

In particular embodiments, antigen sensitivity, in particular “high” antigen sensitivity, of the TCRs described herein, and of the cells expressing/displaying them, can be assessed according to any one of the aspects described in i. to vi. above, taken alone or in combination thereof, especially as shown in the Examples.

In a particular embodiment, antigen affinity of a TCR of the invention may be considered to define such TCR in addition or alternatively to the sensitivity.

By “affinity”, reference is made to the physical strength of the interaction between a TCR and its antigen, especially in the context of its presentation by MHC molecules, especially an antigen complexed with a class II MHC molecule.

High affinity of the TCR for the epitope defined herein may be measured by a Kd value that is equal or less than 20 μM measured by SPR (Surface Plasmon Resonance) analysis.

In a particular embodiment, the affinity of the TCR for said a Gag293 epitope is equal or less than 19, 18, 17, 16, 15, 14, 13, 12, 11, 10, 9, 8, 7, 6, 5, 4, 3, 2 or 1 μM, more particularly in the ranges of: 0.5 to 7 μM, 0.5 to 4 μM, 0.5 to 3 μM or 0.5 to 1 μM, by SPR analysis. According to a particular embodiment, the affinity is less than 1 μM, in particular is 0.86 μM.

These values define Gag293-specific TCRs i.e., TCR which have high-affinity for the epitope, being observed that such affinity values are not commonly encountered for TCRs recognizing epitopes presented by class II MHC molecules.

The affinity can be defined by a Kd_{eq} value (also designated Kd) and can be measured by methods conventional in the art, in particular methods reported in the experimental section below, especially by SPR analysis.

In addition, it is observed that physiological TCR affinities are usually in the 1-100 μM range. As an immunotherapeutic tool, increasing TCR affinity to supra-physiological levels may raise the risks of non-specific cross-reactivity with cellular proteins. For instance, it has been reported, in a TCR transfer therapy trial, a TCR targeting the MAGE-A3 cancer antigen with supra-physiological affinity, which was found to unexpectedly recognize a protein expressed in cardiac tissue, resulting in two fatalities (92). Because of this risk of serious adverse events, adoptive cellular therapy trials involving TCR of supra-physiological affinity may not be the most judicious choice. In this context, provision of TCRs that are in the higher range of physiological affinities may represent a better strategy. Of interest, the TCRs of the invention can reach affinities, which are exactly in the highest range of affinities reported for naturally expressed CD4-derived TCRs (93).

According to the nomenclature used herein, when reference is made to motif(s), the indication [Z1/Z2] means that either amino-acid identified as Z1 or amino-acid identified as Z2 can alternatively be found at that position in the sequence. Motif(s) cover(s) several possibilities of amino-acid sequences, according to all possible combinations of amino-acid residues encompassed by the alternatives suggested between brackets “[” and “]” over the whole length of the motif(s).

In a further aspect of the invention the TCR is specific for the epitope located between positions 293-312 in the GAG protein of HIV-1 having the amino-acid sequence FRDYVDRF[Y/F]KTLRAEQA[S/T]QE (SEQ ID NO: 1, comprising an alpha chain and a beta chain whose variable domains each comprise three complementarity determining regions (CDR1, CDR2, CDR3), wherein

a. the amino-acid sequence of the CDR1 and CDR2 on the alpha variable chain is encoded by the human TRAV24 gene, and

b. the amino-acid sequence of the CDR1 and CDR2 on the beta variable chain is encoded by the human TRBV2 gene, and

c. the amino-acid sequence of the CDR3 on the alpha chain comprises a motif selected amongst: [A/S]X[K/R]AAGNKLT (SEQ ID NO: 2) (motif AV24-1 herein), AXYGGATNKLI (SEQ ID NO: 3) (motif AV24-2), AX[R/N][R/N]AGNMLTF (SEQ ID NO: 4) (motif AV24-3), AXD[N/D]RKLI (SEQ ID NO: 5) (motif AV24-4) or AXE[S/G]X[G/A][A/S][Q/E]KLV (SEQ ID NO: 6) (motif AV24-5), X being any amino-acid, and/or

d. the amino-acid sequence of the CDR3 on the beta chain comprises a motif selected amongst: ASSX[R/G/L][T/A][S/G]GXX[E/D/T][Q/T][F/Y]) (SEQ ID NO: 7) (motif BV2-1), ASSX[R/G/L][T/A][S/G/A]GXX[E/D/T/P][Q/T][F/Y/H] (SEQ ID NO: 8) (motif BV2-2-b), ASSGXXNTEAF (SEQ ID NO: 9) (motif BV2-3) or ASVLMRT[N/R]NEQF (SEQ ID NO: 10) (motif BV2-4), X being any amino-acid.

When a TCR of the invention is further defined through the amino-acid sequence of the CDR3 on its alpha and/or beta chain as disclosed in any embodiment herein, said TCR may further keep the functional properties further described according to any aspect or combination of aspects herein.

According to a particular embodiment, the amino-acid sequence of the CDR3 on the alpha chain comprises either the motif [A/S]X[K/R]AAGNKLT (SEQ ID NO: 2) or AXYGGATNKLI (SEQ ID NO: 3).

According to a particular embodiment, the amino-acid sequence of the CDR3 on the beta chain comprises either the motif ASSX[R/G/L][T/A][S/G]GXX[E/D/T][Q/T][F/Y]) (SEQ ID NO: 7) or ASSX[R/G/L][T/A][S/G/A]GXX[E/D/T/P][Q/T][F/Y/H] (SEQ ID NO: 8)

According to a specific embodiment, the CDR3 on the alpha chain comprises one of the above motifs of SEQ ID NO: 2 or SEQ ID NO: 3 and the CDR3 on the beta chain comprises one of the above motifs of SEQ ID NO: 7 or SEQ ID NO: 8.

According to a particular embodiment, the length of the amino-acid sequence of the CDR3 on the alpha chain of a TCR of the invention is from 9 and 16 amino-acid residues, in particular 12 amino-acid residues, and/or the length of the amino-acid sequence of the CDR3 on the beta chain of a TCR of the invention is from 11 and 18 amino-acid residues, in particular 15 amino-acid residues. CDR3 junctions lengths indicated herein are computed by including the conserved C104 and F/W118 residues in the length of the CDR3 junction, according to the numbering scheme of the International ImMunoGeneTics Information System (IMGT) (34), which excludes these two residues from the CDR3 length calculation. Indeed, according to the IMGT-ONTOLOGY unique numbering system, “CDR3 lengths” correspond to the number of amino-acids comprised between, but not including, the two conserved residues C104 and F/W118 defined by the IMGT-ONTOLOGY numbering system. In contrast, the “CDR3 junctions” referred to herein and the lengths of said CDR3 junctions provided herein include said conserved C104 and F/W118 residues. The IMGT-ONTOLOGY numbering system and “CDR3 lengths” calculation according to this system is however used in the experimental section.

When CDR3 is referred to in the present invention, the definition or feature may be adapted (in terms of amino acid residues) to read on CDR3 junction as an alternative unless technically irrelevant or unless specified otherwise.

The inventors have identified particular relevant motifs for the amino-acid sequence of the CDR3s of a TCR of the invention:

| Motifs | |
|---|---|
| TRAV24 | (SEQ ID NO: 2 to 6) |
| AV24-1 | [A/S]X[K/R]AAGNKLT |
| AV24-2 | AXYGGATNKLI |
| AV24-3 | AX[R/N][R/N]AGNMLTF |
| AV24-4 | AXD[N/D]RKLI |
| AV24-5 | AXE[S/G]X[G/A][A/S][Q/E]KLV |
| Motifs | |
| TRBV2 | (SEQ ID NO: 7 to 10) |
| BV2-1 | ASSX[R/G/L][T/A]SGGXX[E/D/T][Q/T][F/Y] |
| BV2-2-b | ASSX[R/G/L][T/A][S/G/A]GXX[E/D/T/P][Q/T] |
| | [F/Y/H] |
| BV2-3 | ASSGXXNTEAF |
| BV2-4 | ASVLMRT[N/R]NEQF |

In a particular embodiment, the TCR of the invention is an isolated TCR. By “isolated” reference is made to a TCR that has been separated from at least some of the components with which it was associated when initially produced in nature or through a preparation process and/or produced and/or prepared through dedicated non-naturally occurring settings. The TCR can be a non-naturally occurring TCR and/or a recombinant and/or engineered TCR. In a particular embodiment, the TCR of the invention is modified with respect to a naturally occurring TCR so as to differ from the structure of the naturally occurring TCR while retaining the functional properties discussed herein. According to a particular embodiment, the TCR of the invention is a “variant” of a naturally occurring TCR according to the definitions provided herein, in particular is a TCR varying from its naturally occurring counterpart by at least one, in particular one, amino-acid modification, especially substitution.

In a particular embodiment, the TCR is a human TCR. In another particular embodiment, the TCR is a chimeric TCR having constant regions domains from another species than human, especially murine constant regions domains.

According to a particular embodiment, a TCR of the invention recognizes the Gag293 epitope defined herein when presented by a major histocompatibility complex (MHC) molecule. According to a more particular embodiment, such a MHC molecule is a MHC Class II molecule. MHC Class II molecules are normally found only on antigen-presenting cells such as dendritic cells, mononuclear phagocytes, some endothelial cells, thymic epithelial cells, and B cells.

According to a particular embodiment the Gag293 epitope is a peptide from the GAG protein of HIV-1 having the amino-acid sequence FRDYVDRF[Y/F]KTLRAEQA[S/T]QE (SEQ ID NO: 1), optionally presented by a MHC molecule as defined herein, in particular presented by a MHC-II molecule present on an Antigen Presenting Cell.

According to a particular embodiment, a TCR of the invention specifically recognizes a peptide from the GAG protein of HIV1 having the amino-acid sequence FRDYVDRF[Y/F]KTLRAEQA[S/T]QE (SEQ ID NO: 1) when presented by a major histocompatibility complex (MHC) molecule, in particular when said peptide is presented by a peptide-MHC II complex.

According to a particular embodiment, a TCR of the invention displays multiple, especially “broad”, HLA cross-restriction, meaning that a TCR of the invention is MHC II cross-restricted by several HLA-DR alleles, in particular at least 2, 3, 4, 5, 6 or 7 distinct HLA-DR alleles. According to a particular embodiment, multiple restriction is achieved with at least two and up to four or five of HLA alleles selected amongst: HLA-DR11, HLA-DR15, HLA-DRB5, HLA-DR1 and/or HLA-DR7, or several of these alleles, according to all combinations thereof.

A TCR of the invention may recognize all or part of the Gag293 epitope defined herein.

According to a particular embodiment, the antigenic peptide of the Gag293 epitope as defined herein is recognized by a TCR of the invention when presented as a peptide-MHC II complex by antigen-presenting cells.

According to a particular embodiment, the MHC class II molecule is a HLA-DR molecule, especially a HLA-DR molecule selected from the group consisting of: HLA-DR11, HLA-DR15, HLA-DRB5, HLA-DR1, HLA-DR7, or any subcombination thereof. According to a particular embodiment, the MHC-II molecule is a HLA-DR11, HLA-DR15, HLA-DRB5 or HLA-DR1 molecule.

The invention also relates to an antigen-TCR complex formed by the interaction between a TCR of the invention as disclosed herein and peptide of the Gag293 epitope as disclosed herein, said antigenic peptide being presented by a MHC molecule, in particular a class II MHC molecule, as disclosed herein.

According to a particular embodiment, the invention relates to a TCR as defined herein, when presented on a cell, in particular on a primary T cell, such as a PBMC, with HLA-DR restriction, in particular with a restriction to at least one of HLA-DR11, HLA-DR15, HLA-DRB5, HLA-DR1 and/or HLA-DR7, or several of them, according to all combinations thereof, more particularly with a restriction to at least one of HLA-DR11, HLA-DR15, HLA-DRB5 or HLA-DR1 or several of them, according to all combinations thereof.

The specificity of the TRC for its epitope may be assessed by assaying the recognition of Gag293-loaded HLA-DR tetramers, as illustrated in the Examples in tetramer titration experiments. Such specificity may also be inferred from the binding affinity of the obtained TCR or from their sensitivity.

According to a particular embodiment, the TCRs of the invention are characterized as being functional TCRs, especially polyfunctional TCRs.

The functional properties of the TCRs of the invention can be one or several of the functional properties described herein, and/or in the Examples.

In a particular embodiment, these TCRs confer high antigen sensitivity to gag-specific CD4+ and/or CD8+ T cells which express them, in particular to both CD4+ and C8+ T cells, as measured by the capacity of these cells to proliferate and to differentiate into TNF-alpha and/or IFN-gamma secreting effectors upon stimulation with half-maximal responses for TNF-alpha or IFN-gamma production (EC50) that is equal to less than 10⁻⁷ M of Gag293 peptide.

According to a particular embodiment, antigen sensitivity of cells may be assessed by monitoring the induction of the early activation marker CD69 by cells expressing the TCRs.

According to a particular embodiment TCR function according to the invention may be determined assessing markers production including at least one, preferably at least 2 or at least 3 of the following markers: cytokines such as TNF-alpha, IL-2, IFN-gamma, chemokines such as MIP-1beta/CCL4, degranulation marker such as CD107a. TCRs of the invention are in particular very active for the purpose of the invention when they are capable of eliciting the secretion of IFN-gamma in addition to TNF-alpha.

In a particular embodiment of the invention, the TCR is polyfunctional as measured by assaying induction of at least 3 cytokines, in particular 5 cytokines in the group of TNF-alpha, IL-2, IFN-gamma, MIP-1beta and degranulation marker CD107a, and preferably cytokines including IFN-gamma and TNF-alpha by primary CD4+ T cells expressing said TCR upon limiting antigenic stimulation using the Gag293 peptide. Alternatively induction of the activation marker CD69 in J76 cell lines may be used to assess functionality of the TCRs instead of CD4+ T cells.

If assessed in vitro, production of these markers by cells expressing the TCRs of the invention may involve intracellular cytokine staining (ICS) after stimulating the cells with Gag293 peptide.

In a particular embodiment, it has been observed that the TCRs of the invention induce a functional response and in particular a polyfunctional response when expressed on CD4+ T cells, especially CD4+ T cells obtained after transduction with vector particles disclosed herein. Interestingly some TCRs of the invention (such as F24 disclosed hereafter) may also be expressed on transduced CD8+ T cells (by transduction with vector particles as disclosed herein) and induce the secretion of the above cited markers thereby providing CD8+ T cells more effective against HIV replication.

As defined herein, a “public clonotype” corresponds to identical CDR3 amino-acid sequences found in at least two individuals analyzed during the study reported herein.

By extension, a “public clonotype” can also be defined as a corresponding nucleotide sequence encoding such a CDR3 amino-acid sequence, or a set of corresponding nucleotide sequences, given the degeneracy of the genetic code.

According to a particular embodiment, a TCR of the invention has variable domains of the alpha and beta chains each comprising three complementarity determining regions (CDRs), wherein:

a. the amino-acid sequence of the CDR3 on the alpha chain is or comprises a sequence as disclosed in any one or several of SEQ ID NO: 11 to 27 (as disclosed and individualized in Table 3A herein), and/or

b. the amino-acid sequence of the CDR3 on the beta chain is or comprises a sequence as disclosed in any one or several of SEQ ID NO: 28 to 46 (as disclosed and individualized in Table 3B herein), or

c. the amino-acid sequence of the CDR3 on the alpha and/or beta chain comprises a variant having at least 80% amino-acids sequence identity with the sequences disclosed in a. and b. respectively, or a variant as defined herein with respect to identity percentages,

the length of the amino-acid sequence of the CDR3 on the alpha chain being from 9 and 16 amino-acid residues, in particular 12 amino-acid residues, the length of the amino-acid sequence of the CDR3 on the beta chain being from 11 and 18 amino-acid residues, in particular 15 amino-acid residues.

Identity percentages for variants are calculated as disclosed above. The length of the amino-acid sequence of the CDR3 is calculated as indicated above.

According to a particular embodiment, a TCR of the invention has variable domains of the alpha and beta chains each comprising three complementarity determining regions (CDRs), wherein:

a. the amino-acid sequence of the CDR3 on the alpha chain is or comprises a sequence selected from: CAFKAAGNKLTF (SEQ ID NO: 47) (public clonotype TRAV24-F herein), CASKAAGNKLTF (SEQ ID NO: 48) (TRAV24-S), and CSRRAAGNKLTF (SEQ ID NO: 49) (TRAV24-RR), and/or

b. the amino-acid sequence of the CDR3 on the beta chain is or comprises a sequence selected from: CASSRLAGGMDEQF (SEQ ID NO: 50) (TRBV2-24), CATTPGASGISEQF (SEQ ID NO: 51) (TRBV2-25), CASSPGTSGVEQFF (SEQ ID NO: 52) (TRBV2-4), and CASSRRTSGGTDTQYF (SEQ ID NO: 53) (TRBV2-13), or

c. the amino-acid sequence of the CDR3 on the alpha and/or beta chain comprises a variant having at least 80% amino-acids sequence identity with the sequences disclosed in a. and b. respectively, or a variant as defined herein with respect to identity percentages,

wherein the length of the amino-acid sequence of the CDR3 on the alpha chain is from 9 and 16 amino-acid residues, in particular 12 amino-acid residues, the length of the amino-acid sequence of the CDR3 on the beta chain is from 11 and 18 amino-acid residues, in particular 15 amino-acid residues.

Identity percentages for variants are calculated as disclosed above. The length of the amino-acid sequence of the CDR3 is calculated as indicated above.

Other particular embodiments of a TCR of the invention are described with respect to the sequence of the alpha and beta chains of the TCR identified as TCR “F24” herein, as disclosed in FIGS. 15A to 15F and 16A to 16F, which have been recovered by the inventors upon sequencing of the nucleotide sequence (SEQ ID NO: 57) of the plasmid termed “pCDH-F24-TCR” herein (8241 nucleotides), disclosed in the experimental section and part of the present disclosure.

Translation in amino-acids of the nucleotide sequence found in this plasmid provides the following sequences for the expressed chains of the TCR:

amino-acid sequence (SEQ ID NO: 58-274 aa) of the alpha chain of a TCR of the invention, translated from its encoding nucleic acid sequence:

MEKNPLVAPLLILWFHLDCVSSILNVEQSPQSLHVQEGDSTNFTCSFP
SSNFYALHWYRWETAKSPEALFV [symbol] EKKKGRISATLNTKEG
YSYLYIKGSQPEDSATYL [symbol] GGGTRVLVKPNIQKPDPA
ITYQLRDSKSSDKSITCLFTDFDSQTNITSQSKDSDITYITDKTVLDM
RSMDFKSNSAVAWSNKSDFACANAFNNSIIPADTFFPSPESSCDVKL
VEKSFETDTNLNFQNLSVIGFRILLLKVAGFNLLMTLRLWSS

amino-acid sequence (SEQ ID NO: 59-313 aa) of the beta chain of a TCR of the invention, translated from its encoding nucleic acid sequence:

MDTWLVCWAIFSLLKAGLTEPEVTQTPSHQVTQMGQEVILRCVPISNHL
YFYWYRQILGQKVEFLVS [symbol] [symbol] SEKSEIFDDQFSVERPDGS
NFTLKIRSTKLEDSAMYF [symbol] [symbol] GPGTRLTVLEDLKNVFP
PEVAVFEPSEAEISHTQKATLVCLATGFYPDHVELSWWVNGKEVHSGV
STDPQPLKEQPALNDSRYCLSSRLRVSATFWQNPRNHFRCQVQFYGLS
ENDEWTQDRAKPVTQIVSAEAWGRADCGFTSESYQQGVLSATILYEIL
LGKATLYAVLVSALVLMAMVKRKDSRG

Legend for SEQ ID NO: 58 and SEQ ID NO: 59:

| | |
|---|---|
| Alpha chain domains | Beta chain domains |

Leader
CDR1
CDR2

| | |
|---|---|
| TRAV24 sequence is the | TRBV2 sequence is the |
| sequence from amino acid 1 | sequence from amino acid 1 |
| to the amino acid before | to the amino acid before |
| the CDR3 junction | the CDR3 junction |

CDR3-JUNCTION

| | |
|---|---|
| from 3′TRAV24 | from 3′TRBV2 |
| and 3′-TRAJ17 | and 3′-TRBJ2-1 |
| TRAC | TRBC2 |

The positions of the corresponding regions of interest in SEQ ID NO: 58 and 59 are therefore:

Alpha chain (SEQ ID NO: 58):

| Region | Start | End |
|---|---|---|
| TRAV24 | 1 | 111 |
| CDR1-IMGT | 49 | 54 |
| CDR2-IMGT | 72 | 77 |
| CDR3-long | 112 | 123 |
| 3′-TRAJ17 | 124 | 133 |
| TRAC | 134 | 274 |

Beta chain (SEQ ID NO: 59):

| Region | Start | End |
|---|---|---|
| TRBV2 | 1 | 110 |
| CDR1-IMGT | 46 | 50 |
| CDR2-IMGT | 68 | 73 |
| CDR3-long | 111 | 125 |
| 3′-TRBJ2-1 | 126 | 134 |
| TRBC2 | 135 | 313 |

A leader sequence is a sequence at the N-terminus of some eukaryotic proteins that determines their ultimate destination. Rearranged TCR genes contain a short leader sequence upstream of the joined VJ and VDJ sequences. As the corresponding nascent polypeptide enters the endoplasmic reticulum, the amino acid sequences encoded by the leader sequence are cleaved. Mature TCR chains do not encompass amino acids corresponding to a leader sequence.

According to a particular embodiment, a TCR of the invention has an amino acid sequence that is devoid of leader sequences.

Are also part of the present disclosure the amino acid sequences SEQ ID NO: 60 and SEQ ID NO: 61, which correspond respectively to the amino acid sequences SEQ ID NO: 58 and SEQ ID NO: 59 minus the amino acid sequences corresponding to the leader sequences discussed above.

Accordingly, according to a particular embodiment, a TCR of the invention has:

the amino-acid sequence of the CDR1 and CDR2 on the alpha variable chain encoded by the human TRAV24 gene, in particular has an amino-acid sequence for the CDR1alpha corresponding to the positions 49 to 54 in SEQ ID NO: 58 and has an amino-acid sequence for the CDR2alpha corresponding to the positions 72 to 77 in SEQ ID NO: 58 and/or

the amino-acid sequence of the CDR1 and CDR2 on the beta variable chain encoded by the human TRBV2 gene, in particular has an amino-acid sequence for the CDR1beta corresponding to the positions 46 to 50 in SEQ ID NO: 59 and has an amino-acid sequence for the CDR2beta corresponding to the positions 68 to 73 in SEQ ID NO: 59 and/or

the amino-acid sequence of the CDR3 on the alpha chain that is: CAFKAAGNKLTF (SEQ ID NO: 11), and the amino-acid sequence of the CDR3 on the beta chain that is: CASSRLAGGMDEQFF (SEQ ID NO: 512), or

the amino-acid sequence of the CDR3 on the alpha and/or beta chain comprises a variant having at least 80% amino-acids sequence identity with the sequences disclosed above for the CDR3 found on the alpha and beta chains, respectively, or a variant as defined herein with respect to identity percentages, the length of the amino-acid sequence of the CDR3 on the alpha chain being from 9 and 16 amino-acid residues, in particular 12 amino-acid residues, the length of the amino-acid sequence of the CDR3 on the beta chain being from 11 and 18 amino-acid residues, in particular 15 amino-acid residues.

Identity percentages for variants are calculated as disclosed above. The length of the amino-acid sequence of the CDR3 is calculated as indicated above.

According to a particular embodiment, a TCR of the invention has constant domains TRAC and/or TRBC2 as defined in the Table above.

According to a more particular embodiment, a TCR of the invention has:

the amino-acid sequence of its alpha chain is as disclosed in SEQ ID NO: 58 or SEQ ID NO: 60, and/or

the amino-acid sequence of its beta chain is as disclosed in SEQ ID NO: 59 or SEQ ID NO: 61, or

the amino-acid sequence of its alpha and/or beta chain is a variant having at least 80% amino-acids sequence identity with the sequences disclosed above, or a variant as defined herein with respect to identity percentages.

Identity percentages for variants are calculated as disclosed above.

According to a particular embodiment, a TCR of the invention has alpha and/or beta chains and/or variable chains as disclosed herein, especially above, in which the amino-acid sequence of the CDR3 on the alpha and/or beta chains is replaced by any CDR3 junction sequence or motif as disclosed herein, or a variant thereof having at least 80% amino-acids sequence identity according to the definitions provided herein, in particular a variant as defined herein with respect to identity percentages, in which case the length of the amino-acid sequence of the variant CDR3 on the alpha chain is from 9 and 16 amino-acid residues, in particular 12 amino-acid residues, the length of the amino-acid sequence of the variant CDR3 on the beta chain is from 11 and 18 amino-acid residues, in particular 15 amino-acid residues.

According to a particular embodiment, the obtained TCR remains polyfunctional as disclosed herein, and/or retains an affinity for the epitope located between positions 293-312 of the GAG protein of HIV-1 as defined herein.

Identity percentages for variants are calculated as disclosed above. The length of the amino-acid sequence of the CDR3 is calculated as indicated above.

The skilled persons knows how to substitute an amino-acid sequence (and corresponding encoding nucleotide sequence) of a CDR3 of a TCR, or any part of a segment of a TCR as identified herein. Reference is made to the publications regarding the IMGT nomenclature disclosed above.

According to a particular embodiment, the TCR of the invention as disclosed with respect to its amino acid sequences is a human TCR. According to another particular embodiment the TCR of the invention as disclosed by its amino acid sequences is a chimeric TCR, in particular a human-murine TCR.

According to a particular embodiment, a TCR of the invention has constant domains encoded by the TRAC and TRBC genes for the alpha and beta chain respectively.

Part of the present disclosure, sequences for germline TRAC and TRBC genes are respectively disclosed under:

a. SEQ ID NO: 64 for the TRAC01 allelic sequence corresponding to the accession number X02883 in the IMGT database at www.imgt.org

NATATCCAGAACCCTGACCCTGCCGTGTACCAGCTGAGAGACTCTAAATC
CAGTGACAAGTCTGTCTGCCTATTCACCGATTTTGATTCTCAAACAAATG
TGTCACAAAGTAAGGATTCTGATGTGTATATCACAGACAAAACTGTGCTA
GACATGAGGTCTATGGACTTCAAGAGCAACAGTGCTGTGGCCTGGAGCAA
CAAATCTGACTTTGCATGTGCAAACGCCTTCAACAACAGCATTATTCCAG
AAGACACCTTCTTCCCCAGCCCAGAAAGTTCCTGTGATGTCAAGCTGGTC
GAGAAAAGCTTTGAAACAGATACGAACCTAAACTTTCAAAACCTGTCAGT
GATTGGGTTCCGAATCCTCCTCCTGAAAGTGGCCGGGTTTAATCTGCTCA
TGACGCTGCGGCTGTGGTCCAGC

b. SEQ ID NO: 65 for the TRBC02-2 allelic sequence corresponding to the accession number L36092 in the IMGT;

GAGGACCTGAAAAACGTGTTCCCACCCGAGGTCGCTGTGTTTGAGCCATC
AGAAGCAGAGATCTCCCACACCCAAAAGGCCACACTGGTATGCCTGGCCA
CAGGCTTCTACCCCGACCACGTGGAGCTGAGCTGGTGGGTGAATGGGAAG
GAGGTGCACAGTGGGGTCAGCACAGACCCGCAGCCCCTCAAGGAGCAGCC
CGCCCTCAATGACTCCAGATACTGCCTGAGCAGCCGCCTGAGGGTCTCGG
CCACCTTCTGGCAGAACCCCCGCAACCACTTCCGCTGTCAAGTCCAGTTC
TACGGGCTCTCGGAGAATGACGAGTGGACCCAGGATAGGGCCAAACCCGT
CACCCAGATCGTCAGCGCCGAGGCCTGGGGTAGAGCAGACTGTGGCTTCA
CCTCCGAGTCTTACCAGCAAGGGGTCCTGTCTGCCACCATCCTCTATGAG
ATCTTGCTAGGGAAGGCCACCTTGTATGCCGTGCTGGTCAGTGCCCTCGT
GCTGATGGCCATGGTCAAGAGAAAGGATTCCAGAGGC

The present disclosure also encompasses other alleles which have been reported for these genes or sequences varying in 1, 2, 3, 4 or 5 nucleotides positions. Slight variations in constant sequences do not affect or do not substantially affect TCR function.

According to a particular embodiment, a TCR of the invention is a recombinant TCR.

According to the invention such recombinant TCR may be obtained by recombination, especially by PCR cloning of the sequences defined herein, in particular by recombination of the sequences encoding the CDR1 and CDR2 together with any of the CDR3 where the combination in the respective resulting alpha and beta chains and the assembly of the alpha and beta chains provides a functional, in particular TCR with high antigen affinity or sensitivity. The positions of the domains of interest of the TCR as disclosed herein, provide in particular the features to design a TCR pattern.

The invention further relates to a TCR having the features disclosed, which is an heterodimeric TCR. By “heterodimeric TCR”, it is meant a TCR composed of two different disulfide-linked chains. Differently said, an heterodimeric TCR as defined herein consists of both an alpha chain and a beta chain, which are associated through disulphide bond(s) at the level of their respective constant domains. TCR constant domains comprise connecting sequences having at least one cysteine residue capable of engaging in disulfide bonds, enabling the formation of a link between the two chains and an heterodimeric structure.

The invention also relates to a chimeric TCR that is a single chain TCR (scTCR) comprising the variable domains of an alpha and a beta chain of the invention (Vα and Vβ) as defined herein, which are connected through a linker (L), an alpha constant domain and/or a beta constant domain fused to their respective variable alpha or beta chain, according to the following scheme VαCα-L-VβCβ.

The invention also relates to a chimeric TCR that is a soluble TCR, meaning a TCR that has lost its capacity to be membrane-bound. The constant domain of an alpha or beta chain of a TCR is encoded by the Trac and Trbc genes, respectively, typically include the following elements: a constant domain sequence Cα or Cβ, a connection sequence (H), a transmembrane region (Tm), and a cytoplasmic tail (CT). A soluble TCR lacks at least the transmembrane region (Tm) and the cytoplasmic tail (CT) region. A soluble TCR may be an heterodimeric TCR or a scTCR, provided it has lost its capacity to be membrane-bound. According to a particular embodiment, the chains of an heterodimeric soluble TCR are further engineered to add additional disulphide bond(s) between the two chains forming the heterodimeric structure.

The invention also relates to a chimeric TCR that is a single chain TCR fragment (scTv) consisting of the variable domains of an alpha and a beta chain of the invention (Vα and Vβ) as defined herein, which are connected through a linker (L), according to any one of the following schemes: Vα-L-Vβ or Vβ-L-Vα. Being devoid of at least a transmembrane region, a scTv is a soluble TCR according to the definitions provided herein. A scTv does not comprise TCR constant domains.

The chimeric TCR of the invention include the features of the alpha and/or beta TCR chains, or fragments thereof, disclosed herein. In particular, a chimeric TCR comprises a variable alpha chain and/or a variable beta chain region of the invention as defined herein.

The invention also relates to a chimeric and/or further engineered TCR, such as a TCR that has undergone murinization its constant region(s), i.e., a TCR having murine constant regions domain(s) instead of human constant regions domain(s).

The invention also relates to a chimeric and/or further engineered TCR, such as a TCR that has undergone cysteine(s) modification(s) of its TCR chains, especially at the level of its constant regions, i.e., a TCR, the chains of which have been modified to have more cysteine residues than the non-engineered TCR counterpart, that are capable of engaging in additional disulfide bonds linking the alpha and beta chains of, in particular, an heterodimeric structure

The invention also encompasses a TCR as disclosed herein, or a fragment thereof, which is associated, optionally through a linker, with a detectable group such as a fluorescent group, a radiolabelled group, an absorbing group, or an enzyme with properties that generate detectable products.

By “rearranged gene product”, it is meant an amino-acid sequence resulting from combinatorial diversity events and junctional diversity events applied to the nucleotide sequence of germline gene segments. The amino-acid sequence of a “rearranged gene product” is therefore defined by the amino-acid sequence produced by the combinatorial selection of particular gene segments, to which junctional diversity is added by V(D)J recombination in the junctional sequence comprised by the CDR3 region.

According to a particular embodiment, a TCR of the invention has:

a. an amino-acid sequence of the CDR3 on the alpha chain comprising a rearranged gene product obtained upon rearrangement of the human TRAV24 gene with a human TRAJ gene selected from: TRAJ17, TRAJ39, TRAJ32, TRAJ38, TRAJ54 or TRAJ57, and/or

b. the amino-acid sequence of the CDR3 on the beta chain comprises a rearranged gene product obtained upon rearrangement of the human TRBV2 gene with a human TRBJ gene selected from: TRBJ2-1, TRBJJ1-2, TRBJJ1-1, TRBJ1-5, TRBJ2-3, TRBJ2-7, and with a human TRBD gene selected from: TRBD1 or TRBD2.

Further to the rearrangement, some nucleotides of the TRAJ or the TRB germline genes may be present in the nucleic acid encoding the resulting alpha, respectively beta chains of the TCR, corresponding to amino residues found at the C-terminal end of the CDR3 domains.

Part of the present disclosure, sequences for germline TRAJ17, TRAJ39, TRAJ32, TRAJ38, TRAJ54, TRAJ57 TRBJ2-1, TRBJ1-2, TRBJ1-1, TRBJ1-5, TRBJ2-3, TRBJ2-7, TRBD1 and TRBD2 are respectively disclosed under:

a. SEQ ID NO: 66 for the TRAJ17-01 allelic sequence corresponding to the accession number X05773 in the IMGT database at www.imgt.org

TGATCAAAGCTGCAGGCAACAAGCTAACTTTTGGAGGAGGAACCAGGGTG
CTAGTTAAACCAA

b. SEQ ID NO: 67 for the TRAJ32-01 allelic sequence corresponding to the accession number M94081 in the IMGT;

TGAATTATGGCGGTGCTACAAACAAGCTCATCTTTGGAACTGGCACTCTG
CTTGCTGTCCAGCCAA

c. SEQ ID NO: 68 for the TRAJ39-01 allelic sequence corresponding to the accession number M94081 in the IMGT;

TGAATAATAATGCAGGCAACATGCTCACCTTTGGAGGGGGAACAAGGTTA
ATGGTCAAACCCC

d. SEQ ID NO: 69 for the TRAJ54-01 allelic sequence corresponding to the accession number M94081 in the IMGT;

TAATTCAGGGAGCCCAGAAGCTGGTATTTGGCCAAGGAACCAGGCTGACT
ATCAACCCAA

e. SEQ ID NO: 70 for the TRAJ57-01 allelic sequence corresponding to the accession number M94081 in the IMGT;

TAACTCAGGGCGGATCTGAAAAGCTGGTCTTTGGAAAGGGAACGAAACTG
ACAGTAAACCCAT

f. SEQ ID NO: 71 for the TRBJ1-1-01allelic sequence corresponding to the accession number K02545 in the IMGT;

TGAACACTGAAGCTTTCTTTGGACAAGGCACCAGACTCACAGTTGTAG

g. SEQ ID NO: 72 for the TRBJ1-2-01 allelic sequence corresponding to the accession number K02545 in the IMGT;

CTAACTATGGCTACACCTTCGGTTCGGGGACCAGGTTAACCGTTGTAG

h. SEQ ID NO: 73 for the TRBJ1-5-01 allelic sequence corresponding to the accession number M14158 in the IMGT;

TAGCAATCAGCCCCAGCATTTTGGTGATGGGACTCGACTCTCCATCCTAG

i. SEQ ID NO: 74 for the TRBJ2-1-01 allelic sequence corresponding to the accession number X02987 in the IMGT;

CTCCTACAATGAGCAGTTCTTCGGGCCAGGGACACGGCTCACCGTGCTAG

j. SEQ ID NO: 75 for the TRBJ2-3-01 allelic sequence corresponding to the accession number X02987 in the IMGT;

AGCACAGATACGCAGTATTTTGGCCCAGGCACCCGGCTGACAGTGCTCG

k. SEQ ID NO: 76 for the TRBJ2-7-01 allelic sequence corresponding to the accession number M14159 in the IMGT;

CTCCTACGAGCAGTACTTCGGGCCGGGCACCAGGCTCACGGTCACAG

l. SEQ ID NO: 77 for the TRBD1-01 allelic sequence corresponding to the accession number K02545 in the IMGT;

GGGACAGGGGGC

m. SEQ ID NO: 78 for the TRBD2-01 allelic sequence corresponding to the accession number X02987 in the IMGT;

GGGACTAGCGGGGGGG

The present disclosure also encompasses other alleles which have been reported for these genes.

Accordingly, the invention also relates to a method for identifying a TCR specific for the epitope located between positions 293-312 of the GAG protein of HIV-1 as defined herein, comprising the steps of:

a. Selecting a cell bearing TCRs specific for the epitope located between positions 293-312 of the GAG protein of HIV-1 as defined herein, and

b. Sequencing the full length or part of the alpha and beta chains of the TCRs of the selected cells, and

c. Expressing TCRs having the sequence determined in step b. in cells, and

d. Measuring an EC50 value for TNF-alpha or IFN-gamma production by CD4+ T cells expressing the said TCR that is equal to less than 10⁻⁷ M of Gag293 peptide, or measuring an EC50 value for TNF-alpha or IFN-gamma production by J76 cells expressing the said TCR that is equal to less than 10⁻⁶ M of Gag293 peptide, or measuring any one or several of the values described herein with respect to the different manners of determining whether a TCRs as disclosed herein has a “high” antigen sensitivity.

According to a particular embodiment, a method for identifying a TCR specific for the epitope located between positions 293-312 of the GAG protein of HIV-1 as defined herein, comprising the steps of

a. Selecting a cell bearing TCRs specific for the epitope located between positions 293-312 of the GAG protein of HIV-1 as defined herein, and

b. Sequencing the full length or part of the alpha and beta chains of the TCRs of the selected cells, and

c. Producing soluble TCRs having the sequence determined in step b. in cells, and

d. Measuring a Kd value for the soluble TCRs and identifying hit TCR(s) displaying a Kd value that is equal or less than 20 μM, in particular equal or less than 19, 18, 17, 16, 15, 14, 13, 12, 11, 10, 9, 8, 7, 6, 5, 4, 3, 2 or 1 μM, more particularly between the ranges of: 0.5 to 7 μM, 0.5 to 4 μM, 0.5 to 3 μM or 0.5 to 1 μM.

According to a particular embodiment, TCR(s) expressed or produced in steps c) above have variable domains of the alpha and beta chains each comprising three complementarity determining regions (CDRs), in which:

i. the amino-acid sequence of the CDR3 on the alpha chain comprises a rearranged gene product obtained upon rearrangement of the human TRAV24 gene with a human TRAJ gene selected from: TRAJ17, TRAJ39, TRAJ32, TRAJ38, TRAJ54 or TRAJ57, and/or

ii. the amino-acid sequence of the CDR3 on the beta chain comprises a rearranged gene product obtained upon rearrangement of the human TRBV2 gene with a human TRBJ gene selected from: TRBJ2-1, TRBJJ1-2, TRBJJ1-1, TRBJ1-5, TRBJ2-3, TRBJ2-7, and with a human TRBD gene selected from: TRBD1 or TRBD2.

Cell selection in steps a) above can be performed according to conventional methods in the art. The experimental section provides an example of sorting of Gag293-specific CD4+ T cells with HLA-DR tetramers.

The invention also relates to a nucleic acid molecule encoding at least one chain of TCR as defined herein, or a fragment thereof, or both alpha and beta chains. The nucleic acid molecule of the invention may encode several TCRs as defined herein, or fragments thereof.

Such a nucleic acid molecule may be obtained by cloning or by synthesis, optionally including steps of recombination of various nucleic acid segments in accordance with well-known methods for the skilled person.

A nucleic acid molecule of the invention may encode the full length of either the alpha chain or the beta chain of a TCR as defined herein, or both chains, or fragment(s) thereof, especially fragments covering the variable domains of said chains. A nucleic acid molecule of the invention may encode the sequence of the alpha variable domain of the alpha chain and/or the sequence of the beta variable domain of the beta chain of a TCR as defined herein, or fragment(s) thereof. A nucleic acid molecule of the invention may comprise a nucleic acid sequence encoding a constant domain. The nucleotide sequence for a constant domain of a TCR, as comprised in TRAC and TRBC genes for respectively the alpha chain and the beta chain, typically include the following elements: a constant domain nucleotide sequence Ca, a nucleotide sequence coding for a connection sequence (H), a nucleotide sequence coding for a transmembrane region (Tm), and a nucleotide sequence coding for a cytoplasmic tail (CT). A nucleic acid molecule encoding the full length of either the alpha chain or the beta chain of a TCR also generally comprises a leader exon nucleotide sequence (L).

According to a particular embodiment, a nucleic acid molecule of the invention encodes at least the amino-acid sequence of a CDR3 domain as defined herein, or a part thereof, or all or part of the variable domain of an alpha and/or a beta chain, comprising all of the three CDRs (CDR1, CDR2, CDR3).

According to a particular embodiment, a nucleic acid molecule of the invention encodes the alpha chain of a TCR as defined herein, or a fragment thereof, and especially comprises the nucleotide sequence of a public clonotype as disclosed in any one of SEQ ID NO: 79 to 120 (as disclosed and individualized in Table 3A herein) encoding the CDR3 region of the alpha chain, or a sequence having at least 80% nucleotide sequence identity with those sequences, identity being as defined herein.

According to a particular embodiment, a nucleic acid molecule of the invention encodes the beta chain of a TCR as defined herein, or a fragment thereof and especially comprises the nucleotide sequence of a public clonotype as disclosed in any one of SEQ ID NO: 121 to 161 (as disclosed and individualized in Table 3B herein) encoding the CDR3 region of the beta chain, or a sequence having at least 80% nucleotide sequence identity with those sequences, identity being as defined herein, in particular a variant as defined herein with respect to identity percentages.

According to a more particular embodiment, such nucleic acid molecules comprise a sequence encoding at least a part of a constant domain, as defined herein.

According to a particular embodiment, the nucleic acid molecule encoding a TCR as defined herein, or fragment thereof, comprises the nucleic acid sequence of both the alpha chain and the beta chain of a TCR as defined herein, or fragment thereof.

The invention thus also relates to a nucleic acid molecule encoding the full length of an alpha chain or the variable domain of the alpha chain of a TCR at least partly encoded by the human TRAV24 gene, as obtained after (i) amplification performed on cDNA obtained from RNA transcripts of cells expressing TCRs of the invention, said amplification, especially PCR amplification, being carried out according to conventional methods accessible to the skilled person with:

a. a forward primer containing the TRAV24 leader sequence with an NheI restriction site and a Kosak sequence added in 5′: 5′-CGG CTA GCC GCC ACC ATG GAG AAG AAT CCT TTG GCA GCC-3′ (SEQ ID NO: 162), and

b. a reverse primer containing the 3′ of TRAC and a NotI site:5′-TTA GCG GCC GCG CTG GAC CAC AGC CGC AGC G-3′ (SEQ ID NO: 163),

(ii) recovery of amplified DNA and possibly cloning the recovered DNA.

The invention also relates to a nucleic acid molecule encoding the full length of a beta chain or the variable domain of the beta chain of a TCR at least partly encoded by the human TRBV2 gene, as obtained after (i) amplification performed on cDNA obtained from RNA transcripts of cells expressing TCRs of the invention, said amplification, especially PCR amplification, being carried out with:

a. a forward primer containing the TRBV2 leader sequence and a BspEI site in 5′: 5′-GGT CCG GAA TGG ATA CCT GGC TCG TAT GCT GGG C-3′ (SEQ ID NO: 164), and

b. a reverse primer containing the 3′ of TRBC and a SalI site: 5′-CCG GTC GAC CTA GCC TCT GGA ATC CTT TCT CTT GAC C-3′ (SEQ ID NO: 165),

(ii) recovery of amplified DNA and possibly cloning the recovered DNA.

Alternatively, the primers used for amplification of the full length of an alpha chain or beta chain or their variable domain(s) of a TCR at least partly encoded by the human TRAV24 or TRBV2 genes respectively, can be chosen amongst:

a. TRAV24 forward primer:
(SEQ ID NO: 166)
5′-CCG AGG CCT TGT TTG TAA TG-3′;
b. TRAC reverse primer:
(SEQ ID NO: 167)
5′GTG AAT AGG CAG ACA GAC TTG T-3′;
c. TRBV2 forward primer:
(SEQ ID NO: 164)
5′-GGT CCG GAA TGG ATA CCT GGC TCG TAT
GCT GGG C-3′;
d. TRBC reverse primer:
(SEQ ID NO: 165)
5′-CCG GTC GAC CTA GCC TCT GGA ATC CTT TCT
CTT GAC C-3′.

These primers do in particular not encompass a Kosak sequence. The skilled person readily knows how to define suitable primers aimed at amplifying nucleotide target sequences of interest.

The invention also relates to a nucleic acid molecule encoding at least a part of the alpha chain of a TCR and/or at least a part of the beta chain of a TCR, in which:

a. the nucleic acid molecule encoding at least a part of the alpha chain of a TCR, especially the variable domain of the alpha chain, comprises at least the nucleotide sequence of the human TRAV24 gene coding the CDR1 and the CDR2 of a TCR and further comprises the junctional rearranged nucleotide sequence disclosed any one of SEQ ID NO: 79 to 120 (as disclosed and individualized in Table 3A herein) or a variant thereof having at least 80% sequence identity with these sequences, in particular a variant as defined herein with respect to identity percentages, and/or

b. the nucleic acid molecule encoding at least a part of the beta chain of a TCR, especially the variable domain of the beta chain, comprises at least the nucleotide sequence of the human TRBV2 gene coding the CDR1 and the CDR2 of a TCR and further comprises the junctional rearranged nucleotide sequence disclosed any one of SEQ ID NO: 121 to 161 (as disclosed and individualized in Table 3B herein) or a variant thereof having at least 80% sequence identity with these sequences, in particular a variant as defined herein with respect to identity percentages.

As an example, FIG. 3G illustrates a junctional rearranged nucleotide sequence corresponding to the CDR3 junction of the most prevalent clonotype CAFKAAGNKLTF (SEQ ID NO: 11) obtained by rearrangements that occurred between the 3′ end of the TRAV24*01 germline sequence and TRAJ17*01 germline sequence, said rearrangements comprising mutations (P and/or N), i.e., random non-contemplated nucleotide(s) addition(s), and/or trimmed nucleotides (trim), i.e., germline-encoded nucleotide(s) deletion(s).

Differently said, according to the particular embodiment defined in the paragraphs above, a nucleic acid molecule of the invention encodes at least a part of either an alpha chain or a beta chain or their variable domain(s) of a TCR, which correspond to gene products based on the human TRAV24 and TRBV2 genes when rearranged with TRAJ and TRBJ genes, said nucleic acid molecule matching one of the clonotypes defined by an amino-acid or nucleotide sequence as set forth in Table 3A or 3B, respectively, or a variant thereof having at least 80% sequence identity with one of the nucleotide sequences set forth in Table 3A or 3B, respectively, in particular a variant as defined herein with respect to identity percentages.

The length and/or boundaries of a junctional rearranged nucleotide sequence corresponding to a CDR3 junction may also be defined by correspondence with the length of an amino-acid sequence of a CDR3 as defined above, when they are defined and computed according to the numbering scheme of the International ImMunoGeneTics Information System (IMGT) (34).

Accordingly, according to a particular embodiment, a junctional rearranged nucleotide sequence corresponding to a CDR3 junction, which therefore encodes the CDR3 of a TCR, has a length from 25 to 60 nucleotides, more particularly from 27 to 54 nucleotides.

According to particular embodiments, rearranged nucleotide junction(s) of a CDR3 (junctional rearranged nucleotide sequence above) may result from:

addition of 0 to 10 random non-contemplated nucleotide(s), and/or

deletion of 0 to 15 germline-encoded nucleotide(s).

Other particular embodiments of nucleic acid molecule encoding at least a part of the alpha chain of a TCR and/or at least a part of the beta chain of a TCR of the invention are described with respect to the sequence of the alpha and beta chains of the TCR identified as TCR “F24” herein, as disclosed in Table S8, which have been recovered by the inventors upon sequencing of the nucleotide sequence (SEQ ID NO: 57) of the plasmid termed “pCDH-F24-TCR” herein (8241 nucleotides), disclosed in the experimental section and part of the present disclosure.

The nucleotide sequence of this plasmid provides the following sequences:

nucleotide sequence (SEQ ID NO: 62-822 nt) of the alpha chain of a TCR of the invention:

ATGGAGAAGAATCCTTTGGTAGCCCCATTACTAATCCTCTGGTTTCATCT
TGACTGCGTGAGCAGCATACTGAACGTGGAACAAAGTCCTCAGTCACTGC
ATGTTCAGGAGGGAGACAGCACCAATTTCACCTGCAGCTTCCCTTCCAGC
AATTTTTATGCCTTACACTGGTACAGATGGGAAACTGCAAAAAGCCCCGA
GGCCTTGTTTGTAATGACTTTAAATGGGGATGAAAAGAAGAAAGGACGAA
TAAGTGCCACTCTTAATACCAAGGAGGGTTACAGCTATTTGTACATCAAA
GGATCCCAGCCTGAAGACTCAGCCACATACCTCTGTGCCTTTAAAGCTGC
AGGCAACAAGCTAACTTTTGGAGGAGGAACCAGGGTGCTAGTTAAACCAA
ATATCCAGAAGCCTGACCCTGCCGTGTACCAGCTGAGAGACTCTAAATCC
AGTGACAAGTCTGTCTGCCTATTCACCGATTTTGATTCTCAAACAAATGT
GTCACAAAGTAAGGATTCTGATGTGTATATCACAGACAAAACTGTGCTAG
ACATGAGGTCTATGGACTTCAAGAGCAACAGTGCTGTGGCCTGGAGCAAC
AAATCTGACTTTGCATGTGCAAACGCCTTCAACAACAGCATTATTCCAGC
AGACACCTTCTTCCCCAGCCCAGAAAGTTCCTGTGATGTCAAGCTGGTCG
AGAAAAGCTTTGAAACAGATACGAACCTAAACTTTCAAAACCTGTCAGTG
ATTGGGTTCCGAATCCTCCTCCTGAAAGTGGCCGGGTTTAATCTGCTCAT
GACGCTGCGGCTGTGGTCCAGC

SEQ ID NO: 62 can be found from positions 2497 to 3318 of the “pCDH-F24-TCR” sequence provided under SEQ ID NO: 57.

nucleotide sequence (SEQ ID NO: 63-939 nt) of the beta chain of a TCR of the invention:

ATGGATACCTGGCTCGTATGCTGGGCAATTTTTAGTCTCTTGAAAGCAGG
ACTCACAGAACCTGAAGTCACCCAGACTCCCAGCCATCAGGTCACACAGA
TGGGACAGGAAGTGATCTTGCGCTGTGTCCCCATCTCTAATCACTTATAC
TTCTATTGGTACAGACAAATCTTGGGGCAGAAAGTCGAGTTTCTGGTTTC
CTTTTATAATAATGAAATCTCAGAGAAGTCTGAAATATTCGATGATCAAT
TCTCAGTTGAAAGGCCTGATGGATCAAATTTCACTCTGAAGATCCGGTCC
ACAAAGCTGGAGGACTCAGCCATGTACTTCTGTGCCAGCAGCCGACTAGC
GGGAGGGATGGATGAGCAGTTCTTCGGGCCAGGGACACGGCTCACCGTGC
TAGAGGACCTGAAAAACGTGTTCCCACCCGAGGTCGCTGTGTTTGAGCCA
TCAGAAGCAGAGATCTCCCACACCCAAAAGGCCACACTGGTGTGCCTGGC
CACAGGCTTCTACCCCGACCACGTGGAGCTGAGCTGGTGGGTGAATGGGA
AGGAGGTGCACAGTGGGGTCAGCACAGACCCGCAGCCCCTCAAGGAGCAG
CCCGCCCTCAATGACTCCAGATACTGCCTGAGCAGCCGCCTGAGGGTCTC
GGCCACCTTCTGGCAGAACCCCCGCAACCACTTCCGCTGTCAAGTCCAGT
TCTACGGGCTCTCGGAGAATGACGAGTGGACCCAGGATAGGGCCAAACCT
GTCACCCAGATCGTCAGCGCCGAGGCCTGGGGTAGAGCAGACTGTGGCTT
CACCTCCGAGTCTTACCAGCAAGGGGTCCTGTCTGCCACCATCCTCTATG
AGATCTTGCTAGGGAAGGCCACCTTGTATGCCGTGCTGGTCAGTGCCCTC
GTGCTGATGGCCATGGTCAAGAGAAAGGATTCCAGAGGC

SEQ ID NO: 63 can be found from positions 3388 to 4326 of the “pCDH-F24-TCR” sequence provided under SEQ ID NO: 57.

The positions of the corresponding regions of interest in SEQ ID NO: 62 and 63 are therefore:

Alpha chain (SEQ ID NO: 62):

| Region | Start | End |
|---|---|---|
| TRAV24 | 1 | 333 |
| CDR1-IMGT | 145 | 162 |
| CDR2-IMGT | 214 | 231 |
| CDR3- | 334 | 369 |
| JUNCTION | | |
| 3′-TRAJ17 | 370 | 399 |
| TRAC | 400 | 822 |

Beta chain (SEQ ID NO: 63):

| Region | Start | End |
|---|---|---|
| TRBV2 | 1 | 330 |
| CDR1-IMGT | 136 | 150 |
| CDR2-IMGT | 202 | 219 |
| CDR3- | 331 | 375 |
| JUNCTION | | |
| 3′-TRBJ2-1 | 376 | 402 |
| TRBC2 | 403 | 939 |

Accordingly, according to a particular embodiment, a nucleic acid molecule of the invention encodes at least a part of the alpha chain of a TCR, especially the variable domain of the alpha chain, and comprises:

a. at least the nucleotide sequence of the human TRAV24 gene coding the CDR1 and the CDR2 of a TCR, in particular has the nucleotide sequence for the CDR1alpha corresponding to the positions 145 to 162 in SEQ ID NO: 62 and has the nucleotide sequence for the CDR2alpha corresponding to the positions 214 to 231 in SEQ ID NO: 62 and

b. a junctional rearranged nucleotide sequence (CDR3 junction) corresponding to the positions 334 to 369 in SEQ ID NO: 62, or a variant having at least 80% nucleotide sequence identity thereof, the length of the nucleotide sequence of the CDR3 junction on the alpha chain being from 25 to 60 nucleotides, more particularly from 27 to 54 nucleotides.

According to a particular embodiment, a nucleic acid molecule of the invention encodes at least a part of the beta chain of a TCR, especially the variable domain of the beta chain, and comprises:

a. at least the nucleotide sequence of the human TRBV2 gene coding the CDR1 and the CDR2 of a TCR, in particular has the nucleotide sequence for the CDR1beta corresponding to the positions 136 to 150 in SEQ ID NO: 63 and has the nucleotide sequence for the CDR2beta corresponding to the positions 202 to 219 in SEQ ID NO: 63 and

b. a junctional rearranged nucleotide sequence (CDR3 junction) corresponding to the positions 331 to 375 in SEQ ID NO: 63, or a variant having at least 80% nucleotide sequence identity thereof, in particular a variant as defined herein with respect to identity percentages, the length of the nucleotide sequence of the CDR3 junction on the alpha chain being from 25 to 60 nucleotides, more particularly from 27 to 54 nucleotides.

According to a particular embodiment, a nucleic acid molecule of the invention encompasses at least a constant domain TRAC and/or TRBC2 as defined in the Table above, or a part thereof.

According to a particular embodiment, a nucleic acid molecule of the invention encodes at least a part of the alpha chain of a TCR, especially the variable domain of the alpha chain, and at least a part of the beta chain of a TCR, especially the variable domain of the beta chain, as defined herein.

According to a more particular embodiment, a nucleic acid molecule of the invention comprises or consists of the sequence disclosed in SEQ ID NO: 62 from position 1 to 399, and/or comprises or consists of the sequence disclosed in SEQ ID NO: 63 from position 1 to 402, or comprises or consists of a variant thereof having at least 80% nucleotide sequence identity with the sequences.

According to a more particular embodiment, a nucleic acid molecule of the invention comprises or consists of the sequence disclosed in SEQ ID NO: 62, and/or comprises or consists of the sequence disclosed in SEQ ID NO: 63, or variant thereof having at least 80% nucleotide sequence identity with the sequences, in particular a variant as defined herein with respect to identity percentages.

According to a particular embodiment however, a nucleic acid molecule of the invention is devoid of a leader sequence, and its size and boundaries are adapted accordingly.

According to a particular embodiment, a nucleic acid molecule of the invention comprises or consists of a sequence encoding alpha and/or beta chains and/or the variable parts of these chains as disclosed herein, especially above, in which the nucleotide sequence of the CDR3 on the alpha and/or beta chains is replaced by any nucleotide CDR3 junction sequence as disclosed herein, or a variant thereof having at least 80% nucleotide sequence identity according to the definitions provided herein, in particular a variant as defined herein with respect to identity percentages, in which case the length of the nucleotide sequence of the CDR3 junction is from 25 to 60 nucleotides, more particularly from 27 to 54 nucleotides.

The skilled person knows how to substitute a nucleotide sequence encoding for the CDR3 of a TCR, or any part of a nucleotide sequence encoding for a segment of a TCR as identified herein, especially in the Tables above. Reference is made to the publications regarding the IMGT nomenclature disclosed above.

Identity percentages for variants are calculated as disclosed above.

According to a particular embodiment, a nucleic acid molecule as defined herein comprises or further comprises a nucleotide sequence encoding for a constant region of an alpha and/or beta chain, as defined herein.

According to a particular embodiment, a nucleic acid molecule of the invention as defined herein is a fragment of a nucleic acid molecule of the invention, especially a fragment encoding the variable domain of an alpha and/or beta chain of a TCR, in particular at least encoding the CDR1, CDR2 and CDR3 regions on said chains, optionally with other nucleic acid sequences of interest.

The invention also relates to a nucleic acid molecule encoding at least a part of the full length of the alpha chain or the variable domain of the alpha chain of a TCR as defined herein, and further encoding a part of the full length of the beta chain or the variable domain of the beta chain of a TCR as defined herein.

According to a particular embodiment, a nucleic acid molecule of the invention is a variant nucleic acid molecule, or a chimeric acid molecule.

Variants of the nucleic acid molecules defined herein may result from genetic code degeneracy. Definition for a “variant” based on percentage identity with respect to a reference sequence is provided above. Variants of the nucleic acid molecules of the invention may also encompass nucleic acid molecules encoding and/or chimeric engineered TCRs of the invention as disclosed herein.

According to a particular embodiment, the variants generally defined herein with respect to the nucleotide sequences, or the nucleic acid molecules defined herein retain the capacity to encode all or part of an alpha and/or beta chain of a TCR that retain the functional capacity to recognize a Gag293 epitope as defined herein, when comprising the appropriate alpha and beta chains, in particular with the affinity defined herein.

According to a particular embodiment, a nucleic acid molecule of the invention is an isolated nucleic acid molecule. The nucleic acid molecule of the invention can be a non-naturally occurring nucleic acid molecule and/or a recombinant and/or engineered nucleic acid molecule. In a particular embodiment, the nucleic acid molecule of the invention is a human nucleic acid molecule. In another particular embodiment, the nucleic acid molecule of the invention is a chimeric nucleic acid molecule having constant regions domains from another species than human, especially murine constant regions domains. The nucleic acid molecule of the invention can be a single-stranded or double-stranded nucleic acid molecule. The nucleic acid molecule of the invention can be a DNA or a corresponding RNA molecule.

The invention also concerns a vector, in particular a plasmid comprising at least one nucleic acid molecule as defined herein.

A plasmid or vector can be used either for cloning, for transfer or for expression purposes.

According to a particular embodiment, a plasmid of the invention is suitable for the cloning of the nucleic acid molecule it contains. Such a cloning plasmid may be a bacterial plasmid, an origin of replication and multiple restriction enzyme cleavage sites allowing the insertion of a transgene insert (transcription unit), e.g., a nucleic acid molecule as defined according to the invention, or a fragment thereof.

According to another particular embodiment, a plasmid of the invention is suitable for the expression of the nucleic acid molecule it contains. Such an expression plasmid, also termed expression vector herein, or expression construct, generally contains a promoter sequence, a transcription terminator sequence, and a transgene insert (transcription unit), e.g., a nucleic acid molecule as defined herein, or a fragment thereof. An expression vector may also contain an enhancer sequence which increases the amount of protein or RNA produced.

According to a particular embodiment, a vector of the invention comprises all or a part of the sequence of the plasmid termed “pCDH-F24-TCR” herein, encoding the alpha and beta chains of the “F24” TCR reported in Table S8 is provided under SEQ ID NO: 57 (8241 nucleotides).

The invention is accordingly also directed to a transfer vector, in particular a viral vector, especially a lentiviral transfer vector, which comprises at least one nucleic acid molecule as defined herein, or a fragment thereof, contained in a transcription unit.

According to a particular embodiment, such a lentiviral transfer vector, is used for the preparation of lentiviral vector particles and accordingly is also termed lentiviral vector genome herein.

Given its nature, such a lentiviral transfer vector or lentiviral vector genome is a recombinant construct.

The invention therefore also relates to a lentiviral transfer vector which comprises lentiviral cis-active elements including long terminal repeats (LTRs) or modified LTRs including partially deleted 3′LTR, psi (P) packaging signal, optionally Rev responsive element (RRE), together with a transcription unit comprising a nucleic acid molecule encoding a TCR as defined herein, or a fragment thereof, or comprising a nucleic acid molecule as defined herein, or a fragment thereof.

According to particular embodiment, the lentiviral transfer vector does not comprise any protein from the parental lentivirus, meaning that the sequences of the original lentivirus genome encoding the lentiviral proteins are essentially deleted in the lentiviral transfer vector, resulting in a lack of expression of any viral protein from the parental lentivirus.

As used herein, the term “encoding” defines the ability of a nucleic acid molecule to be transcribed and where appropriate translated for product expression into selected cells or cell lines, when said molecule is placed under transcription and expression control sequences including promoter for transcription. Accordingly, a “polynucleotide or a nucleic acid molecule encoding” according to the invention designates the nucleic acid molecule having its sequence translated into the amino acid sequence and that may be cloned or placed under the control of expression control sequences, especially a heterologous promoter to provide a transcription unit.

In a particular embodiment, the present invention relates to a recombinant lentiviral transfer vector, which can be derived from an Human Immunodeficiency Virus (HIV), for example HIV-1 or HIV-2, Caprine Arthritis Encephalitis Virus (CAEV), Equine Infectious Anaemia Virus (EIAV), Visna/Maedi Virus (VMV), Simian Immunodeficiency Virus (SIV), Feline Immunodeficiency Virus (FIV) or Bovine Immunodeficiency Virus (BIV).

In a preferred embodiment, the lentiviral transfer vector is derived from the genome of HIV, especially of HIV-1 and is accordingly a HIV-based vector, in particular a HIV-1 based vector.

As indicated above and according to a particular embodiment, the recombinant lentiviral transfer vector is replication-incompetent as a result of lack of expression of any lentiviral protein, i.e. as a result of deletion of all or part of the gag and pol genes of the lentiviral genome or mutation in the gag and pol genes of the lentiviral genome, so that the gag and pol genes are not capable of encoding functional GAG and POL proteins.

The lentiviral transfer vector comprises the psi (p) packaging signal. The packaging signal includes a sequence coding the N-terminal fragment (about 15-30 amino-acids) of the gag ORF. In a particular embodiment, its sequence could be modified by frameshift mutation(s) or a mutation in ATG initiation codon.

In a particular embodiment, in said lentiviral transfer vector genome, the 3′ LTR sequence of the lentiviral transfer vector is devoid of at least the activator (enhancer) and of the promoter of the U3 region. In another particular embodiment, in the lentiviral transfer vector, the U3 region of the LTR 5′ is replaced by a non-lentiviral U3. In another particular preferred embodiment, the 3′ LTR region is devoid of the U3 region (delta U3). In this respect, reference is made to the corresponding description in WO 01/27300 and WO 01/27304.

In a particular embodiment, in the lentiviral transfer vector, the U3 region of the LTR 5′ is replaced by a promoter suitable to drive tat-independent primary transcription. In such a case, the vector is independent of tat transactivator.

According to a particular embodiment, the lentiviral transfer vector further comprises a DNA flap encompassing a DNA sequence of a lentivirus in particular of a HIV-1 genomic sequence which is framed by a central polypurine tract (cPPT) and a central termination sequence (CTS). The DNA flap possibly enhances nuclear import of the transcription unit of the transfer vector and increases transduction efficiency of lentiviral vector particles comprising the transfer vector.

Accordingly, a lentiviral transfer vector may be regarded as a replacement vector in which all the lentiviral protein coding sequences between the 2 LTRs have been deleted and replaced by transcription unit(s) as defined herein, and wherein the DNA flap element has been re-inserted in association with the required cis-acting sequences described herein. Further features relating to the composition of the lentiviral transfer vector are disclosed in relation to the preparation of the lentiviral vector particles.

Nucleotide sequence of a DNA flap of lentiviral origin comprises two essential regions, i.e., the cPPT and the CTS regions, wherein the cPPT and CTS regions induce a three-stranded DNA structure during replication of DNA containing them (previously defined in Zennou et al., Cell, 2000, 101, 173-185; and in the international patent applications WO99/55892 and WO01/27300).

In a particular embodiment, the DNA flap is inserted upstream of the transcription unit(s) of interest, advantageously but not necessarily to be located in an approximate central position in the lentiviral transfer vector. A DNA flap suitable for the invention may be obtained from a lentivirus, in particular a human lentivirus such as HIV-1.

It may be alternatively obtained from the CAEV (Caprine Arthritis Encephalitis Virus) virus, the EIAV (Equine Infectious Anaemia Virus) virus, the VISNA virus, the SIV (Simian Immunodeficiency Virus) virus or the FIV (Feline Immunodeficiency Virus) virus. The DNA flap may be either prepared synthetically (chemical synthesis) or by amplification of the DNA providing the DNA flap from the appropriate source as defined above such as by Polymerase chain reaction (PCR). In a more preferred embodiment, the DNA flap is obtained from an HIV lentivirus, especially HIV-1 including from any isolate or consensus sequence.

The recombinant lentiviral transfer vector further comprises transcription unit(s) as defined herein which is placed under the control of a heterologous promoter (i.e. a promoter which does not derive from the lentiviral genome providing the cis-active sequences), thereby providing a transcription unit. A particular promoter is the cytomegalovirus (CMV) promoter. Other promoters may in particular be selected for their properties as constitutive promoters, tissue-specific promoters, or inducible promoters. Examples of suitable promoters comprise the promoters of the following genes: EF1α, human PGK, PPI (preproinsulin), thiodextrin, Ferritin L chain or Ferritin H chain, Chymosin beta 4, Chymosin beta 10, Cystatin Ribosomal Protein L41, CAG, SV40 or MND.

Accordingly, in another more particular embodiment, the present invention relates to a recombinant lentiviral transfer vector as defined herein which comprises a 3′-LTR in which the promoter and the activator of the U3 region have been deleted (delta U3) and transcription unit(s) are placed under the control of a heterologous promoter, a list of which is provided above.

According to a particular embodiment, the lentiviral transfer vector of the invention enables co-expression of several transcription units, especially two transcription units respectively coding for an alpha chain of a TCR and the beta chain of a TCR, or fragments thereof as defined herein, and is a bicistronic or a multicistronic vector. Such a lentiviral transfer vector accordingly comprises several transcription units, especially two transcriptions units comprising nucleotide sequences encoding a TCR alpha chain or a fragment thereof as defined herein on one end, and a TCR beta chain or a fragment thereof as defined herein on the other end, according to the nucleotide sequences disclosed herein. Multicistronic vectors simultaneously express two or more separate proteins from the same mRNA.

According to a preferred embodiment, the lentiviral transfer vector of the invention enables stoechiometrically equivalent levels of expression of the several nucleic acid molecules it contains, especially stoechiometrically equivalent levels of expression of an alpha chain of a TCR and the beta chain of a TCR of the invention as defined herein, the co-expression of which is sought.

According to a particular preferred embodiment, a lentiviral transfer vector of the invention comprises a polynucleotide encoding a self-cleaving 2A peptide sequence inserted between the sequences encoding said alpha and beta TCR chains.

Self-cleaving 2A peptides are short peptides of about 20 amino acids that enable production of equimolar levels of multiple nucleic acid molecules from the same mRNA.

Sequences of common 2A peptides are provided below.

| | | SEQ |
|---|---|---|
| | | ID |
| Peptide | Amino acid sequence | NO: |
| T2A | E G R G S L L T C G D V E E N P G P | 168 |
| P2A | A T N F S L L K Q A G D V E E N P G P | 169 |
| E2A | Q C T N Y A L L K L A G D V E S N P G P | 170 |
| F2A | V K Q T L N F D L L K L A G D V E S N P | 171 |
| | G P | |

GSG residues can be added to the 5′ end of the sequences coding for the above-peptides to improve cleavage efficiency.

A nucleotide sequence encoding a 2A peptide can be either PCR-cloned between the nucleic acid molecules to be co-expressed or a multicistronic cassette can be inserted into a backbone as a single unit.

2A peptides permit stoechiometrically equivalent levels of expression of the nucleic acid molecules between which they are inserted.

According to a particular embodiment, the nucleotide sequence encoding the 2A peptide is a nucleotide sequence encoding T2A peptide.

The present invention also relates to a host cell either transfected or genetically transformed with a recombinant lentiviral transfer vector of the invention as disclosed herein.

The present invention also relates to a host cell either transfected or genetically transformed with the recombinant lentiviral transfer vector of the invention and with additional plasmid vectors for helper functions, including for packaging and for expression of the envelope for vector particles pseudotyping. According to a particular embodiment, more than two additional plasmid vectors may be used for co-transfection or genetic transformation, including for example a packaging plasmid vector (construct) containing only Gag and Pol genes of a lentivirus, a plasmid (construct) expressing the envelope and optionally a separate plasmid (construct) expressing the Rev gene of a lentivirus. The design of these vectors and plasmids is commonly known by the skilled person in the art.

Such a production cell for the expression of lentiviral vector particles may be either transfected or genetically transformed with a lentiviral transfer vector according to the invention and with plasmids providing helper functions, including a plasmid expressing the envelope which encodes a heterologous (i.e., non lentiviral) envelope protein, in particular an envelope protein of a VSV such as a VSV-G protein selected among VSV-G of Indiana strain, of VSV-G of New Jersey strain and including packaging construct(s) as DNA plasmid(s) encoding the GAG and POL proteins of a lentivirus, in particular of the lentivirus providing the sequences of the transfer plasmid.

The host cell of the invention is transfected with these vectors and plasmids by methods well known to the person skilled in the art, i.e. by chemical transfection (calcium phospate, lipofectamine), lipid-based techniques (liposome), electroporation, photoporation.

As used herein, the term “transfected” refers to a cell comprising a recombinant lentiviral transfer vector of the invention (transient expression), whereas the term “genetically transformed” refers to a cell whose genome has been definitively modified by a polynucleotide of the invention (permanent expression).

Said transitory or stably transformed cells can be prokaryotic (bacteria) or eukaryotic (yeast, insect or animal including mammal especially human) cells. In an embodiment, cells are non-human cells. In a particular embodiment, cells of the invention are isolated human cells, “isolated” meaning outside of their natural environment.

In a particular embodiment of the invention, the cell is from the HEK 293T (human embryonic kidney) cell line, in particular as disclosed in Zennou et al. (Cell, 2000, 101, 173-185).

The invention also relates to a method to produce lentiviral vector particles, which are recombinant lentiviral vector particles, comprising or consisting of:

a) transfecting the recombinant lentiviral transfer vector according to the invention, into a host cell suitable for packaging, for example a HEK-293T cell line or a cell line derived therefrom such as HEK-293T/17 cell line;

b) co-transfecting the cell of step a) with at least (i) a plasmid expressing the envelope, especially a plasmid vector encoding the envelope glycoprotein G of a VSV, in particular the VSV-G of Indiana or of New Jersey VSV strains and (ii) with a packaging plasmid vector encoding the lentiviral GAG and POL or mutated non integrative POL proteins of a lentivirus, in particular of a HIV-1 lentivirus;

c) recovering the recombinant lentiviral particles expressing recombinant TCR.

The transfection steps with the particular plasmid constructs disclosed above may be carried out according to a different sequence with respect to the one described above.

The present invention also relates to recombinant lentiviral vector particles obtained using a lentiviral transfer vector disclosed herein.

According to a particular embodiment, the recombinant lentiviral vector particles comprise a genome consisting of the recombinant lentiviral transfer vector as disclosed herein, and are pseudotyped with a vesicular stomatitis virus glycoprotein G (VSV-G) protein, in particular the VSV-G protein of the VSV Indiana strain or the VSV protein of the New Jersey strain.

The expression “recombinant lentiviral vector particles” defines the obtained particles expressed in a host cell or production cells following transfection by the several plasmid vectors comprising at least the lentiviral transfer vector, the envelope vector encoding the selected envelope protein and the packaging vector providing lentiviral proteins in trans (such as lentiviral GAG and POL proteins, in particular mutated POL protein for the avoidance of integration) according to methods well-known in the art.

The terms “recombinant lentiviral vector particles” encompass recombinant viral particles, and recombinant virus-like particles.

Virus-like particles result from incomplete assembly of the proteins present for encapsidation of the recombinant lentiviral transfer vector in a way that does not enable the formation of true viral particles.

According to the invention, lentiviral vector particles are the product recovered from co-transfection of host cells, for example from a HEK-293T cell line, with at least:
a recombinant lentiviral transfer vector according to the invention, as described herein;
a plasmid expressing an envelope protein, especially a plasmid vector encoding the envelope glycoprotein G of a VSV, in particular the VSV-G of Indiana or of New Jersey VSV strains;
a packaging plasmid vector encoding the lentiviral GAG and POL proteins or mutated non integrative POL protein of a lentivirus, in particular of a HIV-1 lentivirus.

According to a particular embodiment of the invention, the recombinant lentiviral vector particles are integration defective (or non-integrative) as a result of mutation or deletion in the pol gene of the lentivirus present on the packaging plasmid vector. Suitable mutations enabling formation of integration defective particles are well-known in the art and illustrated in WO 2009/019612.

According to a particular embodiment, the lentiviral vector particles encompass a recombinant lentiviral transfer vector which is multicistronic, especially biscistronic, in that it encodes at least a TCR alpha chain and a TCR beta chain of the invention or fragment(s) thereof, as disclosed herein. Accordingly, the lentiviral vector particles can include lentiviral transfer vector plasmids that may comprise bicistronic or multicistronic expression cassettes where the polynucleotides encoding the various polypeptides are separated by a nucleotide sequence encoding a 2A peptide, and/or may also further comprise several expression cassettes for the expression of various polypeptides, where the polynucleotides encoding the additional various polypeptides and distinct from the polynucleotides separated by a polynucleotide encoding a 2A peptide, are separated either by another polynucleotide encoding a 2A peptide, or by an IRES sequence of viral origin (Internal Ribosome Entry Site). Conversely, lentiviral transfer vector plasmids may encode fusion protein(s).

The invention also relates to a method for ex vivo obtaining a collection also designated as a population of recombinant cells expressing a TCR or a recombinant TCR as defined herein, comprising the steps of:

a. Transducing cells capable of expressing a functional TCR, with recombinant lentiviral vector particles of the invention, as disclosed herein, and

b. Culturing the transduced cells in conditions that permit the TCR of the invention as defined herein to be expressed, and

c. Obtaining and/or recovering a recombinant cell collection or population expressing said recombinant TCR and optionally further isolating said expressed recombinant TCR.

“Cells capable of expressing a TCR” are preferably mammalian cells, particularly human cells, especially human non dividing cells.

They can be peripheral blood mononuclear cell (PBMC), especially appropriately pre-activated according to methods known by the skilled person. They encompass T cells in particular primary T-cells, in particular primary CD4+ T cells or CD8+ cells and in particular primary CD8+ T cells or cells amplified from such cells.

According to a particular embodiment, the recovered recombinant cell collection expressing said recombinant TCR comprises or consists of or consists essentially of lymphocyte T cells, in particular primary or mature immunocompetent T cells, especially CD4+ T cells or CD8+ T cells or both.

The invention further relates to a collection or a population of recombinant human cells expressing a TCR of the invention as defined herein at their surface, or a collection or a population of cells obtained after transduction with the lentiviral particles of the invention or by the above method for obtaining a collection of recombinant cells, in particular a collection or a population of ex vivo transduced T-cells, which is formulated for administration to a human host. The transduced T cells may have integrated in their genome the nucleic acid molecule encoding the TCR, together with remaining nucleotides sequences of the LTR of the lentiviral transfer vector such as the R segment or part thereof.

The invention also relates to a collection of cells that comprises distinct populations of cells wherein the difference in the cells lies in the expression of distinct TCRs of the invention, i.e., TCRs having various sequences among those disclosed herein and/or different affinities for the Gag293 epitope of HIV-1.

The invention further relates to a composition, especially a composition comprising recombinant lentiviral vector particles of the invention as defined herein together with one or more pharmaceutically acceptable carrier(s).

The invention also relates to a composition comprising a population of ex vivo transduced cells of the invention as defined herein together with one or more pharmaceutically acceptable carrier(s).

The invention further relates to the use of a recombinant lentiviral transfer vector of the invention as defined herein or recombinant lentiviral vector particles of the invention as defined herein, or population of cells of the invention as defined herein, for the preparation of a medicament, and/or for the ex vivo preparation of an immunotherapeutically active composition, in particular a composition suitable for the treatment of patients seropositive for HIV infection.

The invention also concerns recombinant lentiviral vector particles of the invention as defined herein, or collection of cells of the invention as defined herein, for use as a medicament in a human patient infected with HIV, in particular in an immunotherapeutic treatment (therapeutic vaccine) against HIV-related disease in patients infected with HIV, in particular HIV-1.

The invention also concerns recombinant lentiviral vector particles of the invention as defined herein or collection of cells of the invention as defined herein, for use as a medicament in a human patient infected with HIV, for eliciting a CD4+ and/or CD8+ T cell response in a patient infected with HIV, in particular HIV-1, especially a patient having developed a HIV-related disease.

The invention also concerns recombinant lentiviral vector particles as defined herein or collection of cells as defined herein, for use in adoptive cell therapy, especially adoptive T cell therapy, in a human patient infected with HIV. According to a particular embodiment, said adoptive cell therapy improves immune response in treated patient, in particular, but not only, elicits a CD4+ and/or CD8+ T cell response in a patient infected with HIV, in particular HIV-1, especially a patient having developed a HIV-related disease, and/or achieves antiviral status showing maintenance of an undetectable viral load according to standard assay. According to a particular embodiment, control of HIV infection is achieved. According to a particular embodiment, adoptive T cell therapy may be CD4+ or CD8+ T cell therapy, or both, meaning that administered T cells may be CD4+ or CD8+ T cells, or both, respectively.

The invention accordingly also relates to a method of treatment of a human patient infected with HIV, or a disease as defined herein, comprising the administration to the patient of TCRs of the invention, vectors or cells of the invention, as defined in any one of the embodiments disclosed herein, or composition(s) as defined herein, especially a composition comprising population of cells of the invention as defined herein, for treating HIV, in particular HIV-1, or related disease(s), and/or for eliciting a CD4+ and/or CD8+ T cell response in said patient.

The invention also relates to a method for adoptive cell therapy of a human patient infected with HIV, or a disease as defined herein, comprising the administration to the patient of cells, especially T cell(s), as defined in any one of the embodiments disclosed herein, or composition(s) as defined herein, especially a composition comprising population of cells of the invention as defined herein, for improving the immune response of the patient and/or for eliciting a CD4+ and/or CD8+ T cell response in said patient, especially for treating HIV, in particular HIV-1, or related disease(s) in said patient.

According to a particular embodiment, treatment enables to achieve an immune protective response, e.g., an antiviral status showing maintenance of an undetectable viral load according to standard assay. According to a particular embodiment, the control of HIV infection is obtained.

The human patient treated according to the invention is in particular a patient seropositive for HIV, in particular for HIV-1, who has been treated by antiretroviral therapy and possibly is still undergoing such treatment, especially by HAART, in particular such a patient who is pharmacologically controlling the HIV infection, especially who is controlling viral load under antiretroviral therapy.

Antiretroviral therapy (ART) is treatment of patients infected with human immunodeficiency virus (HIV) using anti-HIV drugs. The standard treatment consists of a combination of at least three drugs (often called “highly active antiretroviral therapy” or HAART) that suppress HIV replication. Three drugs are used in order to reduce the likelihood of the virus developing resistance. ART and HAART have the potential both to reduce mortality and morbidity rates among HIV-infected people, and to improve their quality of life.

Antiretroviral therapy (ART) is definitively beneficial to society. However, the cost of medical care for HIV-infected patients remains at least 10 times higher than for the general population, mounting at an estimated annual 14,500 $ per patient, where the majority of the cost is directly related to ART itself (84). Many studies have concluded that the patient CD4+ T cell count is a powerful indicator of the burden of care costs, with patients who initiate treatment at a lower CD4+ T cell count showing increased non-infectious comorbidities (84, 85). For patients who have access to life-long ART, AIDS-related illnesses are no longer the primary threat, but comorbidities associated to persisting inflammation, such as cardiovascular disease, renal failure, neurocognitive disease, osteoporosis, and some types of cancers, have emerged as important complications (86). These issues are of particular concern in the group of patients called “Immunological non-responders”, or INRs, who show only limited recovery of CD4+ T cell numbers in spite of effective control of the viral load by ART. It is estimated that 10 to 20% of patients can be categorized as INRs, based on a CD4+ T cell count that remains persistently below 200 or 350 cell/mm3 after several years of suppressive ART (87, 88). INRs status is more frequent in patients who initiated ART at a low CD4+ T cell count and who were older at ART initiation (89). Large cohort studies indicate that INRs show substantially increased morbidity and mortality rates, with an increased risk of non-AIDS events of 1.96 in the EuroSIDA cohort (90), and an increased risk of mortality of 2.6 in the COHERE and ART-CC cohorts (91).

According to a particular embodiment, the human patient has been under antiretroviral therapy, including highly active antiretroviral therapy.

According to a particular embodiment, the human patient has been under antiretroviral therapy, including highly active antiretroviral therapy for at least 6 or 7 or 8, or 9 or 10 months. According to another particular embodiment, the human patient has been under antiretroviral therapy, including highly active antiretroviral therapy for at least 1 year.

According to a particular embodiment, the human patient is as defined in any one of the embodiments above, in particular is a patient still undergoing antiretroviral therapy as defined above, and is defined as an immunological non-responder patient, especially according to any one of the definitions provided herein.

According to another embodiment however, the human patient is conversely a “responder” patient.

In a particular embodiment of the invention, the treatment of a patient seropositive for HIV, in particular HIV-1, enables active control of HIV infection. Such active control may involve immune reconstitution in treated patient, sustained antiviral response and in particular maintenance of an undetectable viral load according to standard assay. Such response may in particular be designed to involve one of the following pathways: inducing CD4+ T cell expressing TCRs of the invention in particular CD4+ T cell having a central memory phenotype allowing persistence of immunological memory, or CD4+ T cell having an effector differentiation status (in particular Th1) even when associated with a low viremia, eliciting a cytotoxic response toward HIV infected cells or eliciting a helper response that will enable said cytotoxic response to take place, preventing HIV replication,

In a particular embodiment of the invention, the treatment of a patient seropositive for HIV, in particular HV-1 is designed to enable preventing risk of progressing to AIDS in the treated patient.

In a particular embodiment the patient receiving the disclosed treatment is HLA-DR matched and especially is HLA-DR11, HLA-DR15, HLA-DRB5, HLA-DR1, HLA-DR7, HLA-DR4 and/or HLA-DR3 restricted, more particularly with HLA-DR11, HLA-DR15, HLA-DRB5 or HLA-DR1 restricted.

The invention also relates to the use of active ingredient(s) as defined herein in the preparation of a medicament and/or composition for the treatment of the disease(s) discussed according to any one of the embodiment(s) described herein.

Other examples and features of the invention will be apparent when reading the examples and the figures, which illustrate the experiments conducted by the inventors, in complement to the features and definitions given in the present description.

### LEGEND OF THE FIGURES

FIGS. 1A to 1E: Immunoscope analysis of Gag293-specific CD4+ T cells

(A) Sorting of Gag293-specific CD4+ T cells with HLA-DR tetramers. Examples of primary CD4+ T cell lines labeled with DRB5 (top) and DR1 (bottom) tetramers. The percentage of tetramer positive cells (Tet+) in the total CD4+ T cell population (middle plots) and in the sorted Tet+ population (right plots) is reported in the top right corner of the graph. Samples labeled with tetramers loaded with an irrelevant peptide (CLIP or Annexin II) were used as negative controls (left plots).

(B, C) The percentages of Tet+ cells expressing the TRAV24 (B) or TRBV2 family (C) were analyzed by qPCR in the HIC and the HAART groups and compared by the Mann-Whitney test.

(D, E) CDR3 length profiles for the TRAV24 (D) and TRBV2 families (E) are shown for each patient analyzed. Healthy donor PBMC were used as control (bottom left). The percentage of the TRAV24 or TRBV2 family in the total TRAV or TRBV PCR product is reported above each profile.

FIGS. 2A to 2G: Clonotypic diversity of Gag293-specific TCRs

(A) The number of unique CDR3 amino acid sequences (clonotype AA) obtained per 100 TRAV24 (left) or TRBV2 (right) nucleotide sequences were compared in the HIC and HAART groups with the Mann-Whitney test.

(B) Simpson's diversity indexes computed for TRAV24 (left) and TRBV2 (right) clonotypes AA obtained in each patient were compared in the HIC and HAART groups with the Mann-Whitney test.

(C to E) Frequencies of TRAJ genes (D), TRBJ genes (E), and TRBD genes (F) in Gag293-specific TRA or TRB sequences. Frequencies were compared between the HIC (light grey bars) and HAART groups (dark bars).

(F): the distribution of CDR3 lengths were compared for TRAV24 between the sets of HIV Controller sequences (HIC, n=584) and treated patient sequences (HAART, n=496). CDR3 lengths are reported in number of a. a.

(G): the distribution of CDR3 lengths were compared for TRBV2 between the sets of HIV Controller sequences (HIC, n=716) and treated patient sequences (HAART, n=566).

FIGS. 3A to 3G: Quantification of public motifs and clonotypes in the Gag293-specific TCR repertoire

(A, B) The Meme motif discovery program was used to identify a.a. motifs enriched in Controller TRAV24 sequences (A) and TRBV2 sequences (B) compared to corresponding sequences in treated patients. The Meme program was used in discriminative mode, which highlights differences between sequence datasets. The relative size of each a.a. symbol is proportional to its frequency in the HIC dataset, while the total height of a.a. symbols indicates the information content of the position, in bits.

(C, D) Frequency of public clonotypes per 100 TRAV24 sequences (C) or per 100 TRBV2 sequences (D) for each of the 8 Controllers (HIC) and 8 treated patients (HAART) studied.

(E, F) Frequency of nucleotide sequences coding for a public clonotype per 100 TRAV24 sequences (E) and per 100 TRBV2 sequences (F). (C to F) Significant differences (P<0.05) obtained by the Mann-Whitney test are reported.

(G) Structure of the CDR3 junction for the most prevalent TRAV24 public clonotype AFKAAGNKLT. The number of N mutations (N), of trimmed nucleotides (trim), and the frequency (% AFKAAGNKLT) of the 4 nucleotide sequences coding for this clonotype are reported (SEQ ID NO: 172 to 174 on FIG. 3G).

FIGS. 4A to 4E: Ex vivo analysis of the Gag293-specific TCR repertoire

(A) Gating strategy for tetramer analysis in Controller PBMC. An example of PBMC staining with a control tetramer (AnnII; left plot) and a Gag293-loaded DRB5 tetramer (right plot) is shown.

(B) Frequency of TRAV24 and TRBV2 families in Gag293-Tet+ cells sorted ex vivo. The percentages of TRAV24 expression in total TRAV products (left) and TRBV2 expression in total TRBV products (right) are reported. Dotted lines indicate the mean percentage of TRAV24 and TRBV2 families in CD4+ T cell from 7 healthy donors.

(C) Representation of Gag293-specific clonotypes found ex vivo in the cell line obtained from the same patient. The percentage of sequences matching a TRAV24 (left) or TRBV2 ex vivo clonotype (right) in the corresponding cell line is reported, with medians indicated by horizontal lines.

(D, E) The percentages of public motifs are compared in sequences obtained ex vivo (left) and in the matched cell line (right) for TRAV24 (D) and TRBV2 (E), using the paired Student t test.

FIGS. 5A to 5G: Public TCRs confer MHC II cross-restriction and high-affinity Gag293-MHC binding

(A) Expression of TCR(and CD3 in J76 cells transduced with the F24, F25, and F5 TCRs. Solid grey histograms correspond to mock-transduced J76 cells.

(B) Staining of F24-transduced J76 cells with CLIP-loaded tetramers (top) and Gag293-loaded tetramers (bottom). The percentage of Tet+ cells is reported on each plot.

(C) The percentage of Tet+ cells after transfer of F24 (black), F25 (medium grey), and F5 (light grey) is reported for each of the 4 tetramers tested after subtraction of CLIP-tetramer background (mean of 2 experiments).

(D) Example of SPR sensorgrams. The soluble F24 TCR (concentrations 0.3 to 100 μM) was flown over immobilized DR11-Gag293 monomers to measure the SPR response. RU, response units.

(E) Affinity measurement of the F24, F25, and F5 TCRs for Gag293 complexed to DR11, DRB5, or DR1 monomers. Each soluble TCR was flown over Gag293-DR complexes at different concentrations (x axis) to measure binding RU (y axis).

(F) Summary of affinities (Kdeq) of the F24, F25 or F5 TCRs for Gag293-loaded HLA-DRB monomers. Each Kdeq value represent the mean±SEM from at least two independent experiments performed in duplicate.

(G) Correlation between TCR affinity and tetramer binding. TCR affinities (log Kdeq) of the F24, F25 and F5 TCRs for the 3 Gag293-DR complexes are plotted in function of the percentage of Gag293-Tet+ cells (log % Tet+) for the corresponding TCR/HLA-DR combination (y axis). R: Spearman correlation coefficient.

FIGS. 6A to 6F: Public TCR transfer confers high antigen sensitivity to J76 cells

(A, B) Antigen sensitivity assay in TCR-transduced J76 cells. Percentages of CD69 expression in J76 cells transduced with TCRs F24, F25, F5 (A) or F4 and F13 (B), after coculture with L-cells expressing different HLA-DR alleles (DR11, DR15, DRB5, or DR11) and loaded with decreasing Gag293 concentrations. Experiments were conducted in triplicate, with curves corresponding to one experiment shown for clarity.

(C) Antigen sensitivity assay of TCRs HD5 and HY9 from HAART patients.

(D) Correlation between binding affinity (log Kdeq) for Gag293-loaded HLA-DR monomers (DR11, DRB5, and DR1) and antigen sensitivity (log EC50 for CD69 induction) of the F24, F25 and F5 TCRs.

(E) Correlation between the antigen sensitivity (log EC50) measured for 8 TCRs in the presence of DR11 APC and the number of HLA-DR alleles restricting these TCRs. (E, F). R: Spearman correlation coefficient.

(F) TCR reactivity to native HIV-1 capsid antigens. CD69 induction was quantified in TCR-transduced J76 cells cocultured with dendritic cells infected with the VSV-pseudotyped virus ΨHIV-1 (+) or left uninfected (−). One representative experiment out of three is shown.

FIGS. 7A to 7F: Public TCR transfer confers high avidity responses and polyfunctionality to primary T cells

(A) Cytokine production in primary CD4+ T cells mock-transduced (1st row) or transduced with the F24, F25, or F5 TCR (rows 2-4) and stimulated with 10-5M Gag293. CD4+ T cells were analyzed by ICS for expression of TNF-α, MIP-1β, IL-2, IFN-γ, and CD107a. One representative experiment out of three is shown.

(B) ICS analysis of CD4+ T cells transduced with F24 and stimulated with decreasing Gag293 doses. Expression of the analyzed markers (% marker+) is reported in function of peptide dose, after subtraction of background measured in unstimulated cells.

(C) Summary of EC50 values measured by ICS in CD4+ T cells after TCR transduction. For each TCR, the Gag293 concentration required to achieve half-maximal expression of the 5 markers studied is reported. Mean+SEM of EC50 values obtained for 3 independent experiments are reported.

(D) Cytokine production in CD8+ T cells that were mock-transduced (1st row) or transduced with the F24 TCR (2nd row) and analyzed as in A.

(E) ICS analysis of CD8+ T cells transduced with F24 and stimulated with decreasing Gag293 doses.

(F) Polyfunctionality of CD4+ T cells transduced with the F24, F25, and F5 TCRs and stimulated with decreasing Gag293 doses. The number of co-expressed markers out of the 5 studied (TNF-α, MIP-1β, IL-2, IFN-γ, CD107a) defines the number of functions reported in legend. Stimulation with PMA/ionomycin is used as a positive control.

FIGS. 8A to 8B: Analysis of nucleotide insertions and deletions in Gag293-specific clonotypes

(A) The number of mutations (P+N) inserted in Gag293-specific clonotypes compared to TRAV24-containing (left) or TRBV2-containing (right) germline sequences is reported.

(B) The number of germline nucleotides trimmed during V(D)J recombination to generate the observed Gag293-specific TRAV24-containing (left) and TRBV2-containing (right) clonotypes is reported.

(A and B): The numbers of mutations and trimmed nucleotides were computed in the IMGT/HighV-QUEST program, using the IMGT database of human germline TRAV and TRBV alleles as a reference (www.imgt.org). Significant differences between means (P<0.05) obtained by the unpaired student t-test are reported.

FIGS. 9A to 9B: Identification of prevalent amino acid motifs in Gag293-specific clonotypes

The Meme motif discovery program (meme-suite.org) was used to identify the most prevalent a.a. motifs in HIV Controller (HIC) and treated patients (HAART) clonotypes. The Meme program was used in normal mode with the «one occurrence per sequence» option, to take all clonotypes into account. The relative sizes of the letters in the logo are proportional to their frequencies, while the total height of the letters indicates the information content of the position, in bits.

(A) Most prevalent motif in TRAV24 clonotypes from the HIC (top; n=584) and HAART (bottom; n=496) groups.

(B) Most prevalent motif in TRBV2 clonotypes from the HIC (top; n=716) and HAART (bottom; n=566) groups.

FIGS. 10A to 10C: Analysis of TCR avidity by MHC II tetramer titration

(A) Gag293-DR11 tetramer titration: J76 cells transduced with the TCRs F24, F25, or F5 were incubated with decreasing concentrations of HLA-DR11 tetramer loaded with the Gag293 peptide. The percentage of Gag293-specific tetramer+ (Tet+) cells minus the percentage of cells labeled with a control CLIP-loaded tetramer is reported. (B and C)

Gag293-DRB5 tetramer (B) and Gag293-DR1 tetramer (C) titration on F24-transduced cells. The dip in binding curves at high tetramer concentrations likely reflects competition effects between multivalent ligands. EC50 computation was based on the sigmoidal part of the response curve. (D) Linear correlation between the maximum % of tetramer+ cells and TCR affinity determined by SPR. (E) The half-maximal tetramer binding values (EC50) are reported. ND: not detectable, i.e. tetramer binding was too low to evaluate the EC50 value.

FIGS. 11A to 11D: Analysis of Gag293-specific CD4+ T cell responses in DR11 patients

(A) Comparison of antigen sensitivity in HIV Controllers (HIC) and treated patients (HAART) carrying at least one DR11 allele. Antigen sensitivity was measured by the last Gag293 peptide dilution (in M) that yielded a specific CD4+ T cell line.

(B) Comparison of the maximal ELIPOT response to Gag293 in DR11 patients. CD4+ T cell lines generated with a 10-5 M Gag293 peptide dose were restimulated with the same high peptide dose and analyzed by IFN-γ ELISpot assay. The number of spot forming cells (SFC) per 106 cells is reported. Values>104 SFC/106 cells reached saturation and are reported as equal to 104 SFC/106 cells.

(A and B) P values obtained by the Mann-Whitney U test are reported.

(C and D) Comparison of TRAV24 (C) and TRBV2 (D) expression in Gag293-specific cells of DR11 patients.

The percentage of TRAV24 or TRBV2 expression among tetramer-positive (Tet+) cells is reported.

FIGS. 12A to 12C: TCR transfer in primary T cells from healthy donors confers Gag293-MHC II tetramer recognition

(A) Example of TCR transduction in primary CD4+ T cells from a healthy donor. PBMCs were mock transduced, or transduced to express the F24, F25, or F5 TCRs, and stained with anti-TRBV2 mAb.

(B) MHC II tetramer staining in CD4+ T cells transduced with the F24 TCR (second row), or mock transduced (first row). The percentage of CD4+ T cells stained with Gag293-loaded HLA-DR tetramers (DR11, DR15, DRB5, and DR1) is reported in the top right corner. For these experiments, the DR15 tetramer used corresponded to the HLA DRB1*1502 rather than the HLA DRB1*1501 allele.

(C) Quantification of MHC II tetramer staining in TCR-transduced primary CD4+ T cells. For each tetramer, the mean percentage of tetramer-positive (Tet+) CD4+ T cells obtained from 4 independent experiments is shown. The percentage of Gag293-specific Tet+ cells was computed by subtracting the percentage of CLIP-Tet+ cells from that of Gag293-Tet+ cells. Light grey bars: mock-transduced; black bars: F24-transduced; grey bars: F25-transduced; light grey bars: F5-transduced.

FIGS. 13A and 13B: Analysis of cytokine production in CD8+ T cells transduced with the F24 TCR.

(A) Blocking of the cytokine response in CD8+ T cells with anti-HLA antibodies.

Cytokine induction was measured in CD8+ T cells transduced with the F24 TCR after stimulation with 10-5 M Gag293 peptide or in non-stimulated cells (NS). Cells were pretreated with an isotypic IgG2a control antibody (black bars), an HLA-DR blocking antibody (grey bars) or a pan-MHC I blocking antibody (light grey bars) at 10 μg/ml prior to peptide stimulation. >75% of the response was blocked by HLA-DR antibody treatment for each cytokine tested, indicating that F24-expressing CD8+ T cells were predominantly restricted by MHC II.

(B) Analysis of the polyfunctionality of CD8+ T cells transduced with the Gag293-specific TCR F24. Polyfunctionality was defined as the capacity for specific cells to co-express at least 3 markers among the 5 studied (TNF-α, MIP-1β, IL-2, IFN-γ, and CD107a) after Gag293 peptide stimulation. The number of markers co-expressed defines the number of functions reported in legend. Polyfunctionality is visualized with pie charts in which each slice represents a functional category: white, 5 functions; light grey, 4 functions; middle grey, 3 functions; dark grey, 2 functions; andblack, 1 function. Polyfunctionality was assessed after stimulation at different peptide doses ranging from 10-5 M to 10-8 M. Stimulation with PMA and ionomycin was used as a positive control to induce a highly polyfunctional response (right pie).

FIGS. 14A to 14E: Suppression of HIV-infected dendritic cells by TCR-transduced T cells.

(A) Scheme of the viral suppression assay: immature monocyte-derived dendritic cells (iDC) were infected with a single-cycle pseudotyped HIV-1-GFP virus (ΨHIV-1 NL4-3 GFP) in the presence of Vpx containing virus-like particles (Vpx-VLP), and then cocultivated with TCR-transduced T cells. HIV suppression was quantified by the decrease in GFP+DC in the presence of TCR-transduced T cells at different effector:target (E:T) ratios.

(B) Representative example illustrating the decrease of GFP+ infected DC in the presence of F24-transduced CD4+ T cells.

(C) Quantification of the decrease in GFP+ infected DC in function of the TCR transduced (F24, F25, or F5) and of the E:T ratio.

(D) Comparison of F24 suppressive effect on infection in DC expressing different HLA-DR alleles.

(E) Suppressive effect of the 3 TCRs when transduced in CD8+ T cells.

FIGS. 15A to 15F: TRAV24 clonotypes (SEQ ID NO: 1060 to SEQ ID NO: 1142).

FIG. 16A to 16F: TRBV2 clonotypes (SEQ ID NO: 1143 to SEQ ID NO: 1254).

As a summary of the experiments carried out, it has been observed that the rare patients who spontaneously control HIV replication in the absence of therapy show signs of a particularly efficient cellular immune response. To identify the molecular determinants underlying this response, the inventors characterized the TCR repertoire directed at the most immunoprevalent CD4 epitope in HIV-1 capsid, Gag293. HIV Controllers from the ANRS CODEX cohort showed a highly skewed TCR repertoire characterized by a predominance of TRAV24 and TRBV2 variable genes, shared CDR3 motifs, and a high frequency of public clonotypes. The most prevalent public clonotypes generated TCRs with affinities at the higher end of values reported for naturally occurring TCRs. These high-affinity Gag293-specific TCRs were cross-restricted by up to 5 distinct HLA-DR alleles, accounting for their expression in HIV Controllers of diverse genetic backgrounds. Transfer of these TCRs to healthy donor CD4+ T cells conferred high antigen sensitivity and polyfunctionality, thus recapitulating key features of the Controller CD4 response. Transfer of a high-affinity Gag293-specific TCR could also redirect CD8+ T cells to target HIV-1 capsid via nonconventional MHC II restriction. These findings indicate that TCR clonotypes with superior functions are associated with HIV control. Amplifying or transferring such clonotypes may contribute to immunotherapeutic approaches that aim at a functional HIV cure.

### Results

We set to compare Gag293-specific CD4 responses in HIV Controllers and efficiently treated patients, two groups characterized by long-term viral control. The clinical and immunological characteristics of the studied patients are reported in Table 1. A stringent definition of HIV control was applied, based on an undetectable viral load in standard assays (<50 copies HIV-1 RNA/ml) for over 5 years. The duration of control was actually longer, as patients included in the Controller group (HIC group, n=14) had been infected for a median duration of 19 years. They were compared to HIV-1 infected patients (HAART group, n=15) who had received long-term antiretroviral therapy, with an undetectable viral load for at least 5 years, and a median duration of treatment of 11 years. Thus, both groups were characterized by long-term viral suppression, which ensured that potential differences in CD4 responses were not primarily determined by levels of residual HIV viremia.

TABLE 1
Clinical and immunological characteristics of patients

| | HIV controllers | | Treated patients | | |
|---|---|---|---|---|---|
| Clinical parameters^{a} | (HIC, n = 14) | | (HAART, n = 15) | | P value^{b} |
| Age, years | 50 | [34-61] | 48 | [39-56] | N.S. |
| Duration of HIV-1 infection, years | 19.6 | [9.8-26.0] | 11.9 | [6.8-25.3] | 0.015 |
| Duration of antiretroviral treatment, | N/A | | 11.1 | [5.6-19.3] | |
| years | | | | | |
| Virus load, HIV-1 RNA copies/ml | <50 | | <50 | | N.S. |
| plasma | | | | | |
| CD4+ T cells/mm3 | 875 | [648-1400] | 570 | [266-1534] | 0.008 |

| | | | | |
|---|---|---|---|---|
| Nadir of CD4+ T cells/mm3 | N/A | 216 | [21-589] | — |

| Frequency of Gag293-specific CD4+ T | | | |
|---|---|---|---|
| cell lines generated at different peptide | HIV controllers | Treated patients | |
| doses^{c} | (HIC, n = 14) | (HAART, n = 15) | P value^{d} |
| Gag293 dose: 10⁻⁵ M | 14/14 | 15/15 | N.S. |
| Gag293 dose: 10⁻⁷ M | 14/14 | 8/15 | 0.006 |
| Gag293 dose: 10⁻⁹ M | 8/14 | 1/15 | 0.005 |
| Gag293 dose: 10⁻¹¹ M | 2/14 | 0/15 | N.S. |

^{a}Median values and ranges are reported.
^{b}P values were estimated with the Mann-Whitney test.
^{c}Frequency of viable cell lines that achieved doubling of input cells and gave a specific response by IFN-g ELISPOT assay.
^{d}Differences in frequencies were evaluated with Fisher's exact test.
N.S.: not significant (P ≥ 0.05); N/A: not applicable.

High Antigen Sensitivity of Gag293-Specific CD4 Responses in HIV Controllers

TCR repertoire studies of specific CD4+ T cells have remained scarce in humans due to the limited clonal amplification of CD4+ T cells as compared to CD8+ T cells, and to the generally lower affinity of TCR expressed by CD4+ T cells, which limits MHC II tetramer detection (31). In the case of HIV infection, these factors are compounded by the general decrease of the CD4+ T cell population, the preferential depletion of HIV-specific CD4+ T cells, and their incomplete restoration under antiretroviral therapy (5, 6). To address these issues and analyze the TCR repertoire of CD4+ T cells from treated patients as well as Controllers, we devised a system of short-term primary CD4+ T cell line cultures that allowed the amplification of MHC II tetramer-positive cells prior to sorting. Using this system, we previously reported the presence Gag293-specific CD4+ T cells with high antigen sensitivity and high MHC II tetramer binding capacity in Controllers, while such cells were absent from treated patients (27). To extend these results, we generated primary CD4+ T cells lines by stimulation with decreasing doses of Gag293 peptide. The specificity of CD4+ T cell lines was evaluated at equivalent growth stages (doubling time), by restimulation with Gag293 and analysis by IFN-γ ELISPOT assay. As reported in Table 1, viable Gag293-specific cell lines were obtained for all patients of the HIC and HAART groups when initially stimulated with the highest peptide dose of 10⁻⁵ M. However, a marked difference was observed for cell lines generated at 10⁻⁷ M peptide, with only 8 out of 15 treated patients responding, versus all of the 14 Controllers (P=0.006). The difference was also marked at the 10⁻⁹ M peptide dose (P=0.005). Moreover, 2 of the Controllers, but none of the treated patients responded at the 10⁻¹¹ M peptide dose. These experiments established that Gag293-specific CD4+ T cells had a higher antigen sensitivity in the Controller group, as indicated by the capacity to proliferate and differentiate into IFN-γ-secreting effectors upon stimulation with minimal peptide doses.

Biased TRAV and TRBV Gene Usage in Gag293-Specific CD4+ T Cells

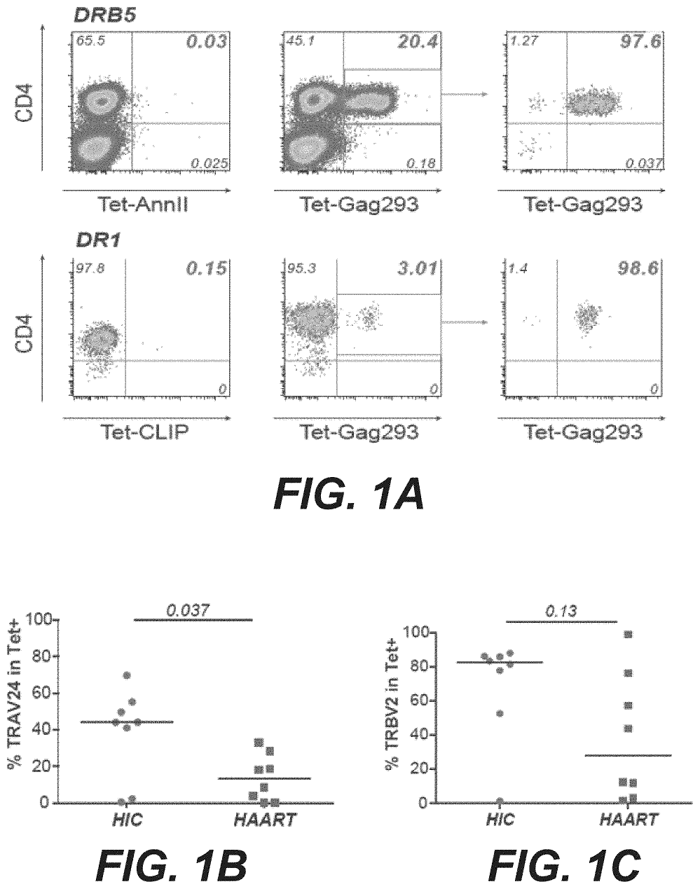

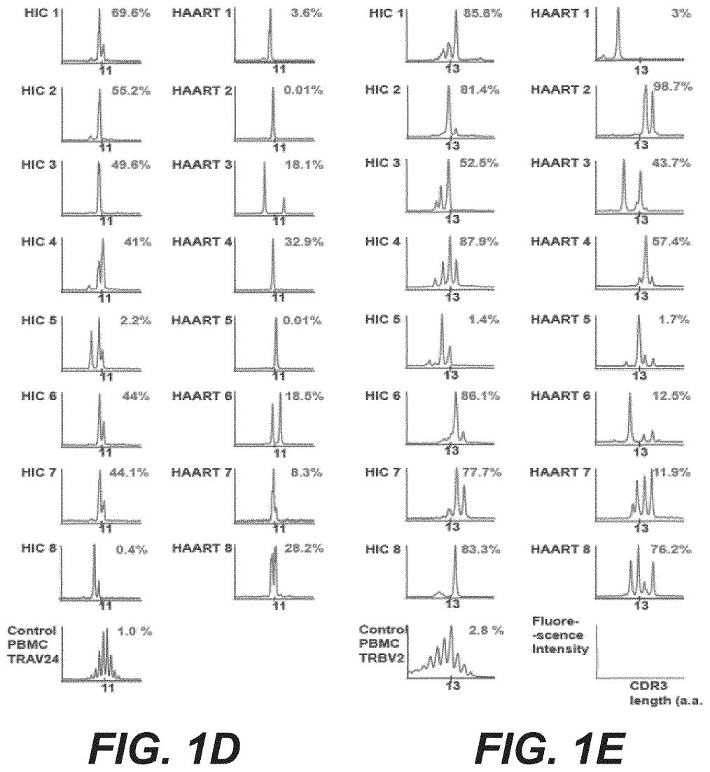

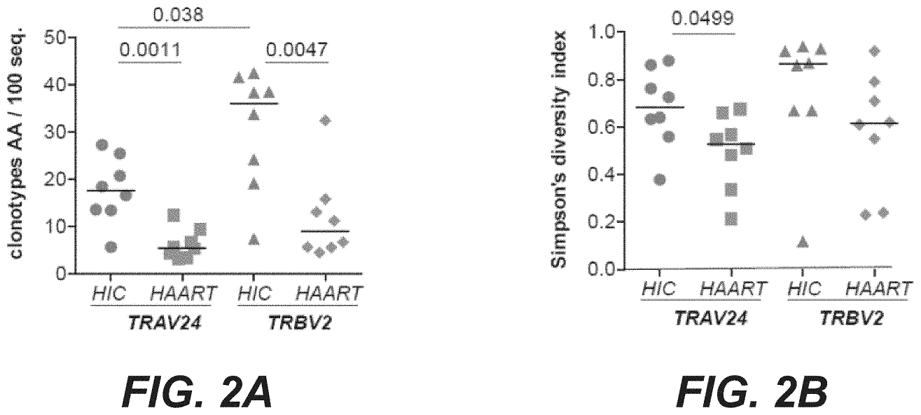

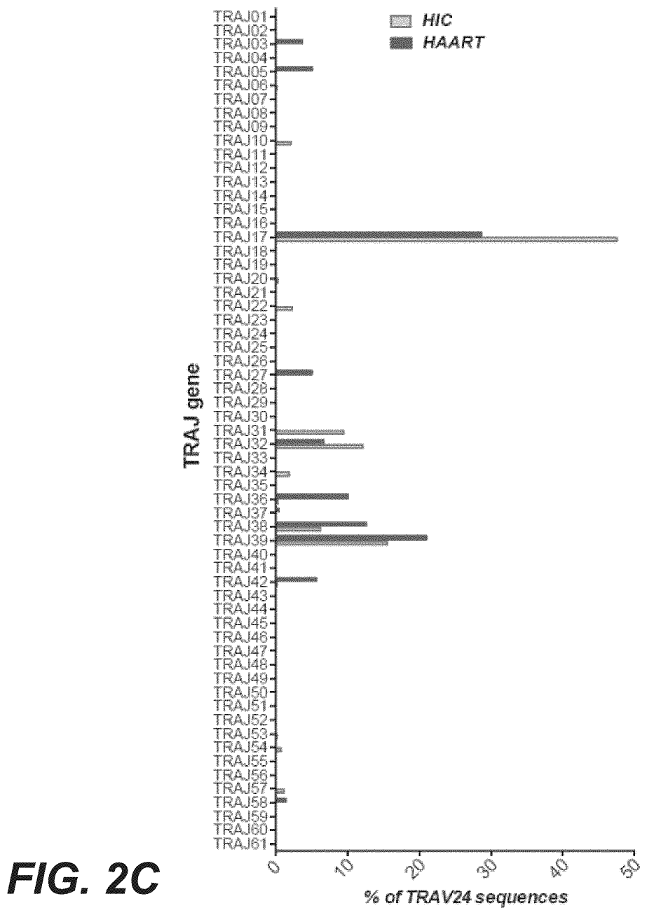

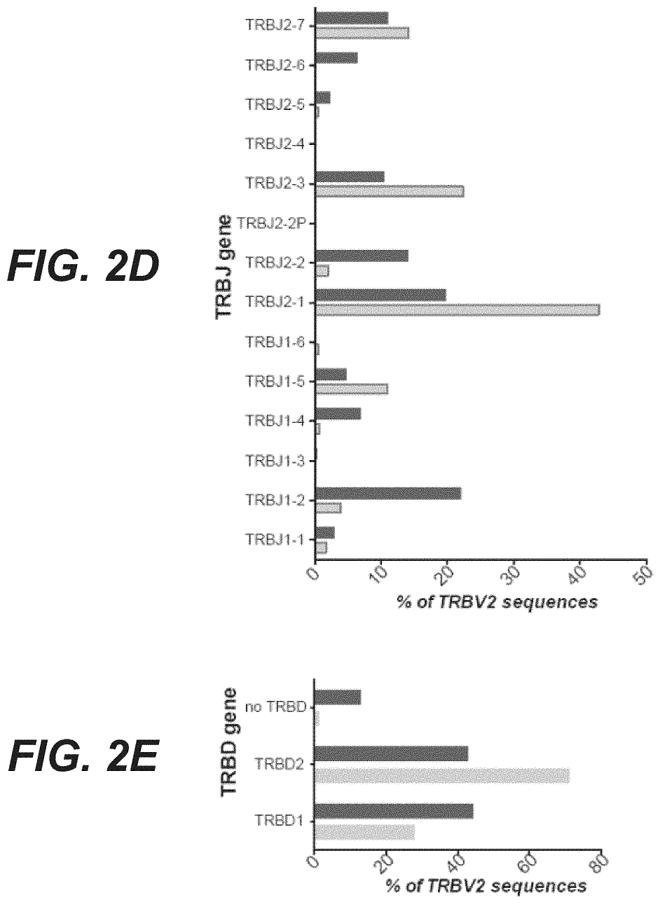

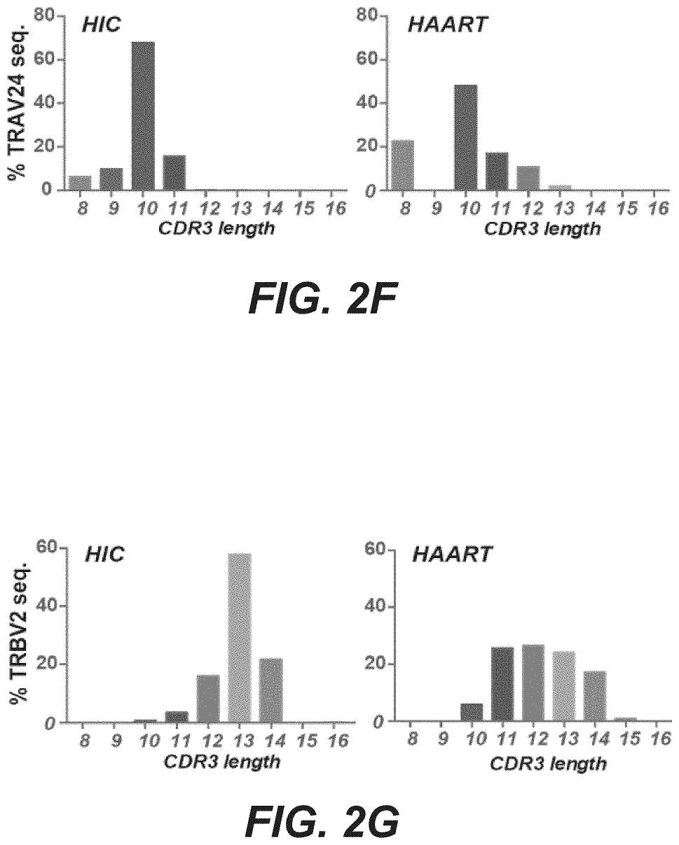

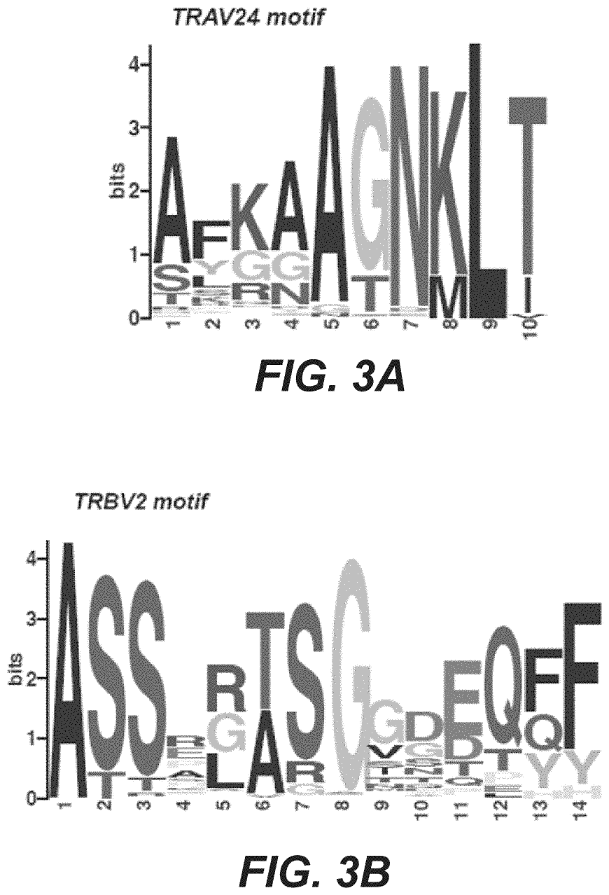

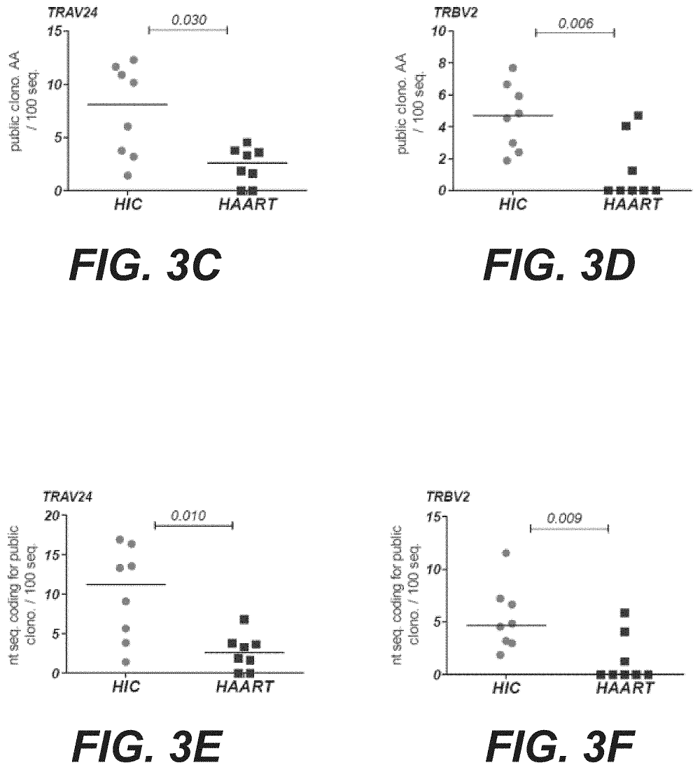

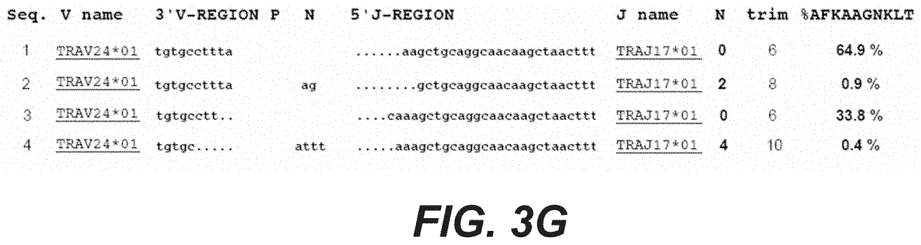

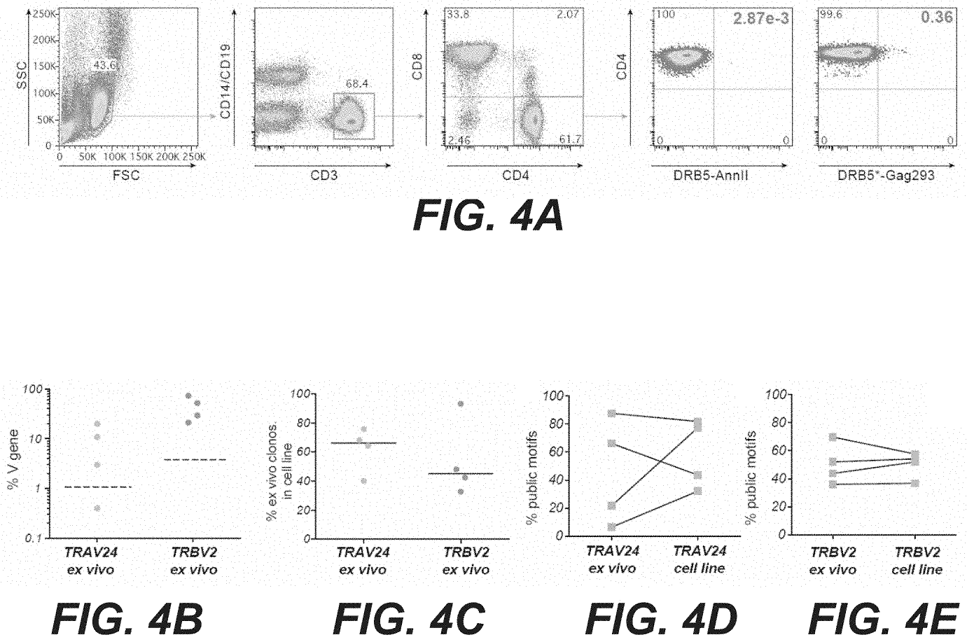

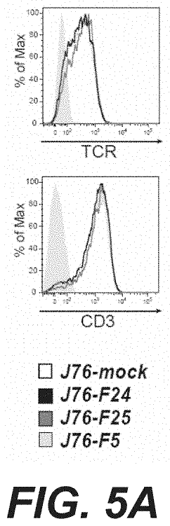

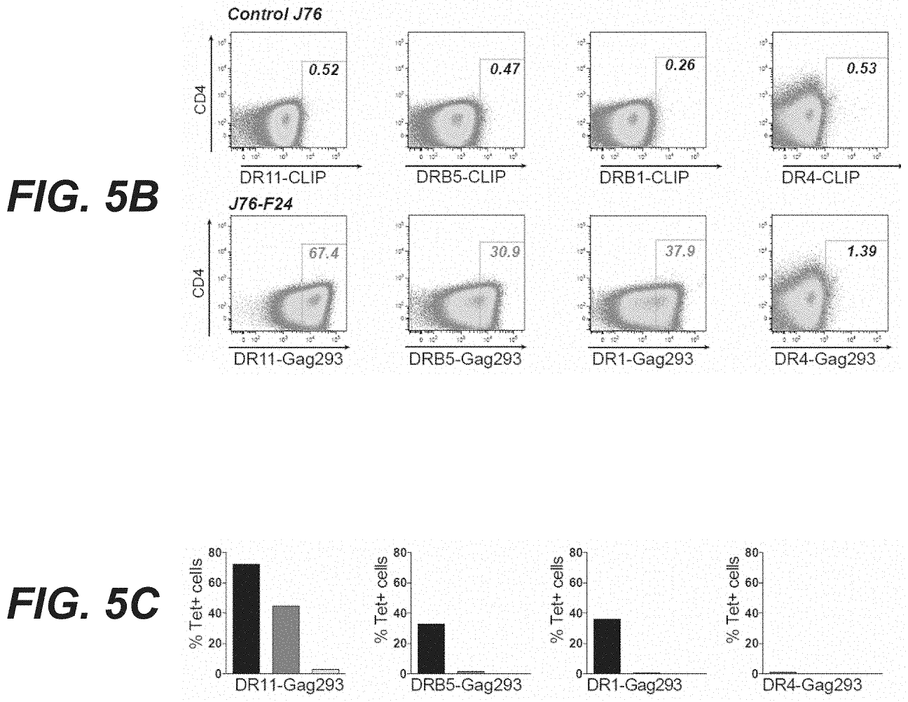

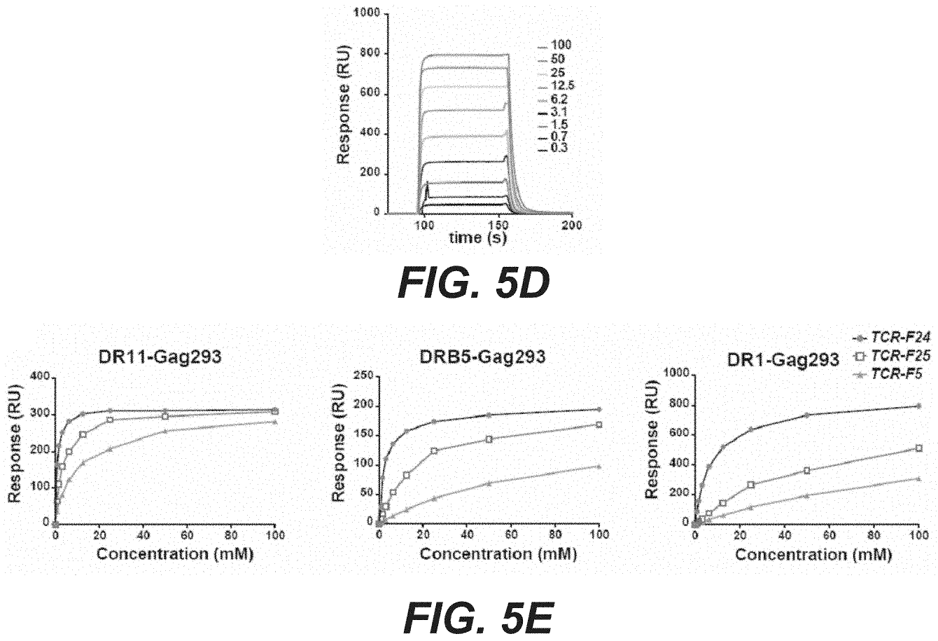

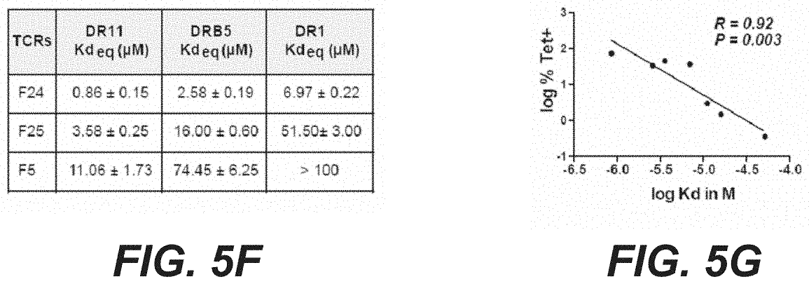

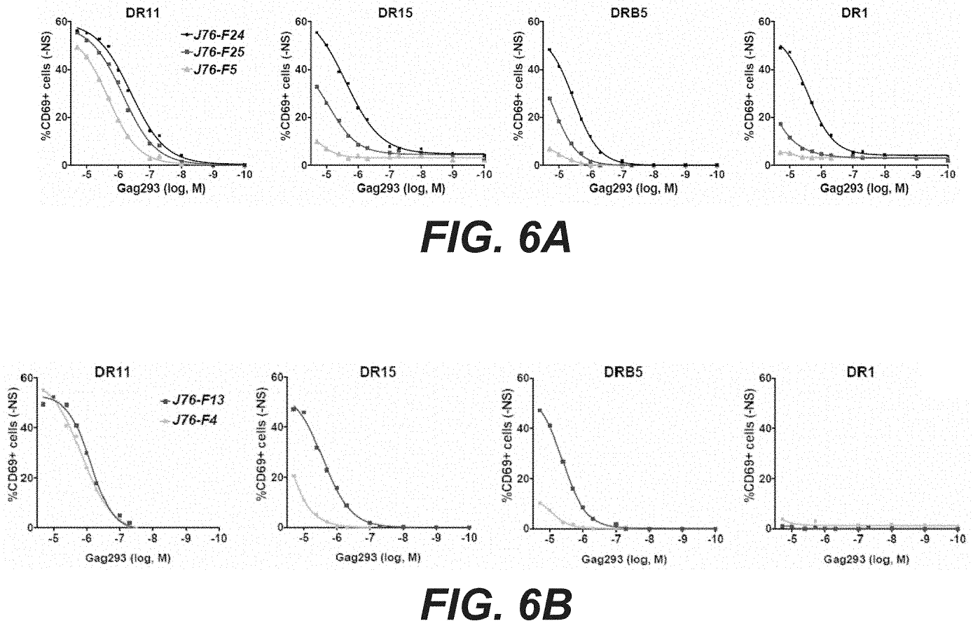

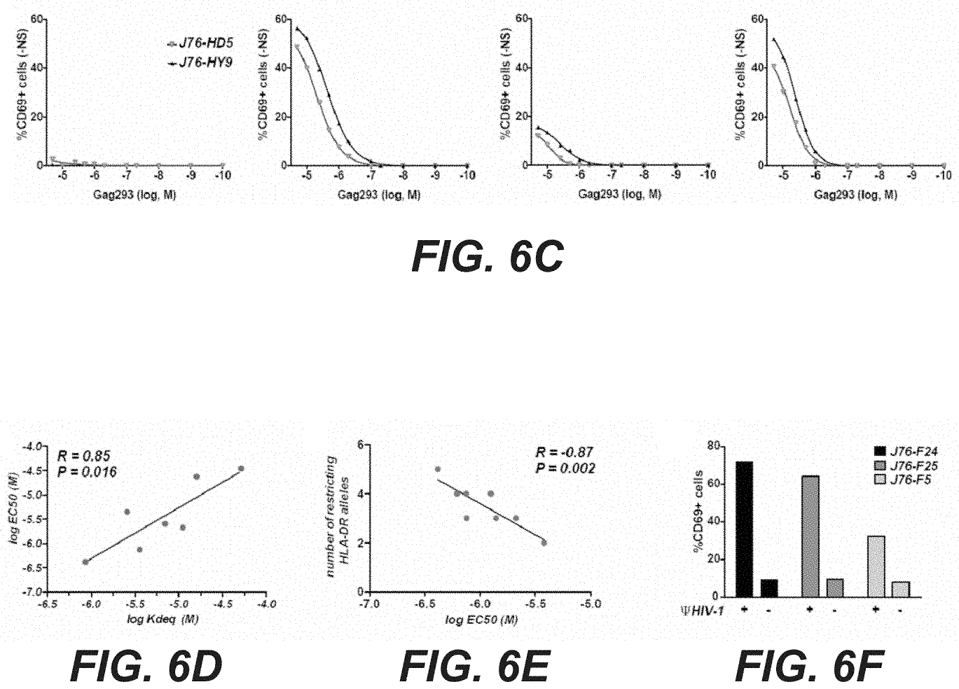

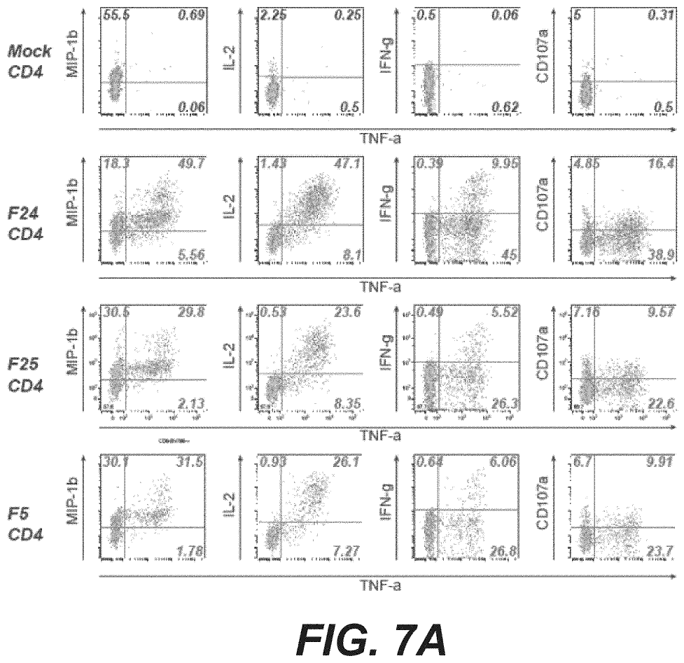

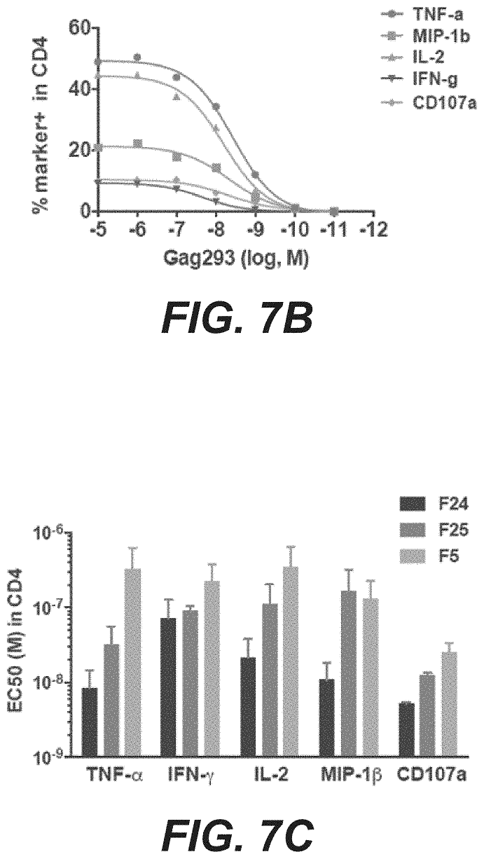

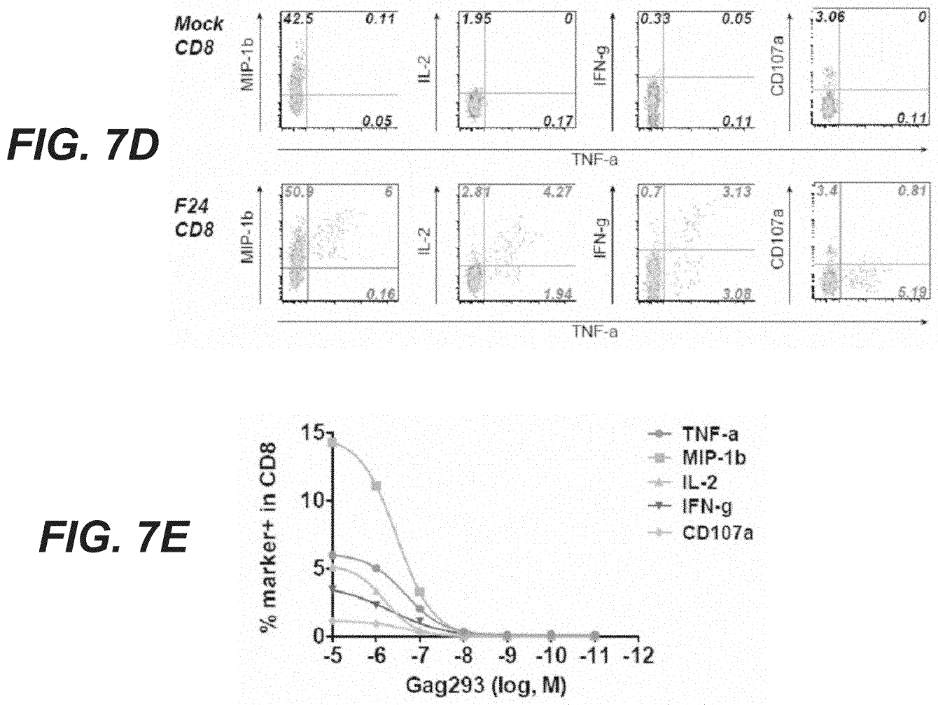

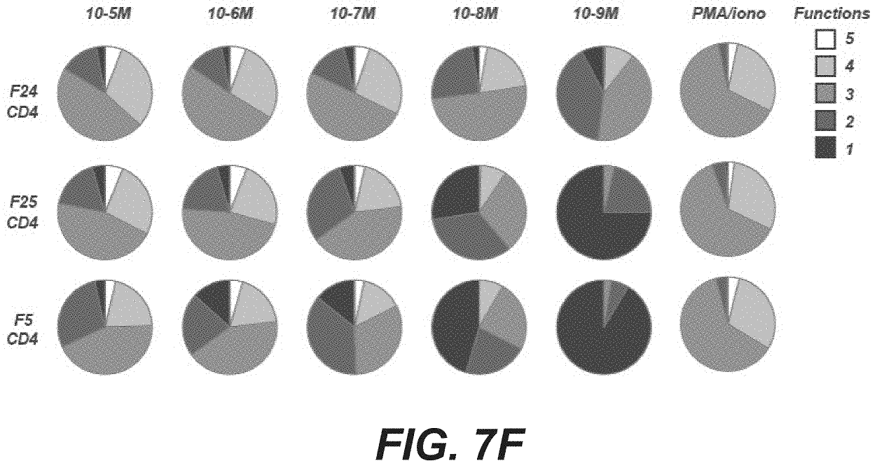

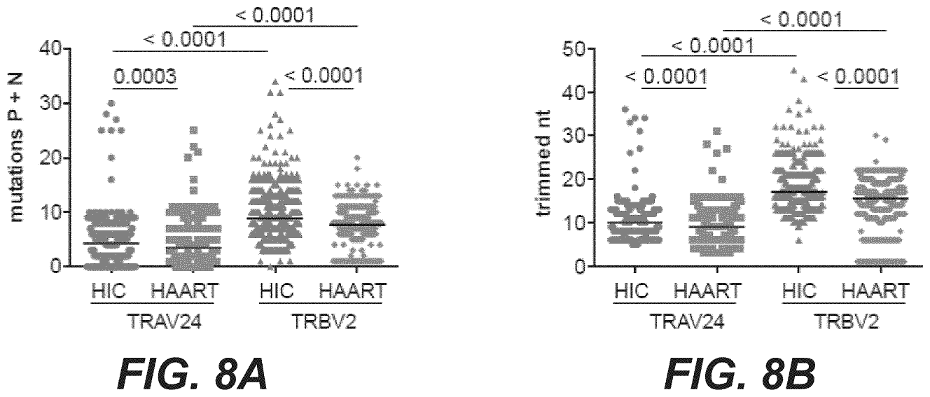

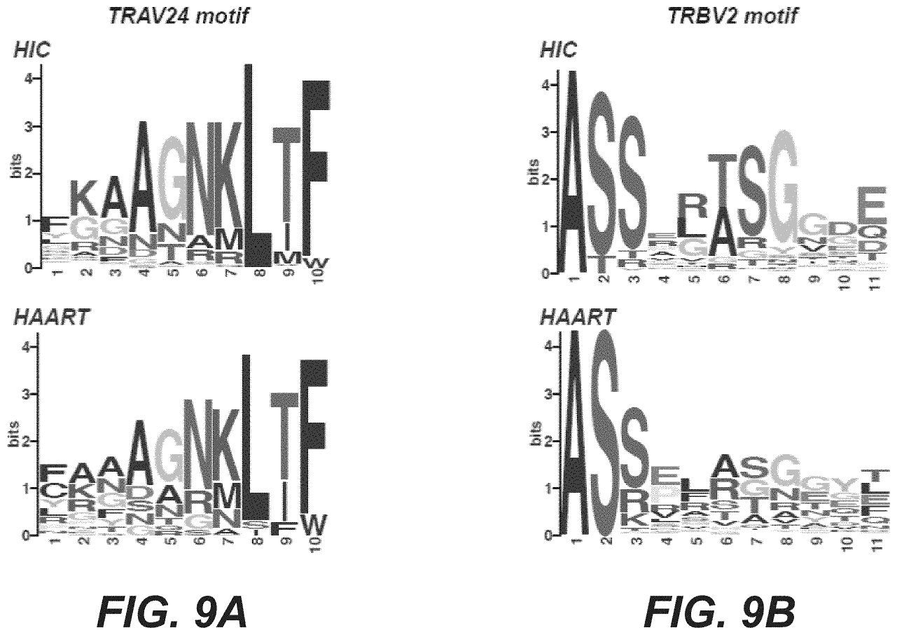

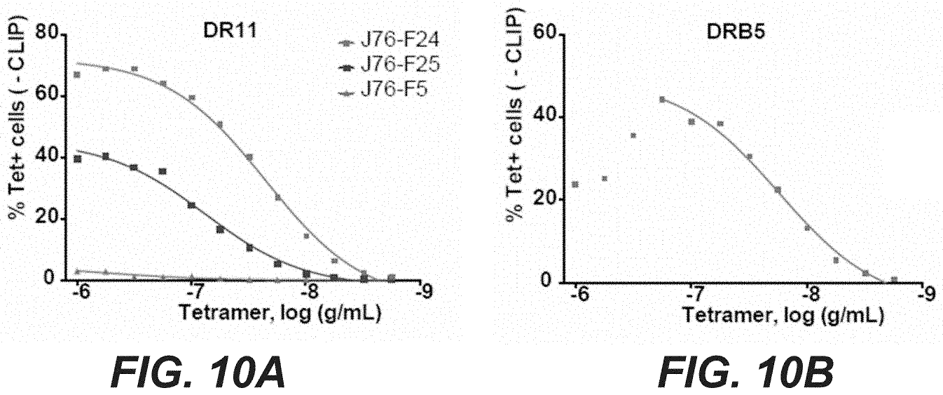

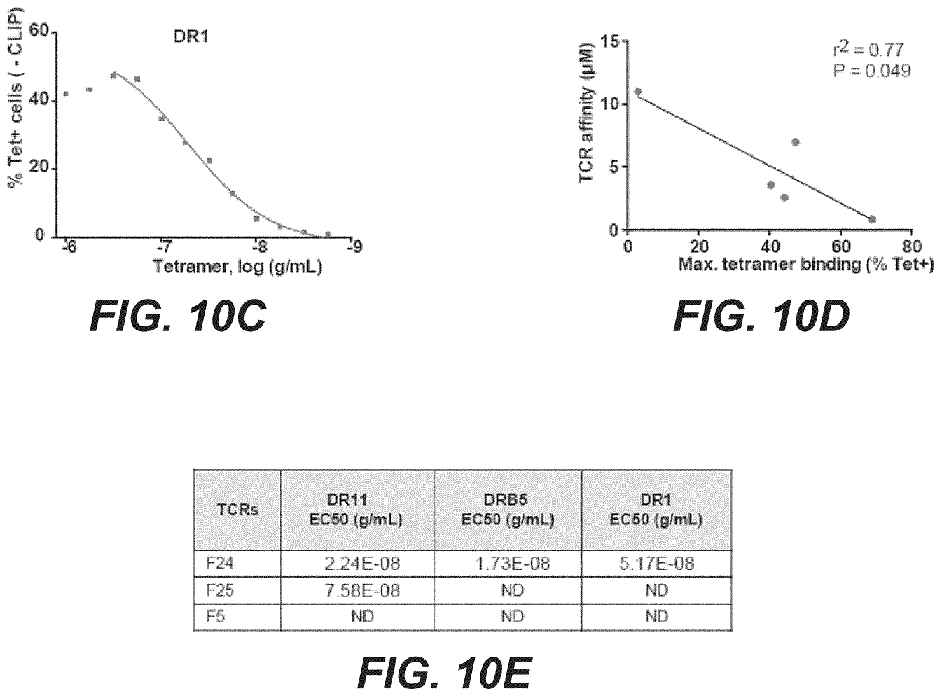

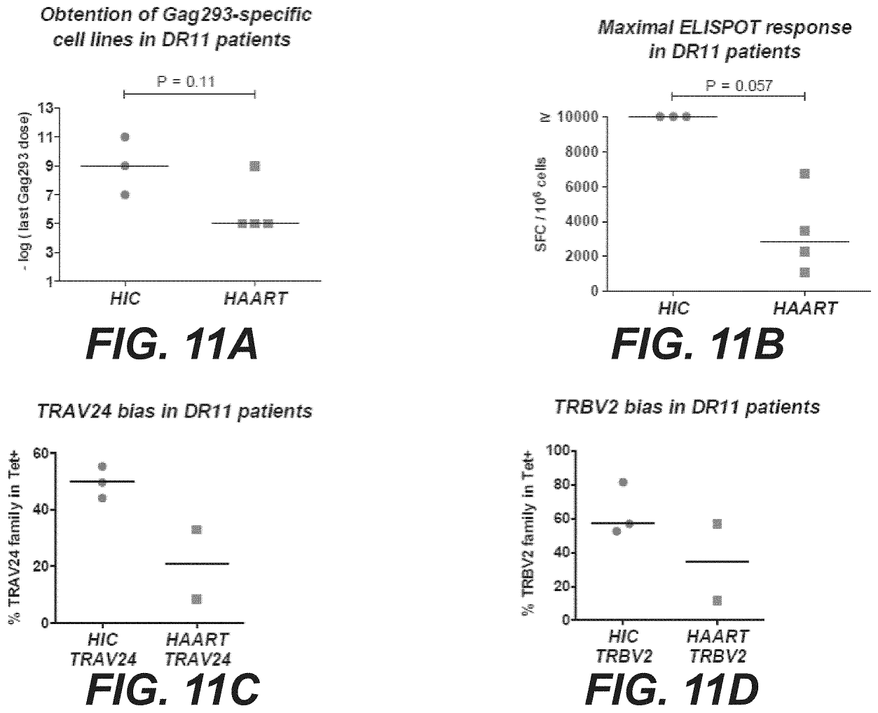

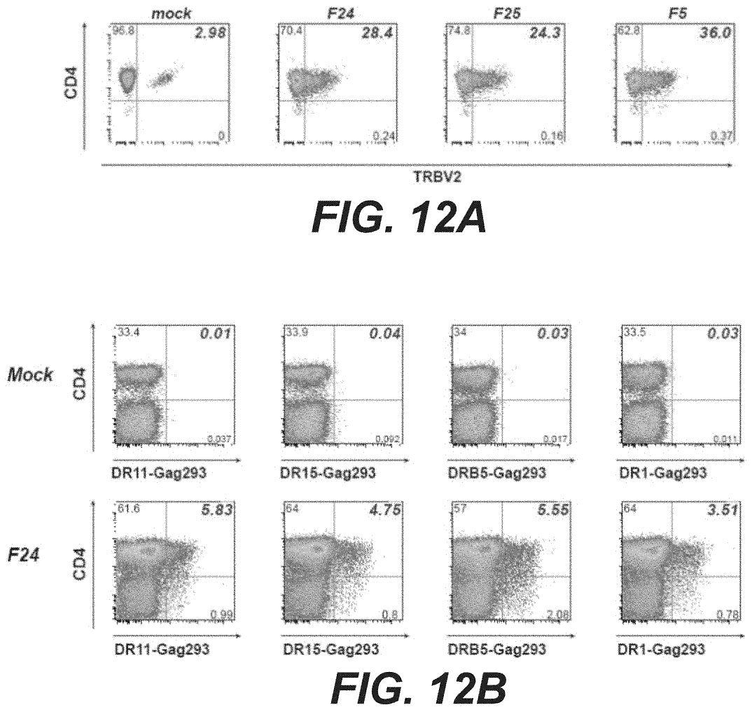

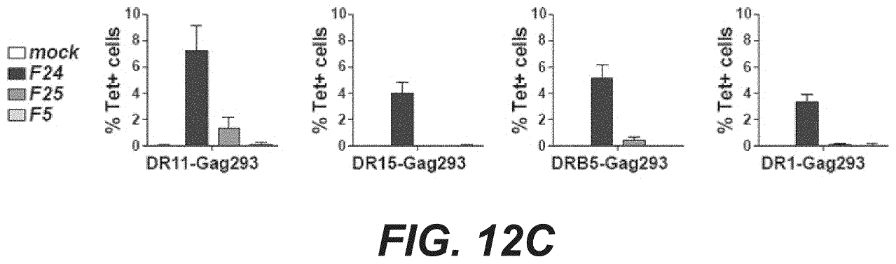

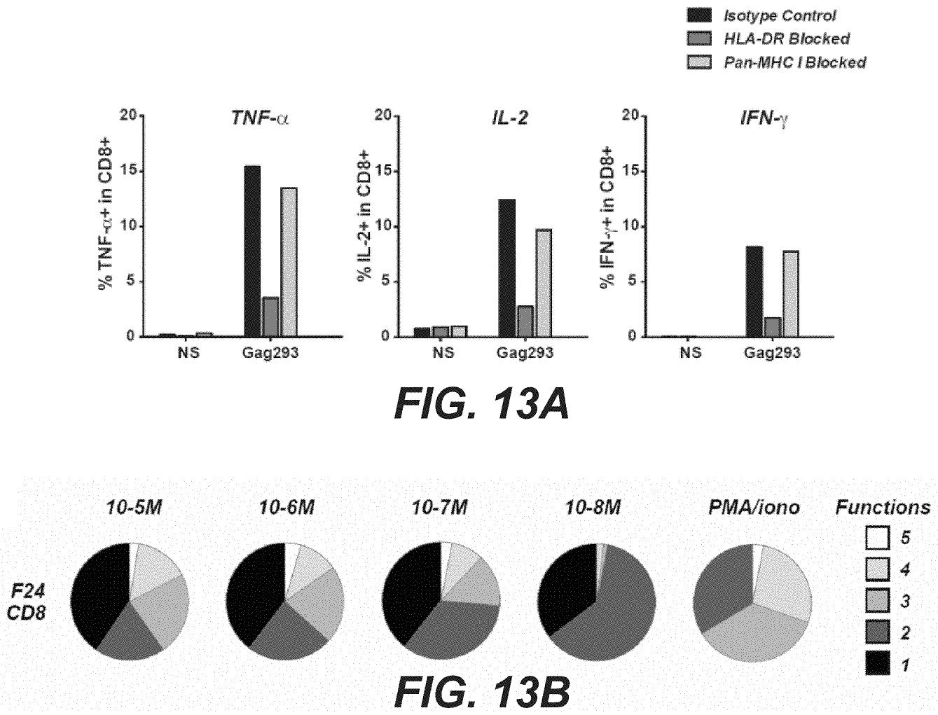

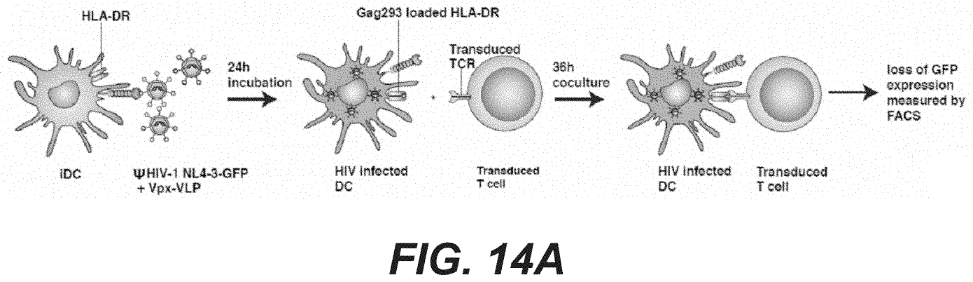

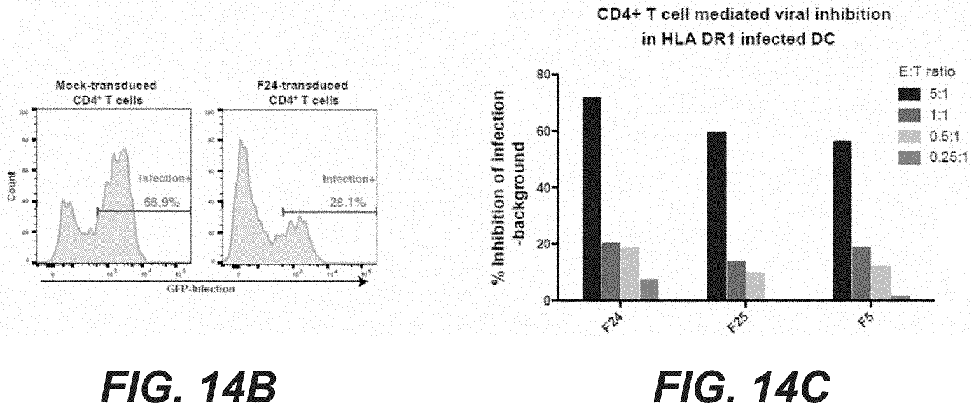

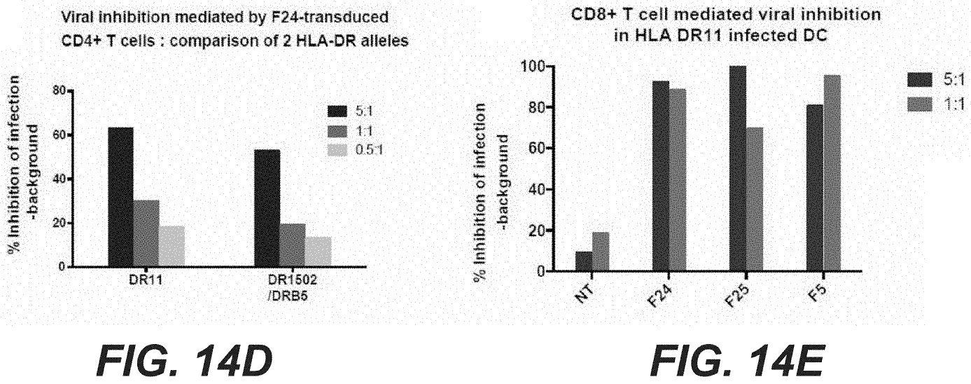

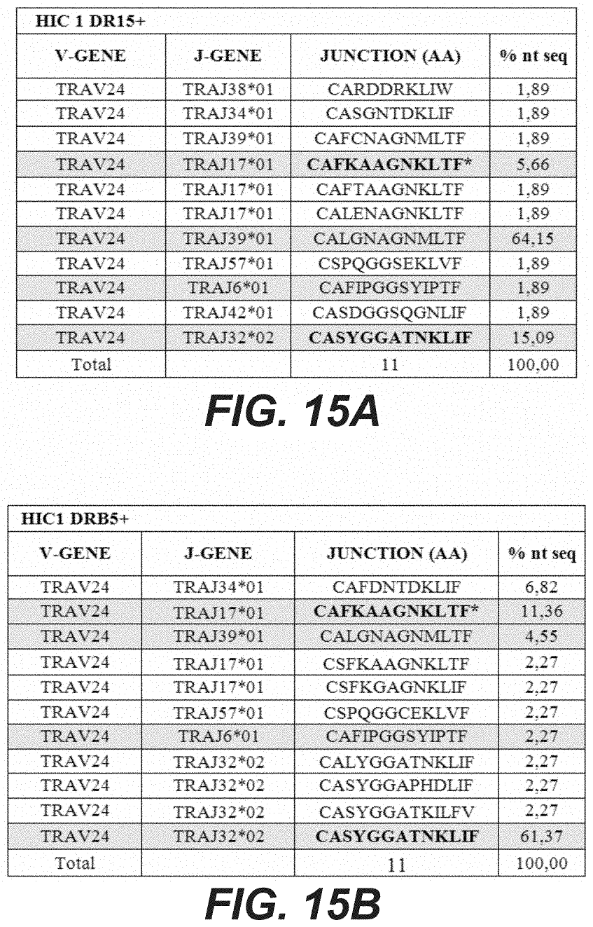

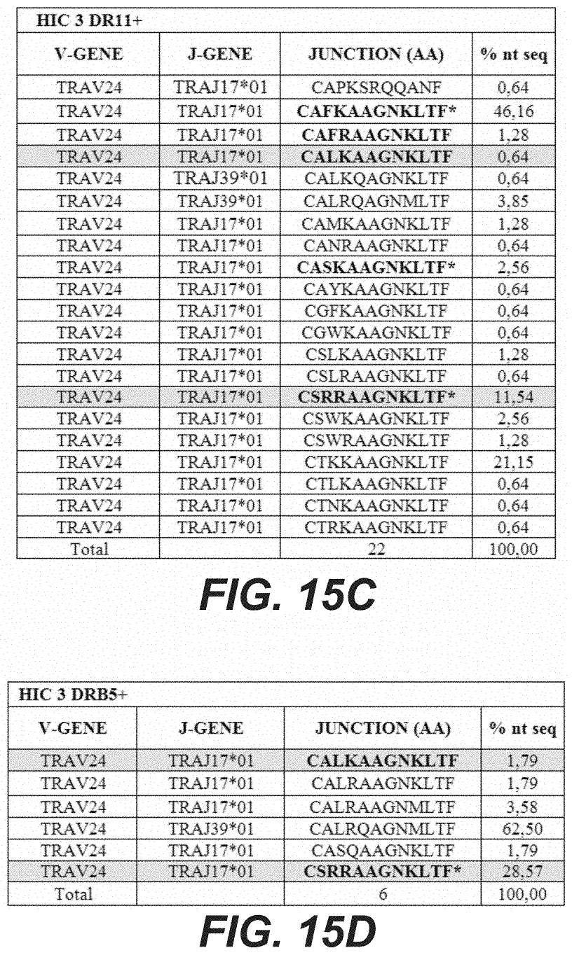

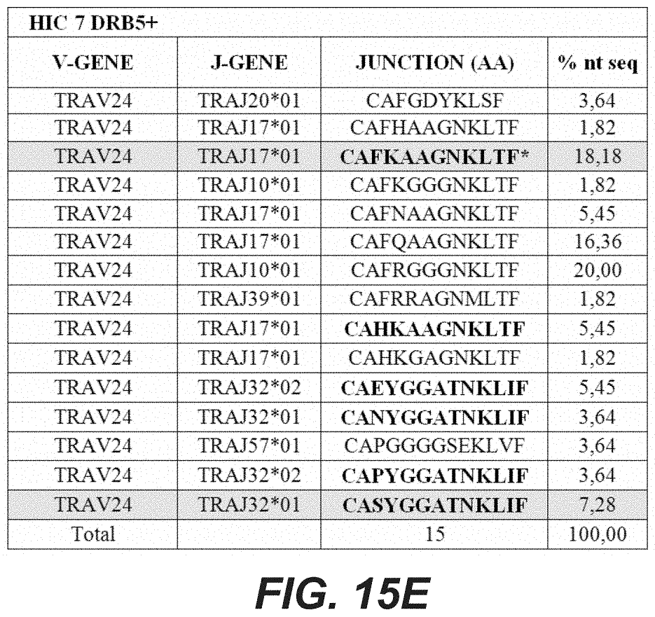

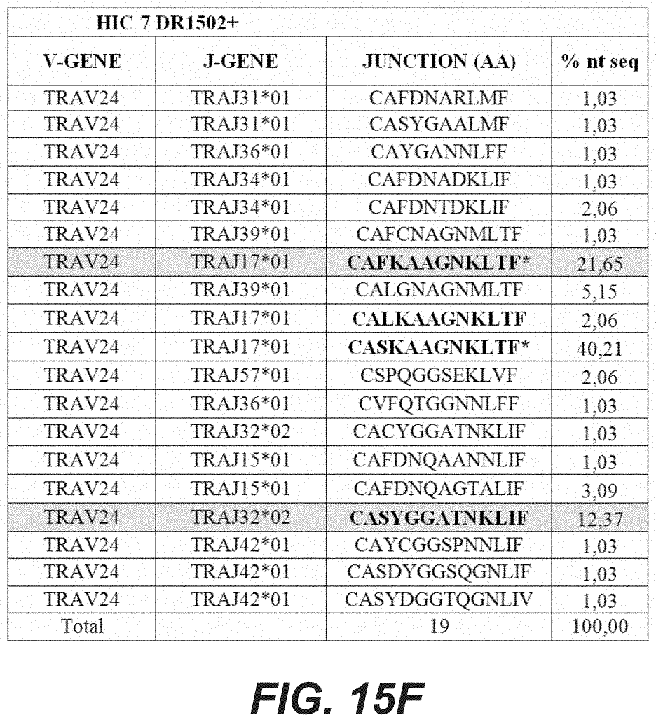

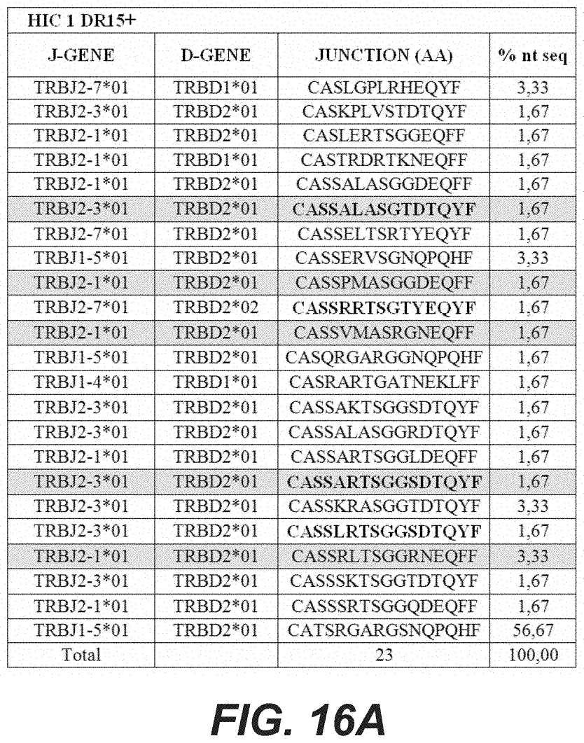

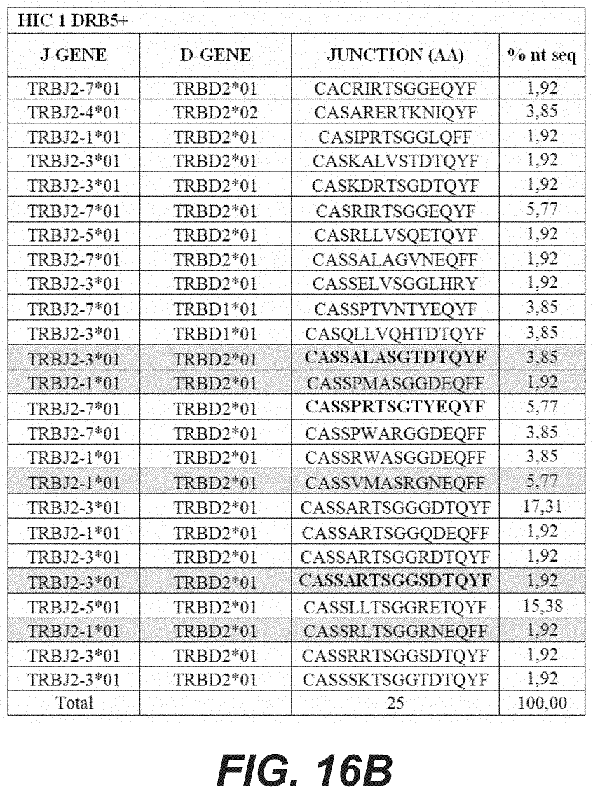

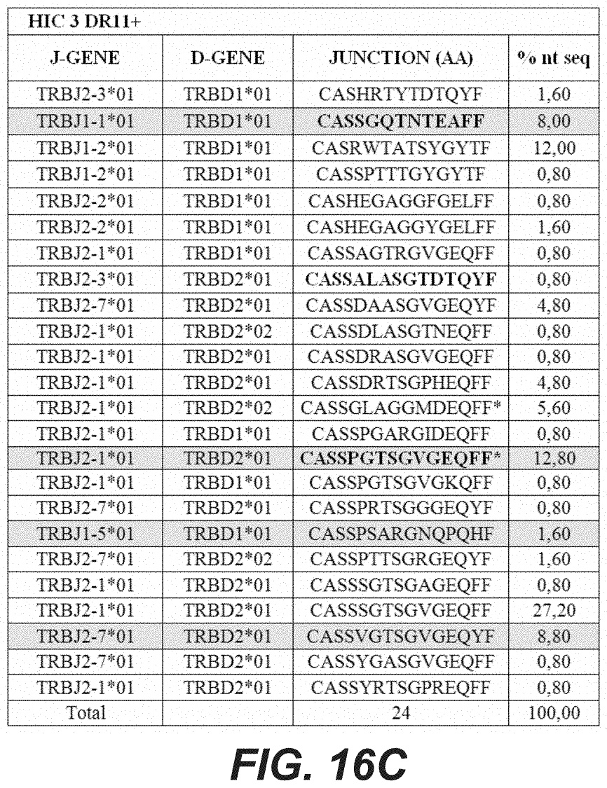

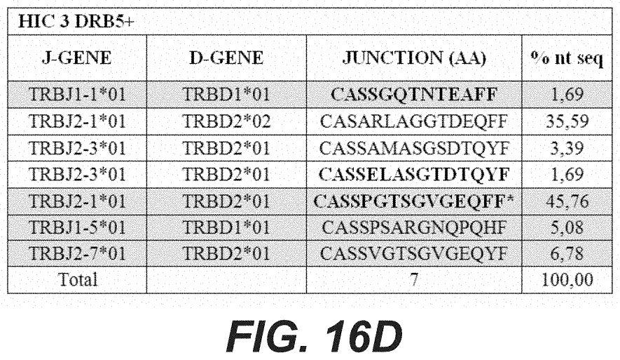

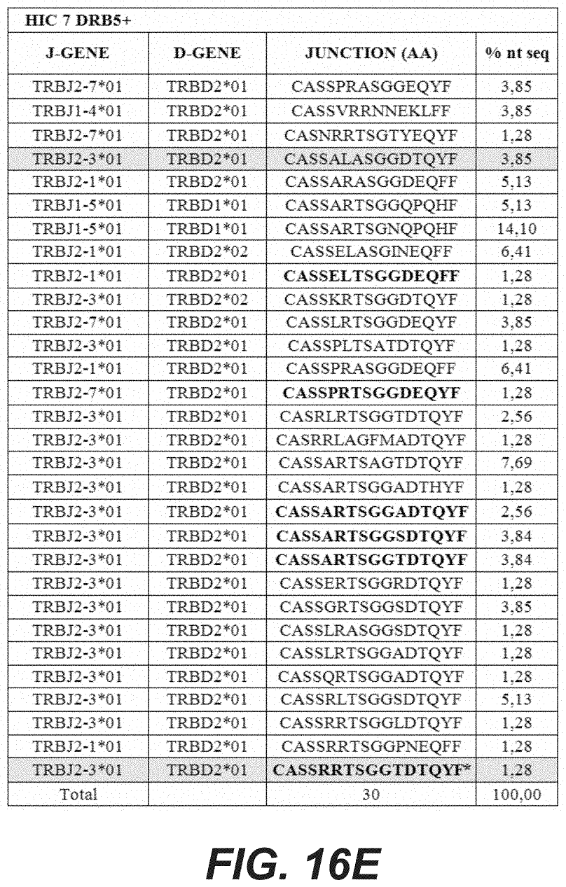

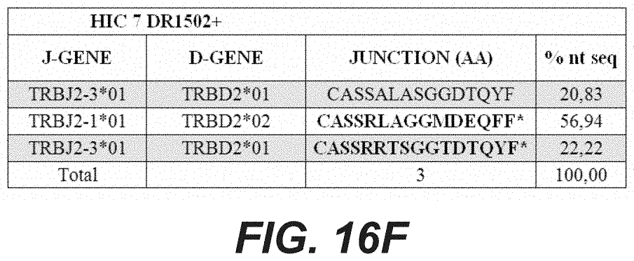

To characterize the Gag93-specific TCR repertoire, we first genotyped patients for the HLA-DRB1 gene. Eight Controllers and eight treated patients who shared at least one of four HLA-DR alleles (DR1, DR11, DR15, or DRB5) were included in the TCR study (Table 5). The frequencies of these four alleles did not differ significantly between the HIC and the HAART groups (P≥0.05 by Fisher's exact test). CD4+ T cell lines from these patients were labeled with HLA-DR matched Gag293-loaded tetramers, and evaluated for the proportion of Tet+ cells in the CD4+CD8-T cell population (FIG. 1A, middle panels). Samples labeled with matched tetramers loaded with an irrelevant peptide (CLIP or Annexin II) were used as negative controls (FIG. 1A, left). The purity of the Tet+ population was controlled post-sorting, as shown in representative examples (FIG. 1A, right). TCR diversity of the sorted cells was evaluated through CDR3 length polymorphism analysis, using the Immunoscope technique (32, 33). The expression of 34 TCRα variable gene (TRAV) families and of 24 TCRβ variable gene (TRBV) families was quantified by real-time RT-PCR, followed by an analysis of the length distribution of the amplified CDR3 products on a capillary sequencer. TRAV gene expression in Controller Tet+ cells proved highly skewed, with a median of 44% of Gag293-specific cells expressing the TRAV24 gene family (FIG. 1B), while this family was amplified at lower levels in specific cells of treated patients (median value: 13%; P=0.037). In comparison the TRAV24 family represented 1±0.2% of T cells in a control group of 7 healthy donors (not shown). Analysis of CDR3 length distribution showed a Gaussian pattern for control PBMC, as expected (FIG. 1D). In contrast, Gag293-specific cells appeared oligoclonal, with a dominant peak corresponding to a CDR3 of 10 aa, according to the numbering scheme of the International ImMunoGeneTics Information System (IMGT) (34). This peak was more prominent in cell lines from Controllers than those of treated patients, suggesting a stronger TCR bias in controlled HIV infection.

Analysis of TRBV distribution also revealed a major bias in Gag293-specific cells, with a marked predominance of the TRBV2 family, expressed at a median value of 82% in the Controller group (FIG. 1C). TRBV2 was amplified in Tet+ cells of 7 out of 8 Controllers in the present study, while this amplification was seen in only 1 out of 4 Controllers in a first study where we tested Gag293-specific TCR profiles with a panel of VB-specific antibodies (27). The lower frequency of TRBV2 (previously noted V1322) amplification detected in the initial study may have resulted from suboptimal V13 antibody binding due to competition with the MHC II tetramer for binding to the TCR. The TRBV2 family also appeared amplified at high levels in some of the treated patient samples (median value=28%), with a difference between the HIC and HAART groups that did not reach significance (P=0.13; FIG. 1C). In comparison, T cells from healthy donors expressed TRBV2 at low levels (3.7±1.0%, not shown). CDR3 length distribution appeared more variable for the TRBV2 than the TRAV24 chain (FIG. 1E), though a trend for a predominant CDR3 of 13 aa was apparent in Controllers Tet+ cells. Taken together, these data provide evidence for a highly skewed Gag293-specific TCR repertoire characterized by the preferential usage of the TRAV24 and TRBV2 variable gene segments. The fact that the bias is more marked in Gag293-specific cells from Controllers than treated patients suggests a contribution of the TRAV24 and TRBV2 variable regions to the high-avidity recognition of Gag293.

High Clonotypic Diversity of Gag293-Specific Cells in Controllers

Clonotypic repertoire analysis was carried out for the two variable gene families amplified in Gag293-specific cells, TRAV24 and TRBV2. PCR products corresponding to these two families were cloned, sequenced, and analyzed with IMGT tools (34). A minimum of 50 productive CDR3 sequences were analyzed for each sample, with the full list of CDR3 sequences provided in Tables 6 and 7. Productive TRAV24 sequences (n=584 for the HIC group; n=496 for the HAART group —HAART group results not shown) were evaluated for diversity by counting the number of distinct clonotypes, i.e. the number of unique CDR3 aa sequences, present in each patient sample. The normalized number of clonotypes proved significantly higher in the HIC than the HAART group (P=0.0011; FIG. 2A). Similarly, analysis of TRBV2 sequences (n=716 for the HIC group; n=566 for the HAART group) showed higher clonotypic diversity in HIC samples (P=0.0047; FIG. 2A). Of note, the number of TRBV2 clonotypes per 100 sequences was remarkably high in the HIC group (median=36), indicating the presence of a diverse TCR repertoire in spite of the pronounced bias for TRBV2. Computation of Simpson's diversity index confirmed a trend for higher clonotypic diversity in the HIC group, which reached significance for TRAV24 but not TRBV2 sequences (FIG. 2B). The number of mutations needed to generate the observed CDR3 sequences from their germline counterparts was also significantly higher in the HIC group, for both the TRAV24 and TRBV2 datasets (FIG. 8). Thus, both the number of clonotypes and that of CDR3 insertions and deletions were consistent with the persistence of a more diverse Gag293-specific repertoire in controlled HIV infection.

Biased J and D Gene Usage in Gag293-Specific TCRs from Controllers

The Gag293-specific CDR3 sequence dataset was analyzed for the distribution of Junction (J) and Diversity (D) gene segments. TRAJ gene usage was restricted, with 15 and 11 distinct TRAJ genes detected in TRAV24 sequences for the HIC and HAART groups, respectively, out of 61 TRAJ genes reported in the IMGT database (FIG. 2C). The TRAJ17 gene predominated in the HIC group (48%), while the next most abundant genes, TRAJ39 and TRAJ32, were present at 16 and 12%, respectively. TRAJ gene usage in the HAART group appeared more evenly distributed among TRAJ17 (29%), TRAJ39 (21%), and TRAJ38 (13%). Analysis of the TRBJ distribution in TRBV2 sequences showed again a more biased repertoire in the HIC group, with a predominant TRBJ2-1 gene present in 43% of sequences, while the most abundant TRBJ gene in the HAART group, TRBJ1-2, represented only 22% of sequences (FIG. 2D). Of note, TRBJ1-2 was only minimally represented in HIC sequences (4%), emphasizing differences between the HIC and HAART Gag293-specific repertoires. In addition, the TRBD2 gene segment was more prevalent in HIC sequences, while the two TRBD genes were equally represented in HAART sequences (FIG. 2E). The CDR3 length was then computed by counting the number of residues comprised between but not including the conserved cysteine (C104) and the conserved phenylalanine or tryptophan (F/W118) that define the boundaries of this hypervariable TCR region (34). For TRAV24 sequences, CDR3 length peaked at 10 a.a., consistent with the Immunoscope data (FIG. 2F). The 10 a.a. peak represented 68% and 48% of HIC and HAART TRAV24 sequences, respectively, pointing to more homogenous CDR3 lengths in Controller sequences (P=5.61e-11; kurtosis: 7.35 for HIC vs 2.91 for HAART). For TRBV2 sequences (FIG. 2G), CDR3 lengths showed a peak at 13 a.a. in HIC sequences, while CDR3 lengths were more evenly distributed in HAART sequences (P<2.2e-16; kurtosis: 4.82 for HIC vs −2.16 for HAART). These analyses indicated that Gag293-specific CDR3 sequences from Controllers were biased in genetic composition, with a predominant use of TRAJ17, TRBJ2-1, and TRBD2, as well as narrowly distributed CDR3 lengths, while sequences from treated patients appeared more heterogeneous. Thus, Controller TCRs showed restricted V(D)J gene usage, indicative of a repertoire shaped under strong selective pressure, while maintaining a high clonotypic diversity.

High Prevalence of Public Motifs in Gag293-Specific TCRs from Controllers

Given the observed biases in V(D)J gene usage, we next evaluated whether Gag293-specific CDR3 sequences shared common aa motifs. The Meme motif discovery software was used in discriminative mode to identify motifs enriched in HIC as compared to HAART TRAV24 sequences. This approach revealed a highly prevalent but complex motif shared by 81% of HIC CDR3 sequences (FIG. 3A). Comparison of the predominant motif in the HIC and HAART datasets highlighted that sequence differences mostly concentrated in the N-terminal part of the CDR3 (FIG. 9A). More detailed analyses identified two simpler motifs of 10 and 11 aa that were significantly more prevalent in HIC than HAART CDR3 sequences (Table 2). CDR3a motif AV24-1, [A/S]x[K/R]AAGNKLT (with x meaning any aa) encompassed the highly conserved AGNKLT sequence derived from the TRAJ17 gene, while motif AV24-2, AxYGGATNKLI, contained a related ATNKLI sequence derived from the TRAJ32 gene. Taken together, these two motifs were found in 49% of HIC versus 29% of HAART TRAV24 sequences (P<0.0001).

TABLE 2
Prevalence of public motifs in Gag293-specific clonotypes

| | | | | | | Public |
|---|---|---|---|---|---|---|
| | | CDR3 | HIC | HAART | P value | clono- |
| | TRAV24 Motif | length | group | group | HIC vs | types |
| | Sequence | (AA) | (% seq.) | (% seq.) | HAART | (% seq.) |
| Motif AV24-1 | [A/S]x[K/R]AAGNKLT | 10 | 37.16 | 22.38 | <0.0001 | 33.33 |
| Motif AV24-2 | AxYGGATNKLI | 11 | 11.64 | 6.65 | 0.0062 | 27.78 |
| Motifs | | — | 48.80 | 29.03 | <0.0001 | 61.11 |
| AV24-1 + | | | | | | |
| AV24-2 | | | | | | |
| | TRBV2 Motif | | | | | Public |
| | Sequence | CDR3 | HIC | HAART | P value | clono- |
| | SEQ ID NO: | length | group | group | HIC vs | types |
| | 196 and 197 | (AA) | (% seq.) | (% seq.) | HAART | (% seq.) |
| Motif BV2-1 | ASSx[R/G/L][T/A][S/G] | 13 | 38.55 | 6.36 | <0.0001 | 50.00 |
| | Gxx[E/T]Q[F/Y] | | | | | |
| Motif BV2-2 | ASSx[R/G/L][T/A]SGGxx | 14 | 11.87 | 3.89 | <0.0001 | 33.33 |
| | [E/T]Q[F/Y] | | | | | |
| Motifs | | — | 50.42 | 10.25 | <0.0001 | 83.33 |
| BV2-1 + | | | | | | |
| BV2-2 | | | | | | |

The percentage of motif occurrence is reported in total sequences from HIC and HAART patients as well as in the set of public clonotypes.
P values for differences between the HIC and HAART groups were computed with Fisher′s exact test.
x = any amino acid.
Seq. = Sequences.

Meme analysis of TRBV2 sequences in discriminative mode identified a complex motif with interspersed highly conserved positions in 79% of HIC sequences (FIG. 3B). A predominant motif was apparent in HIC TRBV2 sequences, while HAART TRBV2 sequences appeared too diverse for motif identification (FIG. 9B). Further analyses of HIC sequences revealed the overlap of two simpler motifs of 13 and 14 aa, respectively (Table 2). CDR33 motifs BV2-1 (ASSx[R/G/L][T/A][S/G]Gxx[E/T]Q[F/Y]) and BV2-2 (ASSx[R/G/L][T/A]SGGxx[E/T]Q[F/Y]) were highly similar and differed only by the presence of an additional G in the latter. Taken together, these two motifs were found in 50% of HIC versus 10% of HAART TRBV2 sequences (P<0.0001), indicating a predominance of conserved CDR3 residues in HIC clonotypes. This bias suggested a requirement of particular CDR3 residues for efficient recognition of the Gag293-MHC II complex (35). It was noteworthy that half of the TRAV24 and TRBV2 clonotypes identified in the HIC group shared the identified public motifs, indicative of a highly constrained Gag293-specific repertoire in controlled HIV infection.

High Frequency of TCR Sharing in HIV Controllers

Public clonotypes were defined as identical CDR3 aa sequences found in at least two individuals, without any mismatch tolerated. A total of 18 public clonotypes were identified in the TRAV24 sequence dataset obtained from the 16 patients studied (Table 3A). The most prevalent public clonotype, AFKAAGNKLT (called TRAV24-F), was found in 6 Controllers and 2 treated patients (75% and 25%, respectively), which are remarkably high frequencies in humans with diverse HLA II backgrounds. Groups of highly related public clonotypes were apparent, with a first group sharing the TRAJ17 chain and motif AxKAAGNKLT (AV24-1), and a second group sharing the TRAJ32 chain and motif AxYGGATNKLI (AV24-2). The 18 public clonotypes showed a high degree of motif sharing (61% positive for motifs AV24-1 or AV24-2, Table 2), confirming their similarity. Of note, public clonotypes were more frequent in the HIC than in the HAART group (P=0.03; FIG. 3C). Counting the number of nucleotide sequences encoding public clonotypes, normalized to 100 sequences, confirmed the higher frequency of TCR sharing in the HIC group (P=0.01; FIG. 3E).

Interestingly, a majority of TRAV24 public clonotypes (67%) were encoded by multiple nucleotide sequences. The mean number of unique nucleotide sequences encoding a TRAV24 public clonotype was 2.33, versus 1.05 for private (i.e. non-public) TRAV24 clonotypes. Detailed analysis of the 4 sequences coding for the most prevalent TRAV24 public clonotype, AFKAAGNKLT, showed that P and N mutations inserted between the TRAV24 and the TRAJ17 genes were few, ranging from 0 to 4 nucleotides (FIG. 3G). The numbers of nucleotides trimmed from germline sequences were also in the low range (6 to 10) compared to the ensemble of HIC sequences (mean of 9.9 trimmed nucleotides). The two predominant sequences, representing 64.9% and 33.8% of AFKAAGNKLT coding sequences, respectively, contained no mutation, and were simply obtained by trimming 6 nucleotides from joined TRAV24-TRAJ17 genes. Thus, the most prevalent TRAV24 public clonotype could be easily generated from germline sequences, suggestive of convergent V(D)J recombination (36).

TABLE 3
Distribution of Gag293-specific public clonotypes

| | | | | | | | | | | | | | | | | | | | Nb | |
|---|---|---|---|---|---|---|---|---|---|---|---|---|---|---|---|---|---|---|---|---|
| | | | HIC1 | HIC2 | HIC3 | HIC4 | | HIC6 | HIC7 | | HAA1 | HAA2 | | HAA4 | | | | HAA8 | of | Nb of |
| | | | DR15 | DRB5 | DR11 | DR15 | HIC5DR1 | DR11 | DRB5 | HIC8DR1 | DRB5 | DRB5 | HAA3DR1 | DR11 | HAA5DR1 | HAA6DR1 | HAA7DR11 | DRB5 | HIC | HAART |
| A-TRAV24 Public Clonotypes | | | | | | | | | | | | | | | | | | | | |
| TRAJ17 | CAFKAAGNKLTF | tgtgcctttaaagct | 2 | 35 | 8 | | | 11 | | | | | | 59 | | | 37 | | 6 | 2 |
| | | gcaggcaacaagcta | | | | | | | | | | | | | | | | | | |
| | | actttt | | | | | | | | | | | | | | | | | | |
| | | tgtgcctttaaggct | 1 | | | | 1 | | | | | | | 1 | | | | | | |
| | | gcaggcaacaagcta | | | | | | | | | | | | | | | | | | |
| | | actttt | | | | | | | | | | | | | | | | | | |
| | | tgtgccttcaaagct | | 2 | 64 | | | 2 | 10 | | | | | | | | | | | |
| | | gcaggcaacaagcta | | | | | | | | | | | | | | | | | | |
| | | actttt | | | | | | | | | | | | | | | | | | |
| | | tgtgcatttaaagct | | | | | | 1 | | | | | | | | | | | | |
| | | gcaggcaacaag | | | | | | | | | | | | | | | | | | |
| | | ctaactttt | | | | | | | | | | | | | | | | | | |
| | CAFRAAGNKLTF | tgtgccttcagggct | | | 2 | | | | | | | | | | | | | | 4 | 1 |
| | | gcaggcaacaagcta | | | | | | | | | | | | | | | | | | |
| | | actttt | | | | | | | | | | | | | | | | | | |
| | | tgtgcctttagagct | | | | 1 | | | | | | | | | | | | | | |
| | | gcaggcaacaagcta | | | | | | | | | | | | | | | | | | |
| | | actttt | | | | | | | | | | | | | | | | | | |
| | | tgtgcctttcgagct | | | | | 2 | | | | | | | | | 3 | | | | |
| | | gcaggcaacaagcta | | | | | | | | | | | | | | | | | | |
| | | actttt | | | | | | | | | | | | | | | | | | |
| | | tgtgcctttagggct | | | | | | 1 | | | | | | | | | | | | |
| | | gcaggcaacaagcta | | | | | | | | | | | | | | | | | | |
| | | actttt | | | | | | | | | | | | | | | | | | |
| | CAHKAAGNKLTF | tgtgcccacaaagct | | 2 | | | | | 3 | | | | | | | | | | 2 | 0 |
| | | gcaggcaacaagcta | | | | | | | | | | | | | | | | | | |
| | | actttt | | | | | | | | | | | | | | | | | | |
| | | tgtgcccataaagct | | 1 | | | | | | | | | | | | | | | | |
| | | gcaggcaacaagcta | | | | | | | | | | | | | | | | | | |
| | | actttt | | | | | | | | | | | | | | | | | | |
| | CALKAAGNKLTF | tgtgccttgaaagct | | | 1 | | | | | | | | | | | | | | 2 | 1 |
| | | gcaggcaacaagcta | | | | | | | | | | | | | | | | | | |
| | | actttt | | | | | | | | | | | | | | | | | | |
| | | tgtgccttaaaagct | | | | | | | | | | | | 1 | | | | | | |
| | | gcaggcaacaagcta | | | | | | | | | | | | | | | | | | |
| | | actttt | | | | | | | | | | | | | | | | | | |
| | | tgtgctctaaaagct | | | | | | 6 | | | | | | | | | | | | |
| | | gcaggcaacaagcta | | | | | | | | | | | | | | | | | | |
| | | actttt | | | | | | | | | | | | | | | | | | |
| | CASKAAGNKLTF | tgtgcctctaaagct | | 13 | | | | | | | | | | | | | | | 2 | 0 |
| | | gcaggcaacaagcta | | | | | | | | | | | | | | | | | | |
| | | actttt | | | | | | | | | | | | | | | | | | |
| | | tgtgcctccaaagct | | | 4 | | | | | | | | | | | | | | | |
| | | gcaggcaacaagcta | | | | | | | | | | | | | | | | | | |
| | | actttt | | | | | | | | | | | | | | | | | | |
| | CSRRAAGNKLTF | tgtagtcggagggct | | | 18 | | | | | | | 10 | | | | | | | 1 | 1 |
| | | gcaggcaacaagcta | | | | | | | | | | | | | | | | | | |
| | | actttt | | | | | | | | | | | | | | | | | | |
| TRAJ32 | CAEYGGATNKLIF | tgtgccgaatatggt | | | | 2 | 1 | | 3 | | | | | | | | | | 3 | 0 |
| | | ggtgctacaaacaag | | | | | | | | | | | | | | | | | | |
| | | ctcatcttt | | | | | | | | | | | | | | | | | | |
| | CANYGGATNKLIF | tgtgccaactatggc | | | | | | | 2 | 5 | | | | | | | | | 2 | 0 |
| | | ggtgctacaaacaag | | | | | | | | | | | | | | | | | | |
| | | ctcatcttt | | | | | | | | | | | | | | | | | | |
| | CAPYGGATNKLIF | tgtgccccttatggt | | | | 2 | | | 1 | | | | | | | | | | 2 | 1 |
| | | ggtgctacaaacaag | | | | | | | | | | | | | | | | | | |
| | | ctcatcttt | | | | | | | | | | | | | | | | | | |
| | | tgtgctccttatggc | | | | | | | 1 | | | | | | | | | | | |
| | | ggtgctacaaacaag | | | | | | | | | | | | | | | | | | |
| | | ctcatcttt | | | | | | | | | | | | | | | | | | |
| | | tgtgccccgtatggt | | | | | | | | | | | | | | | | 1 | | |
| | | ggtgctacaaacaag | | | | | | | | | | | | | | | | | | |
| | | ctcatcttt | | | | | | | | | | | | | | | | | | |
| | CARYGGATNKLIF | tgtgcccgttatggt | | | | 10 | | | | | | | | | | | | | 1 | 1 |
| | | ggtgctacaaacaag | | | | | | | | | | | | | | | | | | |
| | | ctcatcttt | | | | | | | | | | | | | | | | | | |
| | | tgtgctcgttatggt | | | | 1 | | | | | | | | | | | | | | |
| | | ggtgctacaaacaag | | | | | | | | | | | | | | | | | | |
| | | ctcatcttt | | | | | | | | | | | | | | | | | | |
| | | tgtgctcggtatggt | | | | | | | | | | | | | | | | 26 | | |
| | | ggtgctacaaacaag | | | | | | | | | | | | | | | | | | |
| | | ctcatcttt | | | | | | | | | | | | | | | | | | |
| | CASYGGATNKLIF | tgtgcctcttatggt | | 8 | | 1 | | | 1 | | | | | | | | | | 3 | 0 |
| | | ggtgctacaaacaag | | | | | | | | | | | | | | | | | | |
| | | ctcatcttt | | | | | | | | | | | | | | | | | | |
| | | tgtgcctcctatggt | | | | 25 | | | 1 | | | | | | | | | | | |
| | | ggtgctacaaacaag | | | | | | | | | | | | | | | | | | |
| | | ctcatcttt | | | | | | | | | | | | | | | | | | |
| | | tgtgcctcgtatggt | | | | 2 | | | | | | | | | | | | | | |
| | | ggtgctacaaacaag | | | | | | | | | | | | | | | | | | |
| | | ctcatcttt | | | | | | | | | | | | | | | | | | |
| | | tgtgcctcctatggc | | | | | | | 2 | | | | | | | | | | | |
| | | ggtgctacaaacaag | | | | | | | | | | | | | | | | | | |
| | | ctcatcttt | | | | | | | | | | | | | | | | | | |
| TRAJ38 | CAFDNRKLIW | tgtgccttcgacaac | | | | | 31 | | | | 37 | | | | | | | | 1 | 1 |
| | | cgtaagctgatttgg | | | | | | | | | | | | | | | | | | |
| | | tgtgcctttgacaac | | | | | 2 | | | | | | | | | | | | | |
| | | cgtaagctgatttgg | | | | | | | | | | | | | | | | | | |
| TRAJ39 | CAFRNAGNMLTF | tgtgcctttcgtaat | | 1 | | | | | | | | | | | | | | | 2 | 2 |
| | | gcaggcaacatgctc | | | | | | | | | | | | | | | | | | |
| | | accttt | | | | | | | | | | | | | | | | | | |
| | | tgtgcctttcggaat | | | | | | 1 | | | | | | | | | | | | |
| | | gcaggcaacatgctc | | | | | | | | | | | | | | | | | | |
| | | accttt | | | | | | | | | | | | | | | | | | |
| | | tgtgcctttaggaat | | | | | | | | | | | 4 | | | | | | | |
| | | gcaggcaacatgctc | | | | | | | | | | | | | | | | | | |
| | | accttt | | | | | | | | | | | | | | | | | | |
| | | tgtgcctttagaaat | | | | | | | | | | | | | | | 10 | | | |
| | | gcaggcaacatgctc | | | | | | | | | | | | | | | | | | |
| | | accttt | | | | | | | | | | | | | | | | | | |
| | CALNNAGNMLTF | tgtgccctcaataat | | | | | | 1 | | | | | | | | | | | 1 | 1 |
| | | gcaggcaacatgctc | | | | | | | | | | | | | | | | | | |
| | | accttt | | | | | | | | | | | | | | | | | | |
| | | tgtgccttaaataat | | | | | | | | | | | | | | 24 | | | | |
| | | gcaggcaacatgctc | | | | | | | | | | | | | | | | | | |
| | | accttt | | | | | | | | | | | | | | | | | | |
| | CALRNAGNMLTF | tgtgccctgcggaat | | | | | | 3 | | | | | | | | | | | 1 | 1 |
| | | gcaggcaacatgctc | | | | | | | | | | | | | | | | | | |
| | | accttt | | | | | | | | | | | | | | | | | | |
| | | tgtgccttaagaaat | | | | | | | | | | | | 1 | | | | | | |
| | | gcaggcaacatgctc | | | | | | | | | | | | | | | | | | |
| | | accttt | | | | | | | | | | | | | | | | | | |
| | | tgtgccttacgaaat | | | | | | | | | | | | 12 | | | | | | |
| | | gcaggcaacatgctc | | | | | | | | | | | | | | | | | | |
| | | accttt | | | | | | | | | | | | | | | | | | |
| | CATRRAGNMLTF | tgtgcgacgcgccgt | | | | 6 | 5 | | | | | | | | | | | | 2 | 0 |
| | | gcaggcaacatgctc | | | | | | | | | | | | | | | | | | |
| | | accttt | | | | | | | | | | | | | | | | | | |
| TRAJ54 | CASESTGAQKLVF | tgtgcctccgagtcc | | | | 3 | 1 | | | | | | | | | | | | 2 | 0 |
| | | acgggagcccagaag | | | | | | | | | | | | | | | | | | |
| | | ctggtattt | | | | | | | | | | | | | | | | | | |
| TRAJ57 | CAYEGASEKLVF | tgtgcctacgagggg | | | | 3 | 1 | | | | | | | | | | | | 2 | 0 |
| | | gcatctgaaaagctg | | | | | | | | | | | | | | | | | | |
| | | gtcttt | | | | | | | | | | | | | | | | | | |
| B-TRBV2 Public Clonotypes | | | | | | | | | | | | | | | | | | | | |
| TRBJ1-1 | CASSGQTNTEAFF | tgtgccagctcagga | | | 10 | | | | | | | | | | | | 2 | | 1 | 1 |
| | | cagacgaacactgaa | | | | | | | | | | | | | | | | | | |
| | | gctttcttt | | | | | | | | | | | | | | | | | | |
| TRBJ1-5 | CASSEGAAGNQPQHF | tgtgccagcagtgaa | | | | | | 1 | | | | | | | | | | | 1 | 1 |
| | | ggggcggctggcaat | | | | | | | | | | | | | | | | | | |
| | | cagccccagcatttt | | | | | | | | | | | | | | | | | | |
| | | tgtgccagcagcgag | | | | | | | | | | | | 1 | | | | | | |
| | | ggggcggcgggcaat | | | | | | | | | | | | | | | | | | |
| | | cagccccagcatttt | | | | | | | | | | | | | | | | | | |
| TRBJ2-1 | CASSELTSGGDEQFF | tgtgccagcagtgag | | | | | | | 1 | 63 | | | | | | | | | 2 | 0 |
| | | ttgactagcgggggg | | | | | | | | | | | | | | | | | | |
| | | gatgagcagttcttc | | | | | | | | | | | | | | | | | | |
| | CASSPGTSGVGEQFF | tgtgcaagcagcccc | | | 15 | | | | | | | | | | | | | | 2 | 0 |
| | | gggactagcggagtt | | | | | | | | | | | | | | | | | | |
| | | ggtgagcagttcttc | | | | | | | | | | | | | | | | | | |
| | | tgtgcaagcagcccc | | | 1 | | | | | | | | | | | | | | | |
| | | gggactagcggagtt | | | | | | | | | | | | | | | | | | |
| | | ggtgagcagtttttc | | | | | | | | | | | | | | | | | | |
| | | tgtgccagcagtccc | | | | | | 2 | | | | | | | | | | | | |
| | | gggactagcggggtt | | | | | | | | | | | | | | | | | | |
| | | ggtgagcagttcttc | | | | | | | | | | | | | | | | | | |
| | CASVLMRTNNEQFF | tgtgccagtgtatta | | | | 9 | 4 | | | | | | | | | | | | | |
| | | atgaggacgaacaat | | | | | | | | | | | | | | | | | 2 | 0 |
| | | gagcagttcttc | | | | | | | | | | | | | | | | | | |
| TRBJ2-3 | CASSALASGTDTQYF | tgtgccagcagtgcg | 1 | | | | | | | | | | | | | | | | 2 | 1 |
| | | ttagctagcggtaca | | | | | | | | | | | | | | | | | | |
| | | gatacgcagtatttt | | | | | | | | | | | | | | | | | | |
| | | tgtgccagcagtgct | | | 1 | | | | | | | | | | | | | | | |
| | | ttggctagcggcaca | | | | | | | | | | | | | | | | | | |
| | | gatacgcagtatttt | | | | | | | | | | | | | | | | | | |
| | | tgtgccagcagtgcc | | | | | | | | | | | | 2 | | | | | | |
| | | ctggctagtggcaca | | | | | | | | | | | | | | | | | | |
| | | gatacgcagtatttt | | | | | | | | | | | | | | | | | | |
| | CASSARTSGGADTQYF | tgtgccagcagtgca | | | | 1 | | | | | | | | | | | | | 2 | 0 |
| | | aggactagcgggggg | | | | | | | | | | | | | | | | | | |
| | | gccgatacgcagtat | | | | | | | | | | | | | | | | | | |
| | | ttt | | | | | | | | | | | | | | | | | | |
| | | tgtgccagcagtgcc | | | | | | | 1 | | | | | | | | | | | |
| | | aggactagcgggggg | | | | | | | | | | | | | | | | | | |
| | | gcagatacgcagtat | | | | | | | | | | | | | | | | | | |
| | | ttt | | | | | | | | | | | | | | | | | | |
| | | tgtgccagcagtgct | | | | | | | 1 | | | | | | | | | | | |
| | | aggactagcgggggg | | | | | | | | | | | | | | | | | | |
| | | gcagatacgcagtat | | | | | | | | | | | | | | | | | | |
| | | ttt | | | | | | | | | | | | | | | | | | |
| | CASSARTSGGSDTQYF | tgtgccagcagtgcc | 1 | | | | | | | | | | | | | | | | 3 | 0 |
| | | aggactagcgggggc | | | | | | | | | | | | | | | | | | |
| | | tcggatacgcagtat | | | | | | | | | | | | | | | | | | |
| | | ttt | | | | | | | | | | | | | | | | | | |
| | | tgtgccagcagtgct | | | | 3 | | | | | | | | | | | | | | |
| | | cggactagcgggggg | | | | | | | | | | | | | | | | | | |
| | | tcagatacgcagtat | | | | | | | | | | | | | | | | | | |
| | | ttt | | | | | | | | | | | | | | | | | | |
| | | tgtgccagcagcgcc | | | | | | | 1 | | | | | | | | | | | |
| | | cggactagcgggggg | | | | | | | | | | | | | | | | | | |
| | | tcagatacgcagtat | | | | | | | | | | | | | | | | | | |
| | | ttt | | | | | | | | | | | | | | | | | | |
| | | tgtgccagcagtgcc | | | | | | | 2 | | | | | | | | | | | |
| | | aggactagcgggggg | | | | | | | | | | | | | | | | | | |
| | | tcagatacgcagtat | | | | | | | | | | | | | | | | | | |
| | | ttt | | | | | | | | | | | | | | | | | | |
| | CASSARTSGGTDTQYF | tgtgccagcagtgcc | | | | | | | 2 | 1 | | | | | | | | | 2 | 0 |
| | | aggactagcggaggc | | | | | | | | | | | | | | | | | | |
| | | acagatacgcagtat | | | | | | | | | | | | | | | | | | |
| | | ttt | | | | | | | | | | | | | | | | | | |
| | | tgtgccagcagtgct | | | | | | | 1 | | | | | | | | | | | |
| | | aggactagcgggggc | | | | | | | | | | | | | | | | | | |
| | | acagatacgcagtat | | | | | | | | | | | | | | | | | | |
| | | ttt | | | | | | | | | | | | | | | | | | |
| | CASSHRASGGDTQYF | tgtgccagcagtcac | | | | 31 | 1 | | | | | | | | | | | | 2 | 0 |
| | | agggcctcaggcggg | | | | | | | | | | | | | | | | | | |
| | | gatacgcagtatttt | | | | | | | | | | | | | | | | | | |
| | CASSLRTSGGSDTQYF | tgtgccagcagccta | 1 | | | | | | | | | | | | | | | | 1 | 1 |
| | | aggactagcgggggt | | | | | | | | | | | | | | | | | | |
| | | tcagatacgcagtat | | | | | | | | | | | | | | | | | | |
| | | ttt | | | | | | | | | | | | | | | | | | |
| | | tgtgccagcagcctt | | | | | | | | | | | | | | | 1 | | | |
| | | aggactagcgggggt | | | | | | | | | | | | | | | | | | |
| | | tcagatacgcagtat | | | | | | | | | | | | | | | | | | |
| | | ttt | | | | | | | | | | | | | | | | | | |
| | CASSLRTSGGTDTQYF | tgtgccagcagtctt | | | | 1 | | | | | | | | | | | | | 1 | 1 |
| | | aggactagcgggggc | | | | | | | | | | | | | | | | | | |
| | | acagatacgcagtat | | | | | | | | | | | | | | | | | | |
| | | ttt | | | | | | | | | | | | | | | | | | |
| | | tgtgccagcagtctc | | | | | | | | | | | | | | | | 2 | | |
| | | cggactagcgggggc | | | | | | | | | | | | | | | | | | |
| | | acagatacgcagtat | | | | | | | | | | | | | | | | | | |
| | | ttt | | | | | | | | | | | | | | | | | | |
| | | tgtgccagcagtctt | | | | | | | | | | | | | | | | 1 | | |
| | | cggactagcggggga | | | | | | | | | | | | | | | | | | |
| | | acagatacgcagtat | | | | | | | | | | | | | | | | | | |
| | | ttt | | | | | | | | | | | | | | | | | | |
| | CASSPLTSGTDTQYF | tgtgccagcagtccg | | 1 | | | | | | | | | | | | | | | 1 | 1 |
| | | ttgactagcggaaca | | | | | | | | | | | | | | | | | | |
| | | gatacgcagtatttt | | | | | | | | | | | | | | | | | | |
| | | tgtgccagcagtcca | | | | | | | | | | | | 1 | | | | | | |
| | | ttgactagcggcaca | | | | | | | | | | | | | | | | | | |
| | | gatacgcagtatttt | | | | | | | | | | | | | | | | | | |
| | | tgtgccagcagtcgt | | | | | | | | | | | | | | | | | | |
| | | aggactagcgggggc | | 6 | | | | | | | | | | | | | | | | |
| | | acagatacgcagtat | | | | | | | | | | | | | | | | | | |
| | | ttt | | | | | | | | | | | | | | | | | | |
| | CASSRRTSGGTDTQYF | tgtgccagcagtcga | | | | 1 | | | | | | | | | | | | | 4 | 0 |
| | | cggactagcgggggc | | | | | | | | | | | | | | | | | | |
| | | acagatacgcagtat | | | | | | | | | | | | | | | | | | |
| | | ttt | | | | | | | | | | | | | | | | | | |
| | | tgtgccagctcacga | | | | 1 | | | | | | | | | | | | | | |
| | | cggactagcgggggc | | | | | | | | | | | | | | | | | | |
| | | acagatacgcagtat | | | | | | | | | | | | | | | | | | |
| | | ttt | | | | | | | | | | | | | | | | | | |
| | | tgtgccagcagccgc | | | | | | 12 | | | | | | | | | | | | |
| | | cggactagcgggggc | | | | | | | | | | | | | | | | | | |
| | | acagatacgcagtat | | | | | | | | | | | | | | | | | | |
| | | ttt | | | | | | | | | | | | | | | | | | |
| | | tgtgccagcagtcgg | | | | | | | 1 | | | | | | | | | | | |
| | | aggactagcgggggc | | | | | | | | | | | | | | | | | | |
| | | acagatacgcagtat | | | | | | | | | | | | | | | | | | |
| | | ttt | | | | | | | | | | | | | | | | | | |
| TRBJ2-7 | CASSKLASGADEQYF | tgtgccagctcaaaa | | | | 6 | 1 | | | | | | | | | | | | 2 | 0 |
| | | ctggctagcggggcc | | | | | | | | | | | | | | | | | | |
| | | gacgagcagtacttc | | | | | | | | | | | | | | | | | | |
| | | tgtgccagctcaaaa | | | | 1 | | | | | | | | | | | | | | |
| | | ctggctagcggggcc | | | | | | | | | | | | | | | | | | |
| | | gacgagcagtatttc | | | | | | | | | | | | | | | | | | |
| | CASSPRTSGGDEQYF | tgtgccagcagtccc | | | | | | | 1 | | | | | | | | | | 1 | 1 |
| | | cggactagcgggggc | | | | | | | | | | | | | | | | | | |
| | | gacgagcagtacttc | | | | | | | | | | | | | | | | | | |
| | | tgtgccagcagcccc | | | | | | | | | | | | | | | | 1 | | |
| | | cggactagcgggggg | | | | | | | | | | | | | | | | | | |
| | | gacgagcagtacttc | | | | | | | | | | | | | | | | | | |
| | CASSPRTSGTYEQYF | tgtgccagcagtccc | | | | 1 | | | | | | | | | | | | | 1 | 1 |
| | | cggactagcggtacc | | | | | | | | | | | | | | | | | | |
| | | tacgagcagtacttc | | | | | | | | | | | | | | | | | | |
| | | tgtgccagcagtcct | | | | | | | | | | | | | | | | 1 | | |
| | | aggactagcggaacc | | | | | | | | | | | | | | | | | | |
| | | tacgagcagtacttc | | | | | | | | | | | | | | | | | | |
| | CASSRRTSGTYEQYF | tgtgccagcagccgt | 1 | | | | | | | | | | | | | | | | 2 | 0 |
| | | aggactagcgggact | | | | | | | | | | | | | | | | | | |
| | | tacgagcagtacttc | | | | | | | | | | | | | | | | | | |
| | | tgtgccagcagtaga | | | | 1 | | | | | | | | | | | | | | |
| | | cggactagcgggacc | | | | | | | | | | | | | | | | | | |
| | | tacgagcagtacttc | | | | | | | | | | | | | | | | | | |

The number of nucleotide sequences coding for a TRAV24 (A) or TRBV2 (B) public clonotype is reported for each patient studied.
The number of HIC and HAART patients sharing a given public clonotype is reported in the last two columns.
HIC: HIV Controller; HAART or HAA: patient receiving highly active antiretroviral therapy.
The tetramer used for sorting (DR1, DR11, DR15, or DRB5) is reported below each patient code.

Public clonotypes were also abundant in Gag293-specific TCR(I chains, with a total of 18 found in the TRBV2 sequence dataset (Table 3B). Public clonotype sequences derived predominantly from the TRBJ2-3, TRBJ2-1, and TRBJ2-7 genes, which were all more represented in HIC than HAART sequences. TRBV2 public clonotypes showed a remarkably high degree of motif sharing (83%, Table 2), emphasizing the presence of conserved features in spite of relatively high sequence diversity. Again, TRBV2 public clonotypes were clearly more frequent in the HIC than the HAART group, in terms of aa sequences (P=0.006; FIG. 3D) and of nucleotide sequences (P=0.009; FIG. 3F). The most frequent public clonotype, ASSRRTSGGTDTQY (called TRBV2-13), was found in 4/8 Controllers (50%) and absent from treated patients. The majority of TRBV2 public clonotypes (77%) were encoded by more than one nucleotide sequence. The mean number of unique nucleotide sequences encoding a TRBV2 public clonotype was 2.28, versus 1.10 for private TRBV2 clonotypes, again suggestive of convergent V(D)J recombination contributing to the amplification of Gag293-specific public clonotypes. Taken together, TRAV24 and TRBV2 sequence analyses revealed a highly biased Gag293-specific TCR repertoire, characterized by a high degree of TCR sharing in the Controller group. TCR biases were of type III (identical CDR3 sequences), but also of type of type II (conserved CDR3 motifs) (35, 37), and concerned a significant fraction of both TCR chains.

It was noteworthy that the majority of public clonotypes were restricted by multiple HLA DR alleles. Specifically, 83% of TRAV24 public clonotypes and 56% of TRBV2 public clonotypes were identified in samples sorted with at least 2 different HLA DR tetramers (Table 3, A and B). The most prevalent TRAV24 public clonotype, TRAV24-F, was restricted by the 4 HLA DR alleles tested (DR1, DR11, DR15, DRB5), while the most prevalent TRBV2 public clonotype, TRBV2-13, was restricted by 3 of these alleles (DR1, DR15, and DRB5). Thus, highly prevalent public clonotypes showed a high level of HLA cross-restriction, which could help explain their prevalence in patients of diverse genetic backgrounds.

We next evaluated the degree of overlap of the Gag293-specific clonotypic repertoires restricted by two distinct HLA DR alleles in a same patient. This intrapatient comparison was done for 3 HIV Controllers, for whom two cell lines sorted with different tetramers (DR15/DRB5, or DR11/DRB5) were analyzed in parallel (FIGS. 15A to 15F and 16A to 16F). These experiments revealed a consistent but moderate degree of clonotypic repertoire overlap, with a mean of 12% TRAV24 clonotypes and 11% of TRBV2 clonotypes shared between the two Tet+ samples (Table 9). However, the shared clonotypes were highly expressed, as they represented a mean of 44% TRAV24 sequences and 27% TRBV2 sequences analyzed. Public clonotypes were disproportionately represented among shared clonotypes, with a mean of 83% of shared TRAV24 clonotypes and 47% of shared TRBV2 clonotypes. Thus, while the majority of Gag293-specific clonotypes did not show cross-restriction, the subset of clonotypes restricted by multiple HLA DR alleles appeared dominant and more frequently public.

FIGS. 15A to 15F and 16A to 16F show Intrapatient comparison of Gag293-specific clonotypic repertoires obtained with different MHC II tetramers

The Gag293-specific repertoire was compared in two cell lines obtained from the same patient but sorted with two distinct MHC II tetramers.
The TRAV24 clonotypes FIGS. 15A to 15F and TRBV2 clonotypes (B) FIGS. 16A to 16F shared between the two tetramer+ samples are highlighted in grey.
Comparisons are shown for 3 HIV Controllers. Public clonotypes are in bold type. Clonotypes tested functionally are marked by an asterisk.

TABLE 9
Intrapatient comparisons of Gag293-specific clonotypic repertoires
obtained with different MHC II tetramers - Summary

| | | % shared | | % public in |
|---|---|---|---|---|
| | | TRAV24 | % shared | shared |
| | Tetramers | clonotypes | TRAV24 | TRAV24 |
| Patient | used | AA | sequences | clonotypes AA |
| HIC1 | DRB1*1501/ | 22.22 | 83.51 | 50 |
| | DRB5*0101 | | | |
| HIC3 | DRB1*1101/ | 7.69 | 16.98 | 100 |
| | DRB5*0101 | | | |
| HIC7 | DRB5*0101/ | 6.25 | 30.92 | 100 |
| | DRB1*1502 | | | |
| Mean | | 12.05 | 43.80 | 83.33 |
| | | % shared | | % public in |
| | | TRBV2 | % shared | shared |
| | Tetramers | clonotypes | TRBV2 | TRBV2 |
| Patient | used | AA | sequences | clonotypes AA |
| HIC1 | DRB1*1501/ | 11.63 | 12.5 | 40 |
| | DRB5*0101 | | | |
| HIC3 | DRB1*1101/ | 14.81 | 40.22 | 50 |
| | DRB5*0101 | | | |
| HIC7 | DRB5*0101/ | 6.45 | 28.57 | 50 |
| | DRB1*1502 | | | |
| Mean | | 10.96 | 27.10 | 46.67 |

The Gag293-specific repertoire was compared in cell lines obtained from the same patient but sorted with two distinct MHC II tetramers. Comparisons were made for 3 HIV Controllers (HIC1, HIC3, HIC7). Clonotype AA: unique CDR3 sequence, in amino acids.

The % of shared clonotypes AA is obtained from the number of shared clonotypes AA divided by the total number of clonotypes AA obtained for the two tetramer+ samples.

The % of shared sequences is obtained from the number of nt sequences corresponding to shared clonotypes AA divided by the total number of sequences obtained for the two tetramer+ samples.

The % of public clonotypes is computed among the shared clonotypes AA.

Ex Vivo Detection of Gag293-Specific Public Clonotypes in HIV Controllers

The Gag293-specific TCR repertoire was initially analyzed in primary CD4+ T cell lines, as the preferential depletion of specific CD4+ T cells in HIV-infected patients did not allow a direct ex vivo analysis of Tet+ cells. However, the exceptional immunological status of HIV Controllers made the ex vivo analysis feasible for a subset of these patients. Four samples sorted from Controller PBMC containing >2,000 Tet+ cells were analyzed by Immunoscope and sequencing. The DRB5 tetramer was used in these experiments, as it yielded the best signal/noise ratio (example in FIG. 4A). Quantification of TRAV24 expression showed an amplification of this family in 3/4 HIC samples obtained ex vivo, as compared to the percentage of TRAV24 detected in PBMC from 7 healthy donors (1.0±0.2%) (FIG. 4B). Quantification of TRBV2 showed an amplification in 4/4 HIC samples obtained ex vivo, as compared to PBMC from healthy donors (3.7±1.0%) (FIG. 4B).

The frequency of each TRAV24 clonotype found ex vivo was computed relative to total TRAV24 sequences found in the same patient (Table 4, 3rd column) and in the matched cell line (4th column). Ex vivo clonotypes represented in median 66% of sequences found in matched cell lines, indicating a large overlap between the ex vivo and in vitro repertoires (FIG. 4C). Similarly, the TRBV2 clonotypes detected ex vivo (Table 7), represented in median 45% of sequences found in matched cell lines (FIG. 4C). Importantly, the frequency of TRAV24 public motifs (AV24-1+AV24-2) did not differ significantly between ex vivo and cell line derived sequences, and could be as high as 87% (FIG. 4D). Frequencies of TRBV2 public motifs (BV2-1+BV2-2) were also comparable ex vivo and in matched cell lines (FIG. 4E). Public motifs were present at median values of 44% and 48% in the ex vivo TRAV24 and TRBV2 sequence datasets, respectively, emphasizing the high level of motif sharing in the Gag293-specific repertoire.

Detailed analysis of TRAV24 sequences obtained ex vivo revealed the presence of previously identified public clonotypes in each of the patient tested (highlighted in color, Table 4). Public clonotypes were present at median frequencies of 44% and 25% in ex vivo TRAV24 and TRBV2 sequences, respectively, which did not differ significantly from frequencies observed in matched cell lines (not shown). Overall, the ex vivo analysis confirmed the high degree of TCR sharing among HIV Controllers. It was striking that some of the most prevalent public clonotypes, such as AFKAAGNKLT, could represent more than half of TRAV24 sequences in the Gag293-specific repertoire (54.7% in HIC7). This suggested that public clonotypes could shape the properties of the Gag-specific response in controlled HIV infection.

TABLE 4
Listing of TRAV24 clonotypes found in
Gag293-specific CD4+ T cells sorted ex vivo.

| | | | |
|---|---|---|---|
| | | | % in cell |
| J-GENE | JUNCTION (AA) | % ex vivo | line |

HIC1 DRB5+ ex vivo (n = 119 seq.)

| | | | |
|---|---|---|---|
| TRAJ38*01 | CARDDRKLIW | 4.20 | — |
| TRAJ17*01 | CAFTAAGNKLTF | 8.40 | — |
| TRAJ39*01 | CALGNAGNMLTF | 27.73 | 4.65 |
| TRAJ6*01 | CAFIPGGSYIPTF | 3.36 | 2.33 |
| TRAJ32*02 | CASYGGATNKLIF | 21.85 | 62.79 |
| TRAJ45*01 | CALDSGGGADGLTF | 34.45 | — |

HIC2 DRB5+ ex vivo (n = 56 seq.)

| | | | |
|---|---|---|---|
| TRAJ17*01 | CAFKAAGNKLTF | 17.86 | 56.06 |
| TRAJ39*01 | CARRNAGNMLTF | 12.50 | — |
| TRAJ17*01 | CASKAAGNKLTF | 69.64 | 19.70 |

HIC3 DRB5+ ex vivo (n = 76 seq.)

| | | | |
|---|---|---|---|
| TRAJ17*01 | CALKAAGNKLTF | 6.58 | 1.79 |
| TRAJ39*01 | CALRQAGNMLTF | 93.42 | 62.50 |

HIC7 DRB5+ ex vivo (n = 106 seq)

| | | | |
|---|---|---|---|
| TRAJ13*02 | CAPGGYQKVTF | 4.72 | — |
| TRAJ22*01 | CAWGSARQLTF | 4.72 | — |
| TRAJ17*01 | CAFKAAGNKLTF | 54.72 | 18.18 |
| TRAJ17*01 | CAFNAAGNKLTF | 5.66 | 5.45 |
| TRAJ26*01 | CAFVAAGQNFVF | 4.72 | — |
| TRAJ32*02 | CAEYGGATNKLIF | 3.77 | 5.45 |
| TRAJ32*01 | CANYGGATNKLIF | 6.60 | 3.64 |
| TRAJ32*02 | CASYGGATNKLIF | 0.94 | 7.27 |
| TRAJ24*02 | CALMTTDSWGKLQF | 14.15 | — |

The representation of each clonotype in the TRAV24 sequence set obtained ex vivo (3rd column) and in the corresponding cell line (4th column) is reported (SEQ ID NO: 176 to 195).

TCR Transfer Confers Gag293 Recognition in the Context of Multiple HLA-DR Alleles

We set to functionally characterize the most prevalent TRAV24 public clonotype AFKAAGNKLT (clonotype TRAV24-F) by pairing it with different TRBV2 chains and testing the activity of reconstituted TCRs. The chosen TRBV2 clonotypes no. 24 (CASSRLAGGMDEQFF) and 25 (CATTPGASGISEQFF) were the most abundant in cell line of patient HIC2 (10.38% and 33.96% of TRBV2 chains, respectively; Table 7), and were co-expressed at high levels with clonotype TRAV24-F, resulting in a high probability of functional TCR chain pairing. The third TRBV2 clonotype, no. 5 (CASSGLAGGMDEQFF), was derived from patient HIC3, who also expressed TRAV24-F at high level. Clonotype 5 differed by a single residue (R to G) from clonotype 24, and thus provided information on the contribution of the CDR3β arginine to TCR function. TRBV2 clonotypes 24 and 5 carried the public motif BV2-1, while TRBV2 clonotype 25 was private (see Table 10 for a list of all TCR constructs). Full-length TRA and TRB genes were cloned into a T2A lentiviral vector, ensuring equimolar expression of the two chains. The resulting TCRs, F24, F25, and F5, were transduced in J76, a mutant Jurkat cell line defective for endogenous TCR expression, which provides a favorable cellular context for TCR transfer assays. After transduction, J76 cells expressed equivalent levels of the 3 TCRs at the cell surface (≥60% TCR+ cells), and recovered a high level of CD3 surface expression (FIG. 5A).

To test the specificity of the transferred TCRs, transduced J76 were labeled with a panel of Gag293-loaded HLA-DR tetramers. The F24 TCR was found to bind 3 out of the 4 tetramer tested, with an efficiency that was higher for DR11 as compared to DRB5 and DR1 (FIG. 5B). Quantification of tetramer binding (FIG. 5C) showed that the three TCRs recognized Gag293 in the context of DR11, with a labeling that was strong for F24 (72% Tet+), intermediate for F25 (44% Tet+), and low for F5 (3% Tet+). F24 bound the DRB5 tetramer at intermediate levels (33% Tet+), while binding was weak for F25 (1.5%), and undetectable for F5. Binding with the DR1 tetramer showed a similar pattern. The DR4 tetramer provided a negative control, as it did not bind any of the TCRs tested. These experiments indicated that the highly prevalent public clonotype TRAV24-F could confer Gag293 recognition in the context of multiple HLA-DR molecules, with an efficiency that depended both on the presenting HLA-DR allele and on the nature of the TRBV2 chain.

Tetramer titration experiments showed the expected hierarchy between the 3 TCRs (F24>F25>F5) but did not prove sensitive enough to determine TCR avidity for F25 and F5 (FIG. 10). To precisely determine TCR affinity, we expressed the F24, F25, and F5 TCRs as soluble recombinant proteins and measured their binding to Gag293-HLA-DR monomers by surface plasmon resonance (SPR). The soluble TCRs were passed over chips coated with immobilized DR11, DRB5, or DR1 monomers loaded with Gag293 (example of sensorgram in FIG. 5D). In agreement with tetramer binding assays, the three TCRs recognized Gag293-HLA-DR complexes with different affinities (F24>F25>F5). This hierarchy was conserved across the different HLA-DR alleles tested (FIG. 5E). Of note, the F24 TCR recognized the Gag293-DR11 complex with a very high affinity of 0.86 μM (FIG. 5F), which is rarely seen for MHC II restricted TCRs (38). F24 bound the Gag293-DRB5 and Gag293-DR1 complexes with affinities that were three- and eight-fold lower, respectively, but still remained in the high affinity range (<10 μM). The F25 and F5 TCRs also interacted with the Gag293-DR11 complex with relatively high affinities (3 and 11 μM, respectively), while affinities were intermediate or low for the two other allomorphs tested. The percentage of Tet+ cells in TCR-transduced J76 cells, when measurable, correlated well with affinities measured by SPR (R=0.92; FIG. 5G), consistent with the notion that tetramer binding tightly depends on TCR affinity. These results showed that association of the TRAV24-F public clonotype with highly expressed TRBV2 clonotypes could, for certain combinations, generate HLA cross-restricted TCRs of unusually high affinity.

Transfer of TCRs Containing Public TRAV24 and TRBV2 Clonotypes Confers High Antigen Sensitivity to J76 Cells

TCR function was tested by monitoring the induction of the early activation marker CD69 in transduced J76 cells. Murine fibroblastic L cells engineered to express a single human HLA-DR allele were used as APC, allowing a precise control of the restricting HLA molecule. L cells expressing DR1, DR3, DR4, DR7, DR11, DR15, or DRB5 were pulsed with decreasing doses of Gag293 peptide and cocultured with J76 cells transduced with the F24, F25, and F5 TCRs (FIG. 6A). The three TCRs could signal and trigger T cell activation, as indicated by the induction of CD69 in ≥50% of J76 cells in the presence of DR11 APC. The F24 TCR showed the broadest reactivity, as it induced strong T cell activation in the presence of DR11, DR15, DRB5, and DR1 APC, and weak but consistent T cell activation in the presence of DR7 APC (Table 10). When restricted by DR11, F24 antigen sensitivity reached 4×10⁻⁷ M as measured by the EC50 for CD69 induction, which represents a high sensitivity for a cell culture system devoid of costimulatory molecules. The F25 TCR did not react with DR7, and showed intermediate reactivity with the other four HLA-DR alleles. The F5 TCR showed the narrowest restriction, with an antigen sensitivity of 2×10⁻⁶ M in the presence of DR11 APC, low responses in the presence of DR15 and DRB5 APC, and undetectable responses in other cases. TCR affinities measured by SPR for the DR11, DRB5, and DR1 alleles correlated well with antigen sensitivities measured in transduced J76 (R=0.85, P=0.016; FIG. 6D), suggesting that TCR affinity dictated TCR function.

Two other TRAV24 public clonotypes, TRAV24-S(ASKAAGNKLT) and TRAV24-RR (SRRAAGNKLT) conferred cross-restriction by 4 HLA-DR alleles when paired to TRBV2 chains no. 24 and 5, respectively (Table 10), emphasizing that broad HLA cross-restriction was a frequent property of public clonotypes. We then tested the pairing of TRAV24-F with two TRBV2 public clonotypes: TRBV2-4 (ASSPGTSGVGEQF), shared by 2 Controllers, and TRBV2-13 (ASSRRTSGGTDTQY), the most prevalent TRBV2 clonotype, shared by 4 Controllers. The resulting TCRs, F4 and F13, were cross-restricted by 3 HLA-DR alleles (DR11, DR15 and DRB5) (FIG. 6B). While F4 had relatively good antigen sensitivity (10⁻⁶ M) when restricted DR11, it gave only low responses with the two other alleles. In contrast, F13 showed good antigen sensitivity with DR11 (7.5×10⁻⁷ M) and intermediate sensitivity with the two other alleles (Table 10). F13, like F24, carried an R at position 5 of the TRBV2 CDR3 junction, which may contribute to the efficient detection of Gag293 in varied HLA backgrounds. Two TCRs derived from private clonotypes from treated patients were studied for comparison. TCRs HD5 and HY9 comprised the most prevalent TRAV24 and TRBV2 clonotypes from treated patients HAART3 and HAART6, respectively (Table 10). These TCRs yielded responses of medium avidity when restricted by DR15 and DR1 (10⁻⁵ M≤EC50≤10⁻⁶ M), gave only low responses in the presence of DRB5 L cells, and did not react with DR11 L cells (FIG. 6C). While these TCRs from treated patients could react with several HLA-DR alleles, their degree of cross-restriction appeared narrower than that observed for Controller TCRs. Taken together, the analysis of recombinant TCRs specific for Gag293 highlighted that the most prevalent public clonotypes conferred efficient Gag293 recognition in the context of multiple HLA-DR alleles. Interestingly, antigen sensitivity measured for the 8 TCR tested in the presence of DR11 correlated with the number of HLA-DR alleles restricting each of these TCRs (R=−0.87, P=0.002; FIG. 6E), suggesting that both properties depended on the same TCR features.

We next verified that the recombinant TCRs could recognize native HIV capsid antigens naturally processed by APC, in addition to peptide-pulsed APC. To this goal, we used infected monocyte-derived dendritic cells (MDDC) as alternative APC. MDDC from DR11+ healthy donors were infected with an HIV-1 pseudotyped virus and incubated with J76 cells expressing the F24, F25, or F5 TCR. The TCR-transduced cells showed robust CD69 induction in the presence of infected MDDC, while CD69 expression remained moderate in the absence of infection (FIG. 6F). CD69 induction followed the same hierarchy as that observed when L cells were used as APC (F24>F25>F5), indicating that TCR properties were conserved in different antigen presentation systems.

As the public clonotypes consistently generated TCRs with optimal antigen sensitivity in a DR11 context, it was important to verify that differences in antigen sensitivity between the HIC and the HAART group did not depend on a bias in DR11 expression. Among the 29 patients included in the study, 3 Controllers and 4 treated patients expressed a DR11 allele, including HLA DRB1*1101, DRB1*1122, and DRB1*1165 (Table 5). Comparison of the last Gag293 peptide dilution that induced a specific cell line showed that, among DR11 patients, 3 out of 3 Controllers and 1 in 4 treated patient responded at a dilution <10-5 M (FIG. 11A). Trends for a higher Gag293-specific ELISPOT response and for more prominent TRAV24 biases were also noted in DR11 Controllers as compared to DR11 treated patients (FIGS. 11B and C). Taken together, these findings support the notion that particular clonotypes expressed in controlled HIV infection, rather than mere DR11 expression, are associated with high avidity Gag293-specific responses.

TABLE 5
MHC typing and clinical characteristics of patients included in the study

| | | Duration | | | | | |
|---|---|---|---|---|---|---|---|
| | | of | | VL | | | Tetramer |
| Patient | Age | Infection | CD4/ | copies/ | MHC Class II | | used for |
| ID | yrs | yrs | mm3 | mL | DRB1 alleles | | sort |
| HIC 1 | 56.4 | 25.0 | 850 | <50 | DRB1*1501/1507/1510 (DRB5) | | DRB1*1501 |
| HIC 2 | 50.8 | 24.2 | 1020 | <50 | DRB1*1122 | DRB1*1501/ | DRB5*0101 |
| | | | | | | 1507/1510 | |
| | | | | | | (DRB5) | |
| HIC 3 | 54.7 | 22.0 | 1050 | <50 | DRB1*0102 | DRB1*1101 | DRB1*1101 |
| HIC 4 | 61.4 | 26.0 | 852 | <50 | DRB1*1501 | (DRB5) | DRB1*1501 |
| HIC 5 | 53.6 | 11.0 | 742 | <50 | DRB1*0101 | DRB1*1607 | DRB1*0101 |
| HIC 6 | 34.6 | 13.1 | 1048 | <50 | DRB1*1101 | DRB1*1204/ | DRB1*1101 |
| | | | | | | 1201/1202/ | |
| | | | | | | 1307 | |
| HIC 7 | 41.7 | 11.7 | 744 | <50 | DRB1*0701/ | DRB1*1501/ | DRB5*0101 |
| | | | | | 0703/0704/ | 1502 | |
| | | | | | 0705/0706 | (DRB5) | |
| HIC 8 | 48.5 | 21.0 | 799 | <50 | DRB1*0102 | DRB1*1301 | DRB1*0101 |
| HIC 9 | 49.7 | 14.6 | 754 | <50 | DRB1*0101 | DRB1*0701 | — |
| HIC 11 | 45.0 | 26.0 | 1042 | <50 | DRB1*0101 | DRB1*1501 | — |
| | | | | | | (DRB5) | |
| HIC 12 | 50.9 | 25.5 | 648 | <50 | DRB1*0301 | DRB1*1359 | — |
| HIC 13 | 34.0 | 9.8 | 899 | <50 | DRB1*1360/ | DRB1*1401/ | — |
| | | | | | 1402 | 1602 | |
| | | | | | | (DRB5) | |
| HIC 14 | 56.6 | 18.3 | 1400 | <50 | DRB1*1301/ | DRB1*0703 | — |
| | | | | | 1302 | | |
| HIC 15 | 43.6 | 14.7 | 1063 | <50 | DRB1*0101 | DRB1*0703 | — |
| Median | 50.3 | 19.6 | 875 | <50 | | | |
| HIC | | | | | | | |
| HAAR | 41.4 | 7.1 | 516 | <50 | DRB1*0101/ | DRB1*1502/ | DRB5*0101 |
| T 1 | | | | | 0107 | 1504/1506/ | |
| | | | | | | 1514 | |
| | | | | | | (DRB5) | |
| HAAR | 44.5 | 10.2 | 432 | <50 | DRB1*1502 | DRB1*1608 | DRB5*0101 |
| T 2 | | | | | (DRB5) | | |
| HAAR | 52.5 | 25.3 | 682 | <50 | DRB1*0101/ | DRB1*0703 | DRB1*0101 |
| T 3 | | | | | 0107 | | |
| HAAR | 53.0 | 14.5 | 570 | <50 | DRB1*0703 | DRB1*1122 | DRB1*1101 |
| T 4 | | | | | | | |
| HAAR | 48.6 | 9.0 | 948 | <50 | DRB1*0102 | DRB1*1410 | DRB1*0101 |
| T 5 | | | | | | | |
| HAAR | 54.5 | 6.8 | 266 | <50 | DRB1*0101/01 | DRB1*0322 | DRB1*0101 |
| T 6 | | | | | 07 | | |
| HAAR | 55.9 | 18.1 | 846 | <50 | DRB1*0701 | DRB1*1122 | DRB1*1101 |
| T 7 | | | | | | | |
| HAAR | 39.5 | 20.1 | 847 | <50 | DRB1*1501 (DRB5) | | DRB5*0101 |
| T 8 | | | | | | | |
| HAAR | 47.7 | 19.7 | 534 | <50 | N.A. | N.A. | — |
| T 9 | | | | | | | |
| HAAR | 54.0 | 11.2 | 422 | <50 | DRB1*0301 | DRB1*1501 | — |
| T 10 | | | | | | (DRB5) | |
| HAAR | 52.1 | 12.9 | 937 | <50 | DRB1*0701 | DRB1*1001 | — |
| T 11 | | | | | | | |
| HAAR | 43.0 | 12.1 | 558 | <50 | DRB1*1165 | DRB1*1301 | — |
| T 12 | | | | | | | |
| HAAR | 45.0 | 11.9 | 1534 | <50 | DRB1*0401 | DRB1*1101 | — |
| T 13 | | | | | | | |
| HAAR | 47.5 | 11.9 | 557 | <50 | DRB1*0901 | DRB1*1301 | — |
| T 14 | | | | | | | |
| HAAR | 41.4 | 7.1 | 785 | <50 | DRB1*0101 | DRB1*0301 | — |
| T 15 | | | | | | | |
| Median | 47.7 | 11.9 | 570 | <50 | | | |
| HAAR | | | | | | | |
| T | | | | | | | |

Primary CD4+ T cell lines were generated from 14 HIV Controllers (HIC) and 15 treated patients (HAART) genotyped for HLA-DRB1.
For a subgroup of 8 HIC and 8 HAART patients, CD4+ T cell lines were sorted with HLA-matched tetramers loaded with the Gag293 peptide.
The tetramer used for the sort is reported in the rightmost column.
VL: viral load;
N.A. not available.

Transfer of the F24 TCR confers polyfunctionality to primary CD4+ and CD8+ T cells

We next evaluated the properties of the F24, F25, and F5 TCRs when transferred directly into primary T cells, a system physiologically more relevant to potential TCR transfer applications. Healthy donor PBMC were transduced with concentrated TCR lentivector stocks, and analyzed for exogenous TCR expression with a TRBV2-specific mAb. Mock-transduced CD4+ T cells expressed endogenous TRBV2 at levels below 5%, while TCR-transduced CD4+ T cells showed TRBV2 expression rates in the 25%-35% range (FIG. 12A). The hierarchy of HLA-DR tetramer binding was consistent with that observed in J76 cells, with F24 showing significant binding to 4 different tetramers (DR11, DR15, DRB5, and DR1), F25 reacting mostly to the DR11 and DRB5 tetramers and minimally to the DR1 tetramer, and F5 showing barely detectable tetramer binding (FIG. 12B-C).

To evaluate TCR function, PMBC from DR11, DR15, DR1, or DRB5 donors were TCR-transduced and cocultured with autologous MDDC pulsed with Gag293. A panel of 5 markers, including the cytokines TNF-α, IL-2, and IFN-γ, the chemokine MIP-1β/CCL4, and the degranulation marker CD107a, was assayed by intracellular cytokine staining (ICS). CD4+ T cells transduced with the F24, F25, and F5 TCRs showed abundant cytokine production and degranulation capacity after high dose Gag293 peptide stimulation (FIG. 7A). A specific ICS response was detected in ≥50% CD4+ T cells transduced with F24, while in the same experiment TRBV2 expression increased by only 25% post-transduction (FIG. 12A), pointing to efficient cytokine production even in cells expressing limiting amounts of transduced TCR. ICS responses were then measured in the presence of decreasing Gag293 peptide concentrations (as shown for F24 transduced cells, FIG. 7B). The hierarchy of marker induction was conserved across peptide doses, with high expression of TNF-α and IL-2, intermediate expression of MIP-1β, and more limited expression of CD107a and IFN-γ. Of note, specific expression of these 5 markers persisted until very low peptide doses (10⁻⁹ to 10⁻¹⁰ M). Antigen sensitivity was quantified for the 3 TCRs, by measuring the EC50 for induction of the 5 markers tested (FIG. 7C). This analysis confirmed the functional hierarchy between the TCRs (F24>F25>F5), which followed TCR affinity ranking. F24 conferred a highly sensitive detection of Gag293 in primary cells, with EC50 values comprised between 10⁻⁹ and 10^{-s} M when measuring TNF-α, CD107a, or MIP-11 production. IFN-γ responses appeared comparatively less sensitive, with EC50 values comprised between 10⁻⁸ and 10⁻⁷ M, while IL-2 gave intermediate values. Thus, only TCRs with particularly high affinity, such as F24, may lead to full Th1 differentiation with IFN-γ production in situations of low antigen availability.

Polyfunctionality, or the capacity to express multiple cytokines simultaneously, is a hallmark of Controller T cells, and is thought to provide superior effector functions (17, 23, 39). Quantification of the number of functions (or markers) co-expressed by TCR transduced cells revealed a high degree of polyfunctionality (FIG. 7F). Indeed, the majority (>65%) of F24, F25, and F5 expressing cells could be deemed polyfunctional, as they expressed more than 3 functions after high dose Gag293 stimulation. CD4+ T cells co-expressing the full set of markers (5 functions) were detected until the 10⁻⁸M peptide dose for F24, and until the 10⁻⁷ M peptide dose for F25 and F5. After F24 transfer, polyfunctional cells expressing at least 3 functions still represented more than half of CD4+ T cells responding to 10⁻⁹ M peptide, emphasizing that polyfunctional responses could be achieved in response to minimal amounts of Gag antigen.

Interestingly, CD8+ T cells transduced with the F24 TCR also showed a Gag293-specific cytokine response, with detectable induction of the 5 markers tested (FIGS. 7D and 7E). Cytokine response in CD8+ T cells transduced with F25 and F5 remained low or undetectable (not shown). The F24-dependent response in CD8+ T cells could be blocked by treatment with a pan-HLA-DR antibody, indicating that it was restricted by MHC II (FIG. 13A). Thus, the high-affinity TCR F24 was able to confer a Gag293-specific response in absence of CD4 coreceptor expression, in a situation where availability of the Lck kinase may be limiting. F24 transfer induced 3 functions or more in >25% of specific CD8+ T cells until the 10⁻⁷ M peptide dose (FIG. 13B), indicating that this high-affinity TCR could confer polyfunctional responses to CD8+ T cells. Thus, Controller clonotypes could be used to generate TCRs with uncommon properties, including affinities in the micromolar range, broad HLA-DR cross-restriction, high antigen sensitivity, and polyfunctionality in both the CD4+ and CD8+ T cell subsets.

Expression of the High-Affinity F24 TCR Induces the Formation of Mature Immunological Synapses at High Frequency

We set up a system to measure the formation of conjugates between TCR-transduced J76 T cells and antigen presenting cells (APC) pulsed with the Gag293 peptide. EBV-transformed B lymphoblastoid cell lines (B-EBV) of known HLA DR haplotypes were used APC. Conjugate formation was measured by the percentage of B-EBV/TCR-J76 doublets counted by flow cytometry. The high affinity TCR F24 proved markedly more efficient at inducing conjugate formation with B-EBV, compared to the lower affinity TCRs F25 and F5 (not shown). Analysis by imaging flow cytometry showed that the intensity of CD3 relocalization at the T cell/APC interface also strongly depended on TCR affinity (not shown). Therefore, the high affinity TCR F24 proved more efficient at triggering the formation and the maturation of immunological synapses, a key step required for T cell activation and proliferation.

The F24 TCR Confers Cytotoxic Responses to Both CD4+ and CD8+ T Cells

Interestingly, transduction of the F24 TCR resulted in Gag293-specific cytokine responses in CD8+ T cells. Though responses were less sensitive than in CD4+ T cells, polyfunctionality persisted until the 10-7 M peptide dose. The use of blocking HLA antibodies showed that the CD8+ T cells recognized Gag293 through HLA-DR molecules. Thus, transfer of the F24 TCR could redirect CD8+ T cells to target HIV-1 capsid via nonconventional MHC II restriction. This suggests that the F24 TCR is of sufficiently high affinity to function in absence of the CD4 coreceptor and of the CD4-associated Ick molecules.

We developed a primary cell assay to evaluate the capacity of TCR-transduced T cells to kill HIV-infected autologous dendritic cells (DC) (see FIG. 14). Immature DC were infected with single-cycle HIV-1 pseudotyped virus expressing the GFP reporter gene. The infected DC were then cocultivated with autologous CD4+ T cells that had been transduced with the relevant TCRs (FIG. 14A). The viral suppressive effect was evaluated by computing the decrease in infected GFP+ DC in the presence of CD4+ T cells expressing a Gag293-specific TCR, compared to cocultures in the presence of mock-transduced CD4+ T cells. As shown in FIG. 14C, CD4+ T cells expressing the F24, F25, and F5 TCRs appeared capable of cytotoxicity, as indicated by up to 60% of viral suppression at an E:T ratio of 5. CD4+ T cells expressing the TCR of higher affinity, F24, suppressed HIV infection more efficiently, as indicated by a detectable effect at a low E:T ratio of 0.25. In addition, the F24 TCR conferred cytotoxicity in cells restricted by different HLA-DR alleles, including DR11, DR15/DRB5 (FIG. 14D) and DR1 (not shown). Interestingly, The 3 TCRs also conferred efficient cytotoxicity to CD8+ T cells, as indicated by up to 90% viral suppression (FIG. 14E).

These findings have important immunotherapeutic implications, as they open the possibility to target the capsid major homology region with both CD4+ and CD8+ T cells, which should reinforce the immune response and limit the risks of escape mutations. Accordingly, the inventors' findings disclosed herein indicate that transfer of public TCRs encompassed within the present disclosure would enable both CD4+ and CD8+ T cells to lyze HIV-infected target cells with high efficiency. Given the unique set of properties disclosed herein, controller-derived and especially suitably selected public TCRs as disclosed herein represent promising tools for immunotherapy against HIV, especially for HIV control and/or functional cure.

### Discussion

Exploration of the TCR repertoire specific for the most immunoprevalent CD4 epitope in HIV-1 Gag revealed a markedly biased repertoire focused on the TRAV24 and TRBV2 gene families. Highly prevalent public motifs and public clonotypes were preferentially shared by HIV Controllers, suggesting that particular TCR determinants contributed to the efficiency of the antiviral CD4 response in these patients. This notion was reinforced by functional analysis of the most prevalent public clonotypes, as these were able to confer HLA cross-restricted, highly sensitive, polyfunctional responses against Gag antigens, indicative of superior function. These findings, which to our knowledge provide the first assessment of the HIV-specific CD4+ T cell repertoire at the clonotypic level, emphasize that intrinsic TCR determinants, rather than low antigenemia, determine the remarkable properties of the cellular response in controlled HIV infection.

It was noteworthy that both the TRAV24 and TRBV2 repertoires specific for Gag293 showed a high degree of motif and clonotype sharing. In fact, when the inventors characterized the repertoire of TCRs specific for Gag293, they uncovered an unexpected level of TCR sharing among HIV Controllers, which in turn enabled to envision that the features of particular TCR sequences can be associated with HIV control. So far, most studies of the human TCR repertoire have focused on TRB genes, but recent analyses suggest that the human TRA repertoire is even more diverse (40). Our findings provide evidence for a dual bias in controlled HIV infection, with both TCR chains showing a significant degree of conservation. The most prevalent TRAV24 clonotype (AFKAAGNKLT) was present in 6/8 Controllers (75%) studied. In comparison, CD8 TRB public clonotypes reported in previous studies of the HIV-specific repertoire were shared between 2 to 4 patients (14, 15, 41, 42). When the analysis was extended to less stringent definitions of TCR biases, close to half of Controller TRAV24 and TRBV2 clonotypes fitted defined public motifs, while the corresponding frequencies were of 29% and 10% respectively for clonotypes of treated patients. Such strong bias in the Gag293-specific TCR repertoire of Controllers could not be attributed to amplification of a few clonotypes during culture as the repertoire of Controllers remained highly diverse, with a median of 36 distinct TRBV2 clonotypes per 100 CDR3 sequences. In addition, similar frequencies of public motifs were detected in vitro and ex vivo when the TCR repertoire of Controllers could be analyzed in both conditions. Rather, these findings suggest that strong selective pressures shaped the Gag293-specific repertoire during the establishment of viral control.

Public clonotype occurrence should statistically be an extremely rare event, given the huge number of TCRs that can be generated by V(D)J recombination (>10¹⁵) and the extensive genetic diversity observed in human TCR repertoires, with an estimated >10⁷ distinct clonotypes per individual (43). However, public clonotypes have been described in several chronic viral infections of humans, especially those caused by members of the herpesviridae family (32, 35, 37, 43). CD8 public clonotypes specific for conserved regions of Gag have also been identified in the context of Controlled HIV-1 infection, particularly in patients expressing the protective alleles HLA-B57 and B27 (11, 15, 41), though they can also be detected in patients carrying non-protective HLA-B alleles (42). Recent findings indicate that the frequency of HIV-specific public clonotypes correlates with the magnitude of the CD8 response, suggesting an important contribution to antiviral efficacy (42). Whether CD8 public clonotypes are generally more abundant in controlled rather than progressive HIV infection remains debated (44, 45), but it has become clear that the particular set of CD8 public clonotypes present in Controllers is more efficient at suppressing viral replication and at recognizing viral escape mutants than that of progressor patients (11, 12). A high frequency of Gag-specific CD8 public clonotypes has also been associated with a favorable outcome in the SIVmac model, in primary infection as well as post-vaccination (46). The finding that the Gag-specific CD4 repertoire of Controllers is more biased than the corresponding CD8 repertoire and is enriched in highly efficient public clonotypes supports the notion that public clonotypes contribute to HIV/SIV control. A reason for the advantage conferred by public clonotypes in the antiviral response may lie in their intrinsically high frequency. Analyses of the naive TCR repertoire have shown that public clonotypes, which are by definition shared by several individuals, are also present at significantly higher frequencies than private clonotypes in the naive repertoire of each individual (47). The presence of a larger pool of naive T cells ready to respond to a given pathogen is thought to trigger a more rapid initiation of the immune response, which can provide a key advantage to limit viral dissemination at an early stage. This advantage may be particularly critical in the cases of viruses such as HIV and SIV, which can induce a massive CD4+ T cell depletion and ensuing immunosuppression during the first week of infection (48). In effect, abundant public clonotypes endowed with efficient functions may act as a rapid antiviral response force, similar to populations of innate cells expressing conserved TCRs, such as iNKT or MAIT cells (38, 49).

The high prevalence of Gag293 public clonotypes may result from a high probability of being generated during V(D)J recombination. The most prevalent TRAV24 public clonotype, AFKAAGNKLTF, could be simply generated from germline sequences by limited nucleotide trimming, without requiring N/P mutations. In addition, 72% the 36 Gag293 specific public clonotypes were encoded by more than one nucleotide CDR3 sequence, compatible with the notion of convergent recombination (36). Interestingly, a study of the preimmune CD4+ T cell repertoire in healthy individuals identified several clones reactive with HIV antigens, including one that responded to Gag293 with high antigen sensitivity (50), supporting the idea that high-avidity Gag293-specific precursors may be comparatively abundant in the naive repertoire. Another reason for the high prevalence of Gag293-specific public clonotypes may lie in their extensive HLA cross-restriction. The Gag293 peptide shows promiscuous binding to at least 14 HLA-DR and DQ allomorphs, enabling multiple possibilities for cross-restriction (30, 51, 52). Functional analyses indicated that the F24 TCR bound at least 5 distinct Gag293-MHC II complexes, with an efficient recognition of DR1, DR11, DR15, DRB5, and a limited but consistent recognition of DR7. The correlation between the degree of cross-restriction and the antigen sensitivity of the 8 TCRs tested suggests that broad HLA cross-restriction is a characteristic of high-affinity TCRs, which may better tolerate punctual MHC polymorphisms. This notion is supported by a study of human CD4 responses to JC virus, where HLA II cross-restricted clones showed higher antigen sensitivity and greater in vivo expansion than clones with a more narrow restriction (53). Widespread cross-restriction may help explain why HLA II allele frequencies are not strongly biased in carriers of chronic viral infections. HIV Controllers show a highly significant enrichment for protective HLA I alleles, while biases in HLA II allele distribution have been reported but appear less prominent (23, 52, 54). Considering the evidence for broad HLA II cross-restriction of HIV-specific public TCRs, the limited HLA II biases in the Controller population does not rule out a significant contribution of CD4+ T cells to HIV control.

The co-expression of TRAV24 and TRBV2 public clonotypes generated functional TCRs that, for some combinations, displayed a remarkably high affinity for Gag293-MHC II complexes. Human TCRs generally show affinities in the 1 to 100 μM range by SPR analysis (38). MHC II restricted TCRs show a trend for a lower affinity as compared to MHC I restricted TCRs, with average Kd values of 70 versus 35 μM, respectively. With a Kd of 0.86 μM for the Gag293-DR11 complex, F24 demonstrated one of the highest affinities reported so far for an MHC II restricted TCR, ranking second in a list of 22 human and mouse TCRs for which structural data is available (38). The molecular determinants of a high avidity interaction for the Gag293-MHC complex appear to depend on both the TRAV24 and TRBV2 chains, and on the restricting HLA allele. The most conserved CDR3 residues in public motifs represent likely candidates for contact residues with Gag293. However, conserved CDR1/CDR2 residues may also contact the Gag293 peptide, considering the predominance of the TRAV24 and TRBV2 families. Future structural studies should help determine the respective roles of the CDR3 versus the CDR1/2 regions in shaping high-avidity interactions for Gag293-MHC complexes. The expression of high-affinity TCRs for Gag293 in Controllers resulted in high antigen sensitivity as measured by CD69 induction, with a good correlation between the two parameters (R=0.85). Thus, TCR biophysical properties largely accounted for TCR function, consistent with multiple studies in animal models (55, 56). It should be noted, however, that in some studies TCRs with very high or supraphysiological affinities were found to have reduced functions (57). A proposed explanation is that a pMHC that interacts too tightly with a very high affinity TCR cannot disengage to contact additional TCRs, and thus does not achieve serial TCR triggering and T cell response amplification (58). We did not observe decreased functions for the TCR/pMHC II combinations showing the highest affinities by SPR, suggesting that TCR affinities in the order of 1 μM are still in the physiological range for optimal TCR function in human CD4+ T cells.

The reasons for the preferential amplification of high-affinity Gag293-specific TCRs in Controllers compared to treated patients remain to be elucidated. A particular genetic background or a limited duration of acute infection may have promoted the amplification of such TCRs in Controllers (59). Conversely, patients who initially progressed to disease may have lost the high avidity CD4+ T cell population due to preferential infection and depletion, a notion supported by the restricted CD4 TCR repertoire observed in treated patients, and by rapid loss of certain CD4 specificities after the acute infection stage (6). It will be informative to determine whether other immunoprevalent HIV epitopes elicit a response pattern similar to that observed for Gag293, with the induction of a highly biased, high affinity TCR repertoire in controlled infection. The preferential detection of Gag-specific CD4 responses in HIV Controllers (22) suggests that several regions of Gag may be targeted by high affinity TCRs, though this remains to be investigated. High-affinity CD4 TCRs are endowed with strong proliferative capacity (55), which helps explain how they could reach such a dominance in controlled HIV infection. The high affinity of Controller TCRs also translated into efficient cytokine secretion upon Gag293 peptide stimulation. Primary CD4+ T cells transduced with the F24 TCR showed EC50 ranging between 10⁻⁸ M and 10⁻⁹ M peptide for the different cytokines studied, indicative of a remarkably high antigen sensitivity for the CD4+ T cell subset. Polyfunctionality, a property which has been associated with HIV control (39), showed a clear dependence on TCR affinity. Comparison of the F24, F25, and F5 TCRs showed that a difference of 1.1 log in affinity resulted in a 2 log difference in the peptide concentration at which half the specific cells retained 3 functions. Thus, TCR affinity appears critical in determining the range of cytokine produced, particularly at low antigen dose. Of interest, not all cytokines were equally dependent on the strength of the TCR signal, with TNF-α showing the lowest requirements, and IFN-γ the most stringent. The notion of a hierarchy in cytokine production is consistent with a report showing that the affinity of Gag-specific T cell clones dictated their cytokine expression profile (10). It is relevant that for CD4+ T cells, in contrast to CD8+ T cells, IFN-γ is the most “demanding” cytokine, as it can be produced only by high avidity cells upon limiting antigenic stimulation. IFN-γ plays a key role in CD4+T cell helper function, through its capacity to upregulate MHC II in APC and prime them for efficient antigen presentation. In addition, IFN-γ production by tissue CD4+ T cells plays an underappreciated role in the recruitment of immune effectors, through the triggering of chemokine cascades (60). In controlled HIV infection, high avidity Gag-specific CD4+ T cells may play a similar role, by keeping the immune system in constant alert, and rapidly recruiting CD8+ T cells and NK cells to sites of HIV replication upon the occurrence of viral replication blips. In addition, IFN-γ secretion may play a direct antiviral role, through the induction of interferon stimulated genes (ISG) that inhibit HIV replication (61). For instance, Th1 cells are thought to be less infectable than Th2 cells due to a higher expression of the HIV restriction factor APOBEC3G (62). Highly differentiated Th1 effectors that re-express CD45RA appear particularly resistant to HIV infection (63). Gag-specific CD4+ T cells in Controllers maintain a Th1 differentiation status with persistent IFN-γ production (24), raising the possibility that such cells possess a degree of resistance to HIV infection. Thus, high TCR affinity, with the associated capacity for IFN-γ expression at low antigen dose, may contribute to HIV control at several levels, through sensitive immune surveillance, rapid triggering of helper functions, and direct antiviral effector mechanisms.

As a proof of concept, the functionality of the most prevalent public clonotype identified by the inventors was tested by TCR transfer in vitro. The most prevalent TRAV24 clonotype, found in 6/8 Controllers and 2/8 treated patients, was paired with a public TRVB2 chain in a bicistronic lentiviral vector, and the resulting TCR (F24) was transferred to heterologous T cells by lentiviral transduction. Interestingly, the F24 TCR was able to recognize the Gag293 peptide presented in the context of multiple MHC II alleles. This broad MHC II cross-restriction helped explain how TRAV24 and TRBV2 public clonotypes could be shared by Controllers expressing diverse MHC II genotypes. The transfer of the F24 TCR was sufficient to confer high antigen sensitivity to primary CD4+ T cells, as indicated an EC50<10-8 M peptide for cytokine induction (FIG. 14B). These responses showed a high degree of polyfunctionality (i.e. the capacity to secrete multiple cytokines simultaneously), demonstrating that the TCR is a key determinant of the functional properties characteristic of Controller CD4+ T cells. In addition, when measure by surface plasmon resonance, TCR affinity tightly correlated with antigen sensitivity (R=0.85, P=0.16),

Of interest, the F24 TCR was also able to confer polyfunctional cytokine responses when transferred into CD8+ T cells. The responses were MHC II restricted, indicating that the F24 TCR could interact with the Gag293-MHC II complex in the absence of the CD4 coreceptor. The CD4 molecule is not thought to contribute significantly to the affinity of the pMHC/TCR/CD4 complex, but plays an important role in relocating the kinase Lck close to TCR/CD3 signaling complex (64). The comparatively lower cytokine responses in CD8+ than in CD4+ T cells may thus result from a lower number of Lck molecules available for triggering intracellular signals. The transfer of the F24 TCR to CD8+ T cells still conferred the 5 functions tested at the 10⁻⁵ M to 10⁻⁷ M antigen dose, suggesting that transduced CD8+ T cells could become efficient effectors in foci of productive HIV replication. These findings open the possibility of reprogramming CD8+ T cells to target the highly conserved MHR region of capsid, which could be advantageous given the high fitness cost associated with mutations in this region (65). The report that a CMV-based protective SIV vaccine elicited a high frequency of unconventional MHC II-restricted CD8 responses highlights the potential benefits of CD8 T cell reprogramming (66). Studies of TCR transfer for cancer immunotherapy have shown the interest of transferring the same TCR in both CD4+ and CD8+ T cells to enhance tumoricidal activity (67). Similarly, the transfer of a high-avidity Gag-specific TCR in both CD4+ and CD8+ T cell populations may be of interest for adoptive T cell therapies targeting HIV, in order to trigger a full set of antiviral functions.

In conclusion, study of TCRs specific for the immunoprevalent CD4 epitope in capsid revealed that particular clonotypes are associated with HIV control. The TCR repertoire of Controllers was characterized by a high prevalence of public TRAV24 and TRBV2 chains. Reconstituted TCRs showed affinities that reached the micromolar range, at the high end of values obtained for naturally expressed TCRs. Public clonotypes conferred MHC II cross-restriction, high antigen sensitivity and polyfunctionality to CD4+ T cells, suggesting a key role in shaping the properties of an efficient CD4 response. The most prevalent public clonotype also proved functional in CD8+ T cells, suggesting that it could be used to target the highly conserved capsid MHR region in patients of diverse HLA types. Inducing or transferring such clonotypes may contribute to the development of immunotherapeutic approaches that aim at a functional cure of HIV infection.

TABLE 6
List of TRAV24 clonotypes specific for Gag293 (SEQ ID NO: 198 to 314 for Junction (AA)
sequences and SEQ ID NO: 315 to 454 for Junction (nt) sequences

| | | | | | % nt | % AA | JUNCTION |
|---|---|---|---|---|---|---|---|
| V-GENE | J-GENE | JUNCTION | JUNCTION (AA) | frequency | seq | seq | nt number |
| HIC 1 DR15+ | | | | | | | |
| TRAV24*01, or | TRAJ38*01 | tgtgcccgtgacgaccgtaagctgatttgg | CARDDRKLIW | 1 | 1.89 | 1.89 | 30 |
| TRAV24*02 | | | | | | | |
| TRAV24*01 | TRAJ34*01 | tgtgcctctggtaacaccgacaagctcatcttt | CASGNTDKLIF | 1 | 1.89 | 1.89 | 33 |
| TRAV24*01 | TRAJ39*01 | tgtgccttttgtaatgcaggcaacatgctcaccttt | CAFCNAGNMLTF | 1 | 1.89 | 1.89 | 36 |
| TRAV24*01 | TRAJ17*01 | tgtgcctttaaagctgcaggcaacaagctaactttt | CAFKAAGNKLTF* | 2 | 3.77 | 5.66 | |
| TRAV24*01 | TRAJ17*01 | tgtgcctttaaggctgcaggcaacaagctaactttt | | 1 | 1.89 | | |
| TRAV24*01 | TRAJ17*01 | tgtgcctttacggctgcaggcaacaagctaactttt | CAFTAAGNKLTF | 1 | 1.89 | 1.89 | |
| TRAV24*01 | TRAJ17*01 | tgtgccctagaaaatgcaggcaacaagctaactttt | CALENAGNKLTF | 1 | 1.89 | 1.89 | |
| TRAV24*01 | TRAJ39*01 | tgtgccctcggtaatgcaggcaacatgctcaccttt | CALGNAGNMLTF | 33 | 62.26 | 64.15 | |
| TRAV24*01 | TRAJ39*01 | tgtgccctcggtaatgcaggcaacatgctcacgttt | | 1 | 1.89 | | |
| TRAV24*01 | TRAJ57*01 | tgctcgcctcagggcggatctgaaaagctggtcttt | CSPQGGSEKLVF | 1 | 1.89 | 1.89 | |
| TRAV24*01 | TRAJ6*01 | tgtgcctttatcccaggaggaagctacatacctacattt | CAFIPGGSYIPTF | 1 | 1.89 | 1.89 | 39 |
| TRAV24*01 | TRAJ42*01 | tgtgcctctgatggaggaagccaaggcaatctcatcttt | CASDGGSQGNLIF | 1 | 1.89 | 1.89 | |
| TRAV24*01 | TRAJ32*02 | tgtgcctcttatggtggtgctacaaacaagctcatcttt | CASYGGATNKLIF | 8 | 15.09 | 15.09 | |
| | | Total | 11 | 53 | 100.00 | 100.00 | |
| HIC 2 DRB5+ | | | | | | | |
| TRAV24*01 | TRAJ17*01 | tgtgccttcaaagctgcaggcaacaagctaactttt | CAFKAAGNKLTF* | 2 | 3.03 | 56.06 | 36 |
| TRAV24*01 | TRAJ17*01 | tgtgcctttaaagctgcaggcaacaagctaactttt | | 35 | 53.03 | | |
| TRAV24*01 | TRAJ17*01 | tgtgcctttaaggttgcaggcaacaagctaactttt | CAFKVAGNKLTF | 1 | 1.52 | 1.52 | |
| TRAV24*01 | TRAJ39*01 | tgtgcctttaggatggcaggcaacatgctcaccttt | CAFRMAGNMLTF | 1 | 1.52 | 1.52 | |
| TRAV24*01 | TRAJ39*01 | tgtgcctttcgtaatgcaggcaacatgctcaccttt | CAFRNAGNMLTF | 1 | 1.52 | 1.52 | |
| TRAV24*01 | TRAJ17*01 | tgtgcccacaaagctgcaggcaacaagctaactttt | CAHKAAGNKLTF | 2 | 3.03 | 4.55 | |
| TRAV24*01 | TRAJ17*01 | tgtgcccataaagctgcaggcaacaagctaactttt | | 1 | 1.52 | | |
| TRAV24*01 | TRAJ39*01 | tgtgcccctataaatgcaggcaacatgctcaccttt | CAPINAGNMLTF | 6 | 9.09 | 9.09 | |
| TRAV24*01 | TRAJ17*01 | tgtgcccgcaaagctgcaggcaacaagctaactttt | CARKAAGNKLTF | 1 | 1.52 | 1.52 | |
| TRAV24*01 | TRAJ17*01 | tgtgcctctaaagctgcaggcaacaagctaactttt | CASKAAGNKLTF* | 13 | 19.70 | 19.70 | |
| TRAV24*01 | TRAJ17*01 | tgtgcctcacgaactgcaggcaacaagctaactttt | CASRTAGNKLTF | 3 | 4.55 | 4.55 | |
| | | Total | 9 | 66 | 100.00 | 100.00 | |
| HIC 3 DR11+ | | | | | | | |
| TRAV24*01 | TRAJ17*01 | tgtgctccaaagagcaggcaacaagctaacttt | CAPKSRQQANF | 1 | 0.64 | 0.64 | 33 |
| TRAV24*01 | TRAJ17*01 | tgtgccttcaaagctgcaggcaacaagctaactttt | CAFKAAGNKLTF* | 64 | 41.03 | 46.15 | 36 |
| TRAV24*01, or | TRAJ17*01 | tgtgcctttaaagctgcaggcaacaagctaactttt | | 8 | 5.13 | | |
| TRAV24*02 | | | | | | | |
| TRAV24*01, or | TRAJ17*01 | tgtgccttcagggctgcaggcaacaagctaactttt | CAFRAAGNKLTF | 2 | 1.28 | 1.28 | |
| TRAV24*02 | | | | | | | |
| TRAV24*01, or | TRAJ17*01 | tgtgccttgaaagctgcaggcaacaagctaactttt | CALKAAGNKLTF | 1 | 0.64 | 0.64 | |
| TRAV24*02 | | | | | | | |
| TRAV24*01 | TRAJ39*01 | tgtgccttgaaacaagcaggcaacaagctcactttt | CALKQAGNKLTF | 1 | 0.64 | 0.64 | |
| TRAV24*01 | TRAJ39*01 | tgtgccttgaggcaagcaggcaacatgctcaccttt | CALRQAGNMLTF | 6 | 3.85 | 3.85 | |
| TRAV24*01, or | TRAJ17*01 | tgtgccatgaaagctgcaggcaacaagctaactttt | CAMKAAGNKLTF | 1 | 0.64 | 1.28 | |
| TRAV24*02 | | | | | | | |
| TRAV24*01, or | TRAJ17*01 | tgtgcgatgaaagctgcaggcaacaagctaactttt | | 1 | 0.64 | | |
| TRAV24*02 | | | | | | | |
| TRAV24*01, or | TRAJ17*01 | tgtgccaacagagctgcaggcaacaagctaactttt | CANRAAGNKLTF | 1 | 0.64 | 0.64 | |
| TRAV24*02 | | | | | | | |
| TRAV24*01, or | TRAJ17*01 | tgtgcctccaaagctgcaggcaacaagctaactttt | CASKAAGNKLTF* | 4 | 2.56 | 2.56 | |
| TRAV24*02 | | | | | | | |
| TRAV24*01, or | TRAJ17*01 | tgtgcctacaaagctgcaggcaacaagctaactttt | CAYKAAGNKLTF | 1 | 0.64 | 0.64 | |
| TRAV24*02 | | | | | | | |
| TRAV24*01, or | TRAJ17*01 | tgtggtttcaaagctgcaggcaacaagctaactttt | CGFKAAGNKLTF | 1 | 0.64 | 0.64 | |
| TRAV24*02 | | | | | | | |
| TRAV24*01, or | TRAJ17*01 | tgtggttggaaagctgcaggcaacaagctaactttt | CGWKAAGNKLTF | 1 | 0.64 | 0.64 | |
| TRAV24*02 | | | | | | | |
| TRAV24*01, or | TRAJ17*01 | tgtagtttgaaagctgcaggcaacaagctaactttt | CSLKAAGNKLTF | 2 | 1.28 | 1.28 | |
| TRAV24*02 | | | | | | | |
| TRAV24*01, or | TRAJ17*01 | tgtagtttgagagctgcaggcaacaagctaactttt | CSLRAAGNKLTF | 1 | 0.64 | 0.64 | |
| TRAV24*02 | | | | | | | |
| TRAV24*01 | TRAJ17*01 | tgtagtcggagggctgcaggcaacaagctaactttt | CSRRAAGNKLTF* | 18 | 11.54 | 11.54 | |
| TRAV24*01, or | TRAJ17*01 | tgtagttggaaagctgcaggcaacaagctaactttt | CSWKAAGNKLTF | 4 | 2.56 | 2.56 | |
| TRAV24*02 | | | | | | | |
| TRAV24*01, or | TRAJ17*01 | tgtagttggagagctgcaggcaacaagctaactttt | CSWRAAGNKLTF | 1 | 0.64 | 1.28 | |
| TRAV24*02 | | | | | | | |
| TRAV24*01, or | TRAJ17*01 | tgtagttggagggctgcaggcaacaagctaactttt | | 1 | 0.64 | | |
| TRAV24*02 | | | | | | | |
| TRAV24*01, or | TRAJ17*01 | tgtaccaagaaagctgcaggcaacaagctaactttt | CTKKAAGNKLTF | 1 | 0.64 | 21.15 | |
| TRAV24*02 | | | | | | | |
| TRAV24*01 | TRAJ17*01 | tgtacgaagaaagctgcaggcaacaagctaactttt | | 32 | 20.51 | | |
| TRAV24*01, or | TRAJ17*01 | tgtaccttgaaagctgcaggcaacaagctaactttt | CTLKAAGNKLTF | 1 | 0.64 | 0.64 | |
| TRAV24*02 | | | | | | | |
| TRAV24*01, or | TRAJ17*01 | tgtacgaacaaagctgcaggcaacaagctaactttt | CTNKAAGNKLTF | 1 | 0.64 | 0.64 | |
| TRAV24*02 | | | | | | | |
| TRAV24*01, or | TRAJ17*01 | tgtactaggaaagctgcaggcaacaagctaactttt | CTRKAAGNKLTF | 1 | 0.64 | 0.64 | |
| TRAV24*02 | | | | | | | |
| | | Total | 22 | 156 | 100.00 | 100.00 | |
| HIC 4 DR15+ | | | | | | | |
| TRAV24*01 | TRAJ39*01 | tgtgccttccaaaatgcaggcaacatgctcaccttt | CAFQNAGNMLTF | 6 | 9.23 | 9.23 | 36 |
| TRAV24*01 | TRAJ17*01 | tgtgcctttagagctgcaggcaacaagctaactttt | CAFRAAGNKLTF | 1 | 1.54 | 1.54 | |
| TRAV24*01 | TRAJ39*01 | tgtgcgacgcgccgtgcaggcaacatgctcaccttt | CATRRAGNMLTF | 6 | 9.23 | 9.23 | |
| TRAV24*01 | TRAJ57*01 | tgtgcctacgagggggcatctgaaaagctggtcttt | CAYEGASEKLVF | 3 | 4.62 | 4.62 | |
| TRAV24*01 | TRAJ32*02 | tgtgccgaatatggtggtgctacaaacaagctcatcttt | CAEYGGATNKLIF | 2 | 3.08 | 3.08 | 39 |
| TRAV24*01 | TRAJ32*02 | tgtgccccttatggtggtgctacaaacaagctcatcttt | CAPYGGATNKLIF | 2 | 3.08 | 3.08 | |
| TRAV24*01 | TRAJ32*02 | tgtgcccgttatggtggtgctgcaaacaagctcatcttt | CARYGGAANKLIF | 1 | 1.54 | 1.54 | |
| TRAV24*01 | TRAJ32*02 | tgtgcccgttatggtggtgctacaaacaagctcatcttt | CARYGGATNKLIF | 10 | 15.38 | 16.92 | |
| TRAV24*01 | TRAJ32*02 | tgtgctcgttatggtggtgctacaaacaagctcatcttt | | 1 | 1.54 | | |
| TRAV24*01, or | TRAJ32*02 | tgtgcccgctatggtggtggtacaaacaagctcattttt | CARYGGGTNKLIF | 1 | 1.54 | 1.54 | |
| TRAV24*02 | | | | | | | |
| TRAV24*01 | TRAJ54*01 | tgtgcctccgagtccacgggagcccagaagctggtattt | CASESTGAQKLVF | 3 | 4.62 | 4.62 | |
| TRAV24*01 | TRAJ32*02 | tgtgcctcctatggtggtgctacaaacaagctcatcttt | CASYGGATNKLIF | 25 | 38.46 | 43.08 | |
| TRAV24*01 | TRAJ32*02 | tgtgcctcgtatggtggtgctacaaacaagctcatcttt | | 2 | 3.08 | | |
| TRAV24*01 | TRAJ32*02 | tgtgcctcttatggtggtgctacaaacaagctcatcttt | | 1 | 1.54 | | |
| TRAV24*01 | TRAJ32*02 | tgtgcctcctatgttggtgctacaaacaagctcatcttt | CASYVGATNKLIF | 1 | 1.54 | 1.54 | |
| | | Total | 12 | 65 | 100.00 | 100.00 | |
| HIC 5 DR1+ | | | | | | | |
| TRAV24*02 | TRAJ38*01 | tgtgccttcgacaaccgtaagctgatttgg | CAFDNRKLIW | 31 | 51.67 | 55.00 | 30 |
| TRAV24*01 | TRAJ38*01 | tgtgcctttgacaaccgtaagctgatttgg | | 2 | 3.33 | | |
| TRAV24*01 | TRAJ17*01 | tgtgcctttaaggctgcaggcaacaagctaactttt | CAFKAAGNKLTF* | 1 | 1.67 | 1.67 | 36 |
| TRAV24*01 | TRAJ17*01 | tgtgcctttcgagctgcaggcaacaagctaactttt | CAFRAAGNKLTF | 2 | 3.33 | 3.33 | |
| TRAV24*01 | TRAJ36*01 | tgtgcctcagaaactggggcaaacaacctcttcttt | CASETGANNLFF | 2 | 3.33 | 3.33 | |
| TRAV24*02 | TRAJ39*01 | tgtgcgacgcgccgtgcaggcaacatgctcaccttt | CATRRAGNMLTF | 5 | 8.33 | 8.33 | |
| TRAV24*01 | TRAJ57*01 | tgtgcctacgagggggcatctgaaaagctggtcttt | CAYEGASEKLVF | 1 | 1.67 | 1.67 | |
| TRAV24*01 | TRAJ32*02 | tgtgccgaatatggtggtgctacaaacaagctcatcttt | CAEYGGATNKLIF | 1 | 1.67 | 1.67 | 39 |
| TRAV24*01 | TRAJ22*01 | tgtgccttcgcttctggttctgcaaggcaactgaccttt | CAFASGSARQLTF | 13 | 21.67 | 21.67 | |
| TRAV24*01 | TRAJ54*01 | tgtgcctccgagtccacgggagcccagaagctggtattt | CASESTGAQKLVF | 1 | 1.67 | 1.67 | |
| TRAV24*02 | TRAJ38*01 | tgtgccttttacccccctggcaacaaccgtaagctgatttgg | CAFYPPGNNRKLIW | 1 | 1.67 | 1.67 | 42 |
| | | Total | 10 | 60 | 100.00 | 100.00 | |
| HIC 6 DR11+ | | | | | | | |
| TRAV24*01 | TRAJ17*01 | tgtgcatttaaagctgcaggcaacaagctaactttt | CAFKAAGNKLTF* | 1 | 1.69 | 23.73 | 36 |
| TRAV24*01 | TRAJ17*01 | tgtgccttcaaagctgcaggcaacaagctaactttt | | 2 | 3.39 | | |
| TRAV24*01 | TRAJ17*01 | tgtgcctttaaagctgcaggcaacaagctaactttt | | 11 | 18.64 | | |
| TRAV24*01 | TRAJ17*01 | tgtgcctttaaagatgcaggcaacaagctaactttt | CAFKDAGNKLTF | 1 | 1.69 | 1.69 | |
| TRAV24*01 | TRAJ39*01 | tgtgcctttcctaatgcaggcaacatgctcaccttt | CAFPNAGNMLTF | 5 | 8.47 | 8.47 | |
| TRAV24*01 | TRAJ17*01 | tgtgcctttagggctgcaggcaacaagctaactttt | CAFRAAGNKLTF | 1 | 1.69 | 1.69 | |
| TRAV24*01 | TRAJ39*01 | tgtgcctttcggaatgcaggcaacatgctcaccttt | CAFRNAGNMLTF | 1 | 1.69 | 1.69 | |
| TRAV24*01 | TRAJ17*01 | tgtgctctaaaagctgcaggcaacaagctaactttt | CALKAAGNKLTF | 6 | 10.17 | 10.17 | |
| TRAV24*01 | TRAJ17*01 | tgtgctctaaaagatgcaggcaacaagctaactttt | CALKDAGNKLTF | 1 | 1.69 | 1.69 | |
| TRAV24*01 | TRAJ39*01 | tgtgccttaaaaaatgcaggcaacatgctcaccttt | CALKNAGNMLTF | 4 | 6.78 | 6.78 | |
| TRAV24*01 | TRAJ39*01 | tgtgccctcaataatgcaggcaacatgctcaccttt | CALNNAGNMLTF | 1 | 1.69 | 1.69 | |
| TRAV24*01 | TRAJ39*01 | tgtgccctgcggaatgcaggcaacatgctcaccttt | CALRNAGNMLTF | 3 | 5.08 | 5.08 | |
| TRAV24*01 | TRAJ17*01 | tgtgcccccaaagctgcaggcaacaagctaactttt | CAPKAAGNKLTF | 12 | 20.34 | 20.34 | |
| TRAV24*01 | TRAJ17*01 | tgtgcccccaaagatgcaggcaacaagctaactttt | CAPKDAGNKLTF | 1 | 1.69 | 1.69 | |
| TRAV24*01 | TRAJ39*01 | tgtgcctccaagaatgcaggcaacatgctcaccttt | CASKNAGNMLTF | 1 | 1.69 | 1.69 | |
| TRAV24*01 | TRAJ39*01 | tgtgcctccatgaatgcaggcaacatgctcaccttt | CASMNAGNMLTF | 7 | 11.86 | 11.86 | |
| TRAV24*01 | TRAJ53*01 | tgtgcctttaagggaggtggaggtagcaactataaactgacattt | CAFKGGGGSNYKLTF | 1 | 1.69 | 1.69 | 45 |
| | | Total | 15 | 59 | 100.00 | 100.00 | |
| HIC 7 DRB5+ | | | | | | | |
| TRAV24*01 | TRAJ20*01 | tgtgcctttggcgactacaagctcagcttt | CAFGDYKLSF | 2 | 3.64 | 3.64 | 30 |
| TRAV24*01 | TRAJ17*01 | tgtgcctttcacgctgcaggcaacaagctaactttt | CAFHAAGNKLTF | 1 | 1.82 | 1.82 | 36 |
| TRAV24*01 | TRAJ17*01 | tgtgccttcaaagctgcaggcaacaagctaactttt | CAFKAAGNKLTF* | 10 | 18.18 | 18.18 | |
| TRAV24*01 | TRAJ10*01 | tgtgcctttaagggaggaggaaacaaactcaccttt | CAFKGGGNKLTF | 1 | 1.82 | 1.82 | |
| TRAV24*01 | TRAJ17*01 | tgtgcctttaacgctgcaggcaacaagctaactttt | CAFNAAGNKLTF | 3 | 5.45 | 5.45 | |
| TRAV24*01 | TRAJ17*01 | tgtgccttccaggctgcaggcaacaagctaactttt | CAFQAAGNKLTF | 9 | 16.36 | 16.36 | |
| TRAV24*01 | TRAJ10*01 | tgtgcctttaggggaggaggaaacaaactcaccttt | CAFRGGGNKLTF | 11 | 20.00 | 20.00 | |
| TRAV24*01 | TRAJ39*01 | tgtgcctttcgaagagcaggcaacatgctcaccttt | CAFRRAGNMLTF | 1 | 1.82 | 1.82 | |
| TRAV24*01 | TRAJ17*01 | tgtgcccacaaagctgcaggcaacaagctaactttt | CAHKAAGNKLTF | 3 | 5.45 | 5.45 | |
| TRAV24*01 | TRAJ17*01 | tgtgcccacaaaggagcaggcaacaagctaactttt | CAHKGAGNKLTF | 1 | 1.82 | 1.82 | |
| TRAV24*01 | TRAJ32*02 | tgtgccgaatatggtggtgctacaaacaagctcatcttt | CAEYGGATNKLIF | 3 | 5.45 | 5.45 | 39 |
| TRAV24*01 | TRAJ32*01 | tgtgccaactatggcggtgctacaaacaagctcatcttt | CANYGGATNKLIF | 2 | 3.64 | 3.64 | |
| TRAV24*01 | TRAJ57*01 | tgtgcccccggggggggcggatctgaaaagctggtcttt | CAPGGGGSEKLVF | 2 | 3.64 | 3.64 | |
| TRAV24*01 | TRAJ32*02 | tgtgccccttatggtggtgctacaaacaagctcatcttt | CAPYGGATNKLIF | 1 | 1.82 | 3.64 | |
| TRAV24*01 | TRAJ32*01 | tgtgctccttatggcggtgctacaaacaagctcatcttt | | 1 | 1.82 | | |
| TRAV24*01 | TRAJ32*01 | tgtgcctcctatggcggtgctacaaacaagctcatcttt | CASYGGATNKLIF | 2 | 3.64 | 7.27 | |
| TRAV24*01 | TRAJ32*02 | tgtgcctcctatggtggtgctacaaacaagctcatcttt | | 1 | 1.82 | | |
| TRAV24*01 | TRAJ32*02 | tgtgcctcttatggtggtgctacaaacaagctcatcttt | | 1 | 1.82 | | |
| | | Total | 15 | 55 | 100.00 | 100.00 | |
| HIC 8 DR1+ | | | | | | | |
| TRAV24*01 | TRAJ31*01 | tgtgggggggataacaatgccagactcatgttt | CGGDNNARLMF | 54 | 77.14 | 77.14 | 33 |
| TRAV24*01 | TRAJ31*01 | tgtgggggggataacaatgccagactcacgttt | CGGDNNARLTF | 1 | 1.43 | 1.43 | |
| TRAV24*01 | TRAJ34*01 | tgtgcctctctctataacaccgacaagctcatcttt | CASLYNTDKLIF | 10 | 14.29 | 14.29 | 36 |
| TRAV24*01 | TRAJ32*01 | tgtgccaactatggcggtgctacaaacaagctcatcttt | CANYGGATNKLIF | 5 | 7.14 | 7.14 | 39 |
| | | Total | 4 | 70 | 100.00 | 100.00 | |

TRAV24 clonotypes obtained from 8 Controller cell lines, 8 treated patient cell lines, and 4 ex vivo Controller samples are listed.
The V(D)J gene nomenclature is that of the IMGT database (www.imgt.org).
Public clonotypes are in bold type.
Clonotypes tested functionally are marked by an asterisk.

TABLE 7
List of TRBV2 clonotypes specific for Gag293 (SEQ ID NO: 455 to 749 for Junction (AA)
sequences and SEQ ID NO: 750 to 1059 for Junction (nt) sequences
TRBV2 clonotypes obtained from 8 Controller cell lines, 8 treated patient cell lines,
and 4 ex vivo Controller samples are listed. The V(D)J gene nomenclature is that of the
IMGT database (www.imgt.org). Public clonotypes are in bold type. Clonotypes tested
functionally are marked by an asterisk.

| | | | | | fre- | % nt | % AA | nt |
|---|---|---|---|---|---|---|---|---|
| V-GENE | J-GENE | D-GENE | JUNCTION | JUNCTION (AA) | quency | seq | seq | number |
| HIC 1 DR15+ | | | | | | | | |
| TRBV2*01 | TRBJ2-7*01 | TRBD1*01 | tgtgccagcttagggcccctacggcacgagcagtacttc | CASLGPLRHEQYF | 2 | 3.33 | 3.33 | 39 |
| TRBV2*01 | TRBJ2-3*01 | TRBD2*01 | tgtgccagcaaaccactagttagcacagatacgcagtatttt | CASKPLVSTDTQYF | 1 | 1.67 | 1.67 | 42 |
| TRBV2*01, or | TRBJ2-1*01 | TRBD2*01 | tgtgccagccttgaaaggactagcgggggtgagcagttcttc | CASLERTSGGEQFF | 1 | 1.67 | 1.67 | |
| TRBV2*02 or | | | | | | | | |
| TRBV2*03 | | | | | | | | |
| TRBV2*01 | TRBJ2-1*01 | TRBD1*01 | tgtgccagcacccgggacaggacaaagaatgagcagttcttc | CASTRDRTKNEQFF | 1 | 1.67 | 1.67 | 45 |
| TRBV2*01 | TRBJ2-1*01 | TRBD2*01 | tgtgccagcagtgcgttggctagcgggggagatgagcagttcttc | CASSALASGGDEQFF | 1 | 1.67 | 1.67 | |
| TRBV2*01 | TRBJ2-3*01 | TRBD2*01 | tgtgccagcagtgcgttagctagcggtacagatacgcagtatttt | CASSALASGTDTQYF | 1 | 1.67 | 1.67 | |
| TRBV2*01 | TRBJ2-7*01 | TRBD2*01 | tgtgccagcagtgaactgacctctagaacctacgagcagtacttc | CASSELTSRTYEQYF | 1 | 1.67 | 1.67 | |
| TRBV2*01, or | TRBJ1-5*01 | TRBD2*01 | tgtgccagcagtgaacgggtttcgggcaatcagccccagcatttt | CASSERVSGNQPQHF | 2 | 3.33 | 3.33 | |
| TRBV2*02 or | | | | | | | | |
| TRBV2*03 | | | | | | | | |
| TRBV2*01 | TRBJ2-1*01 | TRBD2*01 | tgtgccagcagccctatggctagcgggggggatgagcagttcttc | CASSPMASGGDEQFF | 1 | 1.67 | 1.67 | |
| TRBV2*01, or | TRBJ2-7*01 | TRBD2*02 | tgtgccagcagccgtaggactagcgggacttacgagcagtacttc | CASSRRTSGTYEQYF | 1 | 1.67 | 1.67 | |
| TRBV2*02 or | | | | | | | | |
| TRBV2*03 | | | | | | | | |
| TRBV2*01 | TRBJ2-1*01 | TRBD2*01 | tgtgccagcagtgtgatggctagccgtgggaatgagcagttcttc | CASSVMASRGNEQFF | 1 | 1.67 | 1.67 | |
| TRBV2*01 | TRBJ1-5*01 | TRBD2*01 | tgtgccagccaaaggggggctcgggggggcaatcagccccagcatttt | CASQRGARGGNQPQHF | 1 | 1.67 | 1.67 | 48 |
| TRBV2*01, or | TRBJ1-4*01 | TRBD1*01 | tgtgccagcagggctcgaacaggggcaactaatgaaaaactgtttttt | CASRARTGATNEKLFF | 1 | 1.67 | 1.67 | |
| TRBV2*02 or | | | | | | | | |
| TRBV2*03 | | | | | | | | |
| TRBV2*01 | TRBJ2-3*01 | TRBD2*01 | tgtgccagcagtgccaagactagcgggggctcagatacgcagtatttt | CASSAKTSGGSDTQYF | 1 | 1.67 | 1.67 | |
| TRBV2*01 | TRBJ2-3*01 | TRBD2*01 | tgtgccagcagcgccctggctagcgggggccgggatacgcagtatttt | CASSALASGGRDTQYF | 1 | 1.67 | 1.67 | |
| TRBV2*01 | TRBJ2-1*01 | TRBD2*01 | tgtgccagcagtgcccggactagcgggggactcgatgagcagttcttc | CASSARTSGGLDEQFF | 1 | 1.67 | 1.67 | |
| TRBV2*01 | TRBJ2-3*01 | TRBD2*01 | tgtgccagcagtgccaggactagcgggggctcggatacgcagtatttt | CASSARTSGGSDTQYF | 1 | 1.67 | 1.67 | |
| TRBV2*01, or | TRBJ2-3*01 | TRBD2*01 | tgtgccagcagtaagagggctagcgggggcacagatacgcagtatttt | CASSKRASGGTDTQYF | 2 | 3.33 | 3.33 | |
| TRBV2*02 | | | | | | | | |
| TRBV2*01, or | TRBJ2-3*01 | TRBD2*01 | tgtgccagcagcctaaggactagcgggggttcagatacgcagtatttt | CASSLRTSGGSDTQYF | 1 | 1.67 | 1.67 | |
| TRBV2*02 or | | | | | | | | |
| TRBV2*03 | | | | | | | | |
| TRBV2*01 | TRBJ2-1*01 | TRBD2*01 | tgtgccagcagccgcttgacgagcggggggcggaatgagcagttcttc | CASSRLTSGGRNEQFF | 2 | 3.33 | 3.33 | |
| TRBV2*01 | TRBJ2-3*01 | TRBD2*01 | tgtgccagcagttctaagactagcgggggcacagatacgcagtatttt | CASSSKTSGGTDTQYF | 1 | 1.67 | 1.67 | |
| TRBV2*01 | TRBJ2-1*01 | TRBD2*01 | tgtgccagcagttcaaggactagcgggggccaagatgagcagttcttc | CASSSRTSGGQDEQFF | 1 | 1.67 | 1.67 | |
| TRBV2*01 | TRBJ1-5*01 | TRBD2*01 | tgtgccacctccagaggagcgcggggaagcaatcagccccagcatttt | CATSRGARGSNQPQHF | 34 | 56.67 | 56.67 | |
| | | | Total | 23 | 60 | 100.00 | 100.00 | |
| HIC 2 DRB5+ | | | | | | | | |
| TRBV2*01, or | TRBJ1-2*01 | TRBD1*01 | tgtgccagcgccaggacagggggcgttggctacaccttc | CASARTGGVGYTF | 1 | 0.94 | 0.94 | 39 |
| TRBV2*02 or | | | | | | | | |
| TRBV2*03 | | | | | | | | |
| TRBV2*01, or | TRBJ2-7*01 | TRBD2*02 | tgtgccagcaggactagcgggacctacgagcagtacttc | CASRTSGTYEQYF | 1 | 0.94 | 0.94 | |
| TRBV2*02 or | | | | | | | | |
| TRBV2*03 | | | | | | | | |
| TRBV2*01, or | TRBJ2-7*01 | TRBD1*01 | tgtgccagcaaagcaaaaacggtaacctacaagcagtacttc | CASKAKTVTYKQYF | 1 | 0.94 | 0.94 | 42 |
| TRBV2*02 or | | | | | | | | |
| TRBV2*03 | | | | | | | | |
| TRBV2*01, or | TRBJ2-7*01 | TRBD2*01 | tgtgccagcaaaccaaaagcggtaacctacgagcagtacttc | CASKPKAVTYEQYF | 1 | 0.94 | 0.94 | |
| TRBV2*02 or | | | | | | | | |
| TRBV2*03 | | | | | | | | |
| TRBV2*01, or | TRBJ1-3*01 | TRBD1*01 | tgtgccagcagagggacagcgactggaaacaccatatatttt | CASRGTATGNTIYF | 1 | 0.94 | 0.94 | |
| TRBV2*02 or | | | | | | | | |
| TRBV2*03 | | | | | | | | |
| TRBV2*01, or | TRBJ1-4*01 | TRBD1*01 | tgtgccagcagaccgacagcaactaatgaaaaactgtttttt | CASRPTATNEKLFF | 1 | 0.94 | 0.94 | |
| TRBV2*02 or | | | | | | | | |
| TRBV2*03 | | | | | | | | |
| TRBV2*01, or | TRBJ2-1*01 | TRBD2*01 | tgtgccagcagtgaatatgcgactagcaatgagcagttcttc | CASSEYATSNEQFF | 1 | 0.94 | 0.94 | |
| TRBV2*02 or | | | | | | | | |
| TRBV2*03 | | | | | | | | |
| TRBV2*01, or | TRBJ1-1*01 | TRBD1*01 | tgtgccagcagtcgtggacagcggcacagatacgcagtattt | CASSRGQRHRYAVF | 1 | 0.94 | 0.94 | |
| TRBV2*02 or | | | | | | | | |
| TRBV2*03 | | | | | | | | |
| TRBV2*01, or | TRBJ2-7*01 | TRBD2*02 | tgtgccatctcgcgtctagcgggaggcatggacgagcagtacttc | CAISRLAGGMDEQYF | 1 | 0.94 | 0.94 | 45 |
| TRBV2*02 or | | | | | | | | |
| TRBV2*03 | | | | | | | | |
| TRBV2*01, or | TRBJ2-3*01 | TRBD2*01 | tgtgccagcggccgactagcatcgggcacagatacgcagtatttt | CASGRLASGTDTQYF | 2 | 1.89 | 1.89 | |
| TRBV2*02 or | | | | | | | | |
| TRBV2*03 | | | | | | | | |
| TRBV2*01, or | TRBJ2-2*01 | TRBD2*01 | tgtgccagcaggagggggactagcggcaccggggagctgtttttt | CASRRGTSGTGELFF | 1 | 0.94 | 0.94 | |
| TRBV2*02 or | | | | | | | | |
| TRBV2*03 | | | | | | | | |
| TRBV2*01, or | TRBJ2-7*01 | TRBD2*01 | tgtgccagcagtgatggggctagcggggtgggagagcagtacttc | CASSDGASGVGEQYF | 2 | 1.89 | 1.89 | |
| TRBV2*02 or | | | | | | | | |
| TRBV2*03 | | | | | | | | |
| TRBV2*01, or | TRBJ1-6*02 | TRBD1*01 | tgtgccagcagtgaagctgccaggggtaattcacccctccacttt | CASSEAARGNSPLHF | 2 | 1.89 | 1.89 | |
| TRBV2*02 or | | | | | | | | |
| TRBV2*03 | | | | | | | | |
| TRBV2*01, or | TRBJ2-7*01 | TRBD2*01 | tgtgccagcagtgaaggggctagcgggctcggggagcagtacttc | CASSEGASGLGEQYF | 1 | 0.94 | 0.94 | |
| TRBV2*02 or | | | | | | | | |
| TRBV2*03 | | | | | | | | |
| TRBV2*01, or | TRBJ2-1*01 | TRBD2*02 | tgtgccagcagtgaactggctagcgggataagtgagcagttcttc | CASSELASGISEQFF | 1 | 0.94 | 0.94 | |
| TRBV2*02 or | | | | | | | | |
| TRBV2*03 | | | | | | | | |
| TRBV2*01, or | TRBJ2-1*01 | TRBD2*01 | tgtgccagcagtgaacttgctagcgggctcgcagagcagttcttc | CASSELASGLAEQFF | 2 | 1.89 | 1.89 | |
| TRBV2*02 or | | | | | | | | |
| TRBV2*03 | | | | | | | | |
| TRBV2*01, or | TRBJ2-1*01 | TRBD2*02 | tgtgccagcagtgaactggctagcgggacgggtgagcagttcttc | CASSELASGTGEQFF | 1 | 0.94 | 0.94 | |
| TRBV2*02 or | | | | | | | | |
| TRBV2*03 | | | | | | | | |
| TRBV2*01, or | TRBJ2-7*01 | TRBD2*02 | tgtgccagcagtgaacgggctagcgggaccgacgagcagtacttc | CASSERASGTDEQYF | 2 | 1.89 | 1.89 | |
| TRBV2*02 or | | | | | | | | |
| TRBV2*03 | | | | | | | | |
| TRBV2*01, or | TRBJ2-2*01 | TRBD2*01 | tgtgccagcagtgaaagggctagcggggtcggggagctgtttttt | CASSERASGVGELFF | 2 | 1.89 | 1.89 | |
| TRBV2*02 or | | | | | | | | |
| TRBV2*03 | | | | | | | | |
| TRBV2*01, or | TRBJ2-3*01 | TRBD2*01 | tgtgccagcagcggactggctagcggcacagatacgcagtatttt | CASSGLASGTDTQYF | 2 | 1.89 | 1.89 | |
| TRBV2*02 or | | | | | | | | |
| TRBV2*03 | | | | | | | | |
| TRBV2*01, or | TRBJ2-2*01 | TRBD2*01 | tgtgccagttcgggactagcgtcgggcaccggggagctgtttttt | CASSGLASGTGELFF | 1 | 0.94 | 0.94 | |
| TRBV2*02 or | | | | | | | | |
| TRBV2*03 | | | | | | | | |
| TRBV2*01, or | TRBJ2-7*01 | TRBD2*01 | tgtgccagcagcgggatgactagccgatcctacgagcagtacttc | CASSGMTSRSYEQYF | 3 | 2.83 | 2.83 | |
| TRBV2*02 or | | | | | | | | |
| TRBV2*03 | | | | | | | | |
| TRBV2*01, or | TRBJ2-2*01 | TRBD2*01 | tgtgccagcagcccgggactagccggcaccggggagctgtttttt | CASSPGLAGTGELFF | 1 | 0.94 | 0.94 | |
| TRBV2*02 or | | | | | | | | |
| TRBV2*03 | | | | | | | | |
| TRBV2*01, or | TRBJ2-3*01 | TRBD2*01 | tgtgccagcagtccgttgactagcggaacagatacgcagtatttt | CASSPLTSGTDTQYF | 1 | 0.94 | 0.94 | |
| TRBV2*02 or | | | | | | | | |
| TRBV2*03 | | | | | | | | |
| TRBV2*01, or | TRBJ1-5*01 | TRBD1*01 | tgtgccagcagtcctcgggccagggggaatcagccccagcatttt | CASSPRARGNQPQHF | 2 | 1.89 | 1.89 | |
| TRBV2*02 or | | | | | | | | |
| TRBV2*03 | | | | | | | | |
| TRBV2*01, or | TRBJ2-7*01 | TRBD2*01 | tgtgccagcagccctcggactagcggtccctacgagcagtacttc | CASSPRTSGPYEQYF | 1 | 0.94 | 0.94 | |
| TRBV2*02 or | | | | | | | | |
| TRBV2*03 | | | | | | | | |
| TRBV2*01, or | TRBJ2-7*01 | TRBD2*02 | tgtgccagcagtcctcggactagcgggagttacgagcagtacttc | CASSPRTSGSYEQYF | 1 | 0.94 | 0.94 | |
| TRBV2*02 or | | | | | | | | |
| TRBV2*03 | | | | | | | | |
| TRBV2*01, or | TRBJ2-2*01 | TRBD2*01 | tgtgccagcagccaagggctagcgggcaccggggagctgtttttt | CASSQGLAGTGELFF | 1 | 0.94 | 0.94 | |
| TRBV2*02 or | | | | | | | | |
| TRBV2*03 | | | | | | | | |
| TRBV2*01, or | TRBJ2-3*01 | TRBD1*01 | tgtgccagcagtcaattagctaggggcacagatacgcagtatttt | CASSQLARGTDTQYF | 1 | 0.94 | 0.94 | |
| TRBV2*02 or | | | | | | | | |
| TRBV2*03 | | | | | | | | |
| TRBV2*01, or | TRBJ2-7*01 | TRBD1*01 | tgtgccagcagtcaacttgtatcgctgaggggggagcagtacttc | CASSQLVSLRGEQYF | 1 | 0.94 | 0.94 | |
| TRBV2*02 or | | | | | | | | |
| TRBV2*03 | | | | | | | | |
| TRBV2*01, or | TRBJ2-7*01 | TRBD2*02 | tgtgccagcagtcaacggactagcgggagcgacgagcagtacttc | CASSQRTSGSDEQYF | 1 | 0.94 | 0.94 | |
| TRBV2*02 or | | | | | | | | |
| TRBV2*03 | | | | | | | | |
| TRBV2*01, or | TRBJ2-3*01 | TRBD2*01 | tgtgccagcagccaagttgcggggggtacagctacgcagtatttt | CASSQVAGGTATQYF | 1 | 0.94 | 0.94 | |
| TRBV2*02 or | | | | | | | | |
| TRBV2*03 | | | | | | | | |
| TRBV2*01, or | TRBJ1-5*01 | TRBD2*01 | tgtgccagctcccggggggctcggggcaatcagccccagcatttt | CASSRGARGNQPQHF | 1 | 0.94 | 0.94 | |
| TRBV2*02 or | | | | | | | | |
| TRBV2*03 | | | | | | | | |
| TRBV2*01, or | TRBJ2-1*01 | TRBD2*02 | tgtgccagcagccgactagcgggaggtttaggtgagcagttcttc | CASSRLAGGLGEQFF | 2 | 1.89 | 1.89 | |
| TRBV2*02 or | | | | | | | | |
| TRBV2*03 | | | | | | | | |
| TRBV2*01, or | TRBJ2-1*01 | TRBD2*02 | tgtgccagcagccgactagcgggagggatggatgagcagttcttc | CASSRLAGGMDEQFF* | 11 | 10.38 | 10.38 | |
| TRBV2*02 or | | | | | | | | |
| TRBV2*03 | | | | | | | | |
| TRBV2*01, or | TRBJ2-1*01 | TRBD2*02 | tgtgccagcagccgactagcgggagggacggatgagcagttcttc | CASSRLAGGTDEQFF | 1 | 0.94 | 0.94 | |
| TRBV2*02 or | | | | | | | | |
| TRBV2*03 | | | | | | | | |
| TRBV2*01, or | TRBJ2-3*01 | TRBD2*01 | tgtgccagcagtcgtctgactagcggcacagatacgcagtatttt | CASSRLTSGTDTQYF | 2 | 1.89 | 1.89 | |
| TRBV2*02 or | | | | | | | | |
| TRBV2*03 | | | | | | | | |
| TRBV2*01, or | TRBJ2-1*01 | TRBD2*02 | tgtgccagcagccgagtgacgggagggatggatgagcagttcttc | CASSRVTGGMDEQFF | 1 | 0.94 | 0.94 | |
| TRBV2*02 or | | | | | | | | |
| TRBV2*03 | | | | | | | | |
| TRBV2*01, or | TRBJ2-1*01 | TRBD2*01 | tgtgccagcaccaaactagcggggggtacatctgagcagttcttc | CASTKLAGGTSEQFF | 1 | 0.94 | 0.94 | |
| TRBV2*02 or | | | | | | | | |
| TRBV2*03 | | | | | | | | |
| TRBV2*01, or | TRBJ2-3*01 | TRBD2*01 | tgtgccagcaccaaactagcgtggggcacatatacgcagtatttt | CASTKLAWGTYTQYF | 1 | 0.94 | 0.94 | |
| TRBV2*02 or | | | | | | | | |
| TRBV2*03 | | | | | | | | |
| TRBV2*01, or | TRBJ2-1*01 | TRBD2*02 | tgtgccaccacccccggggctagcgggataagtgagcagttcttc | CATTPGASGISEQFF* | 35 | 33.02 | 33.96 | |
| TRBV2*02 or | | | | | | | | |
| TRBV2*03 | | | | | | | | |
| TRBV2*01, or | TRBJ2-1*01 | TRBD1*01 | tgtgccaccacccccggggctagtgggataagtgagcagttcttc | | 1 | 0.94 | | |
| TRBV2*02 or | | | | | | | | |
| TRBV2*03 | | | | | | | | |
| TRBV2*01, or | TRBJ2-7*01 | TRBD1*01 | tgtgccagcagtgaaaggggacagggggcgcggtacgagcagtacttc | CASSERGQGARYEQYF | 1 | 0.94 | 0.94 | 48 |
| TRBV2*02 or | | | | | | | | |
| TRBV2*03 | | | | | | | | |
| TRBV2*01, or | TRBJ2-3*01 | TRBD2*01 | tgtgccagcagtcgtatgactggcgggggcacagatacgcagtatttt | CASSRMTGGGTDTQYF | 1 | 0.94 | 0.94 | |
| TRBV2*02 or | | | | | | | | |
| TRBV2*03 | | | | | | | | |
| TRBV2*01, or | TRBJ2-3*01 | TRBD2*01 | tgtgccagcagtcgtaggactagcgggggcacagatacgcagtatttt | CASSRRTSGGTDTQYF* | 6 | 5.66 | 5.66 | |
| TRBV2*02 or | | | | | | | | |
| TRBV2*03 | | | | | | | | |
| | | | Total | 44 | 106 | 100.00 | 100.00 | |
| HIC 3 DR11+ | | | | | | | | |
| TRBV2*01, or | TRBJ2-3*01 | TRBD1*01 | tgtgccagtcaccggacatacacagatacgcagtatttt | CASHRTYTDTQYF | 2 | 1.60 | 1.60 | 39 |
| TRBV2*02 or | | | | | | | | |
| TRBV2*03 | | | | | | | | |
| TRBV2*01, or | TRBJ1-1*01 | TRBD1*01 | tgtgccagctcaggacagacgaacactgaagctttcttt | CASSGQTNTEAFF | 10 | 8.00 | 8.00 | |
| TRBV2*02 or | | | | | | | | |
| TRBV2*03 | | | | | | | | |
| TRBV2*01, or | TRBJ1-2*01 | TRBD1*01 | tgtgccagcagatggacagcaacctcttatggctacaccttc | CASRWTATSYGYTF | 15 | 12.00 | 12.00 | 42 |
| TRBV2*02 or | | | | | | | | |
| TRBV2*03 | | | | | | | | |
| TRBV2*01, or | TRBJ1-2*01 | TRBD1*01 | tgtgccagcagtcccacaacgacagggtatggctacaccttc | CASSPTTTGYGYTF | 1 | 0.80 | 0.80 | |
| TRBV2*02 or | | | | | | | | |
| TRBV2*03 | | | | | | | | |
| TRBV2*01, or | TRBJ2-2*01 | TRBD1*01 | tgtgcctcccacgaaggggccgggggcttcggggagctgtttttt | CASHEGAGGFGELFF | 1 | 0.80 | 0.80 | 45 |
| TRBV2*02 or | | | | | | | | |
| TRBV2*03 | | | | | | | | |
| TRBV2*01, or | TRBJ2-2*01 | TRBD1*01 | tgtgcctcccacgaaggggccgggggctacggggagctgtttttt | CASHEGAGGYGELFF | 2 | 1.60 | 1.60 | |
| TRBV2*02 or | | | | | | | | |
| TRBV2*03 | | | | | | | | |
| TRBV2*01, or | TRBJ2-1*01 | TRBD1*01 | tgtgccagcagtgccggaactagaggggtgggggagcagttcttc | CASSAGTRGVGEQFF | 1 | 0.80 | 0.80 | |
| TRBV2*02 or | | | | | | | | |
| TRBV2*03 | | | | | | | | |
| TRBV2*01, or | TRBJ2-3*01 | TRBD2*01 | tgtgccagcagtgctttggctagcggcacagatacgcagtatttt | CASSALASGTDTQYF | 1 | 0.80 | 0.80 | |
| TRBV2*02 or | | | | | | | | |
| TRBV2*03 | | | | | | | | |
| TRBV2*01, or | TRBJ2-7*01 | TRBD2*01 | tgtgccagcagtgacgcggctagcggtgtgggcgagcagtacttc | CASSDAASGVGEQYF | 6 | 4.80 | 4.80 | |
| TRBV2*02 or | | | | | | | | |
| TRBV2*03 | | | | | | | | |
| TRBV2*01, or | TRBJ2-1*01 | TRBD2*02 | tgtgccagcagtgatctggctagcgggacgaatgagcagttcttc | CASSDLASGTNEQFF | 1 | 0.80 | 0.80 | |
| TRBV2*02 or | | | | | | | | |
| TRBV2*03 | | | | | | | | |
| TRBV2*01, or | TRBJ2-1*01 | TRBD2*01 | tgtgccagcagtgaccgggctagcggggtcggggagcagttcttc | CASSDRASGVGEQFF | 1 | 0.80 | 0.80 | |
| TRBV2*02 or | | | | | | | | |
| TRBV2*03 | | | | | | | | |
| TRBV2*01, or | TRBJ2-1*01 | TRBD2*01 | tgtgccagcagtgacaggactagcggtccccatgagcagttcttc | CASSDRTSGPHEQFF | 6 | 4.80 | 4.80 | |
| TRBV2*02 or | | | | | | | | |
| TRBV2*03 | | | | | | | | |
| TRBV2*01, or | TRBJ2-1*01 | TRBD2*02 | tgtgccagcagcggactagcgggaggaatggatgagcagttcttc | CASSGLAGGMDEQFF* | 7 | 5.60 | 5.60 | |
| TRBV2*02 or | | | | | | | | |
| TRBV2*03 | | | | | | | | |
| TRBV2*01, or | TRBJ2-1*01 | TRBD1*01 | tgtgccagcagccccggggcgagaggaattgatgagcagttcttc | CASSPGARGIDEQFF | 1 | 0.80 | 0.80 | |
| TRBV2*02 or | | | | | | | | |
| TRBV2*03 | | | | | | | | |
| TRBV2*01, or | TRBJ2-1*01 | TRBD2*01 | tgtgcaagcagccccgggactagcggagttggtgagcagttcttc | CASSPGTSGVGEQFF* | 15 | 12.00 | 12.80 | |
| TRBV2*02 or | | | | | | | | |
| TRBV2*03 | | | | | | | | |
| TRBV2*01, or | TRBJ2-1*01 | TRBD2*01 | tgtgcaagcagccccgggactagcggagttggtgagcagtttttc | | 1 | 0.80 | | |
| TRBV2*02 or | | | | | | | | |
| TRBV2*03 | | | | | | | | |
| TRBV2*01, or | TRBJ2-1*01 | TRBD1*01 | tgtgcaagcagccccgggacgagcggagttggtaagcagtttttc | CASSPGTSGVGKQFF | 1 | 0.80 | 0.80 | |
| TRBV2*02 or | | | | | | | | |
| TRBV2*03 | | | | | | | | |
| TRBV2*01, or | TRBJ2-7*01 | TRBD2*01 | tgtgccagcagccccaggactagcgggggaggcgagcagtacttc | CASSPRTSGGGEQYF | 1 | 0.80 | 0.80 | |
| TRBV2*02 or | | | | | | | | |
| TRBV2*03 | | | | | | | | |
| TRBV2*01, or | TRBJ1-5*01 | TRBD1*01 | tgtgccagcagtcccagtgctcgcggcaatcagccccagcatttt | CASSPSARGNQPQHF | 2 | 1.60 | 1.60 | |
| TRBV2*02 or | | | | | | | | |
| TRBV2*03 | | | | | | | | |
| TRBV2*01, or | TRBJ2-7*01 | TRBD2*02 | tgtgccagcagcccgacgactagcgggagaggcgagcagtacttc | CASSPTTSGRGEQYF | 2 | 1.60 | 1.60 | |
| TRBV2*02 or | | | | | | | | |
| TRBV2*03 | | | | | | | | |
| TRBV2*01, or | TRBJ2-1*01 | TRBD2*01 | tgtgccagcagttccgggactagcggggccggggagcagttcttc | CASSSGTSGAGEQFF | 1 | 0.80 | 0.80 | |
| TRBV2*02 or | | | | | | | | |
| TRBV2*03 | | | | | | | | |
| TRBV2*01, or | TRBJ2-1*01 | TRBD2*01 | tgtgccagcagttccgggactagcggagttggtgagcagttcttc | CASSSGTSGVGEQFF | 1 | 0.80 | 27.20 | |
| TRBV2*02 or | | | | | | | | |
| TRBV2*03 | | | | | | | | |
| TRBV2*01, or | TRBJ2-1*01 | TRBD2*01 | tgtgccagcagttccgggactagcggggtcggggagcagttcttc | CASSSGTSGVGEQFF | 33 | 26.40 | | |
| TRBV2*02 or | | | | | | | | |
| TRBV2*03 | | | | | | | | |
| TRBV2*01, or | TRBJ2-7*01 | TRBD2*01 | tgtgccagcagtgtcgggactagcggggtgggcgagcagtacttc | CASSVGTSGVGEQYF | 11 | 8.80 | 8.80 | |
| TRBV2*02 or | | | | | | | | |
| TRBV2*03 | | | | | | | | |
| TRBV2*01, or | TRBJ2-7*01 | TRBD2*01 | tgtgccagcagttacggggctagcggggtgggggagcagttcttc | CASSYGASGVGEQFF | 1 | 0.80 | 0.80 | |
| TRBV2*02 or | | | | | | | | |
| TRBV2*03 | | | | | | | | |
| TRBV2*01, or | TRBJ2-1*01 | TRBD2*01 | tgtgccagcagttacaggactagcgggccccgggagcagttcttc | CASSYRTSGPREQFF | 1 | 0.80 | 0.80 | |
| TRBV2*02 or | | | | | | | | |
| TRBV2*03 | | | | | | | | |
| | | | Total | 24 | 125 | 100.00 | 100.00 | |
| HIC 4 DR15+ | | | | | | | | |
| TRBV2*01, or | TRBJ1-2*01 | TRBD2*01 | tgtgccagcaggaaagaaggatctaggctacacctc | CASRKEGSRLHL | 1 | 0.66 | 0.66 | 36 |
| TRBV2*02 or | | | | | | | | |
| TRBV2*03 | | | | | | | | |
| TRBV2*01, or | TRBJ1-2*01 | TRBD1*01 | tgtgccagcagtgacagaacgacatgtggctacaccttc | CASSDRTTCGYTF | 1 | 0.66 | 0.66 | 39 |
| TRBV2*02 or | | | | | | | | |
| TRBV2*03 | | | | | | | | |
| TRBV2*01, or | TRBJ1-2*01 | TRBD1*01 | tgtgccagcagtgaaagaaggatctatggctacaccttc | CASSERRIYGYTF | 5 | 3.29 | 3.29 | |
| TRBV2*02 or | | | | | | | | |
| TRBV2*03 | | | | | | | | |
| TRBV2*01, or | TRBJ2-1*01 | TRBD2*01 | tgtgccagcagagccctagcgtcggggggtgagcagttcttc | CASRALASGGEQFF | 5 | 3.29 | 3.29 | 42 |
| TRBV2*02 or | | | | | | | | |
| TRBV2*03 | | | | | | | | |
| TRBV2*01, or | TRBJ2-3*01 | TRBD2*01 | tgtgccagcagtgctttagctagcggagataagcagtatttt | CASSALASGDKQYF | 1 | 0.66 | 0.66 | |
| TRBV2*02 or | | | | | | | | |
| TRBV2*03 | | | | | | | | |
| TRBV2*01, or | TRBJ2-3*01 | TRBD2*01 | tgtgccagcagtgctttggctagcggagatacgcagtatttt | CASSALASGDTQYF | 1 | 0.66 | 0.66 | |
| TRBV2*02 or | | | | | | | | |
| TRBV2*03 | | | | | | | | |
| TRBV2*01, or | TRBJ2-1*01 | TRBD1*01 | tgtgccagcagtgacgacagggtcggcgatgagcagttcttc | CASSDDRVGDEQFF | 1 | 0.66 | 0.66 | |
| TRBV2*02 or | | | | | | | | |
| TRBV2*03 | | | | | | | | |
| TRBV2*01, or | TRBJ2-1*01 | TRBD2*01 | tgtgccagcagcaaactagcgtctggagatgagcagttcttc | CASSKLASGDEQFF | 4 | 2.63 | 2.63 | |
| TRBV2*02 or | | | | | | | | |
| TRBV2*03 | | | | | | | | |
| TRBV2*01, or | TRBJ2-1*01 | TRBD2*02 | tgtgccagcagtgttttacctggaggcaatgatccgctcttc | CASSVLPGGNDPLF | 1 | 0.66 | 0.66 | |
| TRBV2*02 or | | | | | | | | |
| TRBV2*03 | | | | | | | | |
| TRBV2*01, or | TRBJ2-1*01 | TRBD2*01 | tgtgccagcagtgttttacctggtcgcaatgagccgttcttc | CASSVLPGRNEPFF | 1 | 0.66 | 0.66 | |
| TRBV2*02 or | | | | | | | | |
| TRBV2*03 | | | | | | | | |
| TRBV2*01, or | TRBJ2-1*01 | TRBD1*01 | tgcgccagcagtgttttacgtggtggcaatgagcagtttttc | CASSVLRGGNEQFF | 1 | 0.66 | 0.66 | |
| TRBV2*02 or | | | | | | | | |
| TRBV2*03 | | | | | | | | |
| TRBV2*01, or | TRBJ2-1*01 | TRBD2*01 | tgtgccagcagtgttttacgtggtcgcaatgagccgttcttc | CASSVLRGRNEPFF | 1 | 0.66 | 0.66 | |
| TRBV2*02 or | | | | | | | | |
| TRBV2*03 | | | | | | | | |
| TRBV2*01, or | TRBJ2-1*01 | | tgtgccagcagtgttttacgtggtcgcaatgagcagttcttc | CASSVLRGRNEQFF | 8 | 5.26 | 5.26 | |
| TRBV2*02 or | | | | | | | | |
| TRBV2*03 | | | | | | | | |
| TRBV2*01, or | TRBJ2-1*01 | TRBD2*02 | tgtgccagcagtgtttcacgaggtggcaataagcagtttttc | CASSVSRGGNKQFF | 1 | 0.66 | 0.66 | |
| TRBV2*02 or | | | | | | | | |
| TRBV2*03 | | | | | | | | |
| TRBV2*01, or | TRBJ2-1*01 | TRBD1*01 | tgtgccagtgtattaatgaggacgaacaatgagcagttcttc | CASVLMRTNNEQFF | 9 | 5.92 | 5.92 | |
| TRBV2*02 or | | | | | | | | |
| TRBV2*03 | | | | | | | | |
| TRBV2*01, or | TRBJ2-1*01 | TRBD2*01 | tgctccagcagagctagagggtgcgcgggtaagcagtatttc | CSSRARGCAGKQYF | 1 | 0.66 | 0.66 | |
| TRBV2*02 or | | | | | | | | |
| TRBV2*03 | | | | | | | | |
| TRBV2*01, or | TRBJ2-1*01 | TRBD2*01 | tgtgccagcagtgccctgactagcgggggcgatgagcagttcttc | CASSALTSGGDEQFF | 1 | 0.66 | 0.66 | 45 |
| TRBV2*02 or | | | | | | | | |
| TRBV2*03 | | | | | | | | |
| TRBV2*01, or | TRBJ2-1*01 | TRBD2*01 | tgtgccagcagtgcaaggactagcgggggatccgagcagttcttc | CASSARTSGGSEQFF | 2 | 1.32 | 1.32 | |
| TRBV2*02 or | | | | | | | | |
| TRBV2*03 | | | | | | | | |
| TRBV2*01, or | TRBJ2-7*01 | TRBD2*02 | tgtgccagcagtgctaggactagcgggagtgacgagcagtacttc | CASSARTSGSDEQYF | 2 | 1.32 | 1.32 | |
| TRBV2*02 or | | | | | | | | |
| TRBV2*03 | | | | | | | | |
| TRBV2*01, or | TRBJ2-3*01 | TRBD1*01 | tgtgccagcagtgacagggcctcaggcggggatacgcagtatttt | CASSDRASGGDTQYF | 1 | 0.66 | 0.66 | |
| TRBV2*02 or | | | | | | | | |
| TRBV2*03 | | | | | | | | |
| TRBV2*01, or | TRBJ2-3*01 | TRBD2*01 | tgtgccagcagtgatcgggctagcgggggggatacgcagtatttt | CASSDRASGGDTQYF | 1 | 0.66 | 0.66 | |
| TRBV2*02 or | | | | | | | | |
| TRBV2*03 | | | | | | | | |
| TRBV2*01, or | TRBJ2-3*01 | TRBD1*01 | tgtgccagcagtgatcgggctacagggggggatacgcagtatttt | CASSDRATGGDTQYF | 1 | 0.66 | 0.66 | |
| TRBV2*02 or | | | | | | | | |
| TRBV2*03 | | | | | | | | |
| TRBV2*01, or | TRBJ2-3*01 | TRBD2*01 | tgtgccagcagtgaaaaggctagcgggggggatacgcagtatttt | CASSEKASGGDTQYF | 1 | 0.66 | 0.66 | |
| TRBV2*02 or | | | | | | | | |
| TRBV2*03 | | | | | | | | |
| TRBV2*01, or | TRBJ2-1*01 | TRBD2*01 | tgtgccagcagtgaattggctagcgggggggatgagcagttcttc | CASSELASGGDEQFF | 2 | 1.32 | 1.32 | |
| TRBV2*02 or | | | | | | | | |
| TRBV2*03 | | | | | | | | |
| TRBV2*01, or | TRBJ2-3*01 | TRBD1*01 | tgtgccagcagtcacaaggcttcagggggggataagcagtatttt | CASSHKASGGDKQYF | 1 | 0.66 | 0.66 | |
| TRBV2*02 or | | | | | | | | |
| TRBV2*03 | | | | | | | | |
| TRBV2*01, or | TRBJ2-3*01 | TRBD1*01 | tgtgccagctctcacatggcctcaggcggcgatacgcagtatttt | CASSHMASGGDTQYF | 1 | 0.66 | 0.66 | |
| TRBV2*02 or | | | | | | | | |
| TRBV2*03 | | | | | | | | |
| TRBV2*01, or | TRBJ2-3*01 | TRBD1*01 | tgtgccagcagtcacagggcctcaggcggggctactccgtatttt | CASSHRASGGATPYF | 1 | 0.66 | 0.66 | |
| TRBV2*02 or | | | | | | | | |
| TRBV2*03 | | | | | | | | |
| TRBV2*01, or | TRBJ2-3*01 | TRBD1*01 | tgtgccagcagtcaccgggcctcaggtggggatactccgcatttt | CASSHRASGGDTPHF | 1 | 0.66 | 0.66 | |
| TRBV2*02 or | | | | | | | | |
| TRBV2*03 | | | | | | | | |
| TRBV2*01, or | TRBJ2-3*01 | TRBD1*01 | tgtgccagcagtcacagggcctcaggcggggatacgcagtatttt | CASSHRASGGDTQYF | 31 | 20.39 | 20.39 | |
| TRBV2*02 or | | | | | | | | |
| TRBV2*03 | | | | | | | | |
| TRBV2*01, or | TRBJ2-7*01 | TRBD2*01 | tgtgccagctcaaaactggctagcggggccgacgagcagtacttc | CASSKLASGADEQYF | 6 | 3.95 | 4.61 | |
| TRBV2*02 or | | | | | | | | |
| TRBV2*03 | | | | | | | | |
| TRBV2*01, or | TRBJ2-7*01 | TRBD2*01 | tgtgccagctcaaaactggctagcggggccgacgagcagtatttc | | 1 | 0.66 | | |
| TRBV2*02 or | | | | | | | | |
| TRBV2*03 | | | | | | | | |
| TRBV2*01, or | TRBD1*01 | | tgtgccagctcaaaacttactaggggcgcagataagcagtatttc | CASSKLTRGADKQYF | 1 | 0.66 | 0.66 | |
| TRBJ2-7*01 | | | | | | | | |
| TRBV2*02 | | | | | | | | |
| TRBV2*01, or | TRBJ2-7*01 | TRBD2*01 | tgtgccagcagtaagaggactagcggtacctacgagcagtacttc | CASSKRTSGTYEQYF | 1 | 0.66 | 0.66 | |
| TRBV2*02 or | | | | | | | | |
| TRBV2*03 | | | | | | | | |
| TRBV2*01, or | TRBJ2-7*01 | TRBD2*01 | tgtgccagcagcctccggactagcggctcctacgagcagtacttc | CASSLRTSGSYEQYF | 2 | 1.32 | 1.32 | |
| TRBV2*02 or | | | | | | | | |
| TRBV2*03 | | | | | | | | |
| TRBV2*01, or | TRBJ2-7*01 | TRBD2*01 | tgtgccagcagtccccggactagcggtacctacgagcagtacttc | CASSPRTSGTYEQYF | 1 | 0.66 | 0.66 | |
| TRBV2*02 or | | | | | | | | |
| TRBV2*03 | | | | | | | | |
| TRBV2*01, or | TRBJ2-2*01 | TRBD2*01 | tgtgccagctcgccgcgagtcttctctgtcggggagctgtttttt | CASSPRVFSVGELFF | 4 | 2.63 | 2.63 | |
| TRBV2*02 or | | | | | | | | |
| TRBV2*03 | | | | | | | | |
| TRBV2*01, or | TRBJ2-1*01 | TRBD2*01 | tgtgccagcagccaaagggctagcgggggggacgagcagttcttc | CASSQRASGGDEQFF | 2 | 1.32 | 1.32 | |
| TRBV2*02 or | | | | | | | | |
| TRBV2*03 | | | | | | | | |
| TRBV2*01, or | TRBJ2-7*01 | TRBD2*02 | tgtgccagcagtagacggactagcgggacctacgagcagtacttc | CASSRRTSGTYEQYF | 1 | 0.66 | 0.66 | |
| TRBV2*02 or | | | | | | | | |
| TRBV2*03 | | | | | | | | |
| TRBV2*01, or | TRBJ2-7*01 | TRBD2*01 | tgtgccagcagcgtccggactagcgggtcctacgagcagtacttc | CASSVRTSGSYEQYF | 1 | 0.66 | 0.66 | |
| TRBV2*02 or | | | | | | | | |
| TRBV2*03 | | | | | | | | |
| TRBV2*01, or | TRBJ2-7*01 | TRBD2*02 | tgcaccagcagtggtaggactagcgggagggataagcagtatttc | CTSSGRTSGRDKQYF | 1 | 0.66 | 0.66 | |
| TRBV2*02 or | | | | | | | | |
| TRBV2*03 | | | | | | | | |
| TRBV2*01, or | TRBJ2-3*01 | TRBD1*01 | tgtaccagcagtcacagggcctcaggcggggatacgcagtatttt | CTSSHRASGGDTQYF | 1 | 0.66 | 0.66 | |
| TRBV2*02 or | | | | | | | | |
| TRBV2*03 | | | | | | | | |
| TRBV2*01, or | TRBJ2-7*01 | TRBD2*01 | tgtgccagcagtgccaggattagcggggggctcaacgagcagtacttc | CASSARISGGLNEQYF | 1 | 0.66 | 0.66 | 48 |
| TRBV2*02 or | | | | | | | | |
| TRBV2*03 | | | | | | | | |
| TRBV2*01, or | TRBJ2-3*01 | TRBD2*01 | tgtgccagcagtgcaaggactagcgggggggccgatacgcagtatttt | CASSARTSGGADTQYF | 1 | 0.66 | 0.66 | |
| TRBV2*02 or | | | | | | | | |
| TRBV2*03 | | | | | | | | |
| TRBV2*01, or | TRBJ2-7*01 | TRBD2*01 | tgtgccagcagtgccaggactagcggggggcttgacgagcagtacttc | CASSARTSGGLDEQYF | 19 | 12.50 | 12.50 | |
| TRBV2*02 or | | | | | | | | |
| TRBV2*03 | | | | | | | | |
| TRBV2*01, or | TRBJ2-3*01 | TRBD2*01 | tgtgccagcagtgctcggactagcggggggtcagatacgcagtatttt | CASSARTSGGSDTQYF | 3 | 1.97 | 1.97 | |
| TRBV2*02 or | | | | | | | | |
| TRBV2*03 | | | | | | | | |
| TRBV2*01, or | TRBJ2-3*01 | TRBD2*01 | tgtgccagcagtaaaaggactagcgggggggccgatacgcagtatttt | CASSKRTSGGADTQYF | 2 | 1.32 | 1.32 | |
| TRBV2*02 or | | | | | | | | |
| TRBV2*03 | | | | | | | | |
| TRBV2*01, or | TRBJ2-3*01 | TRBD2*01 | tgtgccagcagtcttaggactagcgggggcacagatacgcagtatttt | CASSLRTSGGTDTQYF | 1 | 0.66 | 0.66 | |
| TRBV2*02 or | | | | | | | | |
| TRBV2*03 | | | | | | | | |
| TRBV2*01, or | TRBJ2-3*01 | TRBD2*01 | tgtgccagcagccccaggactagcgggggcacagatacgcagtatttt | CASSPRTSGGTDTQYF | 5 | 3.29 | 3.29 | |
| TRBV2*02 or | | | | | | | | |
| TRBV2*03 | | | | | | | | |
| TRBV2*01, or | TRBJ2-3*01 | TRBD1*01 | tgtgccagctcacgacgggcttccgggggcactactccgcattatttt | CASSRRASGGTTPHYF | 1 | 0.66 | 0.66 | |
| TRBV2*02 or | | | | | | | | |
| TRBV2*03 | | | | | | | | |
| TRBV2*01, or | TRBJ2-3*01 | TRBD2*01 | tgtgccagcagtcgacggactagcgggggcacagatacgcagtatttt | CASSRRTSGGTDTQYF* | 1 | 0.66 | 1.32 | |
| TRBV2*02 or | | | | | | | | |
| TRBV2*03 | | | | | | | | |
| TRBV2*01, or | TRBJ2-3*01 | TRBD2*01 | tgtgccagctcacgacggactagcgggggcacagatacgcagtatttt | | 1 | 0.66 | | |
| TRBV2*02 or | | | | | | | | |
| TRBV2*03 | | | | | | | | |
| TRBV2*01, or | TRBJ2-3*01 | TRBD2*02 | tgtgccagcagtcgtcggactagcgggagggcggatacgcagtatttt | CASSRRTSGRADTQYF | 4 | 2.63 | 2.63 | |
| TRBV2*02 or | | | | | | | | |
| TRBV2*03 | | | | | | | | |
| TRBV2*01, or | TRBJ2-3*01 | TRBD2*02 | tgtgccagcagtcgtaggactagcgggagtctagatacgcagtatttt | CASSRRTSGSLDTQYF | 1 | 0.66 | 0.66 | |
| TRBV2*02 or | | | | | | | | |
| TRBV2*03 | | | | | | | | |
| TRBV2*01, or | TRBJ2-3*01 | TRBD1*01 | tgtgccagcagcaccaggattagagggggcacagataagcagtatttt | CASSTRIRGGTDKQYF | 1 | 0.66 | 0.66 | |
| TRBV2*02 or | | | | | | | | |
| TRBV2*03 | | | | | | | | |
| TRBV2*01, or | TRBJ2-3*01 | TRBD2*01 | tgtgccagcagtgttaggactagcgggggcacagatacgcagtatttt | CASSVRTSGGTDTQYF | 1 | 0.66 | 0.66 | |
| TRBV2*02 or | | | | | | | | |
| TRBV2*03 | | | | | | | | |
| | | | Total | 52 | 152 | 100.00 | 100.00 | |
| HIC 5 DR1+ | | | | | | | | |
| TRBV2*01 | TRBJ1-2*01 | TRBD1*01 | tgtgccagcagagacagtaactatggctacaccttc | CASRDSNYGYTF | 1 | 1.61 | 1.61 | 36 |
| TRBV2*02 | TRBJ2-5*01 | TRBD1*01 | tgtgccagcagtcgacggacggagacccagtacttc | CASSRRTETQYF | 3 | 4.84 | 4.84 | |
| TRBV2*02 | TRBJ2-4*01 | TRBD1*01 | tgtgccagcagtgaaaccagggccaacattcagtacttc | CASSETRANIQYF | 1 | 1.61 | 1.61 | 39 |
| TRBV2*01 | TRBJ2-1*01 | TRBD2*01 | tgtgccagcagcggactagcggcgaatgagcagttcttc | CASSGLAANEQFF | 1 | 1.61 | 1.61 | |
| TRBV2*01, or | TRBD1*01 | | tgtgcctcaagggcagggtcggtggccactgaagctttcttt | CASRAGSVATEAFF | 1 | 1.61 | 1.61 | 42 |
| TRBV2*02 | | | | | | | | |
| TRBV2*01 | TRBJ2-1*01 | TRBD2*01 | tgtgccagcagtgggaggactagcggcaatgagcagttcttc | CASSGRTSGNEQFF | 2 | 3.23 | 3.23 | |
| TRBV2*02 | TRBJ2-1*01 | TRBD1*01 | tgtgccagcagtgtcgtgggcagttacaatgagcagttcttc | CASSVVGSYNEQFF | 34 | 54.84 | 54.84 | |
| TRBV2*01 | TRBJ2-1*01 | TRBD1*01 | tgtgccagtgtattaatgaggacgaacaatgagcagttcttc | CASVLMRTNNEQFF | 4 | 6.45 | 6.45 | |
| TRBV2*02 | TRBJ2-2*01 | TRBD1*01 | tgtgccagcagggccgggacctcgggcaccggggagctgtttttt | CASRAGTSGTGELFF | 1 | 1.61 | 1.61 | 45 |
| TRBV2*01, or | TRBJ2-7*01 | TRBD2*02 | tgtgccagcaggaaggggactagcgggagtggcaagcagtacttc | CASRKGTSGSGKQYF | 2 | 3.23 | 3.23 | |
| TRBV2*02 | | | | | | | | |
| TRBV2*02 | TRBJ2-1*01 | TRBD2*01 | tgtgccagcagtgaaaaggctagcggggtggatgagcagttcttc | CASSEKASGVDEQFF | 1 | 1.61 | 1.61 | |
| TRBV2*01 | TRBJ2-3*01 | TRBD2*01 | tgtgccagcagtgaaagggctagcgggcacgatacgcagtatttt | CASSERASGHDTQYF | 1 | 1.61 | 1.61 | |
| TRBV2*01 | TRBJ2-3*01 | TRBD1*01 | tgtgccagcagtcacagggcctcaggcggggatacgcagtatttt | CASSHRASGGDTQYF | 1 | 1.61 | 1.61 | |
| TRBV2*01, or | TRBJ2-7*01 | TRBD2*01 | tgtgccagctcaaaactggctagcggggccgacgagcagtacttc | CASSKLASGADEQYF | 1 | 1.61 | 1.61 | |
| TRBV2*02 | | | | | | | | |
| TRBV2*01 | TRBJ2-7*01 | TRBD2*01 | tgtgccagcagtaaacaggctagcgggggggacgagcagtacttc | CASSKQASGGDEQYF | 8 | 12.90 | 12.90 | |
| | | | Total | 15 | 62 | 100.00 | 100.00 | |
| HIC 6 DR11+ | | | | | | | | |
| TRBV2*01, or | TRBJ2-7*01 | TRBD2*01 | tgtgccagcagagaatacgcgactagcaacgagcagtacttc | CASREYATSNEQYF | 1 | 1.52 | 1.52 | 42 |
| TRBV2*02 or | | | | | | | | |
| TRBV2*03 | | | | | | | | |
| TRBV2*01, or | TRBJ1-2*01 | TRBD2*01 | tgtgccagcagtgaaatggcgaccgggttgcgctacaccttc | CASSEMATGLRYTF | 1 | 1.52 | 1.52 | |
| TRBV2*02 or | | | | | | | | |
| TRBV2*03 | | | | | | | | |
| TRBV2*01, or | TRBJ1-2*01 | TRBD1*01 | tgtgccagcagtgaaacggcgacagggttgcgctacaccttc | CASSETATGLRYTF | 4 | 6.06 | 6.06 | |
| TRBV2*02 or | | | | | | | | |
| TRBV2*03 | | | | | | | | |
| TRBV2*01, or | TRBJ1-6*02 | TRBD2*01 | tgtgccagcacgctaacacgggttaattcacccctccacttt | CASTLTRVNSPLHF | 2 | 3.03 | 3.03 | |
| TRBV2*02 or | | | | | | | | |
| TRBV2*03 | | | | | | | | |
| TRBV2*01, or | TRBJ2-7*01 | TRBD2*01 | tgtgccagcaatcaacggactagcgggccttacgagcagtacttc | CASNQRTSGPYEQYF | 1 | 1.52 | 1.52 | 45 |
| TRBV2*02 or | | | | | | | | |
| TRBV2*03 | | | | | | | | |
| TRBV2*01, or | TRBJ2-1*01 | TRBD2*01 | tgtgccagcagtgatttggctagcggcacaggggagcagttcttc | CASSDLASGTGEQFF | 2 | 3.03 | 3.03 | |
| TRBV2*02 or | | | | | | | | |
| TRBV2*03 | | | | | | | | |
| TRBV2*01, or | TRBJ2-1*01 | TRBD1*01 | tgtgccagcagtgaagctgcagggggctacggtgagcagttcttc | CASSEAAGGYGEQFF | 1 | 1.52 | 1.52 | |
| TRBV2*02 or | | | | | | | | |
| TRBV2*03 | | | | | | | | |
| TRBV2*01, or | TRBJ1-5*01 | TRBD1*01 | tgtgccagcagtgaatttgccaggggcaatcagccccagcatttt | CASSEFARGNQPQHF | 1 | 1.52 | 1.52 | |
| TRBV2*02 or | | | | | | | | |
| TRBV2*03 | | | | | | | | |
| TRBV2*01, or | TRBJ1-5*01 | TRBD1*01 | tgtgccagcagtgaattcgtaagggacaatcagccccagcatttt | CASSEFVRDNQPQHF | 1 | 1.52 | 1.52 | |
| TRBV2*02 or | | | | | | | | |
| TRBV2*03 | | | | | | | | |
| TRBV2*01, or | TRBJ1-5*01 | TRBD1*01 | tgtgccagcagtgaattcgtaaggggcaatcagccccagcatttt | CASSEFVRGNQPQHF | 4 | 6.06 | 6.06 | |
| TRBV2*02 or | | | | | | | | |
| TRBV2*03 | | | | | | | | |
| TRBV2*01, or | TRBJ1-5*01 | TRBD1*01 | tgtgccagcagtgaaggggcggctggcaatcagccccagcatttt | CASSEGAAGNQPQHF | 1 | 1.52 | 1.52 | |
| TRBV2*02 or | | | | | | | | |
| TRBV2*03 | | | | | | | | |
| TRBV2*01, or | TRBJ2-1*01 | TRBD1*01 | tgtgccagcagtgaaggagctaggggcgtgggggagcagttcttc | CASSEGARGVGEQFF | 1 | 1.52 | 1.52 | |
| TRBV2*02 or | | | | | | | | |
| TRBV2*03 | | | | | | | | |
| TRBV2*01, or | TRBJ2-5*01 | TRBD2*01 | tgtgccagcagtgaaggggctagcggtacgggggcccagtacttc | CASSEGASGTGAQYF | 1 | 1.52 | 1.52 | |
| TRBV2*02 or | | | | | | | | |
| TRBV2*03 | | | | | | | | |
| TRBV2*01, or | TRBJ2-1*01 | TRBD1*01 | tgtgccagcagtgaaggcgctagtggcgtaggggagcagttcttc | CASSEGASGVGEQFF | 1 | 1.52 | 1.52 | |
| TRBV2*02 or | | | | | | | | |
| TRBV2*03 | | | | | | | | |
| TRBV2*01, or | TRBJ2-1*01 | TRBD2*01 | tgtgccagcagtgagggtactagcacctttcgtgagcagttcttc | CASSEGTSTFREQFF | 2 | 3.03 | 3.03 | |
| TRBV2*02 or | | | | | | | | |
| TRBV2*03 | | | | | | | | |
| TRBV2*01, or | TRBJ2-2*01 | TRBD2*01 | tgtgccagcagtgaattagcgagcggcaccggggagctgtttttt | CASSELASGTGELFF | 1 | 1.52 | 1.52 | |
| TRBV2*02 or | | | | | | | | |
| TRBV2*03 | | | | | | | | |
| TRBV2*01, or | TRBJ1-5*01 | TRBD2*02 | tgtgccagcagtggggcagcgaggggcaatcagccccagcatttt | CASSGAARGNQPQHF | 5 | 7.58 | 7.58 | |
| TRBV2*02 or | | | | | | | | |
| TRBV2*03 | | | | | | | | |
| TRBV2*01, or | TRBJ2-7*01 | TRBD2*01 | tgtgccagcagcaaactgactagcgggggatacgagcagtacttc | CASSKLTSGGYEQYF | 2 | 3.03 | 3.03 | |
| TRBV2*02 or | | | | | | | | |
| TRBV2*03 | | | | | | | | |
| TRBV2*01, or | TRBJ2-1*01 | TRBD2*01 | tgtgccagcagtcccgggactagcggggttggtgagcagttcttc | CASSPGTSGVGEQFF* | 2 | 3.03 | 3.03 | |
| TRBV2*02 or | | | | | | | | |
| TRBV2*03 | | | | | | | | |
| TRBV2*01, or | TRBJ2-1*01 | TRBD2*01 | tgtgccagcagtcgtctagcggggggtttcgatgagcagttcttc | CASSRLAGGFDEQFF | 2 | 3.03 | 3.03 | |
| TRBV2*02 or | | | | | | | | |
| TRBV2*03 | | | | | | | | |
| TRBV2*01, or | TRBJ2-3*01 | TRBD2*01 | tgtgccagcagtcgactagcggggggcacagatacgcagtatttt | CASSRLAGGTDTQYF | 2 | 3.03 | 3.03 | |
| TRBV2*02 or | | | | | | | | |
| TRBV2*03 | | | | | | | | |
| TRBV2*01, or | TRBJ2-3*01 | TRBD2*01 | tgtgccagcagtagactagcgtctggcacagatacgcagtatttt | CASSRLASGTDTQYF | 5 | 7.58 | 7.58 | |
| TRBV2*02 or | | | | | | | | |
| TRBV2*03 | | | | | | | | |
| TRBV2*01, or | | | | | | | | |
| TRBV2*02 or | TRBJ2-7*01 | TRBD2*01 | tgtgccagcagtgtattggctagcgggctgggtgagcagtacttc | CASSVLASGLGEQYF | 1 | 1.52 | 1.52 | 48 |
| TRBV2*03 | | | | | | | | |
| TRBV2*01, or | TRBJ1-5*01 | TRBD1*01 | tgtgccagcagacagggggcgaggggaggcaatcagccccagcatttt | CASRQGARGGNQPQHF | 6 | 9.09 | 9.09 | |
| TRBV2*02 or | | | | | | | | |
| TRBV2*03 | | | | | | | | |
| TRBV2*01, or | TRBJ1-5*01 | TRBD1*01 | tgtgccagctcacagggggcgcgggggggaaatcagccccagcatttt | CASSQGARGGNQPQHF | 1 | 1.52 | 1.52 | |
| TRBV2*02 or | | | | | | | | |
| TRBV2*03 | | | | | | | | |
| TRBV2*01, or | TRBJ2-7*01 | TRBD2*01 | tgtgccagcagtcgattagcgggggggagctcctacgagcagtacttc | CASSRLAGGSSYEQYF | 2 | 3.03 | 3.03 | |
| TRBV2*02 or | | | | | | | | |
| TRBV2*03 | | | | | | | | |
| TRBV2*01, or | TRBJ2-3*01 | TRBD2*01 | tgtgccagcagccgcctgactagcgggggggcagatacgcagtatttt | CASSRLTSGGADTQYF | 1 | 1.52 | 1.52 | |
| TRBV2*02 or | | | | | | | | |
| TRBV2*03 | | | | | | | | |
| TRBV2*01, or | TRBJ2-3*01 | TRBD2*01 | tgtgccagcagccgccggactagcgggggcacagatacgcagtatttt | CASSRRTSGGTDTQYF* | 12 | 18.18 | 18.18 | |
| TRBV2*02 or | | | | | | | | |
| TRBV2*03 | | | | | | | | |
| | | | Total | 28 | 66 | 100.00 | 100.00 | |
| HIC 7 DRB5+ | | | | | | | | |
| TRBV2*01, or | TRBJ2-7*01 | TRBD2*01 | tgtgccagcagtcctcgggcctcggggggagagcagtacttc | CASSPRASGGEQYF | 3 | 3.85 | 3.85 | 42 |
| TRBV2*02 or | | | | | | | | |
| TRBV2*03 | | | | | | | | |
| TRBV2*01, or | TRBJ1-4*01 | TRBD2*01 | tgtgccagcagtgtgcggcggaataatgaaaaactgtttttt | CASSVRRNNEKLFF | 3 | 3.85 | 3.85 | |
| TRBV2*02 or | | | | | | | | |
| TRBV2*03 | | | | | | | | |
| TRBV2*01, or | TRBJ2-7*01 | TRBD2*01 | tgtgccagcaaccgaaggactagcggaacctacgagcagtacttc | CASNRRTSGTYEQYF | 1 | 1.28 | 1.28 | 45 |
| TRBV2*02 or | | | | | | | | |
| TRBV2*03 | | | | | | | | |
| TRBV2*01, or | TRBJ2-3*01 | TRBD2*01 | tgtgccagcagcgcactagcgtccgggggagatacgcagtatttt | CASSALASGGDTQYF | 3 | 3.85 | 3.85 | |
| TRBV2*02 or | | | | | | | | |
| TRBV2*03 | | | | | | | | |
| TRBV2*01, or | TRBJ2-1*01 | TRBD2*01 | tgtgccagcagtgcacgggctagcgggggggatgagcagttcttc | CASSARASGGDEQFF | 3 | 3.85 | 5.13 | |
| TRBV2*02 or | | | | | | | | |
| TRBV2*03 | | | | | | | | |
| TRBV2*01, or | TRBJ2-1*01 | TRBD2*01 | tgtgccagcagtgcacgggctagcgggggggatgagcagtttttc | | 1 | 1.28 | | |
| TRBV2*02 or | | | | | | | | |
| TRBV2*03 | | | | | | | | |
| TRBV2*01, or | TRBJ1-5*01 | TRBD1*01 | tgtgccagcagtgcccggacgagtgggggtcagccccagcatttt | CASSARTSGGQPQHF | 4 | 5.13 | 5.13 | |
| TRBV2*02 or | | | | | | | | |
| TRBV2*03 | | | | | | | | |
| TRBV2*01, or | TRBJ1-5*01 | TRBD1*01 | tgtgccagcagcgcccggacatcgggcaatcagccccagcatttt | CASSARTSGNQPQHF | 11 | 14.10 | 14.10 | |
| TRBV2*02 or | | | | | | | | |
| TRBV2*03 | | | | | | | | |
| TRBV2*01, or | TRBJ2-1*01 | TRBD2*02 | tgtgccagcagtgaactggctagcgggatcaatgagcagttcttc | CASSELASGINEQFF | 5 | 6.41 | 6.41 | |
| TRBV2*02 or | | | | | | | | |
| TRBV2*03 | | | | | | | | |
| TRBV2*01, or | TRBJ2-1*01 | TRBD2*01 | tgtgccagcagtgagttgactagcgggggggatgagcagttcttc | CASSELTSGGDEQFF | 1 | 1.28 | 1.28 | |
| TRBV2*02 or | | | | | | | | |
| TRBV2*03 | | | | | | | | |
| TRBV2*01, or | TRBJ2-3*01 | TRBD2*02 | tgtgccagcagtaagaggacctctggaggagatacgcagtatttt | CASSKRTSGGDTQYF | 1 | 1.28 | 1.28 | |
| TRBV2*02 or | | | | | | | | |
| TRBV2*03 | | | | | | | | |
| TRBV2*01, or | TRBJ2-7*01 | TRBD2*01 | tgtgccagcagcctaaggactagcgggggggatgagcagtacttc | CASSLRTSGGDEQYF | 3 | 3.85 | 3.85 | |
| TRBV2*02 or | | | | | | | | |
| TRBV2*03 | | | | | | | | |
| TRBV2*01, or | TRBJ2-3*01 | TRBD2*01 | tgtgccagcagccccctgactagcgccacagatacgcagtatttt | CASSPLTSATDTQYF | 1 | 1.28 | 1.28 | |
| TRBV2*02 or | | | | | | | | |
| TRBV2*03 | | | | | | | | |
| TRBV2*01, or | TRBJ2-1*01 | TRBD2*01 | tgtgccagcagtcctcgggctagcgggggcgatgagcagttcttc | CASSPRASGGDEQFF | 5 | 6.41 | 6.41 | |
| TRBV2*02 or | | | | | | | | |
| TRBV2*03 | | | | | | | | |
| TRBV2*01, or | TRBJ2-7*01 | TRBD2*01 | tgtgccagcagtccccggactagcgggggcgacgagcagtacttc | CASSPRTSGGDEQYF | 1 | 1.28 | 1.28 | |
| TRBV2*02 or | | | | | | | | |
| TRBV2*03 | | | | | | | | |
| TRBV2*01, or | TRBJ2-3*01 | TRBD2*01 | tgtgccagcaggctccggactagcgggggtacagatacgcagtatttt | CASRLRTSGGTDTQYF | 2 | 2.56 | 2.56 | 48 |
| TRBV2*02 or | | | | | | | | |
| TRBV2*03 | | | | | | | | |
| TRBV2*01, or | TRBJ2-3*01 | TRBD2*01 | tgtgccagcagacgactagcggggtttatggcagatacgcagtatttt | CASRRLAGFMADTQYF | 1 | 1.28 | 1.28 | |
| TRBV2*02 or | | | | | | | | |
| TRBV2*03 | | | | | | | | |
| TRBV2*01, or | TRBJ2-3*01 | TRBD2*01 | tgtgccagcagtgcacggactagcgcgggcacagatacgcagtatttt | CASSARTSAGTDTQYF | 1 | 1.28 | 7.69 | |
| TRBV2*02 or | | | | | | | | |
| TRBV2*03 | | | | | | | | |
| TRBV2*01, or | TRBJ2-3*01 | TRBD2*01 | tgtgccagcagtgcacggactagcgctggcacagatacgcagtatttt | | 5 | 6.41 | | |
| TRBV2*02 or | | | | | | | | |
| TRBV2*03 | | | | | | | | |
| TRBV2*01, or | TRBJ2-3*01 | TRBD2*01 | tgtgccagcagtgccaggactagcgggggggcagatacgcattatttt | CASSARTSGGADTHYF | 1 | 1.28 | 1.28 | |
| TRBV2*02 or | | | | | | | | |
| TRBV2*03 | | | | | | | | |
| TRBV2*01, or | TRBJ2-3*01 | TRBD2*01 | tgtgccagcagtgccaggactagcgggggggcagatacgcagtatttt | CASSARTSGGADTQYF | 1 | 1.28 | 2.56 | |
| TRBV2*02 or | | | | | | | | |
| TRBV2*03 | | | | | | | | |
| TRBV2*01, or | TRBJ2-3*01 | TRBD2*01 | tgtgccagcagtgctaggactagcgggggggcagatacgcagtatttt | | 1 | 1.28 | | |
| TRBV2*02 or | | | | | | | | |
| TRBV2*03 | | | | | | | | |
| TRBV2*01, or | TRBJ2-3*01 | TRBD2*01 | tgtgccagcagcgcccggactagcggggggtcagatacgcagtatttt | CASSARTSGGSDTQYF | 1 | 1.28 | 3.85 | |
| TRBV2*02 or | | | | | | | | |
| TRBV2*03 | | | | | | | | |
| TRBV2*01, or | TRBJ2-3*01 | TRBD2*01 | tgtgccagcagtgccaggactagcggggggtcagatacgcagtatttt | | 2 | 2.56 | | |
| TRBV2*02 or | | | | | | | | |
| TRBV2*03 | | | | | | | | |
| TRBV2*01, or | TRBJ2-3*01 | TRBD2*01 | tgtgccagcagtgccaggactagcggaggcacagatacgcagtatttt | CASSARTSGGTDTQYF | 2 | 2.56 | 3.85 | |
| TRBV2*02 or | | | | | | | | |
| TRBV2*03 | | | | | | | | |
| TRBV2*01, or | TRBJ2-3*01 | TRBD2*01 | tgtgccagcagtgctaggactagogggggcacagatacgcagtatttt | | 1 | 1.28 | | |
| TRBV2*02 or | | | | | | | | |
| TRBV2*03 | | | | | | | | |
| TRBV2*01, or | TRBJ2-3*01 | TRBD2*01 | tgtgccagcagtgaacggactagcgggggacgcgatacgcagtatttt | CASSERTSGGRDTQYF | 1 | 1.28 | 1.28 | |
| TRBV2*02 or | | | | | | | | |
| TRBV2*03 | | | | | | | | |
| TRBV2*01, or | TRBJ2-3*01 | TRBD2*01 | tgtgccagcagcggtaggactagcgggggatcggatacgcagtatttt | CASSGRTSGGSDTQYF | 3 | 3.85 | 3.85 | |
| TRBV2*02 or | | | | | | | | |
| TRBV2*03 | | | | | | | | |
| TRBV2*01, or | TRBJ2-3*01 | TRBD2*01 | tgtgccagcagtcttagggctagcggggggtcagatacgcagtatttt | CASSLRASGGSDTQYF | 1 | 1.28 | 1.28 | |
| TRBV2*02 or | | | | | | | | |
| TRBV2*03 | | | | | | | | |
| TRBV2*01, or | TRBJ2-3*01 | TRBD2*01 | tgtgccagcagtctgaggactagcgggggggcagatacgcagtatttt | CASSLRTSGGADTQYF | 1 | 1.28 | 1.28 | |
| TRBV2*02 or | | | | | | | | |
| TRBV2*03 | | | | | | | | |
| TRBV2*01, or | TRBJ2-3*01 | TRBD2*01 | tgtgccagcagccaacggactagcgggggggcagatacgcagtatttt | CASSQRTSGGADTQYF | 1 | 1.28 | 1.28 | |
| TRBV2*02 or | | | | | | | | |
| TRBV2*03 | | | | | | | | |
| TRBV2*01, or | TRBJ2-3*01 | TRBD2*01 | tgtgccagcagccgactgactagcgggggatcagatacgcagtatttt | CASSRLTSGGSDTQYF | 4 | 5.13 | 5.13 | |
| TRBV2*02 or | | | | | | | | |
| TRBV2*03 | | | | | | | | |
| TRBV2*01, or | TRBJ2-3*01 | TRBD2*01 | tgtgccagcagtcgccggactagcgggggtctagatacgcagtatttt | CASSRRTSGGLDTQYF | 1 | 1.28 | 1.28 | |
| TRBV2*02 or | | | | | | | | |
| TRBV2*03 | | | | | | | | |
| TRBV2*01, or | TRBJ2-1*01 | TRBD2*01 | tgtgccagcagtcggcggactagcggggggcccaatgagcagttcttc | CASSRRTSGGPNEQFF | 1 | 1.28 | 1.28 | |
| TRBV2*02 or | | | | | | | | |
| TRBV2*03 | | | | | | | | |
| TRBV2*01, or | TRBJ2-3*01 | TRBD2*01 | tgtgccagcagtcggaggactagcgggggcacagatacgcagtatttt | CASSRRTSGGTDTQYF* | 1 | 1.28 | 1.28 | |
| TRBV2*02 or | | | | | | | | |
| TRBV2*03 | | | | | | | | |
| | | | Total | 30 | 78 | 100.00 | 100.00 | |
| HIC 8 DR1+ | | | | | | | | |
| TRBV2*01, or | TRBJ2-7*01 | TRBD1*01 | tgtgccagcagtgaagggtgggaaccctacgagcagtacttc | CASSEGWEPYEQYF | 1 | 1.49 | 1.49 | 42 |
| TRBV2*02 or | | | | | | | | |
| TRBV2*03 | | | | | | | | |
| TRBV2*01, or | TRBJ2-1*01 | TRBD2*01 | tgtgccagcagtgagttgactagcgggggggatgagcagttcttc | CASSELTSGGDEQFF | 63 | 94.03 | 94.03 | 45 |
| TRBV2*02 or | | | | | | | | |
| TRBV2*03 | | | | | | | | |
| TRBV2*01, or | TRBJ2-1*01 | TRBD2*01 | tgtgccagcagtgagttgactagcgggggggatgagcagttgttc | CASSELTSGGDEQLF | 1 | 1.49 | 1.49 | |
| TRBV2*02 or | | | | | | | | |
| TRBV2*03 | | | | | | | | |
| TRBV2*01, or | TRBJ2-3*01 | TRBD2*01 | tgtgccagcagtgccaggactagcggaggcacagatacgcagtatttt | CASSARTSGGTDTQYF | 1 | 1.49 | 1.49 | 48 |
| TRBV2*02 or | | | | | | | | |
| TRBV2*03 | | | | | | | | |
| TRBV2*01, or | TRBJ1-2*01 | TRBD1*01 | tgtgccaccaccgccgccgggacaggggtagacggaaactatggctacacc | CATTAAGTGVDGNYGYTF | 1 | 1.49 | 1.49 | 54 |
| TRBV2*02 or | | | ttc | | | | | |
| TRBV2*03 | | | | | | | | |
| | | | Total | 5 | 67 | 100 | 100 | |
| HIC 1 DRB5+ | | | | | | | | |
| ex vivo | | | | | | | | |
| TRBV2*01 | TRBJ2-1*01 | TRBD2*01 | tgtgccagccagcggggaaattcctacaatgagcagttcttc | CASQRGNSYNEQFF | 2 | 4.17 | 4.17 | 42 |
| TRBV2*01 | TRBJ2-7*01 | TRBD2*01 | tgtgccagcagaatccggactagcgggggggagcagtacttc | CASRIRTSGGEQYF | 11 | 22.92 | 22.92 | |
| TRBV2*01 | TRBJ2-1*01 | TRBD1*01 | tgtgccagcacccgggacaggacaaagaatgagcagttcttc | CASTRDRTKNEQFF | 1 | 2.08 | 2.08 | |
| TRBV2*01 | TRBJ2-1*01 | TRBD2*01 | tgtgccagcagtgcgttggctagcgggggagatgagcagttcttc | CASSALASGGDEQFF | 1 | 2.08 | 2.08 | 45 |
| TRBV2*01 | TRBJ2-1*01 | TRBD2*01 | tgtgccagcagtctccggactagcgggggagatgagcagttcttc | CASSLRTSGGDEQFF | 2 | 4.17 | 4.17 | |
| TRBV2*01 | TRBJ2-1*01 | TRBD2*01 | tgtgccagcagccctatggctagcgggggggatgagcagttcttc | CASSPMASGGDEQFF | 2 | 4.17 | 4.17 | |
| TRBV2*01 | TRBJ2-3*01 | TRBD2*01 | tgtgccagcagcccccggactagcggcacagatacgcagtatttt | CASSPRTSGTDTQYF | 2 | 4.17 | 4.17 | |
| TRBV2*01 | TRBJ2-7*01 | TRBD2*02 | tgtgccagcagccgtaggactagcgggacttacgagcagtacttc | CASSRRTSGTYEQYF | 1 | 2.08 | 2.08 | |
| TRBV2*01 | TRBJ2-1*01 | TRBD2*01 | tgtgccagcagtgtgatggctagccgtgggaatgagcagttcttc | CASSVMASRGNEQFF | 1 | 2.08 | 2.08 | |
| TRBV2*01 | TRBJ2-1*01 | TRBD2*01 | tgtgtcagcagtgcgttggctagcgggggagatgagcagttcttc | CVSSALASGGDEQFF | 1 | 2.08 | 2.08 | |
| TRBV2*01 | TRBJ2-3*01 | TRBD2*01 | tgtgccagcaataaattgactagcgggggccgggatacgcagtatttt | CASNKLTSGGRDTQYF | 4 | 8.33 | 8.33 | |
| TRBV2*01 | TRBJ2-3*01 | TRBD2*01 | tgtgccagcagtgccaggactagcgggggctcggatacgcagtatttt | CASSARTSGGSDTQYF | 4 | 8.33 | 8.33 | 48 |
| TRBV2*01 | TRBJ2-1*01 | TRBD2*01 | tgtgccagcagcaaaaggactagcggggcgactgatgagcagttcttc | CASSKRTSGATDEQFF | 2 | 4.17 | 4.17 | |
| TRBV2*01 | TRBJ2-5*01 | TRBD2*01 | tgtgccagcagtttgttgactagcgggggacgggagacccagtacttc | CASSLLTSGGRETQYF | 6 | 12.50 | 12.50 | |
| TRBV2*01 | TRBJ2-1*01 | TRBD2*01 | tgtgccagcagccgcttgacgagcggggggcggaatgagcagttcttc | CASSRLTSGGRNEQFF | 2 | 4.17 | 4.17 | |
| TRBV2*01 | TRBJ2-3*01 | TRBD2*01 | tgtgccagcagctccaggactagcgggggcacagatacgcagtatttt | CASSSRTSGGTDTQYF | 3 | 6.25 | 6.25 | |
| TRBV2*01 | TRBJ1-5*01 | TRBD2*01 | tgtgccacctccagaggagcgcggggaagcaatcagccccagcatttt | CATSRGARGSNQPQHF | 3 | 6.25 | 6.25 | |
| | | | Total | 17 | 48 | 100.00 | 100.00 | |
| HIC 2 DRB5+ | | | | | | | | |
| ex vivo | | | | | | | | |
| TRBV2*01, | TRBJ2-2*01 | TRBD1*01 | tgtgccagcagagacggcctcggggagctgtttttt | CASRDGLGELFF | 1 | 2.00 | 2.00 | 36 |
| orTRBV2*02 | | | | | | | | |
| orTRBV2*03 | | | | | | | | |
| TRBV2*01, | TRBJ1-1*01 | TRBD1*01 | tgtgccagctcaggacagacgaacactgaagctttcttt | CASSGQTNTEAFF | 3 | 6.00 | 6.00 | 39 |
| orTRBV2*02 | | | | | | | | |
| orTRBV2*03 | | | | | | | | |
| TRBV2*01 | TRBJ2-3*01 | TRBD2*01 | tgtgccagcagtgcactggctagcggaacagatacgcagtatttt | CASSALASGTDTQYF | 1 | 2.00 | 2.00 | 45 |
| TRBV2*01 | TRBJ2-5*01 | TRBD2*01 | tgtgccagcagtgaattggctagcgggcagggatcccagtacttc | CASSELASGQGSQYF | 5 | 10.00 | 10.00 | |
| TRBV2*01 | TRBJ2-7*01 | TRBD2*02 | tgtgccagcagtgaactggctagcgggagctacgagcagtacttc | CASSELASGSYEQYF | 3 | 6.00 | 6.00 | |
| TRBV2*01, | TRBJ2-1*01 | TRBD2*02 | tgtgccagcagtgaactggctagcgggacgggtgagcagttcttc | CASSELASGTGEQFF | 1 | 2.00 | 2.00 | |
| orTRBV2*02 | | | | | | | | |
| orTRBV2*03 | | | | | | | | |
| TRBV2*01, | TRBJ2-7*01 | TRBD2*01 | tgtgccagcagccctcggactagcggtccctacgagcagtacttc | CASSPRTSGPYEQYF | 3 | 6.00 | 6.00 | |
| orTRBV2*02 | | | | | | | | |
| orTRBV2*03 | | | | | | | | |
| TRBV2*01, | TRBJ2-3*01 | TRBD2*01 | tgtgccagcagccaattgactagcggcacagatacgcagtatttt | CASSQLTSGTDTQYF | 2 | 4.00 | 4.00 | |
| orTRBV2*02 | | | | | | | | |
| orTRBV2*03 | | | | | | | | |
| TRBV2*01, | TRBJ2-1*01 | TRBD2*02 | tgtgccagcagtcgactagcgggaggatttgatgagcagttcttc | CASSRLAGGFDEQFF | 1 | 2.00 | 2.00 | |
| orTRBV2*02 | | | | | | | | |
| orTRBV2*03 | | | | | | | | |
| TRBV2*01 | TRBJ2-3*01 | TRBD2*01 | tgtgccagcagtcggttggctagcggcacagatacgcagtatttt | CASSRLASGTDTQYF | 4 | 8.00 | 8.00 | |
| TRBV2*01, | TRBJ1-5*01 | TRBD1*01 | tgtgccagcagtcggacagtctcgggcaatcagccccagcatttt | CASSRTVSGNQPQHF | 1 | 2.00 | 2.00 | |
| orTRBV2*02 | | | | | | | | |
| orTRBV2*03 | | | | | | | | |
| TRBV2*01, | TRBJ2-7*01 | TRBD2*01 | tgtgccagcagttcgttggctagcagaccctacgagcagtacttc | CASSSLASRPYEQYF | 1 | 2.00 | 2.00 | |
| orTRBV2*02 | | | | | | | | |
| orTRBV2*03 | | | | | | | | |
| TRBV2*01, | TRBJ2-1*01 | TRBD2*01 | tgtgccagcacgaagggcgctagcgggtcgggtgagcagttcttc | CASTKGASGSGEQFF | 2 | 4.00 | 4.00 | |
| orTRBV2*02 | | | | | | | | |
| orTRBV2*03 | | | | | | | | |
| TRBV2*01 | TRBJ2-1*01 | TRBD2*02 | tgtgccaccacccccggggctagcgggataagtgagcagttcttc | CATTPGASGISEQFF* | 14 | 28.00 | 28.00 | |
| TRBV2*01 | TRBJ2-7*01 | TRBD1*01 | tgtgccagcagtgaaaggggacagggggcgcggtacgagcagtacttc | CASSERGQGARYEQYF | 5 | 10.00 | 10.00 | 48 |
| TRBV2*01, | TRBJ2-3*01 | TRBD2*01 | tgtgccagcagccgaaggactagcggaggcacagatacgcagtatttt | CASSRRTSGGTDTQYF* | 2 | 4.00 | 6.00 | |
| orTRBV2*02 | | | | | | | | |
| orTRBV2*03 | | | | | | | | |
| TRBV2*01, | TRBJ2-3*01 | TRBD2*01 | tgtgccagcagccgaaggactagcggaggtacagatacgcagtatttt | | 1 | 2.00 | | |
| orTRBV2*02 | | | | | | | | |
| orTRBV2*03 | | | | | | | | |
| | | | Total | 16 | 50 | 100.00 | 100.00 | |
| HIC 3 DRB5+ | | | | | | | | |
| ex vivo | | | | | | | | |
| TRBV2*01 | TRBJ2-1*01 | TRBD2*02 | tgtgccagcgcccgactagcgggaggtaccgatgagcagttcttc | CASARLAGGTDEQFF | 1 | 4.00 | 4.00 | 45 |
| TRBV2*01 | TRBJ1-5*01 | TRBD2*01 | tgtgccagcagcgcgaaggcccgcgggaatcagccccagcatttt | CASSAKARGNQPQHF | 2 | 8.00 | 8.00 | |
| TRBV2*01 | TRBJ2-3*01 | TRBD2*01 | tgtgccagcagcgcactagcggggggaacagatacgcagtatttt | CASSALAGGTDTQYF | 3 | 12.00 | 12.00 | |
| TRBV2*01 | TRBJ2-3*01 | TRBD2*01 | tgtgccagcagtgctttggctagcggcacagatacgcagtatttt | CASSALASGTDTQYF | 5 | 20.00 | 20.00 | |
| TRBV2*01 | TRBJ2-7*01 | TRBD2*01 | tgtgccagcagtgacgcggctagcggtgtgggcgagcagtacttc | CASSDAASGVGEQYF | 4 | 16.00 | 16.00 | |
| TRBV2*01 | TRBJ1-5*01 | TRBD1*01 | tgtgccagcagcggacaggcgaggggcaatcagccccagcatttt | CASSGQARGNQPQHF | 4 | 16.00 | 16.00 | |
| TRBV2*01 | TRBJ2-1*01 | TRBD2*01 | tgtgcaagcagccccgggactagcggagttggtgagcagttcttc | CASSPGTSGVGEQFF* | 4 | 16.00 | 16.00 | |
| TRBV2*01 | TRBJ1-5*01 | TRBD1*01 | tgtgccagcagtcccagtgctcgcggcaatcagccccagcatttt | CASSPSARGNQPQHF | 1 | 4.00 | 4.00 | |
| TRBV2*01 | TRBJ2-7*01 | TRBD2*01 | tgtgccagcagtgtcgggactagcggggtgggcgagcagtacttc | CASSVGTSGVGEQYF | 1 | 4.00 | 4.00 | |
| | | | Total | 9 | 25 | 100.00 | 100.00 | |
| HIC 7 DRB5+ | | | | | | | | |
| ex vivo | | | | | | | | |
| TRBV2*01, | TRBJ2-7*01 | TRBD2*01 | tgtgccagccaggcaagcggccgatcctacgagcagtacttc | CASQASGRSYEQYF | 4 | 3.67 | 3.67 | 42 |
| orTRBV2*02 | | | | | | | | |
| orTRBV2*03 | | | | | | | | |
| TRBV2*01 | TRBJ2-5*01 | TRBD1*01 | tgtgccagcagtgaattttgggggcaagagacccagtacttc | CASSEFWGQETQYF | 1 | 0.92 | 0.92 | |
| TRBV2*01 | TRBJ2-1*01 | TRBD2*01 | tgtgccagcagtgagctggctagcggggatgagcagttcttc | CASSELASGDEQFF | 9 | 8.26 | 8.26 | |
| TRBV2*01 | TRBJ2-7*01 | TRBD1*01 | tgtgccagcagtgtatcgcaggggagcgacgagcagtacttc | CASSVSQGSDEQYF | 1 | 0.92 | 0.92 | |
| TRBV2*01, | TRBJ2-7*01 | TRBD2*01 | tgtgccagctgtcccatggctagccgatcctacgagcagtacttc | CASCPMASRSYEQYF | 1 | 0.92 | 0.92 | |
| orTRBV2*02 | | | | | | | | |
| orTRBV2*03 | | | | | | | | |
| TRBV2*01 | TRBJ1-2*01 | TRBD1*01 | tgtgccagcattatcggttcccaaggggcctatggctacaccttc | CASIIGSQGAYGYTF | 1 | 0.92 | 0.92 | |
| TRBV2*01 | TRBJ2-3*01 | TRBD2*01 | tgtgccagcagcgcactagcgtccgggggagatacgcagtatttt | CASSALASGGDTQYF | 6 | 5.50 | 5.50 | |
| TRBV2*01, | TRBJ2-1*01 | TRBD2*01 | tgtgccagcagtgcacgggctagcgggggggatgagcagttcttc | CASSARASGGDEQFF | 4 | 3.67 | 3.67 | |
| orTRBV2*02 | | | | | | | | |
| orTRBV2*03 | | | | | | | | |
| TRBV2*01, | TRBJ1-5*01 | TRBD1*01 | tgtgccagcagcgcccggacatcgggcaatcagccccagcatttt | CASSARTSGNQPQHF | 5 | 4.59 | 4.59 | |
| orTRBV2*02 | | | | | | | | |
| orTRBV2*03 | | | | | | | | |
| TRBV2*01, | TRBJ1-1*01 | TRBD1*01 | tgtgccagcagtgaacgcgggacagccaacactgaagctttcttt | CASSERGTANTEAFF | 1 | 0.92 | 0.92 | 45 |
| orTRBV2*02 | | | | | | | | |
| TRBV2*01, | TRBJ2-3*01 | TRBD2*01 | tgtgccagcagcttcaggactagcgggggtgatacgcagtatttt | CASSFRTSGGDTQYF | 2 | 1.83 | 1.83 | |
| orTRBV2*02 | | | | | | | | |
| orTRBV2*03 | | | | | | | | |
| TRBV2*01, | TRBJ2-3*01 | TRBD2*01 | tgtgccagcagtaagcgggctagcgggggagatacgcagtatttt | CASSKRASGGDTQYF | 2 | 1.83 | 1.83 | |
| orTRBV2*02 | | | | | | | | |
| TRBV2*01, | | | | | | | | |
| orTRBV2*02 | TRBJ2-1*01 | TRBD2*01 | tgtgccagcagtcctcgggctagcgggggcgatgagcagttcttc | CASSPRASGGDEQFF | 3 | 2.75 | 2.75 | |
| orTRBV2*03 | | | | | | | | |
| TRBV2*01 | TRBJ2-7*01 | TRBD2*01 | tgtgccagcagtccccggactagcgggggcgacgagcagtacttc | CASSPRTSGGDEQYF | 7 | 6.42 | 6.42 | |
| TRBV2*01, | TRBJ2-7*01 | TRBD2*02 | tgtgccagcagccccaggactagcgggacctacgagcagtacttc | CASSPRTSGTYEQYF | 1 | 0.92 | 15.60 | |
| orTRBV2*02 | | | | | | | | |
| TRBV2*01 | TRBJ2-7*01 | TRBD2*02 | tgtgccagcagtcctcggactagcgggacctacgagcagtacttc | | 16 | 14.68 | | |
| TRBV2*01 | TRBJ2-7*01 | TRBD1*01 | tgtgccagcagccccgtggccagggggccttacgagcagtacttc | CASSPVARGPYEQYF | 1 | 0.92 | 0.92 | |
| TRBV2*01 | TRBJ2-7*01 | TRBD2*01 | tgtgccagcagtcaactgactagcagaacctacgagcagtacttc | CASSQLTSRTYEQYF | 1 | 0.92 | 0.92 | |
| TRBV2*01, | TRBJ2-3*01 | TRBD2*01 | tgtgccagcagtgccaggactagcggggggtcagatacgcagtatttt | CASSARTSGGSDTQYF | 6 | 5.50 | 5.50 | 48 |
| orTRBV2*02 | | | | | | | | |
| TRBV2*01, | TRBJ2-3*01 | TRBD2*01 | tgtgccagcagcgcccggactagcgggggcacagatacgcagtatttt | CASSARTSGGTDTQYF | 1 | 0.92 | 11.01 | |
| orTRBV2*02 | | | | | | | | |
| orTRBV2*03 | | | | | | | | |
| TRBV2*01 | TRBJ2-3*01 | TRBD2*01 | tgtgccagcagtgccaggactagcggaggcacagatacgcagtatttt | | 11 | 10.09 | | |
| TRBV2*01, | TRBJ2-3*01 | TRBD2*01 | tgtgccagcagcggtaggactagcgggggatcggatacgcagtatttt | CASSGRTSGGSDTQYF | 4 | 3.67 | 3.67 | |
| orTRBV2*02 | | | | | | | | |
| TRBV2*01 | TRBJ2-3*01 | TRBD2*01 | tgtgccagcagtctgaggactagcgggggggcagatacgcagtatttt | CASSLRTSGGADTQYF | 1 | 0.92 | 0.92 | |
| TRBV2*01, | TRBJ2-3*01 | TRBD2*01 | tgtgccagcagtctcaggactagcgggggttcagatacgcagtatttt | CASSLRTSGGSDTQYF | 2 | 1.83 | 1.83 | |
| orTRBV2*02 | | | | | | | | |
| orTRBV2*03 | | | | | | | | |
| TRBV2*01, | TRBJ2-3*01 | TRBD2*01 | tgtgccagcagtcctctgactagcgggggcacagatacgcagtatttt | CASSPLTSGGTDTQYF | 1 | 0.92 | 0.92 | |
| orTRBV2*02 | | | | | | | | |
| orTRBV2*03 | | | | | | | | |
| TRBV2*01, | TRBJ2-3*01 | TRBD2*01 | tgtgccagcagtcggaggactagcggggcctcagatacgcagtatttt | CASSRRTSGASDTQYF | 1 | 0.92 | 0.92 | |
| orTRBV2*02 | | | | | | | | |
| TRBV2*01, | TRBJ2-1*01 | TRBD2*01 | tgtgccagcagtcgacgtactagcgggggggccgatgagcagttcttc | CASSRRTSGGADEQFF | 1 | 0.92 | 0.92 | |
| orTRBV2*02 | | | | | | | | |
| orTRBV2*03 | | | | | | | | |
| TRBV2*01, | TRBJ2-3*01 | TRBD2*01 | tgtgccagcagccgacggactagcggggggacggatacgcagtatttt | | 2 | 1.83 | 5.50 | |
| orTRBV2*02 | | | | | | | | |
| orTRBV2*03 | | | | | | | | |
| TRBV2*01 | TRBJ2-3*01 | TRBD2*01 | tgtgccagcagccggcggactagcgggggcacagatacgcagtatttt | CASSRRTSGGTDTQYF* | 2 | 1.83 | | |
| TRBV2*01 | TRBJ2-3*01 | TRBD2*01 | tgtgccagcagtagaaggactagcggtggcacagatacgcagtatttt | | 1 | 0.92 | | |
| TRBV2*01, | TRBJ2-3*01 | TRBD2*01 | tgtgccagcagtagacggactagcggggggacagatacgcagtatttt | | 1 | 0.92 | | |
| orTRBV2*02 | | | | | | | | |
| TRBV2*01, | TRBJ2-1*01 | TRBD2*01 | tgtgccagcagcagacgaactagcgggggatacgatgagcagttcttc | CASSRRTSGGYDEQFF | 2 | 1.83 | 1.83 | |
| orTRBV2*02 | | | | | | | | |
| orTRBV2*03 | | | | | | | | |
| TRBV2*01 | TRBJ2-3*01 | TRBD2*01 | tgtgccagcagtagccggactagcgggggatcagatacgcagtatttt | CASSSRTSGGSDTQYF | 5 | 4.59 | 4.59 | |
| TRBV2*01 | TRBJ2-3*01 | TRBD2*01 | tgtgccagcagtgttcggactagcggggggtcagatacgcagtatttt | CASSVRTSGGSDTQYF | 1 | 0.92 | 0.92 | |
| TRBV2*01 | TRBJ2-7*01 | TRBD1*01 | tgtgccagcagtataagtttacccgggacactttattacgagcagtacttc | CASSISLPGTLYYEQYF | 1 | 0.92 | 0.92 | 51 |
| | | | Total | 30 | 109 | 100.0 | 100.00 | |

TABLE 10
Antigen sensitivity of the TCRs tested by transduction in J76 cells. SEQ ID NOs: 1255 to 1274
The EC50 value for CD69 induction in J76 transduced with 10 different TCRs is reported.
Each TCR was tested with a panel of 7 L cell transfcctants used as APC. Each L cell line expressed a single human HLA-DR allele.
The EC50 corresponds to the Gag293 peptide concentration (in M) at which half maximal CD69 induction was observed.
Responses too low for EC50 determination arc indicated by a dash.
The CDR3 junction sequence is reported for the TRA and TRB chains of each TCR tested. Sequences in bold correspond to public clonotvpes.

| TCR | TCRA CDR3 | TRA | CDR3 | TRB CDR3 | TRB | DR3 | DR11 | DR15 | DRB5 | DR1 | DR7 | DR4 | DR3 |
|---|---|---|---|---|---|---|---|---|---|---|---|---|---|
| name | Junction | motif | length | Junction | motif | length | EC50 | EC50 | EC50 | EC50 | EC50 | EC50 | EC50 |
| F24 | CAFKAAGNKLTF | AV24-1 | 10 | CASSRLAGGMDEQFF | BV2-1 | 13 | 4.15E-07 | 2.17E-06 | 4.46E-06 | 2.53E-06 | 8.69E-06 | — | — |
| F25 | CAFKAAGNKLTF | AV24-1 | 10 | CATTPGASGISEQF | — | 13 | 7.48E-07 | 8.91E-06 | 2.36E-05 | 3.48E-05 | — | — | — |
| F5 | CAFKAAGNKLTF | AV24-1 | 10 | CASSGLAGGMDEQFF | BV2-1 | 13 | 2.12E-06 | 1.60E-05 | — | — | — | — | — |
| S24 | CASKAAGNKLTF | AV24-1 | 10 | CASSRLAGGMDEQFF | BV2-1 | 13 | 6.19E-07 | 3.57E-06 | 9.23E-06 | 9.98E-06 | — | — | — |
| S25 | CASKAAGNKLTF | AV24-1 | 10 | CATTPGASGISEQF | — | 13 | 3.79E-06 | — | — | — | — | — | — |
| RR5 | CSRRAAGNKLTF | AV24-1 | 10 | CASSGLAGGMDEQF | BV2-1 | 13 | 1.25E-06 | 8.46E-06 | 1.10E-05 | 1.11E-05 | — | — | — |
| F4 | CAFKAAGNKLTF | AV24-1 | 10 | CASSPGTSGVGEQFF | BV2-1 | 13 | 1.39E-06 | 6.78E-05 | 8.42E-06 | — | — | — | — |
| F13 | CAFKAAGNKLTF | AV24-1 | 10 | CASSRRTSGGTDTQYF | BV2-2 | 14 | 7.54E-07 | 2.61E-06 | 4.10E-06 | — | — | — | — |
| HD5 | CASDYGGSQGNLI | — | 12 | CASSEFRTRGYTF | — | 11 | — | 8.26E-06 | 5.86E-06 | 1.08E-05 | — | — | — |
| | F | | | | | | | | | | | | |
| HY9 | CAYGANNLFF | — | 8 | CASRPLHSVYEQYF | — | 12 | — | 1.99E-06 | 3.84E-06 | 3.04E-06 | — | — | — |

### Materials and Methods

Study Design

HIV Controllers (HIC group; n=14) were recruited through the CO21 CODEX cohort implemented by Agence Nationale de Recherche sur le SIDA et les hépatites virales (ANRS). HIV Controllers were defined as HIV-1 infected patients who had been seropositive for >5 years, had received no antiretroviral treatment, and for whom >90% of plasma viral load measurements were undetectable by standard assays. All HIV Controllers included in the study had current viral loads <50 copies/mL. The group of efficiently treated patients (HAART group; n=14) had received antiretroviral therapy for a minimum of 5 years and showed long term HIV-1 suppression with viral loads <50 copies/mL. Treated patients were recruited at the Raymond Poincaré and Bicêtre hospitals (France). Patients were included in the TCR study if their genotype matched at least one of the following alleles: DRB1*0101 (DR1), DRB1*1101 (DR11), DRB1*1501 (DR15), or DRB5*0101 (DRB5). Healthy donors were anonymous volunteers who donated blood at Etablissement Francais du Sang.

MHC II Tetramer Labeling of Primary CD4+ T Cell Lines and Patient PBMC

The 20-mer Gag293 peptide (FRDYVDRFYKTLRAEQASQE), spanning amino acids 293-312 in HIV-1 HXB2 Gag, was used to stimulate Gag-specific CD4+ T cell lines, as previously described (27). Cell lines were tested for Gag293 specificity by IFN-γ ELISPOT assay and, if positive, labeled with an HLA-DR-matched Gag293-loaded tetramer and sorted. For ex vivo tetramer labeling, 10⁷ Controller PBMC were tetramer-labeled and sorted, with a minimum of 2,000 Tet+ events acquired for TCR diversity analysis.

Analysis of TCR Genetic Diversity by Immunoscope and Sequencing

TCR diversity was evaluated in sorted Tet+ cell RNA by the Immunoscope technique, as previously described (32). PCR products corresponding to the two amplified familes, TRAV24 and TRBV2, were cloned, sequenced, and analyzed with tools of IMGT database (34). Public motifs enriched in CDR3 sequences were detected with the MEME motif discovery software (http://meme.nbcr.net).

Analysis of TCR Affinity by Surface Plasmon Resonance

Soluble TCRs were produced in bacterial expression vectors as previously described (68). The affinity of the soluble TCRs for immobilized Gag293-loaded HLA-DR monomers (DR11, DRB5, and DR1) was measured on a BIAcore 3000 instrument.

TCR Transfer Experiments

Lentivectors expressing full-length TRAV24 and TRBV2 chains in equimolar amounts were produced by transfecting 293-T cells, concentrated by ultracentrifugation, and used to transduce the Jurkat-derived TCR-negative J76 cell line. TCR function was evaluated by measuring CD69 induction in transduced J76 cells incubated with Gag293-loaded HLA-DR-expressing L cells. Alternatively, monocyte-derived dendritic cells infected with pseudotyped pNL4-3-deltaEnv-EGFP were used as APC. For TCR transfer in primary cells, PBMC were PHA-stimulated for 48 h before transduction with highly concentrated lentivector stocks. TCR function was evaluated by intracellular cytokine staining for IFN-γ, IL-2, MIP-1β, TNF-α and CD107a. All possible combinations of these 5 markers were analyzed by boolean gating, before analysis of polyfunctionality with the SPICE software.

Antibodies

The following antibodies were used for membrane or intracellular staining: CD8-AlexaFluor488 (AF488, clone RPA-T8), CD3-eFluor® 780-Allophycocyanin (eF780-APC, clone UCHT1), TCR-Allophycocyanin (APC, clone IP26) and IL-2-APC (clone MQ1-17H12) (all from eBioscience); CD4 BD Horizon™ R-phycoerythrin-CF594 (PE-CF594, clone RPA-T4), CD4-R-phycoerythrin-cyanine 7 (PE-Cy7, clone SK3), CD69-R-phycoerythrin (PE, clone FN50), CD107a-AlexaFluor700 (AF700, clone H4A3), Perforin-AF488 (clone δG9), IFNγ-PE-Cy7 (clone B27), MIP1β-PE (clone D21-1351) (all from BD Biosciences); CD14-Viogreen (clone TÜK4), and CD19-VioGreen (clone LT19) (from Miltenyi Biotec); TRBV2-PE (clone IMMU 546, Beckman Coulter); CD14-Brilliant Violet 510™ (BV510, clone M5E2), and CD19-BV510 (clone HIB19), CD8-Brilliant Violet 785™ (BV785, clone RPA-T8), HLA-DR-PE-Cy7 (clone LN3), CD45RA-Brilliant Violet 421™ (BV421, clone HI100), CCR7-PE-Cy7 (clone G043H7), and TNFα-BV421 (clone MAb11) (all from Biolegend). The fixable viability dye eFluor® 506 (eF506, eBioscience) was added to restrict the analysis to live cells.

Cell Culture

Transformed cell lines: The mutant Jurkat cell line J76, which lacks endogenous TCR expression, was provided and are as disclosed in (69). J76 cells were maintained in RPMI 1640 medium supplemented with 100 ug/ml penicillin/streptomycin, 1% Hepes buffer, and 2 mM L-glutamine (complete RPMI) in the presence of 10% fetal bovine serum (FBS). Murine fibroblasts (L cells) stably transfected to express a single human HLA-DR allele (DR1, DR3, DR4, DR7, DR11, DR15, or DRB5) were used as antigen presenting cells (70). L cells were maintained in complete RMPI supplemented with 10% FBS and 1% Non-Essential Amino Acids (Life Technologies).

Primary Cells:

Peripheral blood mononuclear cells (PBMC) were isolated from heparinized blood via density gradient centrifugation on Ficoll-Paque PLUS (GE Healthcare Life Sciences) and were either cryopreserved or used freshly for the preparation of monocyte derived dendritic cells (MDDC). The latter were obtained by positive selection of CD14+ monocytes using magnetic Microbeads (Miltenyi Biotec). Monocytes were plated at 2×10⁶ cells per mL in synthetic AIM-V medium (Life Technologies) supplemented with 10 ng/mL GM-CSF and 20 ng/mL IL-4 (both from Miltenyi Biotec) and incubated for 5-7 days at 37° C. in a 5% C02 incubator. Differentiated immature MDDC were collected and cryopreserved until further use.

Primary CD4+ T Cell Lines:

The 20-mer Gag293 peptide (FRD YVD RFY KTL RAE QAS QE), spanning amino acids 293 to 312 in HIV-1 HXB2 Gag, was used in highly purified form (>99% pure; Proteogenix SAS) to stimulate Gag-specific CD4+ T cell lines. PBMCs from HIV-1 infected patients were plated at 2×10⁶ cells per well in 24-well plates in the presence of decreasing amount of Gag293 peptide (10⁻⁵M to 10⁻¹¹M) in complete RPMI supplemented with 10% human AB serum, 0.5 μM AZT, 5 nM Saquinavir and 5 ng/ml recombinant IL-7 (Cytheris). Recombinant IL-2 (Chiron, Novartis) was added to a final concentration of 100 U/ml starting from day 2, and every 2 days afterwards. When CD4+ T cell lines reached doubling time (observed number of cells =2× number of input cells), they were tested for Gag293 specificity by IFN-γ ELISPOT assay, as described previously (71).

MHC Class II Tetramer Labeling

Patients were genotyped for the HLA-DRB1 gene at a 4 digit resolution using the INNO-LiPA HLA-DRB1 Plus kit (Fujirebio). Patients were included in the study if their genotype matched at least one of the following alleles: DR1, DR11, DR15, or DRB5. APC-labeled MHC II tetramers for the DR1, DRB15, DRB5, DRB1*0301 (DR3), and DRB1*0701 (DR7) alleles were obtained through the NIH Tetramer Core Facility at Emory University, USA. HLA-DRB1*0401 (DR4) biotinylated monomers were supplied internally (Institut Pasteur, Paris). DR11 biotinylated monomers were obtained through the tetramer Core laboratory of the Benaroya Research Institute (Seattle, USA). Monomers were loaded with 0.2 mg/ml peptide by incubation at 37° C. for 72 h in the presence of 2.5 mg/ml n-octyl-β-D-glucopyranoside and protease inhibitors. Peptide-loaded monomers were tetramerized using APC-conjugated streptavidin (eBioscience). For each tetramer loaded with the Gag293 peptide, a corresponding control tetramer was loaded with an irrelevant peptide (either the CLIP peptide PVSKMRMATPLLMQA for the DR1, DR15, DR11 tetramers; or an Annexin II peptide DVPKWISIMTERSVPH for the DRB5 tetramer).

The MHC II tetramer labeling protocol was adapted from (72). Primary CD4+ T cells lines were incubated with 1 μg MHC II tetramer/10⁶ cells at a concentration ≥1 μg/mL in complete RPMI supplemented with 15% human AB serum for 90 min at 4° C. Antibodies for surface markers were added for the last 30 min of labeling, using the following combination: CD3-eF780-APC, CD4-PE-CF594, CD8-BV785, CD45RA-BV421, CCR7-PE-Cy7, CD14-VioGreen, and CD19-VioGreen. Gag293-specific tetramer-labeled (Tet+) cells were visualized in the CD3+, CD4+, CD14-, CD19-, CD8-lymphocyte gate, and were sorted using a FACSAria II cell sorter (BD Biosciences) installed in a microbiological safety cabinet. Each Gag293-tetramer labeled sample was matched with a control-tetramer labeled sample processed in identical conditions. Sorted Tet+ cells were resuspended in RLT buffer (Qiagen) and kept frozen at −80° C. until RNA extraction. Sorted samples that yielded a minimum of 20,000 Tet+ events were selected for clonotypic analysis. Patient PBMC were labeled with MHC II tetramer and sorted as described above, with a minimum of 10⁷ cells labeled per sample. Sorted PBMC samples that yielded a minimum of 2,000 Tet+ events were used for clonotypic analysis.

J76 cells expressing recombinant TCRs were labeled as above, except that incubation with the MHC II tetramer was performed in complete RPMI supplemented with 15% FBS for 1 h at 37° C., followed by incubation for 30 min at 4° C. with the following antibody combination: CD3-eF780-APC, CD4-PE-Cy7, αβTCR-APC and eF506-viability dye. The percentage of Tet+ cells was measured in the live CD3+, CD4+ gate after acquisition on an LSR Fortessa cytometer (BD Biosciences).

CDR3 Length Polymorphism Analysis and CDR3 Sequencing

The expression and diversity of 35 TCRα variable gene (TRAV) families and of 24 TCRβ variable gene (TRBV) families were evaluated in Tet+ cells by the Immunoscope technique, as described previously (73, 74). TRAV and TRBV gene expression was measured by quantitative RT-PCR, followed by an analysis of the length distribution of the amplified CDR3 products on a capillary sequencer. Briefly, total RNA was extracted from Tet+ cells using the RNeasy mini or micro kits (Qiagen), depending on available cell number. Next, cDNA was obtained by reverse transcription with 500 g/mL oligo (dT)17 and 200 U of Superscript II reverse transcriptase (Life technologies). A cDNA aliquot was amplified with each of 35 TRAV and 24 TRBV family-specific primers, in combination with a constant region TRAC or a TRBC primer, respectively (73, 74). Amplification was performed in the presence of a family-specific TaqMan probe on an ABI 7300 real time PCR device (Applied Biosystems). An aliquot of each PCR reaction was used as template in a run-off reaction with a nested fluorescent TRAC- or TRBC-specific primer, to generate TRAV- or TRBV-specific single stranded DNA products. These fluorescent DNA samples were separated on an ABI-PRISM 3730 DNA analyzer (Applied Biosystems) and quantified for size and intensity with the Immunoscope software. Fluorescence intensity (in arbitrary units) was plotted in function of the CDR3 length in amino acids.

PCR products corresponding to the TRAV24 and TRBV2 families were cloned and sequenced. The primers used to amplify the full-length chains were: TRAV24 forward primer: 5′-CCG AGG CCT TGT TTG TAA TG-3′; TRAC reverse primer: 5′GTG AAT AGG CAG ACA GAC TTG T-3′; TRBV2 forward primer: 5′-GGT CCG GAA TGG ATA CCT GGC TCG TAT GCT GGG C-3′; TRBC reverse primer: 5′-CCG GTC GAC CTA GCC TCT GGA ATC CTT TCT CTT GAC C-3′. PCR products were cloned into the pCR-Blunt-II-TOPO vector (Life technologies), transformed in E. coli, and analyzed by DNA sequencing (Eurofins Genomics).

Analysis of the TCR Clonotypic Repertoire

TRA and TRB sequences were analyzed with the software suite from the International ImMunoGeneTics (IMGT) Information System (75). The V(D)J gene nomenclature used is that of the IMGT database (www.imgt.org). Clonotypic diversity of the TRAV24 and TRBV2 repertoires was evaluated (i) by counting the number of unique amino acid clonotypes (clonotypes AA) per 100 CDR3 nucleotide sequences (ii) by computing Simpson's diversity index, using the EstimateS software version 9.1.0 (76). Simpson's diversity index takes into account both the number of clonotypes and the frequency of each clonotype in the dataset, and is maximal when all clonotypes have an equal representation. The number of N and P mutations introduced during the V(D)J recombination process was determined by comparing the observed CDR3 sequences to their germline counterparts, using the Junction analysis module of the IMGT/HighV-QUEST software (75). The distribution of TRAV, TRAJ, TRBV, TRBJ, and TRBD genes in the sequence set was computed with the statistics module of IMGT/HighV-QUEST. The CDR3 lengths corresponded to the number of a.a. comprised between, but not including, the two conserved residues C104 and F/W118, as defined by the IMGT-ONTOLOGY unique numbering system. In contrast, the “CDR3 junctions” included the conserved C104 and F/W118 residues. Kurtosis, which measures the “peakedness” of a distribution, was used to evaluate biases in CDR3 lengths. Kurtosis was measured in the Prism v6.0 software (GraphPad). A Gaussian distribution has a kurtosis of zero, while a flatter distribution has negative kurtosis, and a more “peaked” distribution has positive kurtosis.

Motifs enriched in the TRAV24 and TRBV2 CDR3 sequence sets were first identified with the MEME motif discovery software version 4.10.0 (77) available at http://meme-suite.org. The MEME software chooses the width and number of occurrence of each motif automatically in order to minimize the “E-value” of the motif, i.e. the probability of finding an equally well conserved pattern in random sequences. Motifs were searched in discriminative mode, to identify public motifs enriched in the HIC compared to the HAART dataset. Motifs were represented as sequence logos, where the relative sizes of the letters indicate their frequencies in the sequence set, and the total height of the letters represents the information content of the position, in bits. Based on the initial motif analysis, simpler public motifs included within the MEME motifs were identified and counted using the “Protein Pattern Find” module of the Sequence Manipulation Suite (78).

Analysis of Soluble Recombinant TCRs by Surface Plasmon Resonance

Soluble TCRs were engineered by inserting disulfide linkage between the TRAC and TRBC constant domains and truncating the transmembrane and cytoplasmic regions, as previously described (79). The soluble TCRα and TCRβ chains were expressed separately as inclusion bodies, refolded together, and purified before surface plasmon resonance (SPR) analysis. All SPR experiments were conducted at 25° C. on a BIAcore 3000 instrument in the presence of TBS buffer (10 mM Tris-HCl, pH 8, 150 mM NaCl and 0.005% surfactant P20). The TBS buffer was supplemented with 1% BSA to prevent non-specific binding. The pHLA-II complexes were immobilized onto a Streptadivin-coated sensor chip with 1,000-1,200 Response Unit (RU) per flow cell. A flow cell containing a pHLA-I complex was used as negative control. Experiments were conducted as previously described (79), with a concentration range of 0.78-100 μM of pHLA-II complexes. The BIAevaluationVersion 3.1 software was used for data analysis with the 1:1 Langmuir binding model.

TCR Lentivector Construction

Full-length TCRα and TCRβ chains amplified from Gag293-specific Tet+ cells were cloned into lentiviral expression vectors. Full-length TRAV24+ chains were amplified with a forward primer containing the TRAV24 leader sequence with an NheI restriction site and a Kosak sequence added in 5′ (5′-CGG CTA GCC GCC ACC ATG GAG AAG AAT CCT TTG GCA GCC-3′) and a reverse primer containing the 3′ of TRAC and a NotI site (5′-TTA GCG GCC GCG CTG GAC CAC AGC CGC AGC G-3′). Full-length TRBV2+ chains were amplified with a forward primer containing the TRBV2 leader sequence and a BspEI site in 5′ (5′-GGT CCG GAA TGG ATA CCT GGC TCG TAT GCT GGG C-3′) and a reverse primer containing the 3′ of TRBC and a SalI site (5′-CCG GTC GAC CTA GCC TCT GGA ATC CTT TCT CTT GAC C-3′). The TCRα and TCRβ chains were first cloned separately in the pCR-Blunt-II-TOPO vector, and then combined into the pCDH-EF1-MCS-T2A vector (SBI System Bioscience), with a self-cleaving T2A sequence inserted in between, ensuring an equimolar expression of the two chains from the same transcript. All constructs were verified by DNA sequencing.

The sequence of the plasmid termed “pCDH-F24-TCR” herein, encoding the alpha and beta chains of the “F24” TCR reported in Table S8 is provided under SEQ ID NO: 57 (8241 nucleotides)

TCR Lentivector and HIV-1 Pseudotype Preparation

TCR lentivectors were transfected in 293T cells with Lipofectamine 3000 (Life technologies) in the presence of the pPACKH1 Packaging Plasmid Mix (SBI System Bioscience). 48 h after transfection, supernatants were collected, filtered using a 0.45 m filter, and concentrated by ultracentrifugation at 23,000 g for 90 min at 4° C. on a 20% sucrose cushion. Viral particles were resuspended in PBS and frozen in aliquots at −80° C. until use. Gag p24 concentration was measured with the Alliance HIV-1 p24 Antigen ELISA kit (Perkin Elmer).

Single cycle pseudotyped HIV-1 particles (ΨHIV-1) were produced by calcium phosphate transfection of 25 μg of pNL4-3-deltaEnv-EGFP (from the NIH AIDS Reagent Program) and 10 μg of pVSV-G plasmids in 293T cells. Virus-like particles (VLP) expressing Vpx were obtained similarly by co-transfection of the pSIV3+ vector (80) with pVSV-G. Viruses were harvested at 48 h later and concentrated as described above.

TCR Transduction

For TCR transfer, 0.5×10⁶ J76 cells were resuspended in 0.5 mL complete RPMI medium supplemented with 10% FBS in a 24-well plate. TCR lentiviral particles (200 ng of p24) were added to each well and thoroughly resuspended by pipetting. After 3 h, 0.5 mL fresh medium was added to each well. Transduced J76 cells were analyzed at day 3 by staining with CD4-PE-Cy7, TCR-APC, CD3-eF780-APC antibodies and the eF510-viability dye. Samples were acquired on an LSR Fortessa flow cytometer (BD Biosciences) to determine TCR expression and relocalization of the CD3 complex to the cell surface as a measure of transduction efficiency.

To transfer TCRs in primary T cells, healthy donor PBMCs were pre-activated with 5 μg/mL PHA and 50 UI/mL IL-2 for 48 h. PHA blasts were collected and plated at 10⁵ cells/well in a 24-well plate. TCR lentiviral particles (400 ng of p24) were mixed with 5 μL Lentiblast solution A +5 μL Lentiblast solution B (OZ Biosciences), and added to each well. Complete RPMI medium supplemented with 10% FBS and 50 UI/mL IL-2 was added up to a volume of 0.5 mL and plates were centrifuged at 1,000 g for 1 h at 32° C., before incubation o/n at 37° C. The following day, fresh medium and 50 UI/mL IL-2 were added up to a volume of 1 mL. 48 to 72 h later, transduced PBMC were labeled with TRBV2-PE, CD4-PE-CF594, CD8-BV785, CD3-eF780-APC antibodies, and the eF506-viability dye. Samples analyzed by flow cytometry as above, and quantified for an increase in TRBV2 expression level in the live CD3+CD4+CD8-lymphocyte gate to measure transduction efficiency.

Analyses of TCR Functions

J76 Cell Activation Assay:

L cells expressing a single HLA-DR allele were pulsed with serial dilutions (from 2×10⁻⁵ to 10⁻¹¹ M) of Gag293 peptide. 5×10⁴ TCR-transduced J76 cells and 5×10⁴ peptide-pulsed L cells were co-cultured in a 96-well plate o/n at 37° C. On the next day, cells were labeled with CD69-PE, CD4-PE-Cy7, TCR-APC, CD3-eF780-APC antibodies and eF506-viability dye. Samples were acquired in a 96-well plate using a FACSCanto II flow cytometer and analyzed to estimate the induction of CD69 expression as a measure of J76 cell activation upon Gag293-HLA-DR recognition.

To measure J76 cell activation upon stimulation with HIV-1-derived, endogenously processed Gag proteins, MDDC were incubated for 3 h with 50 ng p24 of ΨHIV-1 per 0.15×10⁶ cells in presence of Vpx VLP. Cells were then extensively washed to remove unbound viral particles and co-cultured o/n at 37° C. with TCR-transduced J76 cells at a 1:1 ratio. After co-culture, cells were harvested, washed, treated with FcR Blocker (Miltenyi Biotec), stained with CD69-PE, HLA-DR-PE-Cy7, TCR-APC, CD3-eF780-APC antibodies and eF506-viability dye, and analyzed by flow cytometry as above.

Intracellular Cytokine Staining (ICS) in Primary T Cells:

PBMC from healthy donors expressing at least one of 4 HLA-DR alleles (DR11, DR1, DR15, or DRB5) were transduced with TCR lentivectors and tested 7 to 9 days after transduction. Autologous MDDC (2.5×10⁴) were pulsed with serial dilutions of from 10⁻⁵ to 10⁻¹¹ M of Gag293 peptide and co-cultured at a 1:1 ratio with transduced PBMC. Cells were co-cultured for 1 h at 37° C. before addition of 1 μg/ml Brefeldin A (eBioscience), and further incubated o/n. Negative controls consisted in TCR-transduced PBMC incubated with unpulsed MDDC and in mock-transduced PBMC incubated with peptide-pulsed MDDC. Positive controls were obtained by pulsing MMDC with 1 g/ml Staphylococcal Enterotoxin A (Toxin Technology, Sarasota, Fla.), or by using TCR-transduced PBMC stimulated with 50 ng/mL phorbol 12-myristate 13-acetate (PMA) and 0.25 μg/ml ionomycin in the absence of MDDC.

For ICS, cells were washed, treated with FcR Blocker, and stained for surface antigens with CD4-PE-CF594, CD8-BV785, CD14-BV510, CD3-eF780-APC antibodies, and eF506-viability dye. Cells were fixed and permeabilized using the CytoFix/Cytoperm kit (BD Biosciences) before staining for intracellular cytokines with IL-2-APC, MIP1β-PE, IFNγ-PE-Cy7, TNFα-BV421, and CD107a antibodies. Fluorescence was acquired on an LSR Fortessa flow cytometer. Intracellular cytokine production was evaluated in the live CD14-CD3+CD4+CD8- or CD14-CD3+CD4-CD8+ lymphocyte gates. The percentage of cytokine-producing T cells was determined after subtracting the percentage of cytokine-positive events in unstimulated controls. All flow cytometry experiments were analyzed with the Flowjo v8.8 software (Tree Star). Polyfunctionality was assessed for a panel of 5 functions that included the production of IFN-γ, IL-2, MIP-1β, TNF-α and CD107a. All possible combinations of the 5 markers were analyzed by boolean gating in Flowjo. The resulting data table was converted to the matrix symmetric positive definite (SPD) format, and then analyzed using the SPICE software version 5.3 (81), with a cytokine positivity threshold of 0.1%.

HLA-Blocking Experiments in Primary Cells:

MDDC were brought to a concentration of 5×10⁶/mL in complete RPMI medium supplemented with 10% FBS. MDDC were pretreated with 10 μg/mL of an HLA-DR blocking antibody (Biolegend, clone: L243) or a pan-MHC I blocking antibody (Biolegend, clone: W6/32) for 1 h at 37° C. prior to Gag293 peptide stimulation. Incubation with 10 μg/mL of an isotypic IgG2a control antibody (Biolegend, clone: MOPC-173) was used as a negative control for HLA blocking. MDDC were then pulsed with 10⁻⁵M Gag293 peptide and cocultured at a 1:1 ratio with TCR-transduced PBMC, as described above. Cytokine production was evaluated by ICS in the CD8+ T cell gate.

Statistical Analyses

P values <0.05 were considered statistically significant. Statistics were computed with the Prism v6.0 software (GraphPad) and the R version 3.2.3 software (https://www.r-project.org/). Differences between groups were analyzed with the non parametric Mann-Whitney U test, with the exception of total clonotypic repertoires, for which means where compared with unpaired t tests. Differences in cell line response frequencies and HLA-DR allele frequencies were analyzed in contingency tables with Fisher's exact test. Differences in proportions of CDR3 lengths were computed with a 2-sample test for equality of proportions with continuity correction R. Correlations were analyzed with the non-parametric Spearman's coefficient. Half maximal effective concentrations (EC50) were obtained after non-linear curve fit using a sigmoidal dose response model in Prism. All significant differences between groups (P<0.05) were reported on data plots.

### BIBLIOGRAPHY

1. Deeks S G, et al. Systemic effects of inflammation on health during chronic HIV infection. Immunity. 2013; 39(4):633-45.
2. Connors M, et al. HIV infection induces changes in CD4+ T-cell phenotype and depletions within the CD4+ T-cell repertoire that are not immediately restored by antiviral or immune-based therapies. Nat Med. 1997; 3(5):533-40.
3. Gorochov G, et al. Perturbation of CD4+ and CD8+ T-cell repertoires during progression to AIDS and regulation of the CD4+ repertoire during antiviral therapy. Nat Med. 1998; 4(2):215-21.
4. Rosenberg E S, et al. Vigorous HIV-1-specific CD4+ T cell responses associated with control of viremia. Science. 1997; 278(5342):1447-50.
5. Douek D C, et al. HIV preferentially infects HIV-specific CD4+ T cells. Nature. 2002; 417(6884):95-8.
6. Lubong Sabado R, et al. In vitro priming recapitulates in vivo HIV-1 specific T cell responses, revealing rapid loss of virus reactive CD4 T cells in acute HIV-1 infection. PLoS One. 2009; 4(1):e4256.
7. Lambotte O, et al. HIV controllers: a homogeneous group of HIV-1-infected patients with spontaneous control of viral replication. Clin Infect Dis. 2005; 41(7):1053-6.
8. Chakrabarti L A, and Simon V. Immune mechanisms of HIV control. Curr Opin Immunol. 2010; 22(4):488-96.
9. Saez-Cirion A, et al. HIV controllers exhibit potent CD8 T cell capacity to suppress HIV infection ex vivo and peculiar CTL activation phenotype. Proc Natl Acad Sci USA. 2007; 104(16):6776-81.
10. Almeida J R, et al. Antigen sensitivity is a major determinant of CD8+ T-cell polyfunctionality and HIV-suppressive activity. Blood. 2009; 113(25):6351-60.
11. Ladell K, et al. A molecular basis for the control of preimmune escape variants by HIV-specific CD8+ T cells. Immunity. 2013; 38(3):425-36.
12. Chen H, et al. TCR clonotypes modulate the protective effect of HLA class I molecules in HIV-1 infection. Nat Immunol. 2012; 13(7):691-700.
13. Migueles S A, et al. Lytic granule loading of CD8+ T cells is required for HIV-infected cell elimination associated with immune control. Immunity. 2008; 29(6):1009-21.
14. Almeida J R, et al. Superior control of HIV-1 replication by CD8+ T cells is reflected by their avidity, polyfunctionality, and clonal turnover. J Exp Med. 2007; 204(10):2473-85.
15. Iglesias M C, et al. Escape from highly effective public CD8+ T-cell clonotypes by HIV. Blood. 2011; 118(8):2138-49.
16. Younes S A, et al. HIV-1 viremia prevents the establishment of interleukin 2-producing HIV-specific memory CD4+ T cells endowed with proliferative capacity. J Exp Med. 2003; 198(12): 1909-22.
17. Harari A, et al. Functional heterogeneity of memory CD4 T cell responses in different conditions of antigen exposure and persistence. J Immunol. 2005; 174(2): 1037-45.
18. van Grevenynghe J, et al. Transcription factor FOXO3a controls the persistence of memory CD4(+) T cells during HIV infection. Nat Med. 2008; 14(3):266-74.
19. Potter S J, et al. Preserved central memory and activated effector memory CD4+ T-cell subsets in human immunodeficiency virus controllers: an ANRS EP36 study. J Virol. 2007; 81(24):13904-15.
20. Kaufmann D E, et al. Limited durability of viral control following treated acute HIV infection. PLoS Med. 2004; 1(2):e36.
21. Saez-Cirion A, et al. Restriction of HIV-1 replication in macrophages and CD4+ T cells from HIV controllers. Blood. 2011; 118(4):955-64.
22. Ranasinghe S, et al. HIV-Specific CD4 T Cell Responses to Different Viral Proteins Have Discordant Associations with Viral Load and Clinical Outcome. J Virol. 2012; 86(1):277-83.
23. Ferre A L, et al. HIV controllers with HLA-DRB1*13 and HLA-DQB1*06 alleles have strong, polyfunctional mucosal CD4+ T-cell responses. J Virol. 2010; 84(21):11020-9.
24. Vingert B, et al. HIV controllers maintain a population of highly efficient Th1 effector cells in contrast to patients treated in the long term. J Virol. 2012; 86(19): 10661-74.
25. Kaufmann D E, et al. Upregulation of CTLA-4 by HIV-specific CD4+ T cells correlates with disease progression and defines a reversible immune dysfunction. Nat Immunol. 2007; 8(11):1246-54.
26. Porichis F, et al. Responsiveness of HIV-specific CD4 T cells to PD-1 blockade. Blood. 2011.
27. Vingert B, et al. HIV controller CD4+ T cells respond to minimal amounts of Gag antigen due to high TCR avidity. PLoS Pathog. 2010; 6(2):e1000780.
28. Alexander-Miller M A. High-avidity CD8+ T cells: optimal soldiers in the war against viruses and tumors. Immunol Res. 2005; 31(1): 13-24.
29. Berger C T, et al. High functional avidity CTL responses to HLA-B-restricted Gag-derived epitopes associate with relative HIV control. J Virol. 2011.
30. Kaufmann D E, et al. Comprehensive analysis of human immunodeficiency virus type 1-specific CD4 responses reveals marked immunodominance of gag and nef and the presence of broadly recognized peptides. J Virol. 2004; 78(9):4463-77.
31. Wooldridge L, et al. Tricks with tetramers: how to get the most from multimeric peptide-MHC. Immunology. 2009; 126(2):147-64.
32. Lim A, et al. Frequent contribution of T cell clonotypes with public TCR features to the chronic response against a dominant EBV-derived epitope: application to direct detection of their molecular imprint on the human peripheral T cell repertoire. J Immunol. 2000; 165(4):2001-11.
33. Hacein-Bey-Abina S, et al. Efficacy of gene therapy for X-linked severe combined immunodeficiency. N Engl J Med. 2010; 363(4):355-64.
34. Alamyar E, et al. IMGT((R)) tools for the nucleotide analysis of immunoglobulin (IG) and T cell receptor (TR) V-(D)-J repertoires, polymorphisms, and IG mutations: IMGT/V-QUEST and IMGT/HighV-QUEST for NGS. Methods Mol Biol. 2012; 882:569-604.
35. Turner S J, et al. Structural determinants of T-cell receptor bias in immunity. Nat Rev Immunol. 2006; 6(12):883-94.
36. Venturi V, et al. A mechanism for TCR sharing between T cell subsets and individuals revealed by pyrosequencing. J Immunol. 2011; 186(7):4285-94.
37. Gras S, et al. T-cell receptor bias and immunity. Curr Opin Immunol. 2008; 20(1):119-25.
38. Rossjohn J, et al. T cell antigen receptor recognition of antigen-presenting molecules. Annu Rev Immunol. 2015; 33:169-200.
39. Betts M R, et al. HIV nonprogressors preferentially maintain highly functional HIV-specific CD8+ T cells. Blood. 2006; 107(12):4781-9.
40. Liu P, et al. Characterization of human alphabetaTCR repertoire and discovery of D-D fusion in TCRbeta chains. Protein Cell. 2014; 5(8):603-15.
41. Gillespie G M, et al. Strong TCR conservation and altered T cell cross-reactivity characterize a B*57-restricted immune response in HIV-1 infection. J Immunol. 2006; 177(6):3893-902.
42. Kloverpris H N, et al. CD8+ TCR Bias and Immunodominance in HIV-1 Infection. J Immunol. 2015.
43. Miles J J, et al. Bias in the alphabeta T-cell repertoire: implications for disease pathogenesis and vaccination. Immunol Cell Biol. 2011; 89(3):375-87.
44. Mendoza D, et al. HLA B*5701-positive long-term nonprogressors/elite controllers are not distinguished from progressors by the clonal composition of HIV-specific CD8+ T cells. J Virol. 2012; 86(7):4014-8.
45. Simons B C, et al. Despite biased TRBV gene usage against a dominant HLA B57-restricted epitope, TCR diversity can provide recognition of circulating epitope variants. J Immunol. 2008; 181(7):5137-46.
46. Price D A, et al. Public clonotype usage identifies protective Gag-specific CD8+ T cell responses in SIV infection. J Exp Med. 2009; 206(4):923-36.
47. Warren R L, et al. Exhaustive T-cell repertoire sequencing of human peripheral blood samples reveals signatures of antigen selection and a directly measured repertoire size of at least 1 million clonotypes. Genome Res. 2011; 21(5):790-7.
48. Mattapallil J J, et al. Massive infection and loss of memory CD4+ T cells in multiple tissues during acute SIV infection. Nature. 2005; 434(7037):1093-7.
49. Godfrey D I, et al. The burgeoning family of unconventional T cells. Nat Immunol. 2015; 16(11):1114-23.
50. Campion S L, et al. Proteome-wide analysis of HIV-specific naive and memory CD4(+) T cells in unexposed blood donors. J Exp Med. 2014; 211(7): 1273-80.
51. Jones R B, et al. Human immunodeficiency virus type 1 escapes from interleukin-2-producing CD4+ T-cell responses without high-frequency fixation of mutations. J Virol. 2009; 83(17):8722-32.
52. Ranasinghe S, et al. Association of HLA-DRB1-restricted CD4 T cell responses with HIV immune control. Nature Medicine. 2013; 19(7):930-3.
53. Yousef S, et al. TCR bias and HLA cross-restriction are strategies of human brain-infiltrating J C virus-specific CD4+ T cells during viral infection. J Immunol. 2012; 189(7):3618-30.
54. Malhotra U, et al. Role for HLA class II molecules in HIV-1 suppression and cellular immunity following antiretroviral treatment. J Clin Invest. 2001; 107(4):505-17.
55. Williams M A, et al. Rapid culling of the CD4+ T cell repertoire in the transition from effector to memory. Immunity. 2008; 28(4):533-45.
56. Belyakov I M, et al. Impact of vaccine-induced mucosal high-avidity CD8+ CTLs in delay of AIDS viral dissemination from mucosa. Blood. 2006; 107(8):3258-64.
57. Corse E, et al. Attenuated T cell responses to a high-potency ligand in vivo. PLoS Biol. 2010; 8(9).
58. Valitutti S. The Serial Engagement Model 17 Years After: From TCR Triggering to Immunotherapy. Front Immunol. 2012; 3:272.
59. Thorborn G, et al. Clonotypic composition of the CD4+ T cell response to a vectored retroviral antigen is determined by its speed. J Immunol. 2014; 193(4):1567-77.
60. Swain S L, et al. Expanding roles for CD4(+) T cells in immunity to viruses. Nat Rev Immunol. 2012; 12(2): 136-48.
61. Blanco-Melo D, et al. Intrinsic cellular defenses against human immunodeficiency viruses. Immunity. 2012; 37(3):399-411.
62. Vetter M L, et al. Differences in APOBEC3G expression in CD4+ T helper lymphocyte subtypes modulate HIV-1 infectivity. PLoS Pathog. 2009; 5(2):e1000292.
63. Oswald-Richter K, et al. Identification of a CCR5-expressing T cell subset that is resistant to R5-tropic HIV infection. PLoS Pathog. 2007; 3(4):e58.
64. Reinherz E L, and Wang J H. Codification of bidentate pMHC interaction with TCR and its co-receptor. Trends Immunol. 2015.
65. Rihn S J, et al. Extreme genetic fragility of the HIV-1 capsid. PLoS Pathog. 2013; 9(6):e1003461.
66. Hansen S G, et al. Cytomegalovirus vectors violate CD8+ T cell epitope recognition paradigms. Science. 2013; 340(6135): 1237874.
67. Ghorashian S, et al. CD8 T cell tolerance to a tumor-associated self-antigen is reversed by CD4 T cells engineered to express the same T cell receptor. J Immunol. 2015; 194(3):1080-9.
68. Gras S, et al. The shaping of T cell receptor recognition by self-tolerance. Immunity. 2009; 30(2):193-203.
69. Heemskerk M H, et al. Redirection of antileukemic reactivity of peripheral T lymphocytes using gene transfer of minor histocompatibility antigen HA-2-specific T-cell receptor complexes expressing a conserved alpha joining region. Blood. 2003; 102(10):3530-40.
70. Klohe E P, et al. Analysis of the molecular specificities of anti-class II monoclonal antibodies by using L cell transfectants expressing HLA class II molecules. J Immunol. 1988; 141(6):2158-64.
71. Vingert B, et al. HIV controller CD4+ T cells respond to minimal amounts of Gag antigen due to high TCR avidity. PLoS Pathog. 2010; 6(2):e1000780.
72. Seth N, et al. Expansion and contraction of HIV-specific CD4 T cells with short bursts of viremia, but physical loss of the majority of these cells with sustained viral replication. J Immunol. 2005; 175(10):6948-58.
73. Lim A, et al. Frequent contribution of T cell clonotypes with public TCR features to the chronic response against a dominant EBV-derived epitope: application to direct detection of their molecular imprint on the human peripheral T cell repertoire. J Immunol. 2000; 165(4):2001-11.
74. Hacein-Bey-Abina S, et al. Efficacy of gene therapy for X-linked severe combined immunodeficiency. N Engl J Med. 2010; 363(4):355-64.
75. Alamyar E, et al. IMGT((R)) tools for the nucleotide analysis of immunoglobulin (IG) and T cell receptor (TR) V-(D)-J repertoires, polymorphisms, and IG mutations: IMGT/V-QUEST and IMGT/HighV-QUEST for NGS. Methods Mol Biol. 2012; 882(569-604.
76. Colwell R K. EstimateS: Statistical estimation of species richness and shared species from samples. Version 9. http://purloclcorg/estimates. 2013.
77. Bailey T L, et al. The MEME Suite. Nucleic Acids Res. 2015.
78. Stothard P. The sequence manipulation suite: JavaScript programs for analyzing and formatting protein and DNA sequences. Biotechniques. 2000; 28(6):1102, 4.
79. Gras S, et al. The shaping of T cell receptor recognition by self-tolerance. Immunity. 2009; 30(2):193-203.
80. Negre D, et al. Characterization of novel safe lentiviral vectors derived from simian immunodeficiency virus (SIVmac251) that efficiently transduce mature human dendritic cells. Gene Ther. 2000; 7(19):1613-23.
81. Roederer M, et al. SPICE: exploration and analysis of post-cytometric complex multivariate datasets. Cytometry A. 2011; 79(2): 167-74.
82. Chakrabarti L A, and Simon V. Immune mechanisms of HIV control. Curr Opin Immunol. 2010; 22(4):488-96.
83. Walker B D, and Yu X G. Unravelling the mechanisms of durable control of HIV-1. Nat Rev Immunol. 2013; 13(7):487-98.
84. Guaraldi G, Zona S, Menozzi M, Carli F, Bagni P, Berti A, Rossi E, Orlando G, Zoboli G, and Palella F. Cost of noninfectious comorbidities in patients with HIV. Clinicoecon Outcomes Res. 2013; 5(481-8.
85. Gebo K A, Fleishman J A, Conviser R, Hellinger J, Hellinger F J, Josephs J S, Keiser P, Gaist P, Moore R D, and Network HIVR. Contemporary costs of HIV healthcare in the HAART era. AIDS. 2010; 24(17):2705-15.
86. Deeks S G, Lewin S R, and Havlir D V. The end of AIDS: HIV infection as a chronic disease. Lancet. 2013; 382(9903):1525-33.
87. Gaardbo J C, Hartling H J, Gerstoft J, and Nielsen S D. Incomplete immune recovery in HIV infection: mechanisms, relevance for clinical care, and possible solutions. Clin Dev Immunol. 2012; 2012(670957.
88. Kelley C F, Kitchen C M, Hunt P W, Rodriguez B, Hecht F M, Kitahata M, Crane H M, Willig J, Mugavero M, Saag M, et al. Incomplete peripheral CD4+ cell count restoration in HIV-infected patients receiving long-term antiretroviral treatment. Clin Infect Dis. 2009; 48(6):787-94.
89. Appay V, Fastenackels S, Katlama C, Ait-Mohand H, Schneider L, Guihot A, Keller M, Grubeck-Loebenstein B, Simon A, Lambotte O, et al. Old age and anti-cytomegalovirus immunity are associated with altered T-cell reconstitution in HIV-1-infected patients. AIDS. 2011; 25(15):1813-22.
90. Zoufaly A, Cozzi-Lepri A, Reekie J, Kirk O, Lundgren J, Reiss P, Jevtovic D, Machala L, Zangerle R, Mocroft A, et al. Immuno-virological discordance and the risk of non-AIDS and AIDS events in a large observational cohort of HIV-patients in Europe. PLoS One. 2014; 9(1):e87160.
91. Engsig F N, Zangerle R, Katsarou O, Dabis F, Reiss P, Gill J, Porter K, Sabin C, Riordan A, Fatkenheuer G, et al. Long-term mortality in HIV-positive individuals virally suppressed for >3 years with incomplete CD4 recovery. Clin Infect Dis. 2014; 58(9):1312-21.
92. Linette G P, Stadtmauer E A, Maus M V, Rapoport A P, Levine B L, Emery L, Litzky L, Bagg A, Carreno B M, Cimino P J, et al. Cardiovascular toxicity and titin cross-reactivity of affinity-enhanced T cells in myeloma and melanoma. Blood. 2013; 122(6):863-71.
93. Rossjohn J, Gras S, Miles J J, Turner S J, Godfrey D I, and McCluskey J. T cell antigen receptor recognition of antigen-presenting molecules. Annu Rev Immunol. 2015; 33(169-200.

1-24. (canceled)

25. A T-cell receptor (TCR) which is specific for the epitope located between positions 293-312 in the GAG protein of HIV-1 having the amino-acid sequence FRDYVDRF[Y/F]KTLRAEQA[S/T]QE (SEQ ID NO: 1), comprising an alpha chain and a beta chain whose variable domains each comprise three complementarity determining regions CDR1, CDR2 and CDR3, wherein
a. the amino-acid sequence of the CDR1 and CDR2 on the alpha variable chain is encoded by the human TRAV24 gene, and
b. the amino-acid sequence of the CDR1 and CDR2 on the beta variable chain is encoded by the human TRBV2 gene, and
c. the TCR has a sensitivity for said epitope of the GAG protein of HIV-1 that is measured as the capability of CD4+ T cells which express the TCR, to proliferate and to differentiate into TNF-alpha and/or IFN-gamma secreting effectors upon stimulation with said epitope with half-maximal responses for TNF-alpha or IFN-gamma production (EC50) being achieved.

26. The TCR of claim 25, wherein
d. the amino-acid sequence of the CDR3 on the alpha chain comprises a motif selected amongst: [A/S]X[K/R]AAGNKLT (SEQ ID NO: 2), AXYGGATNKLI (SEQ ID NO: 3), AX[R/N][R/N]AGNMLTF (SEQ ID NO: 4), AXD[N/D]RKLI (SEQ ID NO: 5) or AXE[S/G]X[G/A][A/S][Q/E]KLV (SEQ ID NO: 6), X being any amino-acid, and/or
e. the amino-acid sequence of the CDR3 on the beta chain comprises a motif selected amongst: ASSX[R/G/L][T/A][S/G]GXX[E/D/T][Q/T][F/Y]) (SEQ ID NO: 7), ASSX[R/G/L][T/A][S/G/A]GXX[E/D/T/P][Q/T][F/Y/H] (SEQ ID NO: 8), ASSGXXNTEAF (SEQ ID NO: 9) or ASVLMRT[N/R]NEQF (SEQ ID NO: 10), X being any amino-acid.

27. The TCR of claim 25, which specifically recognizes a peptide from the GAG protein of HIV-1 having the amino-acid sequence FRDYVDRF[Y/F]KTLRAEQA[S/T]QE (SEQ ID NO: 1) when presented by a major histocompatibility complex (MHC) molecule.

28. The TCR of claim 25, which has at least one of the following properties:
a. an affinity for the epitope located between positions 293-312 of the GAG protein of HIV-1 measured by a Kd value m that is equal or less than 20 μM by SPR analysis and/or
b. it is polyfunctional as measured by assaying induction of at least 3 cytokines.

29. The TCR of claim 25, which has sensitivity for the epitope located between positions 293-312 of the GAG protein of HIV-1 that is measured according to at least one or several of:
i. the capability of CD4+ T cells which express the TCR, to proliferate and to differentiate into TNF-alpha and/or IFN-gamma secreting effectors upon stimulation with said epitope, defined through the epitope concentration required for achieving half-maximal responses (EC50) for TNF-alpha or IFN-gamma production, wherein the EC50 value is in the range of 10E-8 to 10E-7 M, and/or
ii. monitoring the induction of the early activation marker CD69 by cells expressing the TCRs, upon stimulation with said epitope, as defined through the epitope concentration required for achieving half-maximal responses (EC50) for CD69 induction, wherein the EC50 is in the range of 10-5 M to 10-7 M, and/or
iii. by MHC-class II tetramers binding/titration experiment, sensitivity being defined by half-maximal tetramer binding values (EC50) in cells transduced with the TCR, wherein the EC50 tetramer binding value is in the range of 10E-9 to 10E-7 M, and/or
iv. by assessing with TCR-transduced cells, upon antigenic stimulation using the Gag293 peptide, whether markers production including at least one of the following markers is achieved: cytokines such as TNF-alpha, IL-2, IFN-gamma, chemokines such as MIP-1 beta/CCL4, degranulation marker such as CD107a, and/or
v. by assessing the polyfunctionality of TCR-transduced cells such as CD4+ or CD8+ cells, upon antigenic stimulation using the Gag293 peptide, though assessment of the presence of a detectable induction of at least 3 cytokines, and/or
vi. by assessing the cytotoxicity of TCR-transduced cells such as CD4+ or CD8+ cells, in the presence of HIV-infected cells, and evaluating the percentage of viral suppression, wherein the the viral suppression observed in HIV-infected cells, especially in the presence of CD4+ transduced cells, is in a range of 40%, to 100%, and/or viral suppression can be detected in HIV-infected cells in the presence of CD4+ transduced cells at a ratio below 2, and/or viral suppression is observed in CD8+ transduced cells and is in a range of 40% to 100%.

30. The TCR of claim 25, wherein:
a. the amino-acid sequence of the CDR3 on the alpha chain is or comprises a sequence as disclosed in any one of SEQ ID NO: 11 to 27, and/or
b. the amino-acid sequence of the CDR3 on the beta chain is or comprises a sequence as disclosed in any one of SEQ ID NO: 28 to 46, or
c. the amino-acid sequence of the CDR3 on the alpha and/or beta chain comprises a variant having at least 80% amino-acids sequence identity with the sequences disclosed in a. and b. respectively,
the length of the amino-acid sequence of the CDR3 on the alpha chain being from 9 and 16 amino-acid residues, the length of the amino-acid sequence of the CDR3 on the beta chain being from 11 and 18 amino-acid residues.

31. The TCR of claim 25, wherein:
a. the amino-acid sequence of the CDR3 on the alpha chain is or comprises a sequence selected from: CAFKAAGNKLTF (SEQ ID NO: 47), CASKAAGNKLTF (SEQ ID NO: 48), and CSRRAAGNKLTF (SEQ ID NO: 49), and/or
b. the amino-acid sequence of the CDR3 on the beta chain is or comprises a sequence selected from: CASSRLAGGMDEQF (SEQ ID NO: 50), CATTPGASGISEQF (SEQ ID NO: 51), CASSPGTSGVEQFF (SEQ ID NO: 52), and CASSRRTSGGTDTQYF (SEQ ID NO: 53), or
c. the amino-acid sequence of the CDR3 on the alpha and/or beta chain comprises a variant having at least 80% amino-acids sequence identity with the sequences disclosed in a. and b. respectively,
the length of the amino-acid sequence of the CDR3 on the alpha chain being from 9 and 16 amino-acid residues, the length of the amino-acid sequence of the CDR3 on the beta chain being from 11 and 18 amino-acid residues.

32. The TCR of claim 25, wherein:
the amino-acid sequence of the CDR1 and CDR2 on the alpha variable chain encoded by the human TRAV24 gene has an amino-acid sequence for the CDR1alpha corresponding to the positions 49 to 54 in SEQ ID NO: 58 and has an amino-acid sequence for the CDR2alpha corresponding to the positions 72 to 77 in SEQ ID NO: 58 and
the amino-acid sequence of the CDR1 and CDR2 on the beta variable chain encoded by the human TRBV2 gene has an amino-acid sequence for the CDR1beta corresponding to the positions 46 to 50 in SEQ ID NO: 59 and has an amino-acid sequence for the CDR2beta corresponding to the positions 68 to 73 in SEQ ID NO: 59 and
the amino-acid sequence of the CDR3 on the alpha chain that is: CAFKAAGNKLTF (SEQ ID NO: 11), and the amino-acid sequence of the CDR3 on the beta chain that is: CASSRLAGGMDEQFF (SEQ ID NO: 512), or
the amino-acid sequence of the CDR3 on the alpha and/or beta chain comprises a variant having at least 80% amino-acids sequence identity with the sequences disclosed above for the CDR3 found on the alpha and beta chains, respectively, the length of the amino-acid sequence of the CDR3 on the alpha chain being from 9 and 16 amino-acid residues, the length of the amino-acid sequence of the CDR3 on the beta chain being from 11 and 18 amino-acid residues.

33. The TCR of claim 25, wherein:
the amino-acid sequence of its alpha chain is as disclosed in SEQ ID NO: 58 or SEQ ID NO: 60, and
the amino-acid sequence of its beta chain is as disclosed in SEQ ID NO: 59 or SEQ ID NO: 61, or
the amino-acid sequence of its alpha and/or beta chain is a variant having at least 80% amino-acids sequence identity with the sequences disclosed above.

34. The TCR of claim 25, which is an isolated, and/or recombinant TCR and/or a chimeric TCR such as a single chain TCR, a soluble TCR, a single chain TCR fragment.

35. A nucleic acid molecule encoding at least one chain of the TCR of claim 25, or a fragment thereof selected in the group of nucleic acid molecules encoding the alpha chain, the variable domain of the alpha chain, the CDR3 domain of the alpha chain, the beta chain, the variable domain of the beta chain, the CDR3 domain of the beta chain, the soluble form of the TCR.

36. A nucleic acid molecule encoding at least a part of the alpha chain of a human TCR and/or at least a part of the beta chain of a TCR, in which:
a. the nucleic acid molecule encoding at least a part of the alpha chain of a TCR comprises at least the nucleotide sequence of the human TRAV24 gene coding the CDR1 and the CDR2 of a TCR and further comprises the junctional rearranged nucleotide sequence disclosed in any one of SEQ ID NO: 79 to SEQ ID NO: 120 or a variant thereof having at least 80% sequence identity with these sequences, and/or
b. the nucleic acid molecule encoding at least a part of the beta chain of a TCR comprises at least the nucleotide sequence of the human TRBV2 gene coding the CDR1 and the CDR2 of a TCR and further comprises the junctional rearranged nucleotide sequence disclosed in any one of SEQ ID NO: 121 to SEQ ID NO: 161 or a variant thereof having at least 80% sequence identity with these sequences.

37. The nucleic acid molecule according to claim 36, which comprises or consists of the sequence disclosed in SEQ ID NO: 62, and/or comprises or consists of the sequence disclosed in SEQ ID NO: 63, or variant thereof having at least 80% nucleotide sequence identity with the sequences.

38. The nucleic acid molecule according to claim 36, which comprises a nucleic acid sequence coding for the alpha chain and/or the beta chain of a TCR.

39. A recombinant nucleic acid molecule which comprises the nucleic acid molecule coding for the alpha chain and the nucleic acid molecule of the beta chain of a TCR wherein both nucleic acid molecules are linked to form a single molecule, optionally are linked by a polynucleotide encoding a 2A peptide, and each of said nucleic acid molecule is as defined in claim 35.

40. A vector comprising at least one nucleic acid molecule according to claim 35.

41. A lentiviral transfer vector, comprising at least one nucleic acid molecule according to claim 35 and lentiviral cis-active elements including long terminal repeats (LTRs) or modified LTRs including partially deleted 3′LTR, psi (Ψ) packaging signal, optionally Rev responsive element (RRE), wherein the nucleic acid molecule is contained in a transcription unit and wherein said vector optionally further comprises a lentiviral DNA flap encompassing the fragment of the lentiviral genome framed by the regions of central polypurine tract (cPPT) and central termination sequence (CTS) and/or wherein the transcription unit optionally comprises a self-cleaving 2A sequence inserted between sequences encoding alpha and beta TCR chains.

42. The lentiviral transfer vector according to claim 41, wherein the lentiviral sequences are from the genomic sequence of a viral species selected from the group consisting of: human immunodeficiency virus (HIV), simian immunodeficiency virus (SIV), visna/maedi virus (VMV), caprine arthritis-encephalitis virus (CAEV), equine infectious anemia virus (EIAV), feline immunodeficiency virus (FIV), and bovine immunodeficiency virus (BIV)

43. A method to produce recombinant lentiviral vector particles, comprising or consisting of:
a) transfecting the recombinant lentiviral transfer vector according to claim 41, into a host cell, for example a HEK-293T cell line;
b) co-transfecting the cell of step a) with (i) a plasmid vector encoding the envelope glycoprotein G of a VSV,
and (ii) with a plasmid vector encoding the lentiviral GAG and POL protein or mutated non integrative POL protein of a lentivirus, as packaging construct;
c) recovering the recombinant lentiviral particles expressing recombinant TCR.

44. Recombinant lentiviral vector particles the genome of which comprises the recombinant lentiviral transfer vector according to claim 41, which are pseudotyped with a vesicular stomatitis virus glycoprotein G (VSV-G) protein.

45. A method for obtaining a collection of recombinant cells expressing a TCR or a recombinant TCR, comprising the steps of:
a. Transducing cells capable of expressing a TCR at their surface with recombinant lentiviral vector particles according to claim 44, and
b. Culturing the transduced cells in conditions that permit the TCR to be expressed at their surface, and
c. Obtaining and/or recovering a recombinant cell collection expressing said recombinant TCR and optionally further isolating said expressed recombinant TCR.

46. A collection of recombinant human T-cells presenting a TCR as claimed in claim 25, formulated for administration to a human host.

47. A method of providing an immunotherapeutic treatment of a human patient seropositive for HIV, comprising administering the recombinant lentiviral vector particles according to claim 44 to the human patient.

48. The method of claim 47, wherein the human patient is under antiretroviral therapy following a HIV infection.


SEQUENCE LISTING (1274 sequences)

SEQ ID NO: 1 - protein, 20 aa, Artificial sequence
  Consensus sequence epitope Gag 293
  MISC_FEATURE (1)..(20): Gag 293 Consensus
  MISC_FEATURE (9)..(9): Xaa can be Tyr or Phe
  MISC_FEATURE (18)..(18): Xaa can be Ser or Thr
FRDYVDRFXKTLRAEQAXQE

SEQ ID NO: 2 - protein, 10 aa, Artificial sequence
  Motif Trav24 AV24-1
  MISC_FEATURE (1)..(10): Motif AV24-1
  MISC_FEATURE (1)..(1): Xaa can be Ala or Ser
  MISC_FEATURE (2)..(2): Xaa can be any amino-acid
  MISC_FEATURE (3)..(3): Xaa can be Lys or Arg
XXXAAGNKLT

SEQ ID NO: 3 - protein, 11 aa, Artificial sequence
  Motif TRAV24-2
  MISC_FEATURE (1)..(11): Motif TRAV24-2
  MISC_FEATURE (2)..(2): Xaa can be any amino-acid
AXYGGATNKLI

SEQ ID NO: 4 - protein, 11 aa, Artificial sequence
  Motif TRAV24-3
  MISC_FEATURE (1)..(11): Motif TRAV24-3
  MISC_FEATURE (2)..(2): Xaa can be any amino-acid
  MISC_FEATURE (3)..(3): Xaa can be Arg or Asn
  MISC_FEATURE (4)..(4): Xaa can be Arg or Asn
AXXXAGNMLTF

SEQ ID NO: 5 - protein, 8 aa, Artificial sequence
  Motif TRAV24-4
  MISC_FEATURE (1)..(8): Motif TRAV24-4
  MISC_FEATURE (2)..(2): Xaa can be any amino-acid
  MISC_FEATURE (4)..(4): Xaa can be Asn or Asp
AXDXRKLI

SEQ ID NO: 6 - protein, 11 aa, Artificial sequence
  Motif TRAV24-5
  MISC_FEATURE (1)..(11): Motif TRAV24-5
  MISC_FEATURE (2)..(2): Xaa can be any amino-acid
  MISC_FEATURE (4)..(4): Xaa can be Ser or Gly
  MISC_FEATURE (5)..(5): Xaa can be any amino-acid
  MISC_FEATURE (6)..(6): Xaa can be Gly or Ala
  MISC_FEATURE (7)..(7): Xaa can be ala or Ser
  MISC_FEATURE (8)..(8): Xaa can be Gln or Glu
AXEXXXXXKLV

SEQ ID NO: 7 - protein, 14 aa, Artificial sequence
  Motif TRBV2-1
  MISC_FEATURE (1)..(14): Motif TRBV2-1
  MISC_FEATURE (4)..(4): Xaa can be any amino-acid
  MISC_FEATURE (5)..(5): Xaa can be Arg or Gly or Leu
  MISC_FEATURE (6)..(6): Xaa can be Thr or Ala
  MISC_FEATURE (10)..(10): Xaa can be any amino-acid
  MISC_FEATURE (11)..(11): Xaa can be any amino-acid
  MISC_FEATURE (12)..(12): Xaa can be Glu or Asp or Thr
  MISC_FEATURE (13)..(13): Xaa can be Gln or Thr
  MISC_FEATURE (14)..(14): Xaa can be Phe or Tyr
ASSXXXSGGXXXXX

SEQ ID NO: 8 - protein, 13 aa, Artificial sequence
  Motif TRBV2-2-b
  MISC_FEATURE (1)..(13): Motif TRBV2-2-b
  MISC_FEATURE (4)..(4): Xaa can be any amino-acid
  MISC_FEATURE (5)..(5): Xaa can be Arg or Gly or Leu
  MISC_FEATURE (6)..(6): Xaa can be Thr or Ala
  MISC_FEATURE (7)..(7): Xaa can be Ser or Gly or Ala
  MISC_FEATURE (9)..(9): Xaa can be any amino-acid
  MISC_FEATURE (10)..(10): Xaa can be any amino-acid
  MISC_FEATURE (11)..(11): Xaa can be Glu or Asp or Thr or Pro
  MISC_FEATURE (12)..(12): Xaa can be Gln or Thr
  MISC_FEATURE (13)..(13): Xaa can be Phe or Tyr or His
ASSXXXXGXXXXX

SEQ ID NO: 9 - protein, 11 aa, Artificial sequence
  Motif TRBV2-3
  MISC_FEATURE (1)..(11): Motif TRBV2-3
  MISC_FEATURE (5)..(5): Xaa can be any amino-acid
  MISC_FEATURE (6)..(6): Xaa can be any amino-acid
ASSGXXNTEAF

SEQ ID NO: 10 - protein, 12 aa, Artificial sequence
  Motif TRBV2-4
  MISC_FEATURE (1)..(12): Motif TRBV2-4
  MISC_FEATURE (8)..(8): Xaa can be Asn or Arg
ASVLMRTXNEQF

SEQ ID NO: 11 - protein, 12 aa, Homo sapiens
CAFKAAGNKLTF

SEQ ID NO: 12 - protein, 12 aa, Homo sapiens
CAFRAAGNKLTF

SEQ ID NO: 13 - protein, 12 aa, Homo sapiens
CAHKAAGNKLTF

SEQ ID NO: 14 - protein, 12 aa, Homo sapiens
CALKAAGNKLTF

SEQ ID NO: 15 - protein, 12 aa, Homo sapiens
CASKAAGNKLTF

SEQ ID NO: 16 - protein, 12 aa, Homo sapiens
CSRRAAGNKLTF

SEQ ID NO: 17 - protein, 13 aa, Homo sapiens
CAEYGGATNKLIF

SEQ ID NO: 18 - protein, 13 aa, Homo sapiens
CANYGGATNKLIF

SEQ ID NO: 19 - protein, 13 aa, Homo sapiens
CAPYGGATNKLIF

SEQ ID NO: 20 - protein, 13 aa, Homo sapiens
CARYGGATNKLIF

SEQ ID NO: 21 - protein, 13 aa, Homo sapiens
CASYGGATNKLIF

SEQ ID NO: 22 - protein, 10 aa, Homo sapiens
CAFDNRKLIW

SEQ ID NO: 23 - protein, 12 aa, Homo sapiens
CAFRNAGNMLTF

SEQ ID NO: 24 - protein, 12 aa, Homo sapiens
CALNNAGNMLTF

SEQ ID NO: 25 - protein, 12 aa, Homo sapiens
CALRNAGNMLTF

SEQ ID NO: 26 - protein, 12 aa, Homo sapiens
CATRRAGNMLTF

SEQ ID NO: 27 - protein, 13 aa, Homo sapiens
CASESTGAQKLVF

SEQ ID NO: 28 - protein, 12 aa, Homo sapiens
CAYEGASEKLVF

SEQ ID NO: 29 - protein, 13 aa, Homo sapiens
CASSGQTNTEAFF

SEQ ID NO: 30 - protein, 15 aa, Homo sapiens
CASSEGAAGNQPQHF

SEQ ID NO: 31 - protein, 15 aa, Homo sapiens
CASSELTSGGDEQFF

SEQ ID NO: 32 - protein, 15 aa, Homo sapiens
CASSPGTSGVGEQFF

SEQ ID NO: 33 - protein, 14 aa, Homo sapiens
CASVLMRTNNEQFF

SEQ ID NO: 34 - protein, 15 aa, Homo sapiens
CASSALASGTDTQYF

SEQ ID NO: 35 - protein, 16 aa, Homo sapiens
CASSARTSGGADTQYF

SEQ ID NO: 36 - protein, 16 aa, Homo sapiens
CASSARTSGGSDTQYF

SEQ ID NO: 37 - protein, 16 aa, Homo sapiens
CASSARTSGGTDTQYF

SEQ ID NO: 38 - protein, 15 aa, Homo sapiens
CASSHRASGGDTQYF

SEQ ID NO: 39 - protein, 16 aa, Homo sapiens
CASSLRTSGGSDTQYF

SEQ ID NO: 40 - protein, 16 aa, Homo sapiens
CASSLRTSGGTDTQYF

SEQ ID NO: 41 - protein, 15 aa, Homo sapiens
CASSPLTSGTDTQYF

SEQ ID NO: 42 - protein, 16 aa, Homo sapiens
CASSRRTSGGTDTQYF

SEQ ID NO: 43 - protein, 15 aa, Homo sapiens
CASSKLASGADEQYF

SEQ ID NO: 44 - protein, 15 aa, Homo sapiens
CASSPRTSGGDEQYF

SEQ ID NO: 45 - protein, 15 aa, Homo sapiens
CASSPRTSGTYEQYF

SEQ ID NO: 46 - protein, 15 aa, Homo sapiens
CASSRRTSGTYEQYF

SEQ ID NO: 47 - protein, 12 aa, Homo sapiens
CAFKAAGNKLTF

SEQ ID NO: 48 - protein, 12 aa, Homo sapiens
CASKAAGNKLTF

SEQ ID NO: 49 - protein, 12 aa, Homo sapiens
CSRRAAGNKLTF

SEQ ID NO: 50 - protein, 14 aa, Homo sapiens
CASSRLAGGMDEQF

SEQ ID NO: 51 - protein, 14 aa, Homo sapiens
CATTPGASGISEQF

SEQ ID NO: 52 - protein, 14 aa, Homo sapiens
CASSPGTSGVEQFF

SEQ ID NO: 53 - protein, 16 aa, Homo sapiens
CASSRRTSGGTDTQYF

SEQ ID NO: 54 - DNA, 60 bp, Human immunodeficiency virus
tttagagactatgtagaccggttctataaaactctaagagccgagcaagcttcacaggag

SEQ ID NO: 55 - DNA, 343 bp, Homo sapiens
atggagaagaatcctttggcagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgccttta

SEQ ID NO: 56 - DNA, 290 bp, Homo sapiens
gaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgaagc

SEQ ID NO: 57 - DNA, 8241 bp, Artificial sequence
  Plasmid pCDH-F24-TCR
  source (1)..(8241): Plasmid pCDH-F24-TCR
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagctagccgccaccatggagaagaatcctttggtagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgcctttaaagctgcaggcaacaagctaacttttggaggaggaaccagggtgctagttaaaccaaatatccagaagcctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagcagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcgcggccgctgagggcagaggaagtcttctaacatgcggtgacgtggaggagaatcccggcccttccggaatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagccgactagcgggagggatggatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 58 - protein, 274 aa, Homo sapiens
MEKNPLVAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCAFKAAGNKLTFGGGTRVLVKPNIQKPDPAVYQLRDSKSSDKSVCLFTDFDSQTNVSQSKDSDVYITDKTVLDMRSMDFKSNSAVAWSNKSDFACANAFNNSIIPADTFFPSPESSCDVKLVEKSFETDTNLNFQNLSVIGFRILLLKVAGFNLLMTLRLWSS

SEQ ID NO: 59 - protein, 313 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSRLAGGMDEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 60 - protein, 263 aa, Homo sapiens
ILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCAFKAAGNKLTFGGGTRVLVKPNIQKPDPAVYQLRDSKSSDKSVCLFTDFDSQTNVSQSKDSDVYITDKTVLDMRSMDFKSNSAVAWSNKSDFACANAFNNSIIPADTFFPSPESSCDVKLVEKSFETDTNLNFQNLSVIGFRILLLKVAGFNLLMTLRLWSS

SEQ ID NO: 61 - protein, 294 aa, Homo sapiens
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSRLAGGMDEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 62 - DNA, 822 bp, Homo sapiens
atggagaagaatcctttggtagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgcctttaaagctgcaggcaacaagctaacttttggaggaggaaccagggtgctagttaaaccaaatatccagaagcctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagcagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 63 - DNA, 939 bp, Homo sapiens
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagccgactagcgggagggatggatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 64 - DNA, 423 bp, Homo sapiens
  misc_feature (1)..(1): n is a, c, g, or t
natatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 65 - DNA, 537 bp, Homo sapiens
gaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 66 - DNA, 63 bp, Homo sapiens
tgatcaaagctgcaggcaacaagctaacttttggaggaggaaccagggtgctagttaaaccaa

SEQ ID NO: 67 - DNA, 66 bp, Homo sapiens
tgaattatggcggtgctacaaacaagctcatctttggaactggcactctgcttgctgtccagccaa

SEQ ID NO: 68 - DNA, 63 bp, Homo sapiens
tgaataataatgcaggcaacatgctcacctttggagggggaacaaggttaatggtcaaacccc

SEQ ID NO: 69 - DNA, 60 bp, Homo sapiens
taattcagggagcccagaagctggtatttggccaaggaaccaggctgactatcaacccaa

SEQ ID NO: 70 - DNA, 63 bp, Homo sapiens
taactcagggcggatctgaaaagctggtctttggaaagggaacgaaactgacagtaaacccat

SEQ ID NO: 71 - DNA, 48 bp, Homo sapiens
tgaacactgaagctttctttggacaaggcaccagactcacagttgtag

SEQ ID NO: 72 - DNA, 48 bp, Homo sapiens
ctaactatggctacaccttcggttcggggaccaggttaaccgttgtag

SEQ ID NO: 73 - DNA, 50 bp, Homo sapiens
tagcaatcagccccagcattttggtgatgggactcgactctccatcctag

SEQ ID NO: 74 - DNA, 50 bp, Homo sapiens
ctcctacaatgagcagttcttcgggccagggacacggctcaccgtgctag

SEQ ID NO: 75 - DNA, 49 bp, Homo sapiens
agcacagatacgcagtattttggcccaggcacccggctgacagtgctcg

SEQ ID NO: 76 - DNA, 47 bp, Homo sapiens
ctcctacgagcagtacttcgggccgggcaccaggctcacggtcacag

SEQ ID NO: 77 - DNA, 12 bp, Homo sapiens
gggacagggggc

SEQ ID NO: 78 - DNA, 16 bp, Homo sapiens
gggactagcggggggg

SEQ ID NO: 79 - DNA, 36 bp, Homo sapiens
tgtgcctttaaagctgcaggcaacaagctaactttt

SEQ ID NO: 80 - DNA, 36 bp, Homo sapiens
tgtgcctttaaggctgcaggcaacaagctaactttt

SEQ ID NO: 81 - DNA, 36 bp, Homo sapiens
tgtgccttcaaagctgcaggcaacaagctaactttt

SEQ ID NO: 82 - DNA, 36 bp, Homo sapiens
tgtgcatttaaagctgcaggcaacaagctaactttt

SEQ ID NO: 83 - DNA, 36 bp, Homo sapiens
tgtgccttcagggctgcaggcaacaagctaactttt

SEQ ID NO: 84 - DNA, 36 bp, Homo sapiens
tgtgcctttagagctgcaggcaacaagctaactttt

SEQ ID NO: 85 - DNA, 36 bp, Homo sapiens
tgtgcctttcgagctgcaggcaacaagctaactttt

SEQ ID NO: 86 - DNA, 36 bp, Homo sapiens
tgtgcctttagggctgcaggcaacaagctaactttt

SEQ ID NO: 87 - DNA, 36 bp, Homo sapiens
tgtgcccacaaagctgcaggcaacaagctaactttt

SEQ ID NO: 88 - DNA, 36 bp, Homo sapiens
tgtgcccataaagctgcaggcaacaagctaactttt

SEQ ID NO: 89 - DNA, 36 bp, Homo sapiens
tgtgccttgaaagctgcaggcaacaagctaactttt

SEQ ID NO: 90 - DNA, 36 bp, Homo sapiens
tgtgccttaaaagctgcaggcaacaagctaactttt

SEQ ID NO: 91 - DNA, 36 bp, Homo sapiens
tgtgctctaaaagctgcaggcaacaagctaactttt

SEQ ID NO: 92 - DNA, 36 bp, Homo sapiens
tgtgcctctaaagctgcaggcaacaagctaactttt

SEQ ID NO: 93 - DNA, 36 bp, Homo sapiens
tgtgcctccaaagctgcaggcaacaagctaactttt

SEQ ID NO: 94 - DNA, 36 bp, Homo sapiens
tgtagtcggagggctgcaggcaacaagctaactttt

SEQ ID NO: 95 - DNA, 39 bp, Homo sapiens
tgtgccgaatatggtggtgctacaaacaagctcatcttt

SEQ ID NO: 96 - DNA, 39 bp, Homo sapiens
tgtgccaactatggcggtgctacaaacaagctcatcttt

SEQ ID NO: 97 - DNA, 39 bp, Homo sapiens
tgtgccccttatggtggtgctacaaacaagctcatcttt

SEQ ID NO: 98 - DNA, 39 bp, Homo sapiens
tgtgctccttatggcggtgctacaaacaagctcatcttt

SEQ ID NO: 99 - DNA, 39 bp, Homo sapiens
tgtgccccgtatggtggtgctacaaacaagctcatcttt

SEQ ID NO: 100 - DNA, 39 bp, Homo sapiens
tgtgcccgttatggtggtgctacaaacaagctcatcttt

SEQ ID NO: 101 - DNA, 39 bp, Homo sapiens
tgtgctcgttatggtggtgctacaaacaagctcatcttt

SEQ ID NO: 102 - DNA, 39 bp, Homo sapiens
tgtgctcggtatggtggtgctacaaacaagctcatcttt

SEQ ID NO: 103 - DNA, 39 bp, Homo sapiens
tgtgcctcttatggtggtgctacaaacaagctcatcttt

SEQ ID NO: 104 - DNA, 39 bp, Homo sapiens
tgtgcctcctatggtggtgctacaaacaagctcatcttt

SEQ ID NO: 105 - DNA, 39 bp, Homo sapiens
tgtgcctcgtatggtggtgctacaaacaagctcatcttt

SEQ ID NO: 106 - DNA, 39 bp, Homo sapiens
tgtgcctcctatggcggtgctacaaacaagctcatcttt

SEQ ID NO: 107 - DNA, 30 bp, Homo sapiens
tgtgccttcgacaaccgtaagctgatttgg

SEQ ID NO: 108 - DNA, 30 bp, Homo sapiens
tgtgcctttgacaaccgtaagctgatttgg

SEQ ID NO: 109 - DNA, 36 bp, Homo sapiens
tgtgcctttcgtaatgcaggcaacatgctcaccttt

SEQ ID NO: 110 - DNA, 36 bp, Homo sapiens
tgtgcctttcggaatgcaggcaacatgctcaccttt

SEQ ID NO: 111 - DNA, 36 bp, Homo sapiens
tgtgcctttaggaatgcaggcaacatgctcaccttt

SEQ ID NO: 112 - DNA, 36 bp, Homo sapiens
tgtgcctttagaaatgcaggcaacatgctcaccttt

SEQ ID NO: 113 - DNA, 36 bp, Homo sapiens
tgtgccctcaataatgcaggcaacatgctcaccttt

SEQ ID NO: 114 - DNA, 36 bp, Homo sapiens
tgtgccttaaataatgcaggcaacatgctcaccttt

SEQ ID NO: 115 - DNA, 36 bp, Homo sapiens
tgtgccctgcggaatgcaggcaacatgctcaccttt

SEQ ID NO: 116 - DNA, 36 bp, Homo sapiens
tgtgccttaagaaatgcaggcaacatgctcaccttt

SEQ ID NO: 117 - DNA, 36 bp, Homo sapiens
tgtgccttacgaaatgcaggcaacatgctcaccttt

SEQ ID NO: 118 - DNA, 36 bp, Homo sapiens
tgtgcgacgcgccgtgcaggcaacatgctcaccttt

SEQ ID NO: 119 - DNA, 39 bp, Homo sapiens
tgtgcctccgagtccacgggagcccagaagctggtattt

SEQ ID NO: 120 - DNA, 36 bp, Homo sapiens
tgtgcctacgagggggcatctgaaaagctggtcttt

SEQ ID NO: 121 - DNA, 39 bp, Homo sapiens
tgtgccagctcaggacagacgaacactgaagctttcttt

SEQ ID NO: 122 - DNA, 45 bp, Homo sapiens
tgtgccagcagtgaaggggcggctggcaatcagccccagcatttt

SEQ ID NO: 123 - DNA, 45 bp, Homo sapiens
tgtgccagcagcgagggggcggcgggcaatcagccccagcatttt

SEQ ID NO: 124 - DNA, 45 bp, Homo sapiens
tgtgccagcagtgagttgactagcgggggggatgagcagttcttc

SEQ ID NO: 125 - DNA, 45 bp, Homo sapiens
tgtgcaagcagccccgggactagcggagttggtgagcagttcttc

SEQ ID NO: 126 - DNA, 45 bp, Homo sapiens
tgtgcaagcagccccgggactagcggagttggtgagcagtttttc

SEQ ID NO: 127 - DNA, 45 bp, Homo sapiens
tgtgccagcagtcccgggactagcggggttggtgagcagttcttc

SEQ ID NO: 128 - DNA, 42 bp, Homo sapiens
tgtgccagtgtattaatgaggacgaacaatgagcagttcttc

SEQ ID NO: 129 - DNA, 45 bp, Homo sapiens
tgtgccagcagtgcgttagctagcggtacagatacgcagtatttt

SEQ ID NO: 130 - DNA, 45 bp, Homo sapiens
tgtgccagcagtgctttggctagcggcacagatacgcagtatttt

SEQ ID NO: 131 - DNA, 45 bp, Homo sapiens
tgtgccagcagtgccctggctagtggcacagatacgcagtatttt

SEQ ID NO: 132 - DNA, 48 bp, Homo sapiens
tgtgccagcagtgcaaggactagcgggggggccgatacgcagtatttt

SEQ ID NO: 133 - DNA, 48 bp, Homo sapiens
tgtgccagcagtgccaggactagcgggggggcagatacgcagtatttt

SEQ ID NO: 134 - DNA, 48 bp, Homo sapiens
tgtgccagcagtgctaggactagcgggggggcagatacgcagtatttt

SEQ ID NO: 135 - DNA, 48 bp, Homo sapiens
tgtgccagcagtgccaggactagcgggggctcggatacgcagtatttt

SEQ ID NO: 136 - DNA, 48 bp, Homo sapiens
tgtgccagcagtgctcggactagcggggggtcagatacgcagtatttt

SEQ ID NO: 137 - DNA, 48 bp, Homo sapiens
tgtgccagcagcgcccggactagcggggggtcagatacgcagtatttt

SEQ ID NO: 138 - DNA, 48 bp, Homo sapiens
tgtgccagcagtgccaggactagcggggggtcagatacgcagtatttt

SEQ ID NO: 139 - DNA, 48 bp, Homo sapiens
tgtgccagcagtgccaggactagcggaggcacagatacgcagtatttt

SEQ ID NO: 140 - DNA, 48 bp, Homo sapiens
tgtgccagcagtgctaggactagcgggggcacagatacgcagtatttt

SEQ ID NO: 141 - DNA, 45 bp, Homo sapiens
tgtgccagcagtcacagggcctcaggcggggatacgcagtatttt

SEQ ID NO: 142 - DNA, 48 bp, Homo sapiens
tgtgccagcagcctaaggactagcgggggttcagatacgcagtatttt

SEQ ID NO: 143 - DNA, 48 bp, Homo sapiens
tgtgccagcagccttaggactagcgggggttcagatacgcagtatttt

SEQ ID NO: 144 - DNA, 48 bp, Homo sapiens
tgtgccagcagtcttaggactagcgggggcacagatacgcagtatttt

SEQ ID NO: 145 - DNA, 48 bp, Homo sapiens
tgtgccagcagtctccggactagcgggggcacagatacgcagtatttt

SEQ ID NO: 146 - DNA, 48 bp, Homo sapiens
tgtgccagcagtcttcggactagcgggggaacagatacgcagtatttt

SEQ ID NO: 147 - DNA, 45 bp, Homo sapiens
tgtgccagcagtccgttgactagcggaacagatacgcagtatttt

SEQ ID NO: 148 - DNA, 45 bp, Homo sapiens
tgtgccagcagtccattgactagcggcacagatacgcagtatttt

SEQ ID NO: 149 - DNA, 48 bp, Homo sapiens
tgtgccagcagtcgtaggactagcgggggcacagatacgcagtatttt

SEQ ID NO: 150 - DNA, 48 bp, Homo sapiens
tgtgccagcagtcgacggactagcgggggcacagatacgcagtatttt

SEQ ID NO: 151 - DNA, 48 bp, Homo sapiens
tgtgccagctcacgacggactagcgggggcacagatacgcagtatttt

SEQ ID NO: 152 - DNA, 48 bp, Homo sapiens
tgtgccagcagccgccggactagcgggggcacagatacgcagtatttt

SEQ ID NO: 153 - DNA, 48 bp, Homo sapiens
tgtgccagcagtcggaggactagcgggggcacagatacgcagtatttt

SEQ ID NO: 154 - DNA, 45 bp, Homo sapiens
tgtgccagctcaaaactggctagcggggccgacgagcagtacttc

SEQ ID NO: 155 - DNA, 45 bp, Homo sapiens
tgtgccagctcaaaactggctagcggggccgacgagcagtatttc

SEQ ID NO: 156 - DNA, 45 bp, Homo sapiens
tgtgccagcagtccccggactagcgggggcgacgagcagtacttc

SEQ ID NO: 157 - DNA, 45 bp, Homo sapiens
tgtgccagcagcccccggactagcgggggggacgagcagtacttc

SEQ ID NO: 158 - DNA, 45 bp, Homo sapiens
tgtgccagcagtccccggactagcggtacctacgagcagtacttc

SEQ ID NO: 159 - DNA, 45 bp, Homo sapiens
tgtgccagcagtcctaggactagcggaacctacgagcagtacttc

SEQ ID NO: 160 - DNA, 45 bp, Homo sapiens
tgtgccagcagccgtaggactagcgggacttacgagcagtacttc

SEQ ID NO: 161 - DNA, 45 bp, Homo sapiens
tgtgccagcagtagacggactagcgggacctacgagcagtacttc

SEQ ID NO: 162 - DNA, 39 bp, Artificial sequence
  forward primer trav24
  source (1)..(39): forward primer trav24
cggctagccgccaccatggagaagaatcctttggcagcc

SEQ ID NO: 163 - DNA, 31 bp, Artificial sequence
  reverse primer trac
  source (1)..(31): reverse primer trac
ttagcggccgcgctggaccacagccgcagcg

SEQ ID NO: 164 - DNA, 34 bp, Artificial sequence
  forward primer trbv2
  source (1)..(34): forward primer trbv2
ggtccggaatggatacctggctcgtatgctgggc

SEQ ID NO: 165 - DNA, 37 bp, Artificial sequence
  reverse primer trbc
  source (1)..(37): reverse primer trbc
ccggtcgacctagcctctggaatcctttctcttgacc

SEQ ID NO: 166 - DNA, 20 bp, Artificial sequence
  forward primer trav24
  source (1)..(20): forward primer trav24
ccgaggccttgtttgtaatg

SEQ ID NO: 167 - DNA, 22 bp, Artificial sequence
  reverse primer trac
  source (1)..(22): reverse primer trac
gtgaataggcagacagacttgt

SEQ ID NO: 168 - protein, 18 aa, Artificial sequence
  T2A peptide
  MISC_FEATURE (1)..(18): T2A peptide
EGRGSLLTCGDVEENPGP

SEQ ID NO: 169 - protein, 19 aa, Artificial sequence
  P2A peptide
  MISC_FEATURE (1)..(19): P2A peptide
ATNFSLLKQAGDVEENPGP

SEQ ID NO: 170 - protein, 20 aa, Artificial sequence
  E2A peptide
  MISC_FEATURE (1)..(20): E2A peptide
QCTNYALLKLAGDVESNPGP

SEQ ID NO: 171 - protein, 22 aa, Artificial sequence
  F2A peptide
  MISC_FEATURE (1)..(22): F2A peptide
VKQTLNFDLLKLAGDVESNPGP

SEQ ID NO: 172 - DNA, 36 bp, Homo sapiens; identical to SEQ ID NO: 79

SEQ ID NO: 173 - DNA, 36 bp, Homo sapiens; identical to SEQ ID NO: 80

SEQ ID NO: 174 - DNA, 36 bp, Homo sapiens; identical to SEQ ID NO: 81

SEQ ID NO: 175 - DNA, 36 bp, Homo sapiens; identical to SEQ ID NO: 82

SEQ ID NO: 176 - protein, 10 aa, Homo sapiens
CARDDRKLIW

SEQ ID NO: 177 - protein, 12 aa, Homo sapiens
CAFTAAGNKLTF

SEQ ID NO: 178 - protein, 12 aa, Homo sapiens
CALGNAGNMLTF

SEQ ID NO: 179 - protein, 13 aa, Homo sapiens
CAFIPGGSYIPTF

SEQ ID NO: 180 - protein, 13 aa, Homo sapiens
CASYGGATNKLIF

SEQ ID NO: 181 - protein, 14 aa, Homo sapiens
CALDSGGGADGLTF

SEQ ID NO: 182 - protein, 12 aa, Homo sapiens
CAFKAAGNKLTF

SEQ ID NO: 183 - protein, 12 aa, Homo sapiens
CARRNAGNMLTF

SEQ ID NO: 184 - protein, 12 aa, Homo sapiens
CASKAAGNKLTF

SEQ ID NO: 185 - protein, 12 aa, Homo sapiens
CALKAAGNKLTF

SEQ ID NO: 186 - protein, 12 aa, Homo sapiens
CALRQAGNMLTF

SEQ ID NO: 187 - protein, 11 aa, Homo sapiens
CAPGGYQKVTF

SEQ ID NO: 188 - protein, 11 aa, Homo sapiens
CAWGSARQLTF

SEQ ID NO: 189 - protein, 12 aa, Homo sapiens
CAFKAAGNKLTF

SEQ ID NO: 190 - protein, 12 aa, Homo sapiens
CAFNAAGNKLTF

SEQ ID NO: 191 - protein, 12 aa, Homo sapiens
CAFVAAGQNFVF

SEQ ID NO: 192 - protein, 13 aa, Homo sapiens
CAEYGGATNKLIF

SEQ ID NO: 193 - protein, 13 aa, Homo sapiens
CANYGGATNKLIF

SEQ ID NO: 194 - protein, 13 aa, Homo sapiens
CASYGGATNKLIF

SEQ ID NO: 195 - protein, 14 aa, Homo sapiens
CALMTTDSWGKLQF

SEQ ID NO: 196 - protein, 13 aa, Artificial sequence
  Motif disclosed in Table 2
  MISC_FEATURE (1)..(13): Motif disclosed in Table 2
  MISC_FEATURE (4)..(4): Xaa is any amino-acid
  MISC_FEATURE (5)..(5): Xaa is Arg or Gly or Leu
  MISC_FEATURE (6)..(6): Xaa is Thr or Ala
  MISC_FEATURE (7)..(7): Xaa is Ser or Gly
  MISC_FEATURE (9)..(9): Xaa is any amino-acid
  MISC_FEATURE (10)..(10): Xaa is any amino-acid
  MISC_FEATURE (11)..(11): Xaa is Glu or Thr
  MISC_FEATURE (13)..(13): Xaa is Phe or Tyr
ASSXXXXGXXXQX

SEQ ID NO: 197 - protein, 14 aa, Artificial sequence
  Motif disclosed in Table 2
  MISC_FEATURE (1)..(14): Motif disclosed in Table 2
  MISC_FEATURE (4)..(4): Xaa is any amino-acid
  MISC_FEATURE (5)..(5): Xaa is Arg or Gly or Leu
  MISC_FEATURE (6)..(6): Xaa is Thr or Ala
  MISC_FEATURE (10)..(10): Xaa is any amino-acid
  MISC_FEATURE (11)..(11): Xaa is any amino-acid
  MISC_FEATURE (12)..(12): Xaa is Glu or Thr
  MISC_FEATURE (14)..(14): Xaa is Phe or Tyr
ASSXXXSGGXXXQX

SEQ ID NO: 198 - protein, 10 aa, Homo sapiens
CARDDRKLIW

SEQ ID NO: 199 - protein, 11 aa, Homo sapiens
CASGNTDKLIF

SEQ ID NO: 200 - protein, 12 aa, Homo sapiens
CAFCNAGNMLTF

SEQ ID NO: 201 - protein, 12 aa, Homo sapiens
CAFKAAGNKLTF

SEQ ID NO: 202 - protein, 12 aa, Homo sapiens
CAFTAAGNKLTF

SEQ ID NO: 203 - protein, 12 aa, Homo sapiens
CALENAGNKLTF

SEQ ID NO: 204 - protein, 12 aa, Homo sapiens
CALGNAGNMLTF

SEQ ID NO: 205 - protein, 12 aa, Homo sapiens
CSPQGGSEKLVF

SEQ ID NO: 206 - protein, 13 aa, Homo sapiens
CAFIPGGSYIPTF

SEQ ID NO: 207 - protein, 13 aa, Homo sapiens
CASDGGSQGNLIF

SEQ ID NO: 208 - protein, 13 aa, Homo sapiens
CASYGGATNKLIF

SEQ ID NO: 209 - protein, 12 aa, Homo sapiens
CAFKAAGNKLTF

SEQ ID NO: 210 - protein, 12 aa, Homo sapiens
CAFKVAGNKLTF

SEQ ID NO: 211 - protein, 12 aa, Homo sapiens
CAFRMAGNMLTF

SEQ ID NO: 212 - protein, 12 aa, Homo sapiens
CAFRNAGNMLTF

SEQ ID NO: 213 - protein, 12 aa, Homo sapiens
CAHKAAGNKLTF

SEQ ID NO: 214 - protein, 12 aa, Homo sapiens
CAPINAGNMLTF

SEQ ID NO: 215 - protein, 12 aa, Homo sapiens
CARKAAGNKLTF

SEQ ID NO: 216 - protein, 12 aa, Homo sapiens
CASKAAGNKLTF

SEQ ID NO: 217 - protein, 12 aa, Homo sapiens
CASRTAGNKLTF

SEQ ID NO: 218 - protein, 11 aa, Homo sapiens
CAPKSRQQANF

SEQ ID NO: 219 - protein, 12 aa, Homo sapiens
CAFKAAGNKLTF

SEQ ID NO: 220 - protein, 12 aa, Homo sapiens
CAFRAAGNKLTF

SEQ ID NO: 221 - protein, 12 aa, Homo sapiens
CALKAAGNKLTF

SEQ ID NO: 222 - protein, 12 aa, Homo sapiens
CALKQAGNKLTF

SEQ ID NO: 223 - protein, 12 aa, Homo sapiens
CALRQAGNMLTF

SEQ ID NO: 224 - protein, 12 aa, Homo sapiens
CAMKAAGNKLTF

SEQ ID NO: 225 - protein, 12 aa, Homo sapiens
CANRAAGNKLTF

SEQ ID NO: 226 - protein, 12 aa, Homo sapiens
CASKAAGNKLTF

SEQ ID NO: 227 - protein, 12 aa, Homo sapiens
CAYKAAGNKLTF

SEQ ID NO: 228 - protein, 12 aa, Homo sapiens
CGFKAAGNKLTF

SEQ ID NO: 229 - protein, 12 aa, Homo sapiens
CGWKAAGNKLTF

SEQ ID NO: 230 - protein, 12 aa, Homo sapiens
CSLKAAGNKLTF

SEQ ID NO: 231 - protein, 12 aa, Homo sapiens
CSLRAAGNKLTF

SEQ ID NO: 232 - protein, 12 aa, Homo sapiens
CSRRAAGNKLTF

SEQ ID NO: 233 - protein, 12 aa, Homo sapiens
CSWKAAGNKLTF

SEQ ID NO: 234 - protein, 12 aa, Homo sapiens
CSWRAAGNKLTF

SEQ ID NO: 235 - protein, 12 aa, Homo sapiens
CTKKAAGNKLTF

SEQ ID NO: 236 - protein, 12 aa, Homo sapiens
CTLKAAGNKLTF

SEQ ID NO: 237 - protein, 12 aa, Homo sapiens
CTNKAAGNKLTF

SEQ ID NO: 238 - protein, 12 aa, Homo sapiens
CTRKAAGNKLTF

SEQ ID NO: 239 - protein, 12 aa, Homo sapiens
CAFQNAGNMLTF

SEQ ID NO: 240 - protein, 12 aa, Homo sapiens
CAFRAAGNKLTF

SEQ ID NO: 241 - protein, 12 aa, Homo sapiens
CATRRAGNMLTF

SEQ ID NO: 242 - protein, 12 aa, Homo sapiens
CAYEGASEKLVF

SEQ ID NO: 243 - protein, 13 aa, Homo sapiens
CAEYGGATNKLIF

SEQ ID NO: 244 - protein, 13 aa, Homo sapiens
CAPYGGATNKLIF

SEQ ID NO: 245 - protein, 13 aa, Homo sapiens
CARYGGAANKLIF

SEQ ID NO: 246 - protein, 13 aa, Homo sapiens
CARYGGATNKLIF

SEQ ID NO: 247 - protein, 13 aa, Homo sapiens
CARYGGGTNKLIF

SEQ ID NO: 248 - protein, 13 aa, Homo sapiens
CASESTGAQKLVF

SEQ ID NO: 249 - protein, 13 aa, Homo sapiens
CASYGGATNKLIF

SEQ ID NO: 250 - protein, 13 aa, Homo sapiens
CASYVGATNKLIF

SEQ ID NO: 251 - protein, 10 aa, Homo sapiens
CAFDNRKLIW

SEQ ID NO: 252 - protein, 12 aa, Homo sapiens
CAFKAAGNKLTF

SEQ ID NO: 253 - protein, 12 aa, Homo sapiens
CAFRAAGNKLTF

SEQ ID NO: 254 - protein, 12 aa, Homo sapiens
CASETGANNLFF

SEQ ID NO: 255 - protein, 12 aa, Homo sapiens
CATRRAGNMLTF

SEQ ID NO: 256 - protein, 12 aa, Homo sapiens
CAYEGASEKLVF

SEQ ID NO: 257 - protein, 13 aa, Homo sapiens
CAEYGGATNKLIF

SEQ ID NO: 258 - protein, 13 aa, Homo sapiens
CAFASGSARQLTF

SEQ ID NO: 259 - protein, 13 aa, Homo sapiens
CASESTGAQKLVF

SEQ ID NO: 260 - protein, 14 aa, Homo sapiens
CAFYPPGNNRKLIW

SEQ ID NO: 261 - protein, 12 aa, Homo sapiens
CAFKAAGNKLTF

SEQ ID NO: 262 - protein, 12 aa, Homo sapiens
CAFKDAGNKLTF

SEQ ID NO: 263 - protein, 12 aa, Homo sapiens
CAFPNAGNMLTF

SEQ ID NO: 264 - protein, 12 aa, Homo sapiens
CAFRAAGNKLTF

SEQ ID NO: 265 - protein, 12 aa, Homo sapiens
CAFRNAGNMLTF

SEQ ID NO: 266 - protein, 12 aa, Homo sapiens
CALKAAGNKLTF

SEQ ID NO: 267 - protein, 12 aa, Homo sapiens
CALKDAGNKLTF

SEQ ID NO: 268 - protein, 12 aa, Homo sapiens
CALKNAGNMLTF

SEQ ID NO: 269 - protein, 12 aa, Homo sapiens
CALNNAGNMLTF

SEQ ID NO: 270 - protein, 12 aa, Homo sapiens
CALRNAGNMLTF

SEQ ID NO: 271 - protein, 12 aa, Homo sapiens
CAPKAAGNKLTF

SEQ ID NO: 272 - protein, 12 aa, Homo sapiens
CAPKDAGNKLTF

SEQ ID NO: 273 - protein, 12 aa, Homo sapiens
CASKNAGNMLTF

SEQ ID NO: 274 - protein, 12 aa, Homo sapiens
CASMNAGNMLTF

SEQ ID NO: 275 - protein, 15 aa, Homo sapiens
CAFKGGGGSNYKLTF

SEQ ID NO: 276 - protein, 10 aa, Homo sapiens
CAFGDYKLSF

SEQ ID NO: 277 - protein, 12 aa, Homo sapiens
CAFHAAGNKLTF

SEQ ID NO: 278 - protein, 12 aa, Homo sapiens
CAFKAAGNKLTF

SEQ ID NO: 279 - protein, 12 aa, Homo sapiens
CAFKGGGNKLTF

SEQ ID NO: 280 - protein, 12 aa, Homo sapiens
CAFNAAGNKLTF

SEQ ID NO: 281 - protein, 12 aa, Homo sapiens
CAFQAAGNKLTF

SEQ ID NO: 282 - protein, 12 aa, Homo sapiens
CAFRGGGNKLTF

SEQ ID NO: 283 - protein, 12 aa, Homo sapiens
CAFRRAGNMLTF

SEQ ID NO: 284 - protein, 12 aa, Homo sapiens
CAHKAAGNKLTF

SEQ ID NO: 285 - protein, 12 aa, Homo sapiens
CAHKGAGNKLTF

SEQ ID NO: 286 - protein, 13 aa, Homo sapiens
CAEYGGATNKLIF

SEQ ID NO: 287 - protein, 13 aa, Homo sapiens
CANYGGATNKLIF

SEQ ID NO: 288 - protein, 13 aa, Homo sapiens
CAPGGGGSEKLVF

SEQ ID NO: 289 - protein, 13 aa, Homo sapiens
CAPYGGATNKLIF

SEQ ID NO: 290 - protein, 13 aa, Homo sapiens
CASYGGATNKLIF

SEQ ID NO: 291 - protein, 11 aa, Homo sapiens
CGGDNNARLMF

SEQ ID NO: 292 - protein, 11 aa, Homo sapiens
CGGDNNARLTF

SEQ ID NO: 293 - protein, 12 aa, Homo sapiens
CASLYNTDKLIF

SEQ ID NO: 294 - protein, 13 aa, Homo sapiens
CANYGGATNKLIF

SEQ ID NO: 295 - protein, 10 aa, Homo sapiens
CARDDRKLIW

SEQ ID NO: 296 - protein, 12 aa, Homo sapiens
CAFTAAGNKLTF

SEQ ID NO: 297 - protein, 12 aa, Homo sapiens
CALGNAGNMLTF

SEQ ID NO: 298 - protein, 13 aa, Homo sapiens
CAFIPGGSYIPTF

SEQ ID NO: 299 - protein, 13 aa, Homo sapiens
CASYGGATNKLIF

SEQ ID NO: 300 - protein, 14 aa, Homo sapiens
CALDSGGGADGLTF

SEQ ID NO: 301 - protein, 12 aa, Homo sapiens
CAFKAAGNKLTF

SEQ ID NO: 302 - protein, 12 aa, Homo sapiens
CARRNAGNMLTF

SEQ ID NO: 303 - protein, 12 aa, Homo sapiens
CASKAAGNKLTF

SEQ ID NO: 304 - protein, 12 aa, Homo sapiens
CALKAAGNKLTF

SEQ ID NO: 305 - protein, 12 aa, Homo sapiens
CALRQAGNMLTF

SEQ ID NO: 306 - protein, 11 aa, Homo sapiens
CAPGGYQKVTF

SEQ ID NO: 307 - protein, 11 aa, Homo sapiens
CAWGSARQLTF

SEQ ID NO: 308 - protein, 12 aa, Homo sapiens
CAFKAAGNKLTF

SEQ ID NO: 309 - protein, 12 aa, Homo sapiens
CAFNAAGNKLTF

SEQ ID NO: 310 - protein, 12 aa, Homo sapiens
CAFVAAGQNFVF

SEQ ID NO: 311 - protein, 13 aa, Homo sapiens
CAEYGGATNKLIF

SEQ ID NO: 312 - protein, 13 aa, Homo sapiens
CANYGGATNKLIF

SEQ ID NO: 313 - protein, 13 aa, Homo sapiens
CASYGGATNKLIF

SEQ ID NO: 314 - protein, 14 aa, Homo sapiens
CALMTTDSWGKLQF

SEQ ID NO: 315 - DNA, 30 bp, Homo sapiens
tgtgcccgtgacgaccgtaagctgatttgg

SEQ ID NO: 316 - DNA, 33 bp, Homo sapiens
tgtgcctctggtaacaccgacaagctcatcttt

SEQ ID NO: 317 - DNA, 36 bp, Homo sapiens
tgtgccttttgtaatgcaggcaacatgctcaccttt

SEQ ID NO: 318 - DNA, 36 bp, Homo sapiens; identical to SEQ ID NO: 79

SEQ ID NO: 319 - DNA, 36 bp, Homo sapiens; identical to SEQ ID NO: 80

SEQ ID NO: 320 - DNA, 36 bp, Homo sapiens
tgtgcctttacggctgcaggcaacaagctaactttt

SEQ ID NO: 321 - DNA, 36 bp, Homo sapiens
tgtgccctagaaaatgcaggcaacaagctaactttt

SEQ ID NO: 322 - DNA, 36 bp, Homo sapiens
tgtgccctcggtaatgcaggcaacatgctcaccttt

SEQ ID NO: 323 - DNA, 36 bp, Homo sapiens
tgtgccctcggtaatgcaggcaacatgctcacgttt

SEQ ID NO: 324 - DNA, 36 bp, Homo sapiens
tgctcgcctcagggcggatctgaaaagctggtcttt

SEQ ID NO: 325 - DNA, 39 bp, Homo sapiens
tgtgcctttatcccaggaggaagctacatacctacattt

SEQ ID NO: 326 - DNA, 39 bp, Homo sapiens
tgtgcctctgatggaggaagccaaggcaatctcatcttt

SEQ ID NO: 327 - DNA, 39 bp, Homo sapiens; identical to SEQ ID NO: 103

SEQ ID NO: 328 - DNA, 36 bp, Homo sapiens; identical to SEQ ID NO: 81

SEQ ID NO: 329 - DNA, 36 bp, Homo sapiens; identical to SEQ ID NO: 79

SEQ ID NO: 330 - DNA, 36 bp, Homo sapiens
tgtgcctttaaggttgcaggcaacaagctaactttt

SEQ ID NO: 331 - DNA, 36 bp, Homo sapiens
tgtgcctttaggatggcaggcaacatgctcaccttt

SEQ ID NO: 332 - DNA, 36 bp, Homo sapiens; identical to SEQ ID NO: 109

SEQ ID NO: 333 - DNA, 36 bp, Homo sapiens; identical to SEQ ID NO: 87

SEQ ID NO: 334 - DNA, 36 bp, Homo sapiens; identical to SEQ ID NO: 88

SEQ ID NO: 335 - DNA, 36 bp, Homo sapiens
tgtgcccctataaatgcaggcaacatgctcaccttt

SEQ ID NO: 336 - DNA, 36 bp, Homo sapiens
tgtgcccgcaaagctgcaggcaacaagctaactttt

SEQ ID NO: 337 - DNA, 36 bp, Homo sapiens; identical to SEQ ID NO: 92

SEQ ID NO: 338 - DNA, 36 bp, Homo sapiens
tgtgcctcacgaactgcaggcaacaagctaactttt

SEQ ID NO: 339 - DNA, 33 bp, Homo sapiens
tgtgctccaaagagcaggcaacaagctaacttt

SEQ ID NO: 340 - DNA, 36 bp, Homo sapiens; identical to SEQ ID NO: 81

SEQ ID NO: 341 - DNA, 36 bp, Homo sapiens; identical to SEQ ID NO: 79

SEQ ID NO: 342 - DNA, 36 bp, Homo sapiens; identical to SEQ ID NO: 83

SEQ ID NO: 343 - DNA, 36 bp, Homo sapiens; identical to SEQ ID NO: 89

SEQ ID NO: 344 - DNA, 36 bp, Homo sapiens
tgtgccttgaaacaagcaggcaacaagctcactttt

SEQ ID NO: 345 - DNA, 36 bp, Homo sapiens
tgtgccttgaggcaagcaggcaacatgctcaccttt

SEQ ID NO: 346 - DNA, 36 bp, Homo sapiens
tgtgccatgaaagctgcaggcaacaagctaactttt

SEQ ID NO: 347 - DNA, 36 bp, Homo sapiens
tgtgcgatgaaagctgcaggcaacaagctaactttt

SEQ ID NO: 348 - DNA, 36 bp, Homo sapiens
tgtgccaacagagctgcaggcaacaagctaactttt

SEQ ID NO: 349 - DNA, 36 bp, Homo sapiens; identical to SEQ ID NO: 93

SEQ ID NO: 350 - DNA, 36 bp, Homo sapiens
tgtgcctacaaagctgcaggcaacaagctaactttt

SEQ ID NO: 351 - DNA, 36 bp, Homo sapiens
tgtggtttcaaagctgcaggcaacaagctaactttt

SEQ ID NO: 352 - DNA, 36 bp, Homo sapiens
tgtggttggaaagctgcaggcaacaagctaactttt

SEQ ID NO: 353 - DNA, 36 bp, Homo sapiens
tgtagtttgaaagctgcaggcaacaagctaactttt

SEQ ID NO: 354 - DNA, 36 bp, Homo sapiens
tgtagtttgagagctgcaggcaacaagctaactttt

SEQ ID NO: 355 - DNA, 36 bp, Homo sapiens; identical to SEQ ID NO: 94

SEQ ID NO: 356 - DNA, 36 bp, Homo sapiens
tgtagttggaaagctgcaggcaacaagctaactttt

SEQ ID NO: 357 - DNA, 36 bp, Homo sapiens
tgtagttggagagctgcaggcaacaagctaactttt

SEQ ID NO: 358 - DNA, 36 bp, Homo sapiens
tgtagttggagggctgcaggcaacaagctaactttt

SEQ ID NO: 359 - DNA, 36 bp, Homo sapiens
tgtaccaagaaagctgcaggcaacaagctaactttt

SEQ ID NO: 360 - DNA, 36 bp, Homo sapiens
tgtacgaagaaagctgcaggcaacaagctaactttt

SEQ ID NO: 361 - DNA, 36 bp, Homo sapiens
tgtaccttgaaagctgcaggcaacaagctaactttt

SEQ ID NO: 362 - DNA, 36 bp, Homo sapiens
tgtacgaacaaagctgcaggcaacaagctaactttt

SEQ ID NO: 363 - DNA, 36 bp, Homo sapiens
tgtactaggaaagctgcaggcaacaagctaactttt

SEQ ID NO: 364 - DNA, 36 bp, Homo sapiens
tgtgccttccaaaatgcaggcaacatgctcaccttt

SEQ ID NO: 365 - DNA, 36 bp, Homo sapiens; identical to SEQ ID NO: 84

SEQ ID NO: 366 - DNA, 36 bp, Homo sapiens; identical to SEQ ID NO: 118

SEQ ID NO: 367 - DNA, 36 bp, Homo sapiens; identical to SEQ ID NO: 120

SEQ ID NO: 368 - DNA, 39 bp, Homo sapiens; identical to SEQ ID NO: 95

SEQ ID NO: 369 - DNA, 39 bp, Homo sapiens; identical to SEQ ID NO: 97

SEQ ID NO: 370 - DNA, 39 bp, Homo sapiens
tgtgcccgttatggtggtgctgcaaacaagctcatcttt

SEQ ID NO: 371 - DNA, 39 bp, Homo sapiens; identical to SEQ ID NO: 100

SEQ ID NO: 372 - DNA, 39 bp, Homo sapiens; identical to SEQ ID NO: 101

SEQ ID NO: 373 - DNA, 39 bp, Homo sapiens
tgtgcccgctatggtggtggtacaaacaagctcattttt

SEQ ID NO: 374 - DNA, 39 bp, Homo sapiens; identical to SEQ ID NO: 119

SEQ ID NO: 375 - DNA, 39 bp, Homo sapiens; identical to SEQ ID NO: 104

SEQ ID NO: 376 - DNA, 39 bp, Homo sapiens; identical to SEQ ID NO: 105

SEQ ID NO: 377 - DNA, 39 bp, Homo sapiens; identical to SEQ ID NO: 103

SEQ ID NO: 378 - DNA, 39 bp, Homo sapiens
tgtgcctcctatgttggtgctacaaacaagctcatcttt

SEQ ID NO: 379 - DNA, 30 bp, Homo sapiens; identical to SEQ ID NO: 107

SEQ ID NO: 380 - DNA, 30 bp, Homo sapiens; identical to SEQ ID NO: 108

SEQ ID NO: 381 - DNA, 36 bp, Homo sapiens; identical to SEQ ID NO: 80

SEQ ID NO: 382 - DNA, 36 bp, Homo sapiens; identical to SEQ ID NO: 85

SEQ ID NO: 383 - DNA, 36 bp, Homo sapiens
tgtgcctcagaaactggggcaaacaacctcttcttt

SEQ ID NO: 384 - DNA, 36 bp, Homo sapiens; identical to SEQ ID NO: 118

SEQ ID NO: 385 - DNA, 36 bp, Homo sapiens; identical to SEQ ID NO: 120

SEQ ID NO: 386 - DNA, 39 bp, Homo sapiens; identical to SEQ ID NO: 95

SEQ ID NO: 387 - DNA, 39 bp, Homo sapiens
tgtgccttcgcttctggttctgcaaggcaactgaccttt

SEQ ID NO: 388 - DNA, 39 bp, Homo sapiens; identical to SEQ ID NO: 119

SEQ ID NO: 389 - DNA, 42 bp, Homo sapiens
tgtgccttttacccccctggcaacaaccgtaagctgatttgg

SEQ ID NO: 390 - DNA, 36 bp, Homo sapiens; identical to SEQ ID NO: 82

SEQ ID NO: 391 - DNA, 36 bp, Homo sapiens; identical to SEQ ID NO: 81

SEQ ID NO: 392 - DNA, 36 bp, Homo sapiens; identical to SEQ ID NO: 79

SEQ ID NO: 393 - DNA, 36 bp, Homo sapiens
tgtgcctttaaagatgcaggcaacaagctaactttt

SEQ ID NO: 394 - DNA, 36 bp, Homo sapiens
tgtgcctttcctaatgcaggcaacatgctcaccttt

SEQ ID NO: 395 - DNA, 36 bp, Homo sapiens; identical to SEQ ID NO: 86

SEQ ID NO: 396 - DNA, 36 bp, Homo sapiens; identical to SEQ ID NO: 110

SEQ ID NO: 397 - DNA, 36 bp, Homo sapiens; identical to SEQ ID NO: 91

SEQ ID NO: 398 - DNA, 36 bp, Homo sapiens
tgtgctctaaaagatgcaggcaacaagctaactttt

SEQ ID NO: 399 - DNA, 36 bp, Homo sapiens
tgtgccttaaaaaatgcaggcaacatgctcaccttt

SEQ ID NO: 400 - DNA, 36 bp, Homo sapiens; identical to SEQ ID NO: 113

SEQ ID NO: 401 - DNA, 36 bp, Homo sapiens; identical to SEQ ID NO: 115

SEQ ID NO: 402 - DNA, 36 bp, Homo sapiens
tgtgcccccaaagctgcaggcaacaagctaactttt

SEQ ID NO: 403 - DNA, 36 bp, Homo sapiens
tgtgcccccaaagatgcaggcaacaagctaactttt

SEQ ID NO: 404 - DNA, 36 bp, Homo sapiens
tgtgcctccaagaatgcaggcaacatgctcaccttt

SEQ ID NO: 405 - DNA, 36 bp, Homo sapiens
tgtgcctccatgaatgcaggcaacatgctcaccttt

SEQ ID NO: 406 - DNA, 45 bp, Homo sapiens
tgtgcctttaagggaggtggaggtagcaactataaactgacattt

SEQ ID NO: 407 - DNA, 30 bp, Homo sapiens
tgtgcctttggcgactacaagctcagcttt

SEQ ID NO: 408 - DNA, 36 bp, Homo sapiens
tgtgcctttcacgctgcaggcaacaagctaactttt

SEQ ID NO: 409 - DNA, 36 bp, Homo sapiens; identical to SEQ ID NO: 81

SEQ ID NO: 410 - DNA, 36 bp, Homo sapiens
tgtgcctttaagggaggaggaaacaaactcaccttt

SEQ ID NO: 411 - DNA, 36 bp, Homo sapiens
tgtgcctttaacgctgcaggcaacaagctaactttt

SEQ ID NO: 412 - DNA, 36 bp, Homo sapiens
tgtgccttccaggctgcaggcaacaagctaactttt

SEQ ID NO: 413 - DNA, 36 bp, Homo sapiens
tgtgcctttaggggaggaggaaacaaactcaccttt

SEQ ID NO: 414 - DNA, 36 bp, Homo sapiens
tgtgcctttcgaagagcaggcaacatgctcaccttt

SEQ ID NO: 415 - DNA, 36 bp, Homo sapiens; identical to SEQ ID NO: 87

SEQ ID NO: 416 - DNA, 36 bp, Homo sapiens
tgtgcccacaaaggagcaggcaacaagctaactttt

SEQ ID NO: 417 - DNA, 39 bp, Homo sapiens; identical to SEQ ID NO: 95

SEQ ID NO: 418 - DNA, 39 bp, Homo sapiens; identical to SEQ ID NO: 96

SEQ ID NO: 419 - DNA, 39 bp, Homo sapiens
tgtgcccccggggggggcggatctgaaaagctggtcttt

SEQ ID NO: 420 - DNA, 39 bp, Homo sapiens; identical to SEQ ID NO: 97

SEQ ID NO: 421 - DNA, 39 bp, Homo sapiens; identical to SEQ ID NO: 98

SEQ ID NO: 422 - DNA, 39 bp, Homo sapiens; identical to SEQ ID NO: 106

SEQ ID NO: 423 - DNA, 39 bp, Homo sapiens; identical to SEQ ID NO: 104

SEQ ID NO: 424 - DNA, 39 bp, Homo sapiens; identical to SEQ ID NO: 103

SEQ ID NO: 425 - DNA, 33 bp, Homo sapiens
tgtgggggggataacaatgccagactcatgttt

SEQ ID NO: 426 - DNA, 33 bp, Homo sapiens
tgtgggggggataacaatgccagactcacgttt

SEQ ID NO: 427 - DNA, 36 bp, Homo sapiens
tgtgcctctctctataacaccgacaagctcatcttt

SEQ ID NO: 428 - DNA, 39 bp, Homo sapiens; identical to SEQ ID NO: 96

SEQ ID NO: 429 - DNA, 30 bp, Homo sapiens; identical to SEQ ID NO: 315

SEQ ID NO: 430 - DNA, 30 bp, Homo sapiens
tgtgctcgtgacgaccgtaagctgatttgg

SEQ ID NO: 431 - DNA, 36 bp, Homo sapiens; identical to SEQ ID NO: 320

SEQ ID NO: 432 - DNA, 36 bp, Homo sapiens; identical to SEQ ID NO: 322

SEQ ID NO: 433 - DNA, 39 bp, Homo sapiens; identical to SEQ ID NO: 325

SEQ ID NO: 434 - DNA, 39 bp, Homo sapiens; identical to SEQ ID NO: 105

SEQ ID NO: 435 - DNA, 39 bp, Homo sapiens; identical to SEQ ID NO: 103

SEQ ID NO: 436 - DNA, 42 bp, Homo sapiens
tgtgctctcgattcaggaggaggtgctgacggactcaccttt

SEQ ID NO: 437 - DNA, 42 bp, Homo sapiens
tgtgctctcgattcgggaggaggtgctgacggactcaccttt

SEQ ID NO: 438 - DNA, 36 bp, Homo sapiens; identical to SEQ ID NO: 79

SEQ ID NO: 439 - DNA, 36 bp, Homo sapiens
tgtgccaggcgtaatgcaggcaacatgctcaccttt

SEQ ID NO: 440 - DNA, 36 bp, Homo sapiens; identical to SEQ ID NO: 92

SEQ ID NO: 441 - DNA, 36 bp, Homo sapiens
tgtgctctcaaagctgcaggcaacaagctaactttt

SEQ ID NO: 442 - DNA, 36 bp, Homo sapiens
tgtgctctcaaagctgctggcaacaagctaactttt

SEQ ID NO: 443 - DNA, 36 bp, Homo sapiens; identical to SEQ ID NO: 345

SEQ ID NO: 444 - DNA, 36 bp, Homo sapiens
tgtgccttgaggcaagcgggcaacatgctcaccttt

SEQ ID NO: 445 - DNA, 33 bp, Homo sapiens
tgtgcccctgggggttaccagaaagttaccttt

SEQ ID NO: 446 - DNA, 33 bp, Homo sapiens
tgtgcctggggttctgcaaggcaactgaccttt

SEQ ID NO: 447 - DNA, 36 bp, Homo sapiens; identical to SEQ ID NO: 81

SEQ ID NO: 448 - DNA, 36 bp, Homo sapiens; identical to SEQ ID NO: 79

SEQ ID NO: 449 - DNA, 36 bp, Homo sapiens; identical to SEQ ID NO: 411

SEQ ID NO: 450 - DNA, 36 bp, Homo sapiens
tgtgcctttgtagctgctggtcagaattttgtcttt

SEQ ID NO: 451 - DNA, 39 bp, Homo sapiens; identical to SEQ ID NO: 95

SEQ ID NO: 452 - DNA, 39 bp, Homo sapiens; identical to SEQ ID NO: 96

SEQ ID NO: 453 - DNA, 39 bp, Homo sapiens; identical to SEQ ID NO: 105

SEQ ID NO: 454 - DNA, 42 bp, Homo sapiens
tgtgccttaatgacaactgacagctgggggaaattgcagttt

SEQ ID NO: 455 - protein, 13 aa, Homo sapiens
CASLGPLRHEQYF

SEQ ID NO: 456 - protein, 14 aa, Homo sapiens
CASKPLVSTDTQYF

SEQ ID NO: 457 - protein, 14 aa, Homo sapiens
CASLERTSGGEQFF

SEQ ID NO: 458 - protein, 14 aa, Homo sapiens
CASTRDRTKNEQFF

SEQ ID NO: 459 - protein, 15 aa, Homo sapiens
CASSALASGGDEQFF

SEQ ID NO: 460 - protein, 15 aa, Homo sapiens
CASSALASGTDTQYF

SEQ ID NO: 461 - protein, 15 aa, Homo sapiens
CASSELTSRTYEQYF

SEQ ID NO: 462 - protein, 15 aa, Homo sapiens
CASSERVSGNQPQHF

SEQ ID NO: 463 - protein, 15 aa, Homo sapiens
CASSPMASGGDEQFF

SEQ ID NO: 464 - protein, 15 aa, Homo sapiens
CASSRRTSGTYEQYF

SEQ ID NO: 465 - protein, 15 aa, Homo sapiens
CASSVMASRGNEQFF

SEQ ID NO: 466 - protein, 16 aa, Homo sapiens
CASQRGARGGNQPQHF

SEQ ID NO: 467 - protein, 16 aa, Homo sapiens
CASRARTGATNEKLFF

SEQ ID NO: 468 - protein, 16 aa, Homo sapiens
CASSAKTSGGSDTQYF

SEQ ID NO: 469 - protein, 16 aa, Homo sapiens
CASSALASGGRDTQYF

SEQ ID NO: 470 - protein, 16 aa, Homo sapiens
CASSARTSGGLDEQFF

SEQ ID NO: 471 - protein, 16 aa, Homo sapiens
CASSARTSGGSDTQYF

SEQ ID NO: 472 - protein, 16 aa, Homo sapiens
CASSKRASGGTDTQYF

SEQ ID NO: 473 - protein, 16 aa, Homo sapiens
CASSLRTSGGSDTQYF

SEQ ID NO: 474 - protein, 16 aa, Homo sapiens
CASSRLTSGGRNEQFF

SEQ ID NO: 475 - protein, 16 aa, Homo sapiens
CASSSKTSGGTDTQYF

SEQ ID NO: 476 - protein, 16 aa, Homo sapiens
CASSSRTSGGQDEQFF

SEQ ID NO: 477 - protein, 16 aa, Homo sapiens
CATSRGARGSNQPQHF

SEQ ID NO: 478 - protein, 13 aa, Homo sapiens
CASARTGGVGYTF

SEQ ID NO: 479 - protein, 13 aa, Homo sapiens
CASRTSGTYEQYF

SEQ ID NO: 480 - protein, 14 aa, Homo sapiens
CASKAKTVTYKQYF

SEQ ID NO: 481 - protein, 14 aa, Homo sapiens
CASKPKAVTYEQYF

SEQ ID NO: 482 - protein, 14 aa, Homo sapiens
CASRGTATGNTIYF

SEQ ID NO: 483 - protein, 14 aa, Homo sapiens
CASRPTATNEKLFF

SEQ ID NO: 484 - protein, 14 aa, Homo sapiens
CASSEYATSNEQFF

SEQ ID NO: 485 - protein, 14 aa, Homo sapiens
CASSRGQRHRYAVF

SEQ ID NO: 486 - protein, 15 aa, Homo sapiens
CAISRLAGGMDEQYF

SEQ ID NO: 487 - protein, 15 aa, Homo sapiens
CASGRLASGTDTQYF

SEQ ID NO: 488 - protein, 15 aa, Homo sapiens
CASRRGTSGTGELFF

SEQ ID NO: 489 - protein, 15 aa, Homo sapiens
CASSDGASGVGEQYF

SEQ ID NO: 490 - protein, 15 aa, Homo sapiens
CASSEAARGNSPLHF

SEQ ID NO: 491 - protein, 15 aa, Homo sapiens
CASSEGASGLGEQYF

SEQ ID NO: 492 - protein, 15 aa, Homo sapiens
CASSELASGISEQFF

SEQ ID NO: 493 - protein, 15 aa, Homo sapiens
CASSELASGLAEQFF

SEQ ID NO: 494 - protein, 15 aa, Homo sapiens
CASSELASGTGEQFF

SEQ ID NO: 495 - protein, 15 aa, Homo sapiens
CASSERASGTDEQYF

SEQ ID NO: 496 - protein, 15 aa, Homo sapiens
CASSERASGVGELFF

SEQ ID NO: 497 - protein, 15 aa, Homo sapiens
CASSGLASGTDTQYF

SEQ ID NO: 498 - protein, 15 aa, Homo sapiens
CASSGLASGTGELFF

SEQ ID NO: 499 - protein, 15 aa, Homo sapiens
CASSGMTSRSYEQYF

SEQ ID NO: 500 - protein, 15 aa, Homo sapiens
CASSPGLAGTGELFF

SEQ ID NO: 501 - protein, 15 aa, Homo sapiens
CASSPLTSGTDTQYF

SEQ ID NO: 502 - protein, 15 aa, Homo sapiens
CASSPRARGNQPQHF

SEQ ID NO: 503 - protein, 15 aa, Homo sapiens
CASSPRTSGPYEQYF

SEQ ID NO: 504 - protein, 15 aa, Homo sapiens
CASSPRTSGSYEQYF

SEQ ID NO: 505 - protein, 15 aa, Homo sapiens
CASSQGLAGTGELFF

SEQ ID NO: 506 - protein, 15 aa, Homo sapiens
CASSQLARGTDTQYF

SEQ ID NO: 507 - protein, 15 aa, Homo sapiens
CASSQLVSLRGEQYF

SEQ ID NO: 508 - protein, 15 aa, Homo sapiens
CASSQRTSGSDEQYF

SEQ ID NO: 509 - protein, 15 aa, Homo sapiens
CASSQVAGGTATQYF

SEQ ID NO: 510 - protein, 15 aa, Homo sapiens
CASSRGARGNQPQHF

SEQ ID NO: 511 - protein, 15 aa, Homo sapiens
CASSRLAGGLGEQFF

SEQ ID NO: 512 - protein, 15 aa, Homo sapiens
CASSRLAGGMDEQFF

SEQ ID NO: 513 - protein, 15 aa, Homo sapiens
CASSRLAGGTDEQFF

SEQ ID NO: 514 - protein, 15 aa, Homo sapiens
CASSRLTSGTDTQYF

SEQ ID NO: 515 - protein, 15 aa, Homo sapiens
CASSRVTGGMDEQFF

SEQ ID NO: 516 - protein, 15 aa, Homo sapiens
CASTKLAGGTSEQFF

SEQ ID NO: 517 - protein, 15 aa, Homo sapiens
CASTKLAWGTYTQYF

SEQ ID NO: 518 - protein, 15 aa, Homo sapiens
CATTPGASGISEQFF

SEQ ID NO: 519 - protein, 16 aa, Homo sapiens
CASSERGQGARYEQYF

SEQ ID NO: 520 - protein, 16 aa, Homo sapiens
CASSRMTGGGTDTQYF

SEQ ID NO: 521 - protein, 16 aa, Homo sapiens
CASSRRTSGGTDTQYF

SEQ ID NO: 522 - protein, 13 aa, Homo sapiens
CASHRTYTDTQYF

SEQ ID NO: 523 - protein, 13 aa, Homo sapiens
CASSGQTNTEAFF

SEQ ID NO: 524 - protein, 14 aa, Homo sapiens
CASRWTATSYGYTF

SEQ ID NO: 525 - protein, 14 aa, Homo sapiens
CASSPTTTGYGYTF

SEQ ID NO: 526 - protein, 15 aa, Homo sapiens
CASHEGAGGFGELFF

SEQ ID NO: 527 - protein, 15 aa, Homo sapiens
CASHEGAGGYGELFF

SEQ ID NO: 528 - protein, 15 aa, Homo sapiens
CASSAGTRGVGEQFF

SEQ ID NO: 529 - protein, 15 aa, Homo sapiens
CASSALASGTDTQYF

SEQ ID NO: 530 - protein, 15 aa, Homo sapiens
CASSDAASGVGEQYF

SEQ ID NO: 531 - protein, 15 aa, Homo sapiens
CASSDLASGTNEQFF

SEQ ID NO: 532 - protein, 15 aa, Homo sapiens
CASSDRASGVGEQFF

SEQ ID NO: 533 - protein, 15 aa, Homo sapiens
CASSDRTSGPHEQFF

SEQ ID NO: 534 - protein, 15 aa, Homo sapiens
CASSGLAGGMDEQFF

SEQ ID NO: 535 - protein, 15 aa, Homo sapiens
CASSPGARGIDEQFF

SEQ ID NO: 536 - protein, 15 aa, Homo sapiens
CASSPGTSGVGEQFF

SEQ ID NO: 537 - protein, 15 aa, Homo sapiens
CASSPGTSGVGKQFF

SEQ ID NO: 538 - protein, 15 aa, Homo sapiens
CASSPRTSGGGEQYF

SEQ ID NO: 539 - protein, 15 aa, Homo sapiens
CASSPSARGNQPQHF

SEQ ID NO: 540 - protein, 15 aa, Homo sapiens
CASSPTTSGRGEQYF

SEQ ID NO: 541 - protein, 15 aa, Homo sapiens
CASSSGTSGAGEQFF

SEQ ID NO: 542 - protein, 15 aa, Homo sapiens
CASSSGTSGVGEQFF

SEQ ID NO: 543 - protein, 15 aa, Homo sapiens
CASSSGTSGVGEQFF

SEQ ID NO: 544 - protein, 15 aa, Homo sapiens
CASSVGTSGVGEQYF

SEQ ID NO: 545 - protein, 15 aa, Homo sapiens
CASSYGASGVGEQFF

SEQ ID NO: 546 - protein, 15 aa, Homo sapiens
CASSYRTSGPREQFF

SEQ ID NO: 547 - protein, 12 aa, Homo sapiens
CASRKEGSRLHL

SEQ ID NO: 548 - protein, 13 aa, Homo sapiens
CASSDRTTCGYTF

SEQ ID NO: 549 - protein, 13 aa, Homo sapiens
CASSERRIYGYTF

SEQ ID NO: 550 - protein, 14 aa, Homo sapiens
CASRALASGGEQFF

SEQ ID NO: 551 - protein, 14 aa, Homo sapiens
CASSALASGDKQYF

SEQ ID NO: 552 - protein, 14 aa, Homo sapiens
CASSALASGDTQYF

SEQ ID NO: 553 - protein, 14 aa, Homo sapiens
CASSDDRVGDEQFF

SEQ ID NO: 554 - protein, 14 aa, Homo sapiens
CASSKLASGDEQFF

SEQ ID NO: 555 - protein, 14 aa, Homo sapiens
CASSVLPGGNDPLF

SEQ ID NO: 556 - protein, 14 aa, Homo sapiens
CASSVLPGRNEPFF

SEQ ID NO: 557 - protein, 14 aa, Homo sapiens
CASSVLRGGNEQFF

SEQ ID NO: 558 - protein, 14 aa, Homo sapiens
CASSVLRGRNEPFF

SEQ ID NO: 559 - protein, 14 aa, Homo sapiens
CASSVLRGRNEQFF

SEQ ID NO: 560 - protein, 14 aa, Homo sapiens
CASSVSRGGNKQFF

SEQ ID NO: 561 - protein, 14 aa, Homo sapiens
CASVLMRTNNEQFF

SEQ ID NO: 562 - protein, 14 aa, Homo sapiens
CSSRARGCAGKQYF

SEQ ID NO: 563 - protein, 15 aa, Homo sapiens
CASSALTSGGDEQFF

SEQ ID NO: 564 - protein, 15 aa, Homo sapiens
CASSARTSGGSEQFF

SEQ ID NO: 565 - protein, 15 aa, Homo sapiens
CASSARTSGSDEQYF

SEQ ID NO: 566 - protein, 15 aa, Homo sapiens
CASSDRASGGDTQYF

SEQ ID NO: 567 - protein, 15 aa, Homo sapiens
CASSDRASGGDTQYF

SEQ ID NO: 568 - protein, 15 aa, Homo sapiens
CASSDRATGGDTQYF

SEQ ID NO: 569 - protein, 15 aa, Homo sapiens
CASSEKASGGDTQYF

SEQ ID NO: 570 - protein, 15 aa, Homo sapiens
CASSELASGGDEQFF

SEQ ID NO: 571 - protein, 15 aa, Homo sapiens
CASSHKASGGDKQYF

SEQ ID NO: 572 - protein, 15 aa, Homo sapiens
CASSHMASGGDTQYF

SEQ ID NO: 573 - protein, 15 aa, Homo sapiens
CASSHRASGGATPYF

SEQ ID NO: 574 - protein, 15 aa, Homo sapiens
CASSHRASGGDTPHF

SEQ ID NO: 575 - protein, 15 aa, Homo sapiens
CASSHRASGGDTQYF

SEQ ID NO: 576 - protein, 15 aa, Homo sapiens
CASSKLASGADEQYF

SEQ ID NO: 577 - protein, 15 aa, Homo sapiens
CASSKLTRGADKQYF

SEQ ID NO: 578 - protein, 15 aa, Homo sapiens
CASSKRTSGTYEQYF

SEQ ID NO: 579 - protein, 15 aa, Homo sapiens
CASSLRTSGSYEQYF

SEQ ID NO: 580 - protein, 15 aa, Homo sapiens
CASSPRTSGTYEQYF

SEQ ID NO: 581 - protein, 15 aa, Homo sapiens
CASSPRVFSVGELFF

SEQ ID NO: 582 - protein, 15 aa, Homo sapiens
CASSQRASGGDEQFF

SEQ ID NO: 583 - protein, 15 aa, Homo sapiens
CASSRRTSGTYEQYF

SEQ ID NO: 584 - protein, 15 aa, Homo sapiens
CASSVRTSGSYEQYF

SEQ ID NO: 585 - protein, 15 aa, Homo sapiens
CTSSGRTSGRDKQYF

SEQ ID NO: 586 - protein, 15 aa, Homo sapiens
CTSSHRASGGDTQYF

SEQ ID NO: 587 - protein, 16 aa, Homo sapiens
CASSARISGGLNEQYF

SEQ ID NO: 588 - protein, 16 aa, Homo sapiens
CASSARTSGGADTQYF

SEQ ID NO: 589 - protein, 16 aa, Homo sapiens
CASSARTSGGLDEQYF

SEQ ID NO: 590 - protein, 16 aa, Homo sapiens
CASSARTSGGSDTQYF

SEQ ID NO: 591 - protein, 16 aa, Homo sapiens
CASSKRTSGGADTQYF

SEQ ID NO: 592 - protein, 16 aa, Homo sapiens
CASSLRTSGGTDTQYF

SEQ ID NO: 593 - protein, 16 aa, Homo sapiens
CASSPRTSGGTDTQYF

SEQ ID NO: 594 - protein, 16 aa, Homo sapiens
CASSRRASGGTTPHYF

SEQ ID NO: 595 - protein, 16 aa, Homo sapiens
CASSRRTSGGTDTQYF

SEQ ID NO: 596 - protein, 16 aa, Homo sapiens
CASSRRTSGRADTQYF

SEQ ID NO: 597 - protein, 16 aa, Homo sapiens
CASSRRTSGSLDTQYF

SEQ ID NO: 598 - protein, 16 aa, Homo sapiens
CASSTRIRGGTDKQYF

SEQ ID NO: 599 - protein, 16 aa, Homo sapiens
CASSVRTSGGTDTQYF

SEQ ID NO: 600 - protein, 12 aa, Homo sapiens
CASRDSNYGYTF

SEQ ID NO: 601 - protein, 12 aa, Homo sapiens
CASSRRTETQYF

SEQ ID NO: 602 - protein, 13 aa, Homo sapiens
CASSETRANIQYF

SEQ ID NO: 603 - protein, 13 aa, Homo sapiens
CASSGLAANEQFF

SEQ ID NO: 604 - protein, 14 aa, Homo sapiens
CASRAGSVATEAFF

SEQ ID NO: 605 - protein, 14 aa, Homo sapiens
CASSGRTSGNEQFF

SEQ ID NO: 606 - protein, 14 aa, Homo sapiens
CASSVVGSYNEQFF

SEQ ID NO: 607 - protein, 14 aa, Homo sapiens
CASVLMRTNNEQFF

SEQ ID NO: 608 - protein, 15 aa, Homo sapiens
CASRAGTSGTGELFF

SEQ ID NO: 609 - protein, 15 aa, Homo sapiens
CASRKGTSGSGKQYF

SEQ ID NO: 610 - protein, 15 aa, Homo sapiens
CASSEKASGVDEQFF

SEQ ID NO: 611 - protein, 15 aa, Homo sapiens
CASSERASGHDTQYF

SEQ ID NO: 612 - protein, 15 aa, Homo sapiens
CASSHRASGGDTQYF

SEQ ID NO: 613 - protein, 15 aa, Homo sapiens
CASSKLASGADEQYF

SEQ ID NO: 614 - protein, 15 aa, Homo sapiens
CASSKQASGGDEQYF

SEQ ID NO: 615 - protein, 14 aa, Homo sapiens
CASREYATSNEQYF

SEQ ID NO: 616 - protein, 14 aa, Homo sapiens
CASSEMATGLRYTF

SEQ ID NO: 617 - protein, 14 aa, Homo sapiens
CASSETATGLRYTF

SEQ ID NO: 618 - protein, 14 aa, Homo sapiens
CASTLTRVNSPLHF

SEQ ID NO: 619 - protein, 15 aa, Homo sapiens
CASNQRTSGPYEQYF

SEQ ID NO: 620 - protein, 15 aa, Homo sapiens
CASSDLASGTGEQFF

SEQ ID NO: 621 - protein, 15 aa, Homo sapiens
CASSEAAGGYGEQFF

SEQ ID NO: 622 - protein, 15 aa, Homo sapiens
CASSEFARGNQPQHF

SEQ ID NO: 623 - protein, 15 aa, Homo sapiens
CASSEFVRDNQPQHF

SEQ ID NO: 624 - protein, 15 aa, Homo sapiens
CASSEFVRGNQPQHF

SEQ ID NO: 625 - protein, 15 aa, Homo sapiens
CASSEGAAGNQPQHF

SEQ ID NO: 626 - protein, 15 aa, Homo sapiens
CASSEGARGVGEQFF

SEQ ID NO: 627 - protein, 15 aa, Homo sapiens
CASSEGASGTGAQYF

SEQ ID NO: 628 - protein, 15 aa, Homo sapiens
CASSEGASGVGEQFF

SEQ ID NO: 629 - protein, 15 aa, Homo sapiens
CASSEGTSTFREQFF

SEQ ID NO: 630 - protein, 15 aa, Homo sapiens
CASSELASGTGELFF

SEQ ID NO: 631 - protein, 15 aa, Homo sapiens
CASSGAARGNQPQHF

SEQ ID NO: 632 - protein, 15 aa, Homo sapiens
CASSKLTSGGYEQYF

SEQ ID NO: 633 - protein, 15 aa, Homo sapiens
CASSPGTSGVGEQFF

SEQ ID NO: 634 - protein, 15 aa, Homo sapiens
CASSRLAGGFDEQFF

SEQ ID NO: 635 - protein, 15 aa, Homo sapiens
CASSRLAGGTDTQYF

SEQ ID NO: 636 - protein, 15 aa, Homo sapiens
CASSRLASGTDTQYF

SEQ ID NO: 637 - protein, 15 aa, Homo sapiens
CASSVLASGLGEQYF

SEQ ID NO: 638 - protein, 16 aa, Homo sapiens
CASRQGARGGNQPQHF

SEQ ID NO: 639 - protein, 16 aa, Homo sapiens
CASSQGARGGNQPQHF

SEQ ID NO: 640 - protein, 16 aa, Homo sapiens
CASSRLAGGSSYEQYF

SEQ ID NO: 641 - protein, 16 aa, Homo sapiens
CASSRLTSGGADTQYF

SEQ ID NO: 642 - protein, 16 aa, Homo sapiens
CASSRRTSGGTDTQYF

SEQ ID NO: 643 - protein, 14 aa, Homo sapiens
CASSPRASGGEQYF

SEQ ID NO: 644 - protein, 14 aa, Homo sapiens
CASSVRRNNEKLFF

SEQ ID NO: 645 - protein, 15 aa, Homo sapiens
CASNRRTSGTYEQYF

SEQ ID NO: 646 - protein, 15 aa, Homo sapiens
CASSALASGGDTQYF

SEQ ID NO: 647 - protein, 15 aa, Homo sapiens
CASSARASGGDEQFF

SEQ ID NO: 648 - protein, 15 aa, Homo sapiens
CASSARTSGGQPQHF

SEQ ID NO: 649 - protein, 15 aa, Homo sapiens
CASSARTSGNQPQHF

SEQ ID NO: 650 - protein, 15 aa, Homo sapiens
CASSELASGINEQFF

SEQ ID NO: 651 - protein, 15 aa, Homo sapiens
CASSELTSGGDEQFF

SEQ ID NO: 652 - protein, 15 aa, Homo sapiens
CASSKRTSGGDTQYF

SEQ ID NO: 653 - protein, 15 aa, Homo sapiens
CASSLRTSGGDEQYF

SEQ ID NO: 654 - protein, 15 aa, Homo sapiens
CASSPLTSATDTQYF

SEQ ID NO: 655 - protein, 15 aa, Homo sapiens
CASSPRASGGDEQFF

SEQ ID NO: 656 - protein, 15 aa, Homo sapiens
CASSPRTSGGDEQYF

SEQ ID NO: 657 - protein, 16 aa, Homo sapiens
CASRLRTSGGTDTQYF

SEQ ID NO: 658 - protein, 16 aa, Homo sapiens
CASRRLAGFMADTQYF

SEQ ID NO: 659 - protein, 16 aa, Homo sapiens
CASSARTSAGTDTQYF

SEQ ID NO: 660 - protein, 16 aa, Homo sapiens
CASSARTSGGADTHYF

SEQ ID NO: 661 - protein, 16 aa, Homo sapiens
CASSARTSGGADTQYF

SEQ ID NO: 662 - protein, 16 aa, Homo sapiens
CASSARTSGGSDTQYF

SEQ ID NO: 663 - protein, 16 aa, Homo sapiens
CASSARTSGGTDTQYF

SEQ ID NO: 664 - protein, 16 aa, Homo sapiens
CASSERTSGGRDTQYF

SEQ ID NO: 665 - protein, 16 aa, Homo sapiens
CASSGRTSGGSDTQYF

SEQ ID NO: 666 - protein, 16 aa, Homo sapiens
CASSLRASGGSDTQYF

SEQ ID NO: 667 - protein, 16 aa, Homo sapiens
CASSLRTSGGADTQYF

SEQ ID NO: 668 - protein, 16 aa, Homo sapiens
CASSQRTSGGADTQYF

SEQ ID NO: 669 - protein, 16 aa, Homo sapiens
CASSRLTSGGSDTQYF

SEQ ID NO: 670 - protein, 16 aa, Homo sapiens
CASSRRTSGGLDTQYF

SEQ ID NO: 671 - protein, 16 aa, Homo sapiens
CASSRRTSGGPNEQFF

SEQ ID NO: 672 - protein, 16 aa, Homo sapiens
CASSRRTSGGTDTQYF

SEQ ID NO: 673 - protein, 14 aa, Homo sapiens
CASSEGWEPYEQYF

SEQ ID NO: 674 - protein, 15 aa, Homo sapiens
CASSELTSGGDEQFF

SEQ ID NO: 675 - protein, 15 aa, Homo sapiens
CASSELTSGGDEQLF

SEQ ID NO: 676 - protein, 16 aa, Homo sapiens
CASSARTSGGTDTQYF

SEQ ID NO: 677 - protein, 18 aa, Homo sapiens
CATTAAGTGVDGNYGYTF

SEQ ID NO: 678 - protein, 14 aa, Homo sapiens
CASQRGNSYNEQFF

SEQ ID NO: 679 - protein, 14 aa, Homo sapiens
CASRIRTSGGEQYF

SEQ ID NO: 680 - protein, 14 aa, Homo sapiens
CASTRDRTKNEQFF

SEQ ID NO: 681 - protein, 15 aa, Homo sapiens
CASSALASGGDEQFF

SEQ ID NO: 682 - protein, 15 aa, Homo sapiens
CASSLRTSGGDEQFF

SEQ ID NO: 683 - protein, 15 aa, Homo sapiens
CASSPMASGGDEQFF

SEQ ID NO: 684 - protein, 15 aa, Homo sapiens
CASSPRTSGTDTQYF

SEQ ID NO: 685 - protein, 15 aa, Homo sapiens
CASSRRTSGTYEQYF

SEQ ID NO: 686 - protein, 15 aa, Homo sapiens
CASSVMASRGNEQFF

SEQ ID NO: 687 - protein, 15 aa, Homo sapiens
CVSSALASGGDEQFF

SEQ ID NO: 688 - protein, 16 aa, Homo sapiens
CASNKLTSGGRDTQYF

SEQ ID NO: 689 - protein, 16 aa, Homo sapiens
CASSARTSGGSDTQYF

SEQ ID NO: 690 - protein, 16 aa, Homo sapiens
CASSKRTSGATDEQFF

SEQ ID NO: 691 - protein, 16 aa, Homo sapiens
CASSLLTSGGRETQYF

SEQ ID NO: 692 - protein, 16 aa, Homo sapiens
CASSRLTSGGRNEQFF

SEQ ID NO: 693 - protein, 16 aa, Homo sapiens
CASSSRTSGGTDTQYF

SEQ ID NO: 694 - protein, 16 aa, Homo sapiens
CATSRGARGSNQPQHF

SEQ ID NO: 695 - protein, 12 aa, Homo sapiens
CASRDGLGELFF

SEQ ID NO: 696 - protein, 13 aa, Homo sapiens
CASSGQTNTEAFF

SEQ ID NO: 697 - protein, 15 aa, Homo sapiens
CASSALASGTDTQYF

SEQ ID NO: 698 - protein, 15 aa, Homo sapiens
CASSELASGQGSQYF

SEQ ID NO: 699 - protein, 15 aa, Homo sapiens
CASSELASGSYEQYF

SEQ ID NO: 700 - protein, 15 aa, Homo sapiens
CASSELASGTGEQFF

SEQ ID NO: 701 - protein, 15 aa, Homo sapiens
CASSPRTSGPYEQYF

SEQ ID NO: 702 - protein, 15 aa, Homo sapiens
CASSQLTSGTDTQYF

SEQ ID NO: 703 - protein, 15 aa, Homo sapiens
CASSRLAGGFDEQFF

SEQ ID NO: 704 - protein, 15 aa, Homo sapiens
CASSRLASGTDTQYF

SEQ ID NO: 705 - protein, 15 aa, Homo sapiens
CASSRTVSGNQPQHF

SEQ ID NO: 706 - protein, 15 aa, Homo sapiens
CASSSLASRPYEQYF

SEQ ID NO: 707 - protein, 15 aa, Homo sapiens
CASTKGASGSGEQFF

SEQ ID NO: 708 - protein, 15 aa, Homo sapiens
CATTPGASGISEQFF

SEQ ID NO: 709 - protein, 16 aa, Homo sapiens
CASSERGQGARYEQYF

SEQ ID NO: 710 - protein, 16 aa, Homo sapiens
CASSRRTSGGTDTQYF

SEQ ID NO: 711 - protein, 15 aa, Homo sapiens
CASARLAGGTDEQFF

SEQ ID NO: 712 - protein, 15 aa, Homo sapiens
CASSAKARGNQPQHF

SEQ ID NO: 713 - protein, 15 aa, Homo sapiens
CASSALAGGTDTQYF

SEQ ID NO: 714 - protein, 15 aa, Homo sapiens
CASSALASGTDTQYF

SEQ ID NO: 715 - protein, 15 aa, Homo sapiens
CASSDAASGVGEQYF

SEQ ID NO: 716 - protein, 15 aa, Homo sapiens
CASSGQARGNQPQHF

SEQ ID NO: 717 - protein, 15 aa, Homo sapiens
CASSPGTSGVGEQFF

SEQ ID NO: 718 - protein, 15 aa, Homo sapiens
CASSPSARGNQPQHF

SEQ ID NO: 719 - protein, 15 aa, Homo sapiens
CASSVGTSGVGEQYF

SEQ ID NO: 720 - protein, 14 aa, Homo sapiens
CASQASGRSYEQYF

SEQ ID NO: 721 - protein, 14 aa, Homo sapiens
CASSEFWGQETQYF

SEQ ID NO: 722 - protein, 14 aa, Homo sapiens
CASSELASGDEQFF

SEQ ID NO: 723 - protein, 14 aa, Homo sapiens
CASSVSQGSDEQYF

SEQ ID NO: 724 - protein, 15 aa, Homo sapiens
CASCPMASRSYEQYF

SEQ ID NO: 725 - protein, 15 aa, Homo sapiens
CASIIGSQGAYGYTF

SEQ ID NO: 726 - protein, 15 aa, Homo sapiens
CASSALASGGDTQYF

SEQ ID NO: 727 - protein, 15 aa, Homo sapiens
CASSARASGGDEQFF

SEQ ID NO: 728 - protein, 15 aa, Homo sapiens
CASSARTSGNQPQHF

SEQ ID NO: 729 - protein, 15 aa, Homo sapiens
CASSERGTANTEAFF

SEQ ID NO: 730 - protein, 15 aa, Homo sapiens
CASSFRTSGGDTQYF

SEQ ID NO: 731 - protein, 15 aa, Homo sapiens
CASSKRASGGDTQYF

SEQ ID NO: 732 - protein, 15 aa, Homo sapiens
CASSPRASGGDEQFF

SEQ ID NO: 733 - protein, 15 aa, Homo sapiens
CASSPRTSGGDEQYF

SEQ ID NO: 734 - protein, 15 aa, Homo sapiens
CASSPRTSGTYEQYF

SEQ ID NO: 735 - protein, 15 aa, Homo sapiens
CASSPVARGPYEQYF

SEQ ID NO: 736 - protein, 15 aa, Homo sapiens
CASSQLTSRTYEQYF

SEQ ID NO: 737 - protein, 16 aa, Homo sapiens
CASSARTSGGSDTQYF

SEQ ID NO: 738 - protein, 16 aa, Homo sapiens
CASSARTSGGTDTQYF

SEQ ID NO: 739 - protein, 16 aa, Homo sapiens
CASSGRTSGGSDTQYF

SEQ ID NO: 740 - protein, 16 aa, Homo sapiens
CASSLRTSGGADTQYF

SEQ ID NO: 741 - protein, 16 aa, Homo sapiens
CASSLRTSGGSDTQYF

SEQ ID NO: 742 - protein, 16 aa, Homo sapiens
CASSPLTSGGTDTQYF

SEQ ID NO: 743 - protein, 16 aa, Homo sapiens
CASSRRTSGASDTQYF

SEQ ID NO: 744 - protein, 16 aa, Homo sapiens
CASSRRTSGGADEQFF

SEQ ID NO: 745 - protein, 16 aa, Homo sapiens
CASSRRTSGGTDTQYF

SEQ ID NO: 746 - protein, 16 aa, Homo sapiens
CASSRRTSGGYDEQFF

SEQ ID NO: 747 - protein, 16 aa, Homo sapiens
CASSSRTSGGSDTQYF

SEQ ID NO: 748 - protein, 16 aa, Homo sapiens
CASSVRTSGGSDTQYF

SEQ ID NO: 749 - protein, 17 aa, Homo sapiens
CASSISLPGTLYYEQYF

SEQ ID NO: 750 - DNA, 39 bp, Homo sapiens
tgtgccagcttagggcccctacggcacgagcagtacttc

SEQ ID NO: 751 - DNA, 42 bp, Homo sapiens
tgtgccagcaaaccactagttagcacagatacgcagtatttt

SEQ ID NO: 752 - DNA, 42 bp, Homo sapiens
tgtgccagccttgaaaggactagcgggggtgagcagttcttc

SEQ ID NO: 753 - DNA, 42 bp, Homo sapiens
tgtgccagcacccgggacaggacaaagaatgagcagttcttc

SEQ ID NO: 754 - DNA, 45 bp, Homo sapiens
tgtgccagcagtgcgttggctagcgggggagatgagcagttcttc

SEQ ID NO: 755 - DNA, 45 bp, Homo sapiens; identical to SEQ ID NO: 129

SEQ ID NO: 756 - DNA, 45 bp, Homo sapiens
tgtgccagcagtgaactgacctctagaacctacgagcagtacttc

SEQ ID NO: 757 - DNA, 45 bp, Homo sapiens
tgtgccagcagtgaacgggtttcgggcaatcagccccagcatttt

SEQ ID NO: 758 - DNA, 45 bp, Homo sapiens
tgtgccagcagccctatggctagcgggggggatgagcagttcttc

SEQ ID NO: 759 - DNA, 45 bp, Homo sapiens; identical to SEQ ID NO: 160

SEQ ID NO: 760 - DNA, 45 bp, Homo sapiens
tgtgccagcagtgtgatggctagccgtgggaatgagcagttcttc

SEQ ID NO: 761 - DNA, 48 bp, Homo sapiens
tgtgccagccaaaggggggctcgggggggcaatcagccccagcatttt

SEQ ID NO: 762 - DNA, 48 bp, Homo sapiens
tgtgccagcagggctcgaacaggggcaactaatgaaaaactgtttttt

SEQ ID NO: 763 - DNA, 48 bp, Homo sapiens
tgtgccagcagtgccaagactagcgggggctcagatacgcagtatttt

SEQ ID NO: 764 - DNA, 48 bp, Homo sapiens
tgtgccagcagcgccctggctagcgggggccgggatacgcagtatttt

SEQ ID NO: 765 - DNA, 48 bp, Homo sapiens
tgtgccagcagtgcccggactagcgggggactcgatgagcagttcttc

SEQ ID NO: 766 - DNA, 48 bp, Homo sapiens; identical to SEQ ID NO: 135

SEQ ID NO: 767 - DNA, 48 bp, Homo sapiens
tgtgccagcagtaagagggctagcgggggcacagatacgcagtatttt

SEQ ID NO: 768 - DNA, 48 bp, Homo sapiens; identical to SEQ ID NO: 142

SEQ ID NO: 769 - DNA, 48 bp, Homo sapiens
tgtgccagcagccgcttgacgagcggggggcggaatgagcagttcttc

SEQ ID NO: 770 - DNA, 48 bp, Homo sapiens
tgtgccagcagttctaagactagcgggggcacagatacgcagtatttt

SEQ ID NO: 771 - DNA, 48 bp, Homo sapiens
tgtgccagcagttcaaggactagcgggggccaagatgagcagttcttc

SEQ ID NO: 772 - DNA, 48 bp, Homo sapiens
tgtgccacctccagaggagcgcggggaagcaatcagccccagcatttt

SEQ ID NO: 773 - DNA, 39 bp, Homo sapiens
tgtgccagcgccaggacagggggcgttggctacaccttc

SEQ ID NO: 774 - DNA, 39 bp, Homo sapiens
tgtgccagcaggactagcgggacctacgagcagtacttc

SEQ ID NO: 775 - DNA, 42 bp, Homo sapiens
tgtgccagcaaagcaaaaacggtaacctacaagcagtacttc

SEQ ID NO: 776 - DNA, 42 bp, Homo sapiens
tgtgccagcaaaccaaaagcggtaacctacgagcagtacttc

SEQ ID NO: 777 - DNA, 42 bp, Homo sapiens
tgtgccagcagagggacagcgactggaaacaccatatatttt

SEQ ID NO: 778 - DNA, 42 bp, Homo sapiens
tgtgccagcagaccgacagcaactaatgaaaaactgtttttt

SEQ ID NO: 779 - DNA, 42 bp, Homo sapiens
tgtgccagcagtgaatatgcgactagcaatgagcagttcttc

SEQ ID NO: 780 - DNA, 42 bp, Homo sapiens
tgtgccagcagtcgtggacagcggcacagatacgcagtattt

SEQ ID NO: 781 - DNA, 45 bp, Homo sapiens
tgtgccatctcgcgtctagcgggaggcatggacgagcagtacttc

SEQ ID NO: 782 - DNA, 45 bp, Homo sapiens
tgtgccagcggccgactagcatcgggcacagatacgcagtatttt

SEQ ID NO: 783 - DNA, 45 bp, Homo sapiens
tgtgccagcaggagggggactagcggcaccggggagctgtttttt

SEQ ID NO: 784 - DNA, 45 bp, Homo sapiens
tgtgccagcagtgatggggctagcggggtgggagagcagtacttc

SEQ ID NO: 785 - DNA, 45 bp, Homo sapiens
tgtgccagcagtgaagctgccaggggtaattcacccctccacttt

SEQ ID NO: 786 - DNA, 45 bp, Homo sapiens
tgtgccagcagtgaaggggctagcgggctcggggagcagtacttc

SEQ ID NO: 787 - DNA, 45 bp, Homo sapiens
tgtgccagcagtgaactggctagcgggataagtgagcagttcttc

SEQ ID NO: 788 - DNA, 45 bp, Homo sapiens
tgtgccagcagtgaacttgctagcgggctcgcagagcagttcttc

SEQ ID NO: 789 - DNA, 45 bp, Homo sapiens
tgtgccagcagtgaactggctagcgggacgggtgagcagttcttc

SEQ ID NO: 790 - DNA, 45 bp, Homo sapiens
tgtgccagcagtgaacgggctagcgggaccgacgagcagtacttc

SEQ ID NO: 791 - DNA, 45 bp, Homo sapiens
tgtgccagcagtgaaagggctagcggggtcggggagctgtttttt

SEQ ID NO: 792 - DNA, 45 bp, Homo sapiens
tgtgccagcagcggactggctagcggcacagatacgcagtatttt

SEQ ID NO: 793 - DNA, 45 bp, Homo sapiens
tgtgccagttcgggactagcgtcgggcaccggggagctgtttttt

SEQ ID NO: 794 - DNA, 45 bp, Homo sapiens
tgtgccagcagcgggatgactagccgatcctacgagcagtacttc

SEQ ID NO: 795 - DNA, 45 bp, Homo sapiens
tgtgccagcagcccgggactagccggcaccggggagctgtttttt

SEQ ID NO: 796 - DNA, 45 bp, Homo sapiens; identical to SEQ ID NO: 147

SEQ ID NO: 797 - DNA, 45 bp, Homo sapiens
tgtgccagcagtcctcgggccagggggaatcagccccagcatttt

SEQ ID NO: 798 - DNA, 45 bp, Homo sapiens
tgtgccagcagccctcggactagcggtccctacgagcagtacttc

SEQ ID NO: 799 - DNA, 45 bp, Homo sapiens
tgtgccagcagtcctcggactagcgggagttacgagcagtacttc

SEQ ID NO: 800 - DNA, 45 bp, Homo sapiens
tgtgccagcagccaagggctagcgggcaccggggagctgtttttt

SEQ ID NO: 801 - DNA, 45 bp, Homo sapiens
tgtgccagcagtcaattagctaggggcacagatacgcagtatttt

SEQ ID NO: 802 - DNA, 45 bp, Homo sapiens
tgtgccagcagtcaacttgtatcgctgaggggggagcagtacttc

SEQ ID NO: 803 - DNA, 45 bp, Homo sapiens
tgtgccagcagtcaacggactagcgggagcgacgagcagtacttc

SEQ ID NO: 804 - DNA, 45 bp, Homo sapiens
tgtgccagcagccaagttgcggggggtacagctacgcagtatttt

SEQ ID NO: 805 - DNA, 45 bp, Homo sapiens
tgtgccagctcccggggggctcggggcaatcagccccagcatttt

SEQ ID NO: 806 - DNA, 45 bp, Homo sapiens
tgtgccagcagccgactagcgggaggtttaggtgagcagttcttc

SEQ ID NO: 807 - DNA, 45 bp, Homo sapiens
tgtgccagcagccgactagcgggagggatggatgagcagttcttc

SEQ ID NO: 808 - DNA, 45 bp, Homo sapiens
tgtgccagcagccgactagcgggagggacggatgagcagttcttc

SEQ ID NO: 809 - DNA, 45 bp, Homo sapiens
tgtgccagcagtcgtctgactagcggcacagatacgcagtatttt

SEQ ID NO: 810 - DNA, 45 bp, Homo sapiens
tgtgccagcagccgagtgacgggagggatggatgagcagttcttc

SEQ ID NO: 811 - DNA, 45 bp, Homo sapiens
tgtgccagcaccaaactagcggggggtacatctgagcagttcttc

SEQ ID NO: 812 - DNA, 45 bp, Homo sapiens
tgtgccagcaccaaactagcgtggggcacatatacgcagtatttt

SEQ ID NO: 813 - DNA, 45 bp, Homo sapiens
tgtgccaccacccccggggctagcgggataagtgagcagttcttc

SEQ ID NO: 814 - DNA, 45 bp, Homo sapiens
tgtgccaccacccccggggctagtgggataagtgagcagttcttc

SEQ ID NO: 815 - DNA, 48 bp, Homo sapiens
tgtgccagcagtgaaaggggacagggggcgcggtacgagcagtacttc

SEQ ID NO: 816 - DNA, 48 bp, Homo sapiens
tgtgccagcagtcgtatgactggcgggggcacagatacgcagtatttt

SEQ ID NO: 817 - DNA, 48 bp, Homo sapiens; identical to SEQ ID NO: 149

SEQ ID NO: 818 - DNA, 39 bp, Homo sapiens
tgtgccagtcaccggacatacacagatacgcagtatttt

SEQ ID NO: 819 - DNA, 39 bp, Homo sapiens; identical to SEQ ID NO: 121

SEQ ID NO: 820 - DNA, 42 bp, Homo sapiens
tgtgccagcagatggacagcaacctcttatggctacaccttc

SEQ ID NO: 821 - DNA, 42 bp, Homo sapiens
tgtgccagcagtcccacaacgacagggtatggctacaccttc

SEQ ID NO: 822 - DNA, 45 bp, Homo sapiens
tgtgcctcccacgaaggggccgggggcttcggggagctgtttttt

SEQ ID NO: 823 - DNA, 45 bp, Homo sapiens
tgtgcctcccacgaaggggccgggggctacggggagctgtttttt

SEQ ID NO: 824 - DNA, 45 bp, Homo sapiens
tgtgccagcagtgccggaactagaggggtgggggagcagttcttc

SEQ ID NO: 825 - DNA, 45 bp, Homo sapiens; identical to SEQ ID NO: 130

SEQ ID NO: 826 - DNA, 45 bp, Homo sapiens
tgtgccagcagtgacgcggctagcggtgtgggcgagcagtacttc

SEQ ID NO: 827 - DNA, 45 bp, Homo sapiens
tgtgccagcagtgatctggctagcgggacgaatgagcagttcttc

SEQ ID NO: 828 - DNA, 45 bp, Homo sapiens
tgtgccagcagtgaccgggctagcggggtcggggagcagttcttc

SEQ ID NO: 829 - DNA, 45 bp, Homo sapiens
tgtgccagcagtgacaggactagcggtccccatgagcagttcttc

SEQ ID NO: 830 - DNA, 45 bp, Homo sapiens
tgtgccagcagcggactagcgggaggaatggatgagcagttcttc

SEQ ID NO: 831 - DNA, 45 bp, Homo sapiens
tgtgccagcagccccggggcgagaggaattgatgagcagttcttc

SEQ ID NO: 832 - DNA, 45 bp, Homo sapiens; identical to SEQ ID NO: 125

SEQ ID NO: 833 - DNA, 45 bp, Homo sapiens; identical to SEQ ID NO: 126

SEQ ID NO: 834 - DNA, 45 bp, Homo sapiens
tgtgcaagcagccccgggacgagcggagttggtaagcagtttttc

SEQ ID NO: 835 - DNA, 45 bp, Homo sapiens
tgtgccagcagccccaggactagcgggggaggcgagcagtacttc

SEQ ID NO: 836 - DNA, 45 bp, Homo sapiens
tgtgccagcagtcccagtgctcgcggcaatcagccccagcatttt

SEQ ID NO: 837 - DNA, 45 bp, Homo sapiens
tgtgccagcagcccgacgactagcgggagaggcgagcagtacttc

SEQ ID NO: 838 - DNA, 45 bp, Homo sapiens
tgtgccagcagttccgggactagcggggccggggagcagttcttc

SEQ ID NO: 839 - DNA, 45 bp, Homo sapiens
tgtgccagcagttccgggactagcggagttggtgagcagttcttc

SEQ ID NO: 840 - DNA, 45 bp, Homo sapiens
tgtgccagcagttccgggactagcggggtcggggagcagttcttc

SEQ ID NO: 841 - DNA, 45 bp, Homo sapiens
tgtgccagcagtgtcgggactagcggggtgggcgagcagtacttc

SEQ ID NO: 842 - DNA, 45 bp, Homo sapiens
tgtgccagcagttacggggctagcggggtgggggagcagttcttc

SEQ ID NO: 843 - DNA, 45 bp, Homo sapiens
tgtgccagcagttacaggactagcgggccccgggagcagttcttc

SEQ ID NO: 844 - DNA, 36 bp, Homo sapiens
tgtgccagcaggaaagaaggatctaggctacacctc

SEQ ID NO: 845 - DNA, 39 bp, Homo sapiens
tgtgccagcagtgacagaacgacatgtggctacaccttc

SEQ ID NO: 846 - DNA, 39 bp, Homo sapiens
tgtgccagcagtgaaagaaggatctatggctacaccttc

SEQ ID NO: 847 - DNA, 42 bp, Homo sapiens
tgtgccagcagagccctagcgtcggggggtgagcagttcttc

SEQ ID NO: 848 - DNA, 42 bp, Homo sapiens
tgtgccagcagtgctttagctagcggagataagcagtatttt

SEQ ID NO: 849 - DNA, 42 bp, Homo sapiens
tgtgccagcagtgctttggctagcggagatacgcagtatttt

SEQ ID NO: 850 - DNA, 42 bp, Homo sapiens
tgtgccagcagtgacgacagggtcggcgatgagcagttcttc

SEQ ID NO: 851 - DNA, 42 bp, Homo sapiens
tgtgccagcagcaaactagcgtctggagatgagcagttcttc

SEQ ID NO: 852 - DNA, 42 bp, Homo sapiens
tgtgccagcagtgttttacctggaggcaatgatccgctcttc

SEQ ID NO: 853 - DNA, 42 bp, Homo sapiens
tgtgccagcagtgttttacctggtcgcaatgagccgttcttc

SEQ ID NO: 854 - DNA, 42 bp, Homo sapiens
tgcgccagcagtgttttacgtggtggcaatgagcagtttttc

SEQ ID NO: 855 - DNA, 42 bp, Homo sapiens
tgtgccagcagtgttttacgtggtcgcaatgagccgttcttc

SEQ ID NO: 856 - DNA, 42 bp, Homo sapiens
tgtgccagcagtgttttacgtggtcgcaatgagcagttcttc

SEQ ID NO: 857 - DNA, 42 bp, Homo sapiens
tgtgccagcagtgtttcacgaggtggcaataagcagtttttc

SEQ ID NO: 858 - DNA, 42 bp, Homo sapiens; identical to SEQ ID NO: 128

SEQ ID NO: 859 - DNA, 42 bp, Homo sapiens
tgctccagcagagctagagggtgcgcgggtaagcagtatttc

SEQ ID NO: 860 - DNA, 45 bp, Homo sapiens
tgtgccagcagtgccctgactagcgggggcgatgagcagttcttc

SEQ ID NO: 861 - DNA, 45 bp, Homo sapiens
tgtgccagcagtgcaaggactagcgggggatccgagcagttcttc

SEQ ID NO: 862 - DNA, 45 bp, Homo sapiens
tgtgccagcagtgctaggactagcgggagtgacgagcagtacttc

SEQ ID NO: 863 - DNA, 45 bp, Homo sapiens
tgtgccagcagtgacagggcctcaggcggggatacgcagtatttt

SEQ ID NO: 864 - DNA, 45 bp, Homo sapiens
tgtgccagcagtgatcgggctagcgggggggatacgcagtatttt

SEQ ID NO: 865 - DNA, 45 bp, Homo sapiens
tgtgccagcagtgatcgggctacagggggggatacgcagtatttt

SEQ ID NO: 866 - DNA, 45 bp, Homo sapiens
tgtgccagcagtgaaaaggctagcgggggggatacgcagtatttt

SEQ ID NO: 867 - DNA, 45 bp, Homo sapiens
tgtgccagcagtgaattggctagcgggggggatgagcagttcttc

SEQ ID NO: 868 - DNA, 45 bp, Homo sapiens
tgtgccagcagtcacaaggcttcagggggggataagcagtatttt

SEQ ID NO: 869 - DNA, 45 bp, Homo sapiens
tgtgccagctctcacatggcctcaggcggcgatacgcagtatttt

SEQ ID NO: 870 - DNA, 45 bp, Homo sapiens
tgtgccagcagtcacagggcctcaggcggggctactccgtatttt

SEQ ID NO: 871 - DNA, 45 bp, Homo sapiens
tgtgccagcagtcaccgggcctcaggtggggatactccgcatttt

SEQ ID NO: 872 - DNA, 45 bp, Homo sapiens; identical to SEQ ID NO: 141

SEQ ID NO: 873 - DNA, 45 bp, Homo sapiens; identical to SEQ ID NO: 154

SEQ ID NO: 874 - DNA, 45 bp, Homo sapiens; identical to SEQ ID NO: 155

SEQ ID NO: 875 - DNA, 45 bp, Homo sapiens
tgtgccagctcaaaacttactaggggcgcagataagcagtatttc

SEQ ID NO: 876 - DNA, 45 bp, Homo sapiens
tgtgccagcagtaagaggactagcggtacctacgagcagtacttc

SEQ ID NO: 877 - DNA, 45 bp, Homo sapiens
tgtgccagcagcctccggactagcggctcctacgagcagtacttc

SEQ ID NO: 878 - DNA, 45 bp, Homo sapiens; identical to SEQ ID NO: 158

SEQ ID NO: 879 - DNA, 45 bp, Homo sapiens
tgtgccagctcgccgcgagtcttctctgtcggggagctgtttttt

SEQ ID NO: 880 - DNA, 45 bp, Homo sapiens
tgtgccagcagccaaagggctagcgggggggacgagcagttcttc

SEQ ID NO: 881 - DNA, 45 bp, Homo sapiens; identical to SEQ ID NO: 161

SEQ ID NO: 882 - DNA, 45 bp, Homo sapiens
tgtgccagcagcgtccggactagcgggtcctacgagcagtacttc

SEQ ID NO: 883 - DNA, 45 bp, Homo sapiens
tgcaccagcagtggtaggactagcgggagggataagcagtatttc

SEQ ID NO: 884 - DNA, 45 bp, Homo sapiens
tgtaccagcagtcacagggcctcaggcggggatacgcagtatttt

SEQ ID NO: 885 - DNA, 48 bp, Homo sapiens
tgtgccagcagtgccaggattagcggggggctcaacgagcagtacttc

SEQ ID NO: 886 - DNA, 48 bp, Homo sapiens; identical to SEQ ID NO: 132

SEQ ID NO: 887 - DNA, 48 bp, Homo sapiens
tgtgccagcagtgccaggactagcggggggcttgacgagcagtacttc

SEQ ID NO: 888 - DNA, 48 bp, Homo sapiens; identical to SEQ ID NO: 136

SEQ ID NO: 889 - DNA, 48 bp, Homo sapiens
tgtgccagcagtaaaaggactagcgggggggccgatacgcagtatttt

SEQ ID NO: 890 - DNA, 48 bp, Homo sapiens; identical to SEQ ID NO: 144

SEQ ID NO: 891 - DNA, 48 bp, Homo sapiens
tgtgccagcagccccaggactagcgggggcacagatacgcagtatttt

SEQ ID NO: 892 - DNA, 48 bp, Homo sapiens
tgtgccagctcacgacgggcttccgggggcactactccgcattatttt

SEQ ID NO: 893 - DNA, 48 bp, Homo sapiens; identical to SEQ ID NO: 150

SEQ ID NO: 894 - DNA, 48 bp, Homo sapiens; identical to SEQ ID NO: 151

SEQ ID NO: 895 - DNA, 48 bp, Homo sapiens
tgtgccagcagtcgtcggactagcgggagggcggatacgcagtatttt

SEQ ID NO: 896 - DNA, 48 bp, Homo sapiens
tgtgccagcagtcgtaggactagcgggagtctagatacgcagtatttt

SEQ ID NO: 897 - DNA, 48 bp, Homo sapiens
tgtgccagcagcaccaggattagagggggcacagataagcagtatttt

SEQ ID NO: 898 - DNA, 48 bp, Homo sapiens
tgtgccagcagtgttaggactagcgggggcacagatacgcagtatttt

SEQ ID NO: 899 - DNA, 36 bp, Homo sapiens
tgtgccagcagagacagtaactatggctacaccttc

SEQ ID NO: 900 - DNA, 36 bp, Homo sapiens
tgtgccagcagtcgacggacggagacccagtacttc

SEQ ID NO: 901 - DNA, 39 bp, Homo sapiens
tgtgccagcagtgaaaccagggccaacattcagtacttc

SEQ ID NO: 902 - DNA, 39 bp, Homo sapiens
tgtgccagcagcggactagcggcgaatgagcagttcttc

SEQ ID NO: 903 - DNA, 42 bp, Homo sapiens
tgtgcctcaagggcagggtcggtggccactgaagctttcttt

SEQ ID NO: 904 - DNA, 42 bp, Homo sapiens
tgtgccagcagtgggaggactagcggcaatgagcagttcttc

SEQ ID NO: 905 - DNA, 42 bp, Homo sapiens
tgtgccagcagtgtcgtgggcagttacaatgagcagttcttc

SEQ ID NO: 906 - DNA, 42 bp, Homo sapiens; identical to SEQ ID NO: 128

SEQ ID NO: 907 - DNA, 45 bp, Homo sapiens
tgtgccagcagggccgggacctcgggcaccggggagctgtttttt

SEQ ID NO: 908 - DNA, 45 bp, Homo sapiens
tgtgccagcaggaaggggactagcgggagtggcaagcagtacttc

SEQ ID NO: 909 - DNA, 45 bp, Homo sapiens
tgtgccagcagtgaaaaggctagcggggtggatgagcagttcttc

SEQ ID NO: 910 - DNA, 45 bp, Homo sapiens
tgtgccagcagtgaaagggctagcgggcacgatacgcagtatttt

SEQ ID NO: 911 - DNA, 45 bp, Homo sapiens; identical to SEQ ID NO: 141

SEQ ID NO: 912 - DNA, 45 bp, Homo sapiens; identical to SEQ ID NO: 154

SEQ ID NO: 913 - DNA, 45 bp, Homo sapiens
tgtgccagcagtaaacaggctagcgggggggacgagcagtacttc

SEQ ID NO: 914 - DNA, 42 bp, Homo sapiens
tgtgccagcagagaatacgcgactagcaacgagcagtacttc

SEQ ID NO: 915 - DNA, 42 bp, Homo sapiens
tgtgccagcagtgaaatggcgaccgggttgcgctacaccttc

SEQ ID NO: 916 - DNA, 42 bp, Homo sapiens
tgtgccagcagtgaaacggcgacagggttgcgctacaccttc

SEQ ID NO: 917 - DNA, 42 bp, Homo sapiens
tgtgccagcacgctaacacgggttaattcacccctccacttt

SEQ ID NO: 918 - DNA, 45 bp, Homo sapiens
tgtgccagcaatcaacggactagcgggccttacgagcagtacttc

SEQ ID NO: 919 - DNA, 45 bp, Homo sapiens
tgtgccagcagtgatttggctagcggcacaggggagcagttcttc

SEQ ID NO: 920 - DNA, 45 bp, Homo sapiens
tgtgccagcagtgaagctgcagggggctacggtgagcagttcttc

SEQ ID NO: 921 - DNA, 45 bp, Homo sapiens
tgtgccagcagtgaatttgccaggggcaatcagccccagcatttt

SEQ ID NO: 922 - DNA, 45 bp, Homo sapiens
tgtgccagcagtgaattcgtaagggacaatcagccccagcatttt

SEQ ID NO: 923 - DNA, 45 bp, Homo sapiens
tgtgccagcagtgaattcgtaaggggcaatcagccccagcatttt

SEQ ID NO: 924 - DNA, 45 bp, Homo sapiens; identical to SEQ ID NO: 122

SEQ ID NO: 925 - DNA, 45 bp, Homo sapiens
tgtgccagcagtgaaggagctaggggcgtgggggagcagttcttc

SEQ ID NO: 926 - DNA, 45 bp, Homo sapiens
tgtgccagcagtgaaggggctagcggtacgggggcccagtacttc

SEQ ID NO: 927 - DNA, 45 bp, Homo sapiens
tgtgccagcagtgaaggcgctagtggcgtaggggagcagttcttc

SEQ ID NO: 928 - DNA, 45 bp, Homo sapiens
tgtgccagcagtgagggtactagcacctttcgtgagcagttcttc

SEQ ID NO: 929 - DNA, 45 bp, Homo sapiens
tgtgccagcagtgaattagcgagcggcaccggggagctgtttttt

SEQ ID NO: 930 - DNA, 45 bp, Homo sapiens
tgtgccagcagtggggcagcgaggggcaatcagccccagcatttt

SEQ ID NO: 931 - DNA, 45 bp, Homo sapiens
tgtgccagcagcaaactgactagcgggggatacgagcagtacttc

SEQ ID NO: 932 - DNA, 45 bp, Homo sapiens; identical to SEQ ID NO: 127

SEQ ID NO: 933 - DNA, 45 bp, Homo sapiens
tgtgccagcagtcgtctagcggggggtttcgatgagcagttcttc

SEQ ID NO: 934 - DNA, 45 bp, Homo sapiens
tgtgccagcagtcgactagcggggggcacagatacgcagtatttt

SEQ ID NO: 935 - DNA, 45 bp, Homo sapiens
tgtgccagcagtagactagcgtctggcacagatacgcagtatttt

SEQ ID NO: 936 - DNA, 45 bp, Homo sapiens
tgtgccagcagtgtattggctagcgggctgggtgagcagtacttc

SEQ ID NO: 937 - DNA, 48 bp, Homo sapiens
tgtgccagcagacagggggcgaggggaggcaatcagccccagcatttt

SEQ ID NO: 938 - DNA, 48 bp, Homo sapiens
tgtgccagctcacagggggcgcgggggggaaatcagccccagcatttt

SEQ ID NO: 939 - DNA, 48 bp, Homo sapiens
tgtgccagcagtcgattagcgggggggagctcctacgagcagtacttc

SEQ ID NO: 940 - DNA, 48 bp, Homo sapiens
tgtgccagcagccgcctgactagcgggggggcagatacgcagtatttt

SEQ ID NO: 941 - DNA, 48 bp, Homo sapiens; identical to SEQ ID NO: 152

SEQ ID NO: 942 - DNA, 42 bp, Homo sapiens
tgtgccagcagtcctcgggcctcggggggagagcagtacttc

SEQ ID NO: 943 - DNA, 42 bp, Homo sapiens
tgtgccagcagtgtgcggcggaataatgaaaaactgtttttt

SEQ ID NO: 944 - DNA, 45 bp, Homo sapiens
tgtgccagcaaccgaaggactagcggaacctacgagcagtacttc

SEQ ID NO: 945 - DNA, 45 bp, Homo sapiens
tgtgccagcagcgcactagcgtccgggggagatacgcagtatttt

SEQ ID NO: 946 - DNA, 45 bp, Homo sapiens
tgtgccagcagtgcacgggctagcgggggggatgagcagttcttc

SEQ ID NO: 947 - DNA, 45 bp, Homo sapiens
tgtgccagcagtgcacgggctagcgggggggatgagcagtttttc

SEQ ID NO: 948 - DNA, 45 bp, Homo sapiens
tgtgccagcagtgcccggacgagtgggggtcagccccagcatttt

SEQ ID NO: 949 - DNA, 45 bp, Homo sapiens
tgtgccagcagcgcccggacatcgggcaatcagccccagcatttt

SEQ ID NO: 950 - DNA, 45 bp, Homo sapiens
tgtgccagcagtgaactggctagcgggatcaatgagcagttcttc

SEQ ID NO: 951 - DNA, 45 bp, Homo sapiens; identical to SEQ ID NO: 124

SEQ ID NO: 952 - DNA, 45 bp, Homo sapiens
tgtgccagcagtaagaggacctctggaggagatacgcagtatttt

SEQ ID NO: 953 - DNA, 45 bp, Homo sapiens
tgtgccagcagcctaaggactagcgggggggatgagcagtacttc

SEQ ID NO: 954 - DNA, 45 bp, Homo sapiens
tgtgccagcagccccctgactagcgccacagatacgcagtatttt

SEQ ID NO: 955 - DNA, 45 bp, Homo sapiens
tgtgccagcagtcctcgggctagcgggggcgatgagcagttcttc

SEQ ID NO: 956 - DNA, 45 bp, Homo sapiens; identical to SEQ ID NO: 156

SEQ ID NO: 957 - DNA, 48 bp, Homo sapiens
tgtgccagcaggctccggactagcgggggtacagatacgcagtatttt

SEQ ID NO: 958 - DNA, 48 bp, Homo sapiens
tgtgccagcagacgactagcggggtttatggcagatacgcagtatttt

SEQ ID NO: 959 - DNA, 48 bp, Homo sapiens
tgtgccagcagtgcacggactagcgcgggcacagatacgcagtatttt

SEQ ID NO: 960 - DNA, 48 bp, Homo sapiens
tgtgccagcagtgcacggactagcgctggcacagatacgcagtatttt

SEQ ID NO: 961 - DNA, 48 bp, Homo sapiens
tgtgccagcagtgccaggactagcgggggggcagatacgcattatttt

SEQ ID NO: 962 - DNA, 48 bp, Homo sapiens; identical to SEQ ID NO: 133

SEQ ID NO: 963 - DNA, 48 bp, Homo sapiens; identical to SEQ ID NO: 134

SEQ ID NO: 964 - DNA, 48 bp, Homo sapiens; identical to SEQ ID NO: 137

SEQ ID NO: 965 - DNA, 48 bp, Homo sapiens; identical to SEQ ID NO: 138

SEQ ID NO: 966 - DNA, 48 bp, Homo sapiens; identical to SEQ ID NO: 139

SEQ ID NO: 967 - DNA, 48 bp, Homo sapiens; identical to SEQ ID NO: 140

SEQ ID NO: 968 - DNA, 48 bp, Homo sapiens
tgtgccagcagtgaacggactagcgggggacgcgatacgcagtatttt

SEQ ID NO: 969 - DNA, 48 bp, Homo sapiens
tgtgccagcagcggtaggactagcgggggatcggatacgcagtatttt

SEQ ID NO: 970 - DNA, 48 bp, Homo sapiens
tgtgccagcagtcttagggctagcggggggtcagatacgcagtatttt

SEQ ID NO: 971 - DNA, 48 bp, Homo sapiens
tgtgccagcagtctgaggactagcgggggggcagatacgcagtatttt

SEQ ID NO: 972 - DNA, 48 bp, Homo sapiens
tgtgccagcagccaacggactagcgggggggcagatacgcagtatttt

SEQ ID NO: 973 - DNA, 48 bp, Homo sapiens
tgtgccagcagccgactgactagcgggggatcagatacgcagtatttt

SEQ ID NO: 974 - DNA, 48 bp, Homo sapiens
tgtgccagcagtcgccggactagcgggggtctagatacgcagtatttt

SEQ ID NO: 975 - DNA, 48 bp, Homo sapiens
tgtgccagcagtcggcggactagcggggggcccaatgagcagttcttc

SEQ ID NO: 976 - DNA, 48 bp, Homo sapiens; identical to SEQ ID NO: 153

SEQ ID NO: 977 - DNA, 42 bp, Homo sapiens
tgtgccagcagtgaagggtgggaaccctacgagcagtacttc

SEQ ID NO: 978 - DNA, 45 bp, Homo sapiens; identical to SEQ ID NO: 124

SEQ ID NO: 979 - DNA, 45 bp, Homo sapiens
tgtgccagcagtgagttgactagcgggggggatgagcagttgttc

SEQ ID NO: 980 - DNA, 48 bp, Homo sapiens; identical to SEQ ID NO: 139

SEQ ID NO: 981 - DNA, 54 bp, Homo sapiens
tgtgccaccaccgccgccgggacaggggtagacggaaactatggctacaccttc

SEQ ID NO: 982 - DNA, 42 bp, Homo sapiens
tgtgccagccagcggggaaattcctacaatgagcagttcttc

SEQ ID NO: 983 - DNA, 42 bp, Homo sapiens
tgtgccagcagaatccggactagcgggggggagcagtacttc

SEQ ID NO: 984 - DNA, 42 bp, Homo sapiens; identical to SEQ ID NO: 753

SEQ ID NO: 985 - DNA, 45 bp, Homo sapiens; identical to SEQ ID NO: 754

SEQ ID NO: 986 - DNA, 45 bp, Homo sapiens
tgtgccagcagtctccggactagcgggggagatgagcagttcttc

SEQ ID NO: 987 - DNA, 45 bp, Homo sapiens; identical to SEQ ID NO: 758

SEQ ID NO: 988 - DNA, 45 bp, Homo sapiens
tgtgccagcagcccccggactagcggcacagatacgcagtatttt

SEQ ID NO: 989 - DNA, 45 bp, Homo sapiens; identical to SEQ ID NO: 160

SEQ ID NO: 990 - DNA, 45 bp, Homo sapiens; identical to SEQ ID NO: 760

SEQ ID NO: 991 - DNA, 45 bp, Homo sapiens
tgtgtcagcagtgcgttggctagcgggggagatgagcagttcttc

SEQ ID NO: 992 - DNA, 48 bp, Homo sapiens
tgtgccagcaataaattgactagcgggggccgggatacgcagtatttt

SEQ ID NO: 993 - DNA, 48 bp, Homo sapiens; identical to SEQ ID NO: 135

SEQ ID NO: 994 - DNA, 48 bp, Homo sapiens
tgtgccagcagcaaaaggactagcggggcgactgatgagcagttcttc

SEQ ID NO: 995 - DNA, 48 bp, Homo sapiens
tgtgccagcagtttgttgactagcgggggacgggagacccagtacttc

SEQ ID NO: 996 - DNA, 48 bp, Homo sapiens; identical to SEQ ID NO: 769

SEQ ID NO: 997 - DNA, 48 bp, Homo sapiens
tgtgccagcagctccaggactagcgggggcacagatacgcagtatttt

SEQ ID NO: 998 - DNA, 48 bp, Homo sapiens; identical to SEQ ID NO: 772

SEQ ID NO: 999 - DNA, 36 bp, Homo sapiens
tgtgccagcagagacggcctcggggagctgtttttt

SEQ ID NO: 1000 - DNA, 39 bp, Homo sapiens; identical to SEQ ID NO: 121

SEQ ID NO: 1001 - DNA, 45 bp, Homo sapiens
tgtgccagcagtgcactggctagcggaacagatacgcagtatttt

SEQ ID NO: 1002 - DNA, 45 bp, Homo sapiens
tgtgccagcagtgaattggctagcgggcagggatcccagtacttc

SEQ ID NO: 1003 - DNA, 45 bp, Homo sapiens
tgtgccagcagtgaactggctagcgggagctacgagcagtacttc

SEQ ID NO: 1004 - DNA, 45 bp, Homo sapiens; identical to SEQ ID NO: 789

SEQ ID NO: 1005 - DNA, 45 bp, Homo sapiens; identical to SEQ ID NO: 798

SEQ ID NO: 1006 - DNA, 45 bp, Homo sapiens
tgtgccagcagccaattgactagcggcacagatacgcagtatttt

SEQ ID NO: 1007 - DNA, 45 bp, Homo sapiens
tgtgccagcagtcgactagcgggaggatttgatgagcagttcttc

SEQ ID NO: 1008 - DNA, 45 bp, Homo sapiens
tgtgccagcagtcggttggctagcggcacagatacgcagtatttt

SEQ ID NO: 1009 - DNA, 45 bp, Homo sapiens
tgtgccagcagtcggacagtctcgggcaatcagccccagcatttt

SEQ ID NO: 1010 - DNA, 45 bp, Homo sapiens
tgtgccagcagttcgttggctagcagaccctacgagcagtacttc

SEQ ID NO: 1011 - DNA, 45 bp, Homo sapiens
tgtgccagcacgaagggcgctagcgggtcgggtgagcagttcttc

SEQ ID NO: 1012 - DNA, 45 bp, Homo sapiens; identical to SEQ ID NO: 813

SEQ ID NO: 1013 - DNA, 48 bp, Homo sapiens; identical to SEQ ID NO: 815

SEQ ID NO: 1014 - DNA, 48 bp, Homo sapiens
tgtgccagcagccgaaggactagcggaggcacagatacgcagtatttt

SEQ ID NO: 1015 - DNA, 48 bp, Homo sapiens
tgtgccagcagccgaaggactagcggaggtacagatacgcagtatttt

SEQ ID NO: 1016 - DNA, 45 bp, Homo sapiens
tgtgccagcgcccgactagcgggaggtaccgatgagcagttcttc

SEQ ID NO: 1017 - DNA, 45 bp, Homo sapiens
tgtgccagcagcgcgaaggcccgcgggaatcagccccagcatttt

SEQ ID NO: 1018 - DNA, 45 bp, Homo sapiens
tgtgccagcagcgcactagcggggggaacagatacgcagtatttt

SEQ ID NO: 1019 - DNA, 45 bp, Homo sapiens; identical to SEQ ID NO: 130

SEQ ID NO: 1020 - DNA, 45 bp, Homo sapiens; identical to SEQ ID NO: 826

SEQ ID NO: 1021 - DNA, 45 bp, Homo sapiens
tgtgccagcagcggacaggcgaggggcaatcagccccagcatttt

SEQ ID NO: 1022 - DNA, 45 bp, Homo sapiens; identical to SEQ ID NO: 125

SEQ ID NO: 1023 - DNA, 45 bp, Homo sapiens; identical to SEQ ID NO: 836

SEQ ID NO: 1024 - DNA, 45 bp, Homo sapiens; identical to SEQ ID NO: 841

SEQ ID NO: 1025 - DNA, 42 bp, Homo sapiens
tgtgccagccaggcaagcggccgatcctacgagcagtacttc

SEQ ID NO: 1026 - DNA, 42 bp, Homo sapiens
tgtgccagcagtgaattttgggggcaagagacccagtacttc

SEQ ID NO: 1027 - DNA, 42 bp, Homo sapiens
tgtgccagcagtgagctggctagcggggatgagcagttcttc

SEQ ID NO: 1028 - DNA, 42 bp, Homo sapiens
tgtgccagcagtgtatcgcaggggagcgacgagcagtacttc

SEQ ID NO: 1029 - DNA, 45 bp, Homo sapiens
tgtgccagctgtcccatggctagccgatcctacgagcagtacttc

SEQ ID NO: 1030 - DNA, 45 bp, Homo sapiens
tgtgccagcattatcggttcccaaggggcctatggctacaccttc

SEQ ID NO: 1031 - DNA, 45 bp, Homo sapiens; identical to SEQ ID NO: 945

SEQ ID NO: 1032 - DNA, 45 bp, Homo sapiens; identical to SEQ ID NO: 946

SEQ ID NO: 1033 - DNA, 45 bp, Homo sapiens; identical to SEQ ID NO: 949

SEQ ID NO: 1034 - DNA, 45 bp, Homo sapiens
tgtgccagcagtgaacgcgggacagccaacactgaagctttcttt

SEQ ID NO: 1035 - DNA, 45 bp, Homo sapiens
tgtgccagcagcttcaggactagcgggggtgatacgcagtatttt

SEQ ID NO: 1036 - DNA, 45 bp, Homo sapiens
tgtgccagcagtaagcgggctagcgggggagatacgcagtatttt

SEQ ID NO: 1037 - DNA, 45 bp, Homo sapiens; identical to SEQ ID NO: 955

SEQ ID NO: 1038 - DNA, 45 bp, Homo sapiens; identical to SEQ ID NO: 156

SEQ ID NO: 1039 - DNA, 45 bp, Homo sapiens
tgtgccagcagccccaggactagcgggacctacgagcagtacttc

SEQ ID NO: 1040 - DNA, 45 bp, Homo sapiens
tgtgccagcagtcctcggactagcgggacctacgagcagtacttc

SEQ ID NO: 1041 - DNA, 45 bp, Homo sapiens
tgtgccagcagccccgtggccagggggccttacgagcagtacttc

SEQ ID NO: 1042 - DNA, 45 bp, Homo sapiens
tgtgccagcagtcaactgactagcagaacctacgagcagtacttc

SEQ ID NO: 1043 - DNA, 48 bp, Homo sapiens; identical to SEQ ID NO: 138

SEQ ID NO: 1044 - DNA, 48 bp, Homo sapiens
tgtgccagcagcgcccggactagcgggggcacagatacgcagtatttt

SEQ ID NO: 1045 - DNA, 48 bp, Homo sapiens; identical to SEQ ID NO: 139

SEQ ID NO: 1046 - DNA, 48 bp, Homo sapiens; identical to SEQ ID NO: 969

SEQ ID NO: 1047 - DNA, 48 bp, Homo sapiens; identical to SEQ ID NO: 971

SEQ ID NO: 1048 - DNA, 48 bp, Homo sapiens
tgtgccagcagtctcaggactagcgggggttcagatacgcagtatttt

SEQ ID NO: 1049 - DNA, 48 bp, Homo sapiens
tgtgccagcagtcctctgactagcgggggcacagatacgcagtatttt

SEQ ID NO: 1050 - DNA, 48 bp, Homo sapiens
tgtgccagcagtcggaggactagcggggcctcagatacgcagtatttt

SEQ ID NO: 1051 - DNA, 48 bp, Homo sapiens
tgtgccagcagtcgacgtactagcgggggggccgatgagcagttcttc

SEQ ID NO: 1052 - DNA, 48 bp, Homo sapiens
tgtgccagcagccgacggactagcggggggacggatacgcagtatttt

SEQ ID NO: 1053 - DNA, 48 bp, Homo sapiens
tgtgccagcagccggcggactagcgggggcacagatacgcagtatttt

SEQ ID NO: 1054 - DNA, 48 bp, Homo sapiens
tgtgccagcagtagaaggactagcggtggcacagatacgcagtatttt

SEQ ID NO: 1055 - DNA, 48 bp, Homo sapiens
tgtgccagcagtagacggactagcggggggacagatacgcagtatttt

SEQ ID NO: 1056 - DNA, 48 bp, Homo sapiens
tgtgccagcagcagacgaactagcgggggatacgatgagcagttcttc

SEQ ID NO: 1057 - DNA, 48 bp, Homo sapiens
tgtgccagcagtagccggactagcgggggatcagatacgcagtatttt

SEQ ID NO: 1058 - DNA, 48 bp, Homo sapiens
tgtgccagcagtgttcggactagcggggggtcagatacgcagtatttt

SEQ ID NO: 1059 - DNA, 51 bp, Homo sapiens
tgtgccagcagtataagtttacccgggacactttattacgagcagtacttc

SEQ ID NO: 1060 - protein, 12 aa, Homo sapiens
CAFKAAGNKLTF

SEQ ID NO: 1061 - protein, 12 aa, Homo sapiens
CAFTAAGNKLTF

SEQ ID NO: 1062 - protein, 12 aa, Homo sapiens
CALENAGNKLTF

SEQ ID NO: 1063 - protein, 13 aa, Homo sapiens
CASYGGATNKLIF

SEQ ID NO: 1064 - protein, 11 aa, Homo sapiens
CASGNTDKLIF

SEQ ID NO: 1065 - protein, 10 aa, Homo sapiens
CARDDRKLIW

SEQ ID NO: 1066 - protein, 12 aa, Homo sapiens
CAFCNAGNMLTF

SEQ ID NO: 1067 - protein, 12 aa, Homo sapiens
CALGNAGNMLTF

SEQ ID NO: 1068 - protein, 13 aa, Homo sapiens
CASDGGSQGNLIF

SEQ ID NO: 1069 - protein, 12 aa, Homo sapiens
CSPQGGSEKLVF

SEQ ID NO: 1070 - protein, 13 aa, Homo sapiens
CAFIPGGSYIPTF

SEQ ID NO: 1071 - protein, 12 aa, Homo sapiens
CAFKAAGNKLTF

SEQ ID NO: 1072 - protein, 12 aa, Homo sapiens
CAFRAAGNKLTF

SEQ ID NO: 1073 - protein, 12 aa, Homo sapiens
CALKAAGNKLTF

SEQ ID NO: 1074 - protein, 12 aa, Homo sapiens
CAMKAAGNKLTF

SEQ ID NO: 1075 - protein, 12 aa, Homo sapiens
CANRAAGNKLTF

SEQ ID NO: 1076 - protein, 11 aa, Homo sapiens
CAPKSRQQANF

SEQ ID NO: 1077 - protein, 12 aa, Homo sapiens
CASKAAGNKLTF

SEQ ID NO: 1078 - protein, 12 aa, Homo sapiens
CAYKAAGNKLTF

SEQ ID NO: 1079 - protein, 12 aa, Homo sapiens
CGFKAAGNKLTF

SEQ ID NO: 1080 - protein, 12 aa, Homo sapiens
CGWKAAGNKLTF

SEQ ID NO: 1081 - protein, 12 aa, Homo sapiens
CSLKAAGNKLTF

SEQ ID NO: 1082 - protein, 12 aa, Homo sapiens
CSLRAAGNKLTF

SEQ ID NO: 1083 - protein, 12 aa, Homo sapiens
CSRRAAGNKLTF

SEQ ID NO: 1084 - protein, 12 aa, Homo sapiens
CSWKAAGNKLTF

SEQ ID NO: 1085 - protein, 12 aa, Homo sapiens
CSWRAAGNKLTF

SEQ ID NO: 1086 - protein, 12 aa, Homo sapiens
CTKKAAGNKLTF

SEQ ID NO: 1087 - protein, 12 aa, Homo sapiens
CTLKAAGNKLTF

SEQ ID NO: 1088 - protein, 12 aa, Homo sapiens
CTNKAAGNKLTF

SEQ ID NO: 1089 - protein, 12 aa, Homo sapiens
CTRKAAGNKLTF

SEQ ID NO: 1090 - protein, 12 aa, Homo sapiens
CALKQAGNKLTF

SEQ ID NO: 1091 - protein, 12 aa, Homo sapiens
CALRQAGNMLTF

SEQ ID NO: 1092 - protein, 12 aa, Homo sapiens
CALKAAGNKLTF

SEQ ID NO: 1093 - protein, 12 aa, Homo sapiens
CALRAAGNKLTF

SEQ ID NO: 1094 - protein, 12 aa, Homo sapiens
CALRAAGNMLTF

SEQ ID NO: 1095 - protein, 12 aa, Homo sapiens
CASQAAGNKLTF

SEQ ID NO: 1096 - protein, 12 aa, Homo sapiens
CSRRAAGNKLTF

SEQ ID NO: 1097 - protein, 12 aa, Homo sapiens
CALRQAGNMLTF

SEQ ID NO: 1098 - protein, 13 aa, Homo sapiens
CAFDNQAANNLIF

SEQ ID NO: 1099 - protein, 13 aa, Homo sapiens
CAFDNQAGTALIF

SEQ ID NO: 1100 - protein, 12 aa, Homo sapiens
CAFKAAGNKLTF

SEQ ID NO: 1101 - protein, 12 aa, Homo sapiens
CALKAAGNKLTF

SEQ ID NO: 1102 - protein, 12 aa, Homo sapiens
CASKAAGNKLTF

SEQ ID NO: 1103 - protein, 10 aa, Homo sapiens
CAFDNARLMF

SEQ ID NO: 1104 - protein, 10 aa, Homo sapiens
CASYGAALMF

SEQ ID NO: 1105 - protein, 13 aa, Homo sapiens
CACYGGATNKLIF

SEQ ID NO: 1106 - protein, 13 aa, Homo sapiens
CASYGGATNKLIF

SEQ ID NO: 1107 - protein, 11 aa, Homo sapiens
CAFDNADKLIF

SEQ ID NO: 1108 - protein, 11 aa, Homo sapiens
CAFDNTDKLIF

SEQ ID NO: 1109 - protein, 10 aa, Homo sapiens
CAYGANNLFF

SEQ ID NO: 1110 - protein, 12 aa, Homo sapiens
CVFQTGGNNLFF

SEQ ID NO: 1111 - protein, 12 aa, Homo sapiens
CAFCNAGNMLTF

SEQ ID NO: 1112 - protein, 12 aa, Homo sapiens
CALGNAGNMLTF

SEQ ID NO: 1113 - protein, 14 aa, Homo sapiens
CASDYGGSQGNLIF

SEQ ID NO: 1114 - protein, 14 aa, Homo sapiens
CASYDGGTQGNLIV

SEQ ID NO: 1115 - protein, 13 aa, Homo sapiens
CAYCGGSPNNLIF

SEQ ID NO: 1116 - protein, 12 aa, Homo sapiens
CSPQGGSEKLVF

SEQ ID NO: 1117 - protein, 12 aa, Homo sapiens
CAFKGGGNKLTF

SEQ ID NO: 1118 - protein, 12 aa, Homo sapiens
CAFRGGGNKLTF

SEQ ID NO: 1119 - protein, 12 aa, Homo sapiens
CAFHAAGNKLTF

SEQ ID NO: 1120 - protein, 12 aa, Homo sapiens
CAFKAAGNKLTF

SEQ ID NO: 1121 - protein, 12 aa, Homo sapiens
CAFNAAGNKLTF

SEQ ID NO: 1122 - protein, 12 aa, Homo sapiens
CAFQAAGNKLTF

SEQ ID NO: 1123 - protein, 12 aa, Homo sapiens
CAHKAAGNKLTF

SEQ ID NO: 1124 - protein, 12 aa, Homo sapiens
CAHKGAGNKLTF

SEQ ID NO: 1125 - protein, 10 aa, Homo sapiens
CAFGDYKLSF

SEQ ID NO: 1126 - protein, 13 aa, Homo sapiens
CANYGGATNKLIF

SEQ ID NO: 1127 - protein, 13 aa, Homo sapiens
CASYGGATNKLIF

SEQ ID NO: 1128 - protein, 13 aa, Homo sapiens
CAEYGGATNKLIF

SEQ ID NO: 1129 - protein, 13 aa, Homo sapiens
CAPYGGATNKLIF

SEQ ID NO: 1130 - protein, 12 aa, Homo sapiens
CAFRRAGNMLTF

SEQ ID NO: 1131 - protein, 13 aa, Homo sapiens
CAPGGGGSEKLVF

SEQ ID NO: 1132 - protein, 12 aa, Homo sapiens
CAFKAAGNKLTF

SEQ ID NO: 1133 - protein, 12 aa, Homo sapiens
CSFKAAGNKLTF

SEQ ID NO: 1134 - protein, 12 aa, Homo sapiens
CSFKGAGNKLIF

SEQ ID NO: 1135 - protein, 13 aa, Homo sapiens
CALYGGATNKLIF

SEQ ID NO: 1136 - protein, 13 aa, Homo sapiens
CASYGGAPHDLIF

SEQ ID NO: 1137 - protein, 13 aa, Homo sapiens
CASYGGATKILFV

SEQ ID NO: 1138 - protein, 13 aa, Homo sapiens
CASYGGATNKLIF

SEQ ID NO: 1139 - protein, 11 aa, Homo sapiens
CAFDNTDKLIF

SEQ ID NO: 1140 - protein, 12 aa, Homo sapiens
CALGNAGNMLTF

SEQ ID NO: 1141 - protein, 12 aa, Homo sapiens
CSPQGGCEKLVF

SEQ ID NO: 1142 - protein, 13 aa, Homo sapiens
CAFIPGGSYIPTF

SEQ ID NO: 1143 - protein, 16 aa, Homo sapiens
CASRARTGATNEKLFF

SEQ ID NO: 1144 - protein, 16 aa, Homo sapiens
CASQRGARGGNQPQHF

SEQ ID NO: 1145 - protein, 15 aa, Homo sapiens
CASSERVSGNQPQHF

SEQ ID NO: 1146 - protein, 16 aa, Homo sapiens
CATSRGARGSNQPQHF

SEQ ID NO: 1147 - protein, 14 aa, Homo sapiens
CASTRDRTKNEQFF

SEQ ID NO: 1148 - protein, 14 aa, Homo sapiens
CASLERTSGGEQFF

SEQ ID NO: 1149 - protein, 15 aa, Homo sapiens
CASSALASGGDEQFF

SEQ ID NO: 1150 - protein, 16 aa, Homo sapiens
CASSARTSGGLDEQFF

SEQ ID NO: 1151 - protein, 15 aa, Homo sapiens
CASSPMASGGDEQFF

SEQ ID NO: 1152 - protein, 16 aa, Homo sapiens
CASSRLTSGGRNEQFF

SEQ ID NO: 1153 - protein, 16 aa, Homo sapiens
CASSSRTSGGQDEQFF

SEQ ID NO: 1154 - protein, 15 aa, Homo sapiens
CASSVMASRGNEQFF

SEQ ID NO: 1155 - protein, 14 aa, Homo sapiens
CASKPLVSTDTQYF

SEQ ID NO: 1156 - protein, 16 aa, Homo sapiens
CASSAKTSGGSDTQYF

SEQ ID NO: 1157 - protein, 16 aa, Homo sapiens
CASSALASGGRDTQYF

SEQ ID NO: 1158 - protein, 15 aa, Homo sapiens
CASSALASGTDTQYF

SEQ ID NO: 1159 - protein, 16 aa, Homo sapiens
CASSARTSGGSDTQYF

SEQ ID NO: 1160 - protein, 16 aa, Homo sapiens
CASSKRASGGTDTQYF

SEQ ID NO: 1161 - protein, 16 aa, Homo sapiens
CASSLRTSGGSDTQYF

SEQ ID NO: 1162 - protein, 16 aa, Homo sapiens
CASSSKTSGGTDTQYF

SEQ ID NO: 1163 - protein, 13 aa, Homo sapiens
CASLGPLRHEQYF

SEQ ID NO: 1164 - protein, 15 aa, Homo sapiens
CASSELTSRTYEQYF

SEQ ID NO: 1165 - protein, 15 aa, Homo sapiens
CASSRRTSGTYEQYF

SEQ ID NO: 1166 - protein, 14 aa, Homo sapiens
CASIPRTSGGLQFF

SEQ ID NO: 1167 - protein, 16 aa, Homo sapiens
CASSARTSGGQDEQFF

SEQ ID NO: 1168 - protein, 15 aa, Homo sapiens
CASSPMASGGDEQFF

SEQ ID NO: 1169 - protein, 16 aa, Homo sapiens
CASSRLTSGGRNEQFF

SEQ ID NO: 1170 - protein, 15 aa, Homo sapiens
CASSRWASGGDEQFF

SEQ ID NO: 1171 - protein, 15 aa, Homo sapiens
CASSVMASRGNEQFF

SEQ ID NO: 1172 - protein, 15 aa, Homo sapiens
CASQLLVQHTDTQYF

SEQ ID NO: 1173 - protein, 14 aa, Homo sapiens
CASKALVSTDTQYF

SEQ ID NO: 1174 - protein, 14 aa, Homo sapiens
CASKDRTSGDTQYF

SEQ ID NO: 1175 - protein, 15 aa, Homo sapiens
CASSALASGTDTQYF

SEQ ID NO: 1176 - protein, 16 aa, Homo sapiens
CASSARTSGGGDTQYF

SEQ ID NO: 1177 - protein, 16 aa, Homo sapiens
CASSARTSGGRDTQYF

SEQ ID NO: 1178 - protein, 16 aa, Homo sapiens
CASSARTSGGSDTQYF

SEQ ID NO: 1179 - protein, 14 aa, Homo sapiens
CASSELVSGGLHRY

SEQ ID NO: 1180 - protein, 16 aa, Homo sapiens
CASSRRTSGGSDTQYF

SEQ ID NO: 1181 - protein, 16 aa, Homo sapiens
CASSSKTSGGTDTQYF

SEQ ID NO: 1182 - protein, 14 aa, Homo sapiens
CASARERTKNIQYF

SEQ ID NO: 1183 - protein, 14 aa, Homo sapiens
CASRLLVSQETQYF

SEQ ID NO: 1184 - protein, 16 aa, Homo sapiens
CASSLLTSGGRETQYF

SEQ ID NO: 1185 - protein, 14 aa, Homo sapiens
CASSPTVNTYEQYF

SEQ ID NO: 1186 - protein, 14 aa, Homo sapiens
CACRIRTSGGEQYF

SEQ ID NO: 1187 - protein, 14 aa, Homo sapiens
CASRIRTSGGEQYF

SEQ ID NO: 1188 - protein, 14 aa, Homo sapiens
CASSALAGVNEQFF

SEQ ID NO: 1189 - protein, 15 aa, Homo sapiens
CASSPRTSGTYEQYF

SEQ ID NO: 1190 - protein, 15 aa, Homo sapiens
CASSPWARGGDEQFF

SEQ ID NO: 1191 - protein, 13 aa, Homo sapiens
CASSGQTNTEAFF

SEQ ID NO: 1192 - protein, 14 aa, Homo sapiens
CASRWTATSYGYTF

SEQ ID NO: 1193 - protein, 14 aa, Homo sapiens
CASSPTTTGYGYTF

SEQ ID NO: 1194 - protein, 15 aa, Homo sapiens
CASSPSARGNQPQHF

SEQ ID NO: 1195 - protein, 15 aa, Homo sapiens
CASSAGTRGVGEQFF

SEQ ID NO: 1196 - protein, 15 aa, Homo sapiens
CASSPGARGIDEQFF

SEQ ID NO: 1197 - protein, 15 aa, Homo sapiens
CASSPGTSGVGKQFF

SEQ ID NO: 1198 - protein, 15 aa, Homo sapiens
CASSDRASGVGEQFF

SEQ ID NO: 1199 - protein, 15 aa, Homo sapiens
CASSDRTSGPHEQFF

SEQ ID NO: 1200 - protein, 15 aa, Homo sapiens
CASSPGTSGVGEQFF

SEQ ID NO: 1201 - protein, 15 aa, Homo sapiens
CASSSGTSGAGEQFF

SEQ ID NO: 1202 - protein, 15 aa, Homo sapiens
CASSSGTSGVGEQFF

SEQ ID NO: 1203 - protein, 15 aa, Homo sapiens
CASSYRTSGPREQFF

SEQ ID NO: 1204 - protein, 15 aa, Homo sapiens
CASSDLASGTNEQFF

SEQ ID NO: 1205 - protein, 15 aa, Homo sapiens
CASSGLAGGMDEQFF

SEQ ID NO: 1206 - protein, 15 aa, Homo sapiens
CASHEGAGGFGELFF

SEQ ID NO: 1207 - protein, 15 aa, Homo sapiens
CASHEGAGGYGELFF

SEQ ID NO: 1208 - protein, 13 aa, Homo sapiens
CASHRTYTDTQYF

SEQ ID NO: 1209 - protein, 15 aa, Homo sapiens
CASSALASGTDTQYF

SEQ ID NO: 1210 - protein, 15 aa, Homo sapiens
CASSDAASGVGEQYF

SEQ ID NO: 1211 - protein, 15 aa, Homo sapiens
CASSPRTSGGGEQYF

SEQ ID NO: 1212 - protein, 15 aa, Homo sapiens
CASSVGTSGVGEQYF

SEQ ID NO: 1213 - protein, 15 aa, Homo sapiens
CASSYGASGVGEQFF

SEQ ID NO: 1214 - protein, 15 aa, Homo sapiens
CASSPTTSGRGEQYF

SEQ ID NO: 1215 - protein, 13 aa, Homo sapiens
CASSGQTNTEAFF

SEQ ID NO: 1216 - protein, 15 aa, Homo sapiens
CASSPSARGNQPQHF

SEQ ID NO: 1217 - protein, 15 aa, Homo sapiens
CASSPGTSGVGEQFF

SEQ ID NO: 1218 - protein, 15 aa, Homo sapiens
CASARLAGGTDEQFF

SEQ ID NO: 1219 - protein, 15 aa, Homo sapiens
CASSAMASGSDTQYF

SEQ ID NO: 1220 - protein, 15 aa, Homo sapiens
CASSELASGTDTQYF

SEQ ID NO: 1221 - protein, 15 aa, Homo sapiens
CASSVGTSGVGEQYF

SEQ ID NO: 1222 - protein, 15 aa, Homo sapiens
CASSRLAGGMDEQFF

SEQ ID NO: 1223 - protein, 15 aa, Homo sapiens
CASSALASGGDTQYF

SEQ ID NO: 1224 - protein, 16 aa, Homo sapiens
CASSRRTSGGTDTQYF

SEQ ID NO: 1225 - protein, 14 aa, Homo sapiens
CASSVRRNNEKLFF

SEQ ID NO: 1226 - protein, 15 aa, Homo sapiens
CASSARTSGGQPQHF

SEQ ID NO: 1227 - protein, 15 aa, Homo sapiens
CASSARTSGNQPQHF

SEQ ID NO: 1228 - protein, 15 aa, Homo sapiens
CASSARASGGDEQFF

SEQ ID NO: 1229 - protein, 15 aa, Homo sapiens
CASSELTSGGDEQFF

SEQ ID NO: 1230 - protein, 15 aa, Homo sapiens
CASSPRASGGDEQFF

SEQ ID NO: 1231 - protein, 16 aa, Homo sapiens
CASSRRTSGGPNEQFF

SEQ ID NO: 1232 - protein, 15 aa, Homo sapiens
CASSELASGINEQFF

SEQ ID NO: 1233 - protein, 16 aa, Homo sapiens
CASRLRTSGGTDTQYF

SEQ ID NO: 1234 - protein, 16 aa, Homo sapiens
CASRRLAGFMADTQYF

SEQ ID NO: 1235 - protein, 15 aa, Homo sapiens
CASSALASGGDTQYF

SEQ ID NO: 1236 - protein, 16 aa, Homo sapiens
CASSARTSAGTDTQYF

SEQ ID NO: 1237 - protein, 16 aa, Homo sapiens
CASSARTSGGADTHYF

SEQ ID NO: 1238 - protein, 16 aa, Homo sapiens
CASSARTSGGADTQYF

SEQ ID NO: 1239 - protein, 16 aa, Homo sapiens
CASSARTSGGSDTQYF

SEQ ID NO: 1240 - protein, 16 aa, Homo sapiens
CASSARTSGGTDTQYF

SEQ ID NO: 1241 - protein, 16 aa, Homo sapiens
CASSERTSGGRDTQYF

SEQ ID NO: 1242 - protein, 16 aa, Homo sapiens
CASSGRTSGGSDTQYF

SEQ ID NO: 1243 - protein, 16 aa, Homo sapiens
CASSLRASGGSDTQYF

SEQ ID NO: 1244 - protein, 16 aa, Homo sapiens
CASSLRTSGGADTQYF

SEQ ID NO: 1245 - protein, 15 aa, Homo sapiens
CASSPLTSATDTQYF

SEQ ID NO: 1246 - protein, 16 aa, Homo sapiens
CASSQRTSGGADTQYF

SEQ ID NO: 1247 - protein, 16 aa, Homo sapiens
CASSRLTSGGSDTQYF

SEQ ID NO: 1248 - protein, 16 aa, Homo sapiens
CASSRRTSGGLDTQYF

SEQ ID NO: 1249 - protein, 16 aa, Homo sapiens
CASSRRTSGGTDTQYF

SEQ ID NO: 1250 - protein, 15 aa, Homo sapiens
CASSKRTSGGDTQYF

SEQ ID NO: 1251 - protein, 15 aa, Homo sapiens
CASNRRTSGTYEQYF

SEQ ID NO: 1252 - protein, 15 aa, Homo sapiens
CASSLRTSGGDEQYF

SEQ ID NO: 1253 - protein, 14 aa, Homo sapiens
CASSPRASGGEQYF

SEQ ID NO: 1254 - protein, 15 aa, Homo sapiens
CASSPRTSGGDEQYF

SEQ ID NO: 1255 - protein, 12 aa, Homo sapiens
CAFKAAGNKLTF

SEQ ID NO: 1256 - protein, 12 aa, Homo sapiens
CAFKAAGNKLTF

SEQ ID NO: 1257 - protein, 12 aa, Homo sapiens
CAFKAAGNKLTF

SEQ ID NO: 1258 - protein, 12 aa, Homo sapiens
CASKAAGNKLTF

SEQ ID NO: 1259 - protein, 12 aa, Homo sapiens
CASKAAGNKLTF

SEQ ID NO: 1260 - protein, 12 aa, Homo sapiens
CSRRAAGNKLTF

SEQ ID NO: 1261 - protein, 12 aa, Homo sapiens
CAFKAAGNKLTF

SEQ ID NO: 1262 - protein, 12 aa, Homo sapiens
CAFKAAGNKLTF

SEQ ID NO: 1263 - protein, 14 aa, Homo sapiens
CASDYGGSQGNLIF

SEQ ID NO: 1264 - protein, 10 aa, Homo sapiens
CAYGANNLFF

SEQ ID NO: 1265 - protein, 14 aa, Homo sapiens
CASSRLAGGMDEQF

SEQ ID NO: 1266 - protein, 14 aa, Homo sapiens
CATTPGASGISEQF

SEQ ID NO: 1267 - protein, 14 aa, Homo sapiens
CASSGLAGGMDEQF

SEQ ID NO: 1268 - protein, 14 aa, Homo sapiens
CASSRLAGGMDEQF

SEQ ID NO: 1269 - protein, 14 aa, Homo sapiens
CATTPGASGISEQF

SEQ ID NO: 1270 - protein, 14 aa, Homo sapiens
CASSGLAGGMDEQF

SEQ ID NO: 1271 - protein, 15 aa, Homo sapiens
CASSPGTSGVGEQFF

SEQ ID NO: 1272 - protein, 16 aa, Homo sapiens
CASSRRTSGGTDTQYF

SEQ ID NO: 1273 - protein, 13 aa, Homo sapiens
CASSEFRTRGYTF

SEQ ID NO: 1274 - protein, 14 aa, Homo sapiens
CASRPLHSVYEQYF